SEQUENCE LISTING (66 sequences)

SEQ ID NO: 1 - protein, 254 aa, synthetic construct
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 223 aa, synthetic construct
MALPVTALLLPLALLLHAARPQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAKPTTTPAPRPPTPAPTIASQPLSLRPEACRPAAGGAVHTRGLDFACDIYIWAPLAGTCGVLLLSLVITLY

SEQ ID NO: 3 - protein, 233 aa, synthetic construct
MSTESMIRDVELAEEALPKKTGGPQGSRRCLFLSLFSFLIVAGATTLFCLLHFGVIGPQREEFPRDLSLISPLAQPVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 183 aa, synthetic construct
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 5 - protein, 41 aa, synthetic construct
RSKRSRLLHSDYMNMTPRRPGPTRKHYQPYAPPRDFAAYRS

SEQ ID NO: 6 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 7 - DNA, 123 bp, synthetic construct
  source 1..123: mol_type = other DNA
cggagcaagaggtcccgcctgctgcacagcgactatatgaacatgaccccacggagacccggccctacacggaaacattaccagccctatgctccaccccgggacttcgcagcttacagaagt

SEQ ID NO: 8 - protein, 22 aa, synthetic construct
ERVQPLEENVGNAARPRFERNK

SEQ ID NO: 9 - protein, 17 aa, synthetic construct
LEENVGNAARPRFERNK

SEQ ID NO: 10 - protein, 8 aa, synthetic construct
RPRFERNK

SEQ ID NO: 11 - DNA, 66 bp, synthetic construct
  source 1..66: mol_type = other DNA
gaaagggtccaacccctggaagagaatgtgggaaatgcagccaggccaagattcgagaggaacaag

SEQ ID NO: 12 - DNA, 66 bp, synthetic construct
  source 1..66: mol_type = other DNA
gaaagagtgcagcccctggaagagaatgtcgggaatgccgctcgcccaagatttgaaaggaacaaa

SEQ ID NO: 13 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 15 - DNA, 336 bp, synthetic construct
  source 1..336: mol_type = other DNA
cgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 16 - protein, 9 aa, synthetic construct
GDVEXNPGP

SEQ ID NO: 17 - protein, 21 aa, synthetic construct
GSGEGRGSLLTCGDVEENPGP

SEQ ID NO: 18 - DNA, 63 bp, synthetic construct
  source 1..63: mol_type = other DNA
ggctcaggtgaggggcgcgggagcctgctgacttgtggggatgtagaggaaaatcctggtcct

SEQ ID NO: 19 - protein, 22 aa, synthetic construct
GSGATNFSLLKQAGDVEENPGP

SEQ ID NO: 20 - protein, 23 aa, synthetic construct
GSGQCTNYALLKLAGDVESNPGP

SEQ ID NO: 21 - protein, 25 aa, synthetic construct
GSGVKQTLNFDLLKLAGDVESNPGP

SEQ ID NO: 22 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - DNA, 489 bp, synthetic construct
  source 1..489: mol_type = other DNA
atgagaatcagcaaaccacacctccggagcatatcaatccagtgttacttgtgccttcttttgaactcccatttcctcaccgaggcaggcattcatgtgttcatattggggtgctttagtgctgggcttccgaaaacggaagctaactgggtaaacgtcatcagtgaccttaaaaaaattgaggatcttatccaatcaatgcacatcgacgcgactctctacacagaatctgacgtacacccgtcatgcaaagtcacggcaatgaagtgttttcttctcgagctccaagtaatttccctggagtctggcgatgcctccatccacgatacggttgaaaatctgattatattggccaacaatagcctcagttctaacggtaacgtgactgaaagtggctgcaaagagtgcgaagagctcgaagaaaagaatatcaaggagttcctccaatcatttgttcacattgtgcaaatgtttatcaacacctcttga

SEQ ID NO: 24 - DNA, 489 bp, synthetic construct
  source 1..489: mol_type = other DNA
atgcgcataagtaagcctcatctgcggtccatttctatacaatgttatctgtgcttgcttttgaactcccactttcttacggaagcaggcattcatgtgttcattctgggttgtttttctgccgggctgcccaaaaccgaggccaactgggtcaacgtgatcagcgacctcaagaagatcgaggatttgattcaaagtatgcatatagacgccacactctatactgagtccgacgttcacccgagttgtaaagttacggctatgaagtgctttttgttggaactccaggtgatttcccttgaatccggcgatgcgagcatccacgatacggtagagaatcttattattctggcgaataattctctgtcttcaaatgggaatgtaactgagagcggttgtaaagaatgcgaagaacttgaagaaaagaatatcaaggaatttcttcagagtttcgtgcatattgttcaaatgttcatcaacacatcctga

SEQ ID NO: 25 - protein, 175 aa, synthetic construct
RSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 358 aa, synthetic construct
RSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 27 - protein, 21 aa, synthetic construct
MALPVTALLLPLALLLHAARP

SEQ ID NO: 28 - DNA, 63 bp, synthetic construct
  source 1..63: mol_type = other DNA
atggccttaccagtgaccgccttgctcctgccgctggccttgctgctccacgccgccaggccg

SEQ ID NO: 29 - DNA, 63 bp, synthetic construct
  source 1..63: mol_type = other DNA
atggcacttcctgttacagccctcctgctcccactggctttgctgctgcatgctgcacgaccg

SEQ ID NO: 30 - protein, 240 aa, synthetic construct
DIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSS

SEQ ID NO: 31 - DNA, 720 bp, synthetic construct
  source 1..720: mol_type = other DNA
gacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaagggtggcggtggatcgggcggtggtggatctggaggaggtggctcccaggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagc

SEQ ID NO: 32 - protein, 107 aa, synthetic construct
DIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIK

SEQ ID NO: 33 - DNA, 321 bp, synthetic construct
  source 1..321: mol_type = other DNA
gacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaag

SEQ ID NO: 34 - protein, 11 aa, synthetic construct
KASQDINSYLS

SEQ ID NO: 35 - DNA, 33 bp, synthetic construct
  source 1..33: mol_type = other DNA
aaggccagccaggacatcaacagctacctgagc

SEQ ID NO: 36 - protein, 7 aa, synthetic construct
RANRLVD

SEQ ID NO: 37 - DNA, 21 bp, synthetic construct
  source 1..21: mol_type = other DNA
agagccaacagactggtggac

SEQ ID NO: 38 - protein, 9 aa, synthetic construct
LQYDEFPWT

SEQ ID NO: 39 - DNA, 27 bp, synthetic construct
  source 1..27: mol_type = other DNA
ctgcagtacgacgagttcccctggacc

SEQ ID NO: 40 - protein, 15 aa, synthetic construct
GGGGSGGGGSGGGGS

SEQ ID NO: 41 - DNA, 45 bp, synthetic construct
  source 1..45: mol_type = other DNA
ggtggcggtggatcgggcggtggtggatctggaggaggtggctcc

SEQ ID NO: 42 - protein, 118 aa, synthetic construct
QVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSS

SEQ ID NO: 43 - DNA, 354 bp, synthetic construct
  source 1..354: mol_type = other DNA
caggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagc

SEQ ID NO: 44 - protein, 5 aa, synthetic construct
NYGVN

SEQ ID NO: 45 - DNA, 15 bp, synthetic construct
  source 1..15: mol_type = other DNA
aactacggcgtgaac

SEQ ID NO: 46 - protein, 17 aa, synthetic construct
WINTHTGEPTYAEEFKG

SEQ ID NO: 47 - DNA, 51 bp, synthetic construct
  source 1..51: mol_type = other DNA
tggatcaacacccacaccggcgagcccacctacgccgaggagttcaagggc

SEQ ID NO: 48 - protein, 9 aa, synthetic construct
DDYYVRVDY

SEQ ID NO: 49 - DNA, 27 bp, synthetic construct
  source 1..27: mol_type = other DNA
gacgactactacgtgagagtggactac

SEQ ID NO: 50 - protein, 48 aa, synthetic construct
AKPTTTPAPRPPTPAPTIASQPLSLRPEACRPAAGGAVHTRGLDFACD

SEQ ID NO: 51 - DNA, 144 bp, synthetic construct
  source 1..144: mol_type = other DNA
gcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 52 - DNA, 144 bp, synthetic construct
  source 1..144: mol_type = other DNA
gcaaaacctaccacaactcctgcaccacgcccccctactccagcacctaccatcgcatctcagccactgagtctgcgaccagaggcctgccggcccgccgccggcggggccgtccataccagagggctggactttgcctgcgat

SEQ ID NO: 53 - protein, 27 aa, synthetic construct
FWVLVVVGGVLACYSLLVTVAFIIFWV

SEQ ID NO: 54 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 55 - DNA, 81 bp, synthetic construct
  source 1..81: mol_type = other DNA
ttttgggtcctggtggtcgtgggaggggtgctggcatgttactcactgctggtcaccgtggccttcatcatcttctgggtg

SEQ ID NO: 56 - protein, 511 aa, synthetic construct
MALPVTALLLPLALLLHAARPDIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - DNA, 1533 bp, synthetic construct
  source 1..1533: mol_type = other DNA
atggccttaccagtgaccgccttgctcctgccgctggccttgctgctccacgccgccaggccggacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaagggtggcggtggatcgggcggtggtggatctggaggaggtggctcccaggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagcgcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccgaaagggtccaacccctggaagagaatgtgggaaatgcagccaggccaagattcgagaggaacaagagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 58 - DNA, 1533 bp, synthetic construct
  source 1..1533: mol_type = other DNA
atggcacttcctgttacagccctcctgctcccactggctttgctgctgcatgctgcacgaccggacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaagggtggcggtggatcgggcggtggtggatctggaggaggtggctcccaggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagcgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtgaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 59 - protein, 694 aa, synthetic construct
MALPVTALLLPLALLLHAARPDIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 60 - DNA, 2082 bp, synthetic construct
  source 1..2082: mol_type = other DNA
atggccttaccagtgaccgccttgctcctgccgctggccttgctgctccacgccgccaggccggacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaagggtggcggtggatcgggcggtggtggatctggaggaggtggctcccaggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagcgcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccgaaagggtccaacccctggaagagaatgtgggaaatgcagccaggccaagattcgagaggaacaagagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctcaggtgaggggcgcgggagcctgctgacttgtggggatgtagaggaaaatcctggtcctatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 61 - DNA, 2082 bp, synthetic construct
  source 1..2082: mol_type = other DNA
atggcacttcctgttacagccctcctgctcccactggctttgctgctgcatgctgcacgaccggacatccagatgacccagagccccagcagcctgagcgccagcgtgggcgacagagtgaccatcacctgcaaggccagccaggacatcaacagctacctgagctggttccagcagaagcccggcaaggcccccaagaccctgatctacagagccaacagactggtggacggcgtgcccagcagattcagcggcagcggcagcggccaggactacaccctgaccatcagcagcctgcagcccgaggacttcgccacctactactgcctgcagtacgacgagttcccctggaccttcggccagggcaccaaggtggagatcaagggtggcggtggatcgggcggtggtggatctggaggaggtggctcccaggtgcagctggtgcagagcggcagcgagctgaagaagcccggcgccagcgtgaaggtgagctgcaaggccagcggctacaccttcaccaactacggcgtgaactgggtgagacaggcccccggccagggcctggagtggatgggctggatcaacacccacaccggcgagcccacctacgccgaggagttcaagggcagattcgtgttcagcctggacaccagcgtgagcaccgcctacctgcagatcagcagcctgaaggccgaggacaccgccgtgtactactgcgccagagacgactactacgtgagagtggactactggggccagggcaccaccgtgaccgtgagcagcgcaaaacctaccacaactcctgcaccacgcccccctactccagcacctaccatcgcatctcagccactgagtctgcgaccagaggcctgccggcccgccgccggcggggccgtccataccagagggctggactttgcctgcgatttttgggtcctggtggtcgtgggaggggtgctggcatgttactcactgctggtcaccgtggccttcatcatcttctgggtgcggagcaagaggtcccgcctgctgcacagcgactatatgaacatgaccccacggagacccggccctacacggaaacattaccagccctatgctccaccccgggacttcgcagcttacagaagtgaaagagtgcagcccctggaagagaatgtcgggaatgccgctcgcccaagatttgaaaggaacaaacgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcggggctcaggtgaggggcgcgggagcctgctgacttgtggggatgtagaggaaaatcctggtcctatgagaatcagcaaaccacacctccggagcatatcaatccagtgttacttgtgccttcttttgaactcccatttcctcaccgaggcaggcattcatgtgttcatattggggtgctttagtgctgggcttccgaaaacggaagctaactgggtaaacgtcatcagtgaccttaaaaaaattgaggatcttatccaatcaatgcacatcgacgcgactctctacacagaatctgacgtacacccgtcatgcaaagtcacggcaatgaagtgttttcttctcgagctccaagtaatttccctggagtctggcgatgcctccatccacgatacggttgaaaatctgattatattggccaacaatagcctcagttctaacggtaacgtgactgaaagtggctgcaaagagtgcgaagagctcgaagaaaagaatatcaaggagttcctccaatcatttgttcacattgtgcaaatgtttatcaacacctct

SEQ ID NO: 62 - protein, 1255 aa, synthetic construct [sequence omitted]

SEQ ID NO: 63 - protein, 609 aa, synthetic construct [sequence omitted]

SEQ ID NO: 64 - protein, 490 aa, synthetic construct
DIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 673 aa, synthetic construct
DIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 66 - protein, 468 aa, synthetic construct
DIQMTQSPSSLSASVGDRVTITCKASQDINSYLSWFQQKPGKAPKTLIYRANRLVDGVPSRFSGSGSGQDYTLTISSLQPEDFATYYCLQYDEFPWTFGQGTKVEIKGGGGSGGGGSGGGGSQVQLVQSGSELKKPGASVKVSCKASGYTFTNYGVNWVRQAPGQGLEWMGWINTHTGEPTYAEEFKGRFVFSLDTSVSTAYLQISSLKAEDTAVYYCARDDYYVRVDYWGQGTTVTVSSAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2026/0048084 A1
(43) Pub. Date: Feb. 19, 2026

(54) METHODS OF ADMINISTERING NATURAL KILLER CELLS COMPRISING AN ANTI-HUMAN EPIDERMAL GROWTH FACTOR RECEPTOR 2 (HER2) CHIMERIC ANTIGEN RECEPTOR (CAR)

(71) Applicants: Artiva Biotherapeutics, Inc., San Diego, CA (US); GC Cell Corporation, Gyeonggi-do (KR)

(72) Inventors: Hoyong LIM, Gyeonggi-do (KR); Sungyoo CHO, Gyeonggi-do (KR); Yu Kyeong HWANG, Gyeonggi-do (KR); Eunji CHOI, Gyeonggi-do (KR); Miyoung JUNG, Gyeonggi-do (KR); Eunsol LEE, Gyeonggi-do (KR); Hansol KIM, Gyeonggi-do (KR); Peter FLYNN, Cardiff by the Sea, CA (US); Jason B. LITTEN, Poway, CA (US); Thomas James FARRELL, La Jolla, CA (US); John Kin Chuan LIM, San Diego, CA (US); Heather Karen RAYMON, La Jolla, CA (US); Lisa GUERRETTAZ, Cardiff, CA (US); Thorsten GRAEF, Los Altos Hills, CA (US)

(21) Appl. No.: 19/104,146

(22) PCT Filed: Aug. 16, 2023
(86) PCT No.: PCT/US2023/072337

§ 371 Date: Feb. 14, 2025

Related U.S. Application Data
(60) Provisional application No. 63/398,834, filed on Aug. 17, 2022.

(51) Int. Cl. A61K 35/17; A61K 31/337; A61K 31/675; A61K 31/7076; A61K 38/20; A61K 40/15; A61K 40/31; A61K 40/42; A61K 45/06; A61K 47/02; A61K 47/20; A61K 47/26; A61K 47/36; A61K 47/42; A61P 35/00; C07K 14/54; C07K 14/705; C07K 14/725; C07K 14/735; C07K 16/28; C07K 16/32

(52) U.S. Cl. CPC A61K 35/17; A61K 31/337; A61K 31/675; A61K 31/7076; A61K 38/2013; A61K 40/15; A61K 40/31; A61K 40/4205; A61K 40/421; A61K 40/4234; A61K 45/06; A61K 47/02; A61K 47/20; A61K 47/26; A61K 47/36; A61K 47/42; A61P 35/00; C07K 14/5443; C07K 14/7051; C07K 14/70517; C07K 14/70521; C07K 14/70535; C07K 14/70575; C07K 16/2818; C07K 16/2827; C07K 16/2863; C07K 16/32; A61K 2239/13; A61K 2239/17; A61K 2239/21; A61K 2239/22; A61K 2239/38; C07K 2317/565; C07K 2317/622; C07K 2319/02

(57) ABSTRACT

Provided herein, among other things, are methods of administering NK cells comprising poly nucleotides comprising a nucleic acid encoding an anti-human epidermal growth factor receptor 2 (HER2) chimeric antigen receptor (CAR).

### CLAIM OF PRIORITY

This application claims the benefit of U.S. Provisional Application Ser. No. 63/398,834, filed on Aug. 17, 2022. The entire contents of the foregoing are incorporated herein by reference.

### BACKGROUND

Targeted therapies, including the use of adoptive cell therapies such as chimeric antigen receptor T cells (CAR Ts), have revolutionized cancer treatment. These cell therapies may be autologous (CAR T cells manufactured using a patient's own T cells) or allogeneic (CAR T cells manufactured using T cells from healthy donors. Currently, there are no autologous CAR T therapies approved to treat HER2-specific cancers.

Whilst transformative in their ability to treat targeted hematologic cancers, challenging obstacles have arisen in the clinic since 2017, when the first CAR-T therapies targeting CD19 B-cell malignancies were approved by the United States Food and Drug Administration, with the use of autologous CAR-T cell products. CAR T cell manufacturing is a resource-intensive process that can result in failure to produce a viable autologous cell therapy for some patients. The average manufacturing time of 3 weeks that is needed for autologous CAR T cell products may be too long for critically ill patients. Finally, due to the complex nature of the manufacture and delivery of CAR-T cell product, which require close monitoring at top-tier cancer and medical centers, access to this treatment option may be out of reach, both financially and geographically, for most patients. Importantly, even for those patients who have access to this innovative treatment, CAR-T cell products confer a risk of serious and potentially deadly adverse effects. These adverse effects include cytokine release syndrome (CRS) and neurotoxicity, which can be difficult to manage or control.

Allogeneic CAR-T cell therapies, which utilize cells from healthy donors, may overcome some of the manufacturing and logistical challenges of autologous CAR-T cell therapies. However, these “off-the-shelf′ CAR T cell therapies also have issues that include a potentially higher risk of graft-versus-host disease (GVHD) and ineffectiveness due to rapid clearance by the patient's immune system.

Natural killer (NK) cells are cytolytic cells of the innate immune system with an intrinsic ability to lyse tumor cells and virus-infected cells. NK cells have the inherent ability to bridge between innate immunity and engender a multi-clonal adaptive immune response resulting in long-term anticancer immune memory. Importantly. NK cells do not require prior antigen exposure to identify and lyse tumor cells. Receptor engagement by NK cells drives effector function through degranulation of lytic granules, activation of programmed cell death receptors on target cells, and secretion of immune modulatory cytokines. Natural killer cell effector function is governed through the balance of activating and inhibitory receptor signaling. Classically, NK cells are defined as CD56′ and CD3” cells that are subdivided in to CD56^{bright}CD16 cytokine secreting cells and CD56dimCD16′ cytolytic cells. Engagement of CD16 with antibody opsonized tumor cells is sufficient to elicit cytotoxicity and cytokine release response by resting NK cells. Activated NK cells secrete cytokines and chemokines, such as interferon gamma (IFNγ); tumor necrosis factor alpha (TNFα); and macrophage inflammatory protein I (MIPI) that signal and recruit T cells to tumors. Through direct killing of tumor cells, NK cells also expose tumor antigens for recognition by the adaptive immune system. Natural killer cells also engage tumor cells through antibody dependent cellular cytotoxicity (ADCC). To initiate ADCC, NK cells engage with antibodies via the CD16 receptor on their surface.

Natural killer cells also engage tumor cells through antibody dependent cellular cytotoxicity (ADCC), a key component of the innate immune system. Antibody-coated target cells are killed by cells with Fc receptors that recognize the constant region of the bound antibody. Engagement of CD16 (FCyRIII) with antibody-opsonized tumor cells is sufficient to elicit cytotoxicity and cytokine release response by resting NK cells. Activated NK cells secrete cytokines and chemokines, such as interferon gamma (IFNγ): tumor necrosis factor alpha (TNFα); and macrophage inflammatory protein 1 (MIP1) that signal and recruit T cells to tumors. Through direct killing of tumor cells, NK cells also expose tumor antigens for recognition by the adaptive immune system. ADCC is recognized as a potent mechanism of NK cell action, particularly in combination with antibodies belonging to immunoglobulin G1 (IgG1) and IG3 subclasses. To initiate ADCC. NK cells engage with antibodies via the CD16 receptor.

Similar to T cells, allogeneic NK cells engineered to express CARs with anti-tumor activity may provide an important treatment option for cancer patients. NK cells do not suffer from some of the shortcomings of allogeneic CAR-T cells, which often retain expression of endogenous T cell receptors in addition to engineered chimeric antigen receptors. As a result. allogeneic CAR-NK cell treatments can be administered safely to patients without many of the risks associated with allogeneic T cell therapies, including GVHD. However, CAR-NK cells face many of the same challenges as other allogeneic cell therapies, including product sourcing, scalability, persistence, and dose-to-dose variability.

HER2, also known as human epidermal growth factor receptor 2 and ErbB2, is a receptor tyrosine kinase that is highly expressed on the surface of many solid tumors. In normal cells, HER2 plays an important role in cell development. However, the mutation or overexpression of HER2 can directly lead to tumorigenesis as well as metastasis.

HER2 amplification, often seen as an important signal of tumorigenesis, is common in several different solid tumor types, including a 20% to 30% overexpression in human breast, ovarian, and gastric cancers.

Currently, there are 8 approved HER2-directed therapies for cancer patients. These FDA-approved drugs include monoclonal antibodies, antibody-drug conjugates (ADCs) and small molecule tyrosine kinase inhibitors (TKIs). In traditional therapies for HER2+ cancers, such as trastuzumab or lapatinib therapy, downstream signaling mutations may result in tumor growth inhibition resistance. One of trastuzumab's mechanisms of action is the inhibition of the MAPK and PI3K/Akt pathways, which leads to cell cycle arrest. Mutations can confer resistance to trastuzumab treatment, including mutations that cause a loss of PTEN (phosphatase and tensin homolog) and activating mutations of PIK3CA (phosphatidylinositol 3-kinase). Such mutations can constitutive activate the PI3K/Akt pathway, which drives cell proliferation. Loss of PTEN was observed in 36% of Her2 positive primary breast tumor specimens (Stage IV disease); these patients had lower overall response rates to trastuzumab. Additionally, about 25% of trastuzumab resistant patients have PIK3CA mutation. Patients with PI3KCA mutations had significantly shorter progression-free survival than those without the mutation following trastuzumab treatment. Thus, while many patients derive meaningful benefit from these therapies, a significant portion will eventually suffer relapse or disease progression. Once all HER2 directed therapies have been exhausted, patients may be offered cytotoxic chemotherapy that provides only a modest benefit. The absence of safe and effective treatments for patients who have exhausted HER2-directed options represents an important and continued unmet medical need.

The present invention addresses these and other deficiencies in the art.

### SUMMARY

NK cells are immune cells that can engage tumor cells through a complex array of receptors on their cell surface, as well as through antibody-dependent cellular cytotoxicity (ADCC). NK cells may have an advantage over other immune cells, such as the T cells used in CAR-T cell therapy and other cell therapies. Autologous CAR-T cells must be engineered from a patient's own cells. Such engineering can take time, during which period the patient's disease may progress significantly. Such patients may require a bridging therapy to sustain them until their autologous CAR-T cells are ready. Not all patients qualify for autologous CAR-T therapy. For example, some patients may be too sick or may not have sufficient numbers of T cells suitable for engineering purposes. Not all manufacturing runs of autologous CAR-T cells result in sufficient cell numbers or sufficiently active cells to be therapeutically effective. When such manufacturing runs are successful, patients typically only receive a single dose of autologous CAR-T treatment. Because the risk of acute side effects like ICANS and CRS are greatest immediately after administering CAR-T cells, repeat dosing is potentially too risky if the patient will only see marginal benefit from a second, third, or further dose. Additionally, because autologous CAR-T treatments must be unique for each patient, the costs of such treatments can make them unaffordable for many patients who would otherwise benefit from them.

In an exemplary advantage. NK cells can be used as allogeneic therapies, meaning that NK cells from one donor can be safely used in one or many patients without the requirement for HLA matching, gene editing, or other genetic manipulations. As a result, allogeneic CAR-NK cells can be manufactured in bulk, cryopreserved, shipped throughout the world, and administered on demand at the point of care. Thus, the allogeneic cell therapies can be administered to a patient immediately, without the need to wait for the patient's own cells to be engineered and administered and without the need for a bridging therapy. Because the allogeneic therapies described herein can be manufactured in bulk using campaign-manufacturing methods, the costs associated with manufacturing and delivering the allogeneic therapies described herein has the promise to be significantly lower than those of autologous CAR-T therapies. Campaign manufacturing also reduces variability between batches and allows a patient to receive multiple doses of CAR-NK cells made from a single batch derived from a single donor where preferable.

The ability to offer repeat dosing may allow patients to experience or maintain a deeper or prolonged response from the therapy. For example, patients can receive response-based dosing, during which the patient continues to receive doses of CAR-NK cell therapy for as long as the patient derives a benefit. The number of doses and the number of cells administered in each dose can also be tailored to the individual patient. In such cases, the patient is not limited by the number of cells he or she can provide during the cell harvests associated with autologous CAR-T therapy. Thus, the CAR-NK cell therapies described herein can be tailored to each patient based on that patient's own response. In some cases, the therapy can also be reinitiated if the patient relapses.

Allogeneic NK cells may provide an important treatment option for cancer patients. In one exemplary advantage, NK cells have been well tolerated without evidence of graft-versus-host disease, neurotoxicity or cytokine release syndrome associated with other cell-based therapies. In another exemplary advantage, NK cells do not require prior antigen exposure to antigens to identify and lyse tumor cells. In another exemplary advantage, NK cells have the inherent ability to bridge between innate immunity and engender a multi-clonal adaptive immune response resulting in long-term anticancer immune memory. All of these features contribute to the potential for NK cell efficacy as cancer treatment options.

For example, NK cells can recruit and activate other components of the immune system. Activated NK cells secrete cytokines and chemokines, such as interferon gamma (IFNγ): tumor necrosis factor alpha (TNFα); and macrophage inflammatory protein 1 (MIP1) that signal and recruit T cells to tumors. Through direct killing of tumor cells. NK cells also expose tumor antigens for recognition by the adaptive immune system.

Additionally, umbilical cord blood units with preferred characteristics for enhanced clinical activity (e.g., high-affinity CD16 and Killer cell Immunoglobulin-like Receptor (KIR) B-haplotype) can be selected by utilizing a diverse umbilical cord blood bank as a source for NK cells.

Engineered NK cells, e.g., the CAR-NK cells described herein, have an advantage over autologous cell therapies, e.g., T cells used in CAR-T cell therapy, because the NK cells can be used as allogeneic therapies. Thus, NK cells from one donor can be safely used in one or many patients.

In traditional therapies for Her2+ cancers, such as trastuzumab or lapatinib therapy, mutations may result in tumor growth inhibition resistance. As discussed above, some mutations can alter downstream signaling pathways, rendering cells resistant to trastuzumab or lapatinib. Unlike trastuzumab or lapatinib, the HER2-directed CAR-NK cells and therapies described herein, such as AB-201, are activated by binding HER2 expressed on the surface of target cells. The activated CAR-NK cells then employ their own cytotoxic pathways to kill the target cells. This killing is, therefore, independent of signaling integrity within the target cells. Thus, the CAR-NK cells and cell therapies described herein retain the ability to kill HER2+ tumor cells, even in the presence of some downstream signaling mutations that might confer resistance to approved HER2 therapeutics. Other mutations may alter the epitope to which trastuzumab or lapatinib bind, rendering those drugs less effective. Because the scFy of the Her2-directed CAR-NKs described herein, including, for example, the scFv of SEQ ID NO: 30, binds to a different domain (domain I) of HER than either trastuzumab (domain IV) or lapatinib (domain II), the CAR-NK cells described herein may remain effective at binding to HER2+ cells even when other mutations reduce or inhibit the ability of trastuzumab or lapatinib to bind.

Moreover, the CAR-NK cells described herein can retain CD16 expression, including expression of the 158 V/V variant of CD16. Thus, in some cases, the CAR-NK cells can be used in combination with traditional antibody therapy. In one example, the antibody therapy can comprise trastuzumab or lapatinib. Alternatively or in combination, the antibody therapy can be targeted to alternative or additional targets, including, for example, EGFR. Examples of anti-EGFR antibodies include cetuximab, panitumumab, nimotuzumab, and necitumumab. Such antibodies can elicit an ADCC response from NK cells by binding to CD16 expressed on the NK cell surface. Thus, in some cases, the method of treatment can include a dual targeting approach comprising combining the use of the CAR-NK cells targeting HER2 described herein with an anti-EGFR antibody therapy.

Thus, provided herein, among other things, methods comprising administering from I million to 100 billion natural killer (NK) cells to a patient in need thereof, wherein the NK cells comprise a polynucleotide comprising: a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and b) a nucleic acid encoding an IL-15.

In some embodiments, the NK cells are administered in a dose comprising from 1 billion to 100 billion NK cells, optionally in a dose comprising at or about 1 million to at or about 20 billion cells (e.g., at or about 5 million cells, at or about 25 million cells, at or about 50 million cells, at or about 75 million cells, at or about 100 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, or a range defined by any two of the foregoing values), at or about 10 million to at or about 20 billion cells (e.g., at or about 25 million cells, at or about 50 million cells, at or about 75 million cells, at or about 100 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, at or about 20 billion cells, or a range defined by any two of the foregoing values), and in some cases at or about 100 million cells to at or about 50 billion cells (e.g., at or about 150 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, at or about 20 billion cells, at or about 30 billion cells, at or about 40 billion cells).

In some embodiments, the NK cells are administered in a dose comprising at least 1×10⁶, 2×10⁶, 3×10⁶, 4×10⁶, 5×10⁶, 6×10⁶, 7×10⁶, 8×10⁶, or 9×10⁶ cells per dose, at or at least 1×10⁷, 2×10⁷, 3×10⁷, 4×10⁷, 5×10⁷, 6×10⁷, 7×10⁷, 8×10⁷, or 9×10⁷ cells per dose, at or at least 1×10⁸, 2×10⁸, 3×10⁸, 4×10⁸, 5×10⁸, 6×10⁸, 7×10⁸, 8×10⁸, or 9×10⁸ cells per dose, at least 1×10⁹, 2×10⁹, 3×10⁹, 4×10⁹, 5×10⁹, 6×10⁹, 7×10⁹, 8×10⁹ , or 9×10⁹ cells per dose, or at least 1×10¹⁰ or 2×10¹⁰ cells per dose.

In some embodiments, the NK cells are administered in a dose comprising about 1×10⁶, 2×10⁶, 3×10⁶, 4×10⁶, 5×10⁶, 6×10⁶, 7×10⁶, 8×10⁶, or 9×10⁶ cells per dose, about 1×10⁷. 2×10⁷, 3×10⁷, 4×10⁷, 5×10⁷, 6×10⁷, 7×10⁷, 8×10⁷, or 9×10⁷ cells per dose, about 1×10⁸, 2×10⁸, 3×10⁸, 4×10⁸, 5×10⁸, 6×10⁸, 7×10⁸, 8×10⁸, or 9×10⁸ cells per dose, about 1×10⁹, 2×10⁹, 3×10⁹, 4×10⁹, 5×10⁹, 6×10⁹, 7×10⁹, 8×10⁹, or 9×10⁹ cells per dose, or about 1×10¹⁰ or 2×10¹⁰ cells per dose.

In some embodiments, the NK cells are administered in a dose comprising at or about 1 million to at or about 20 billion CAR-expressing NK cells (e.g., at or about 5 million cells, at or about 25 million CAR-expressing cells, at or about 50 million CAR-expressing cells, at or about 75 million CAR-expressing cells, at or about 100 million CAR-expressing cells, at or about 200 million CAR-expressing cells, at or about 300 million CAR-expressing cells, at or about 400 million CAR-expressing cells, at or about 500 million CAR-expressing cells, at or about 1 billion CAR-expressing cells, at or about 2 billion CAR-expressing cells, at or about 3 billion CAR-expressing cells, at or about 4 billion CAR-expressing cells, at or about 5 billion CAR-expressing cells, at or about 6 billion CAR-expressing cells, at or about 7 billion CAR-expressing cells, at or about 8 billion CAR-expressing cells, at or about 9 billion CAR-expressing cells, at or about 10 billion CAR-expressing cells, or at or about 20 billion CAR-expressing cells, or a range defined by any two of the foregoing values) per dose.

In some embodiments, the NK cells are administered in a dose comprising at or at about 1×10⁶, 2×10⁶, 3×10⁶, 4×10⁶, 5×10⁶, 6×10⁶, 7×10⁶, 8×10⁶, or 9×10⁶ CAR-expressing cells per dose, a dose comprising at or at about 1×10⁷, 2×10⁷, 3×10⁷, 4×10⁷, 5×107, 6×10⁷, 7×10⁷, 8×10⁷, or 9×10⁷ CAR-expressing cells per dose, a dose comprising at or at about 1×10⁸, 2×10⁸, 3×10⁸, 4×10⁸, 5×10⁸, 6×10⁸, 7×10⁸, 8×10⁸, or 9×10⁸ CAR-expressing cells per dose, a dose comprising at or at about 1×10⁹, 2×10⁹, 3×10⁹, 4×10⁹, 5×10⁹, 6×10⁹, 7×10⁹, 8×10⁹, or 9×10⁹ CAR-expressing cells per dose, or a dose comprising at or at about 1×10¹⁰ or at or at about 2×10¹⁰ CAR-expressing cells per dose.

In some embodiments, wherein the NK cells are administered in a dose comprising at least or at least about 1×10⁶, 2×10⁶, 3×10⁶, 4×10⁶. 5×10⁶, 6×10⁶, 7×10⁶, 8×10⁶, or 9×10⁶ CAR-expressing cells per dose, at least or at least about 1×10⁷, 2×10⁷, 3×10⁷, 4×10⁷, 5×107. 6×10⁷, 7×10⁷, 8×10⁷, or 9×10⁷ CAR-expressing cells per dose, at least or at least about 1×10⁸, 2×10⁸, 3×10⁸, 4×10⁸, 5×10⁸, 6×10⁸, 7×10⁸, 8×10⁸, or 9×10⁸ CAR-expressing cells per dose, at least or at least about 1×10⁹, 2×10⁹, 3×10⁹, 4×10⁹, 5×10⁹, 6×10⁹, 7×10⁹, 8×10⁹, or 9×10⁹ CAR-expressing cells per dose, at least or at least about 1×10¹⁰ CAR-expressing cells per dose.

Also provided herein are methods comprising administering NK cells to a patient in need thereof, wherein the NK cells comprise a polynucleotide comprising: a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and b) a nucleic acid encoding an IL-15, wherein the NK cells are administered in a dose comprising 1×10⁵ to 10×10⁸ cells/kg.

In some embodiments, the dose of cells comprises between at or about 1×10⁵ of the cells/kg and at or about 1×10⁸ of the cells/kg, such as between at or about 1.5×10⁵ of the cells/kg and at or about 1.5×10⁷ of the cells/kg, or between at or about 4×10⁵ of the cells/kg and at or about 4×10⁶ of the cells/kg.

In some embodiments, the NK cells are administered in a dose comprising at or at about 1×10⁵, 1.5×10⁵, 2×10⁵, 2.5×10⁵, 3×10⁵, 3.5×10⁵, 4×10⁵, 4.5×10⁵, 5×10⁵, 5.5×105. 6×10⁵, 6.5×10⁵, 7×10⁵, 7.5×10⁵, 8×10⁵, 8.5×10⁵, 9×10⁵, or 9.5×10⁵ cell/kg, 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶. 7×10⁶, 7.5×10⁶, 8×10⁶, 8.5×10⁶, 9×10⁶, or 9.5×10⁶ cells/kg, at or at about 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷ cells/kg, or 9.5×10⁷, or at or at about 1×10⁸1.5×10⁸, or 2×10⁸ cells/kg.

In some embodiments, the NK cells are administered in a dose comprising at least or at least about 1×10⁵. 1.5×10⁵, 2×10⁵, 2.5×10⁵, 3×10⁵, 3.5×10⁵, 4×10⁵, 4.5×10⁵. 5×10⁵, 5.5×10⁵, 6×10⁵, 6.5×10⁵, 7×10⁵, 7.5×10⁵, 8×10⁵, 8.5×10⁵, 9×10⁵, or 9.5×10⁵ cell/kg, at least or at least about 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, or 9.5×10⁶cells/kg, at least or at least about 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷ cells/kg, or 9.5×10⁷, or at least or at least about 1×10⁸ or 1.5×10⁸ cells/kg.

Also provided herein are methods comprising administering multiple doses of NK cells to a patient in need thereof, wherein the NK cells comprise a polynucleotide comprising: a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and b) a nucleic acid encoding an IL-15, and wherein the doses are administered once every one to sixteen weeks.

In some embodiments, the NK cells are administered bi-weekly, once every three weeks, once every four weeks, once every five weeks, once every six weeks, once every seven weeks, once every eight weeks, once every nine weeks, once every ten weeks, once every 11 weeks, once every 12 weeks, once every 13 weeks, once every 14 weeks, once every 15 weeks, or once every 16 weeks.

Also provided herein are methods comprising administering NK cells to a patient in need thereof, wherein the NK cells comprise a polynucleotide comprising: a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and b) a nucleic acid encoding an IL-15, wherein the patient meets one or more of the following criteria: (i) the patient has an Eastern Cooperative Oncology Group (ECOG) performance status of from 0 to 4 at the time of treatment: (ii) the patient is refractory to or has a recurrence after receiving 1 or more prior systemic therapies: (iii) the patient has an oxygen saturation level measured via pulse oximeter of at least 92%; (iv) the patient has a left ventricular ejection fraction (LVEF) of at least 50%: (v) the patient has an absolute neutrophil count (ANC) of at least 1000/mm³ (1.0×10⁹ /L): (vi) the patient has a platelet count of at least 75000/mm³ (75×10⁹ /L): (vii) the patient's hemoglobin level, with or without prior transfusion, is at least 8.0 g/dL: (viii) the patient's creatinine clearance is at least 45 mL/min using the Cockcraft-Gault equation, or mL/min/1.73 m² as estimated glomerular filtration rate (eGFR) per Modification of Diet in Renal Disease (MDRD) equation: (ix) the patient's total serum bilirubin is less than 5 mg/dL: or (x) the patient's liver transaminases (aspartate aminotransferase/alanine amino transferase/alkaline phosphatase (AST/ALT/ALP)) is less than or equal to 5 times the upper limit of normal (ULN) for the test.

In some embodiments, the patient has an Eastern Cooperative Oncology Group (ECOG) performance status of 0, 1, 2, 3, or 4 at the time of treatment.

In some embodiments, the patient is refractory to or has a recurrence after receiving 2 or more prior systemic therapies.

In some embodiments, the patient has an oxygen saturation level measured via pulse oximeter of at least 92%, 93%, 94%, 95%, 96%, 97%, 98%, or 99%.

In some embodiments, the patient has a left ventricular ejection fraction (LVEF) of at least 50%, 51%, 52%, 53%, 54%, 55%, 56%, 57%, 58%, 59%, 60%, 61%, 62%, 63%, 64%, or 65%.

In some embodiments, the patient has an absolute neutrophil count (ANC) of at least 1000/mm³ (1.0×109/L), 1500/mm³, 2000/mm³, 2500/mm³, 3000/mm³, 3500/mm³, 4000/mm³, 4500/mm³, 5000/mm³, 5500/mm³, or 6000/mm³.

In some embodiments, the patient has a platelet count of at least 75000/mm³ (75×109/L). 100000/mm³. 125000/mm³. 150000/mm³. 175000/mm³. 200000/mm³. 225000/mm³. 250000/mm³, 275000/mm³, 300000/mm³, 325000/mm³, 350000/mm³, 375000/mm³, or 400000/mm³.

In some embodiments, the patient's hemoglobin level, with or without prior transfusion, is at least 8.0, 9.0, 10.0, 11.0, 12.0, 130, 14.0, 15.0, 16.0, 17.0, 18.0, 19.0, or 20.0 g/dL

In some embodiments, the patient's creatinine clearance is at least 45, 50, 55, 60, 65, 70, 75, 80, 85, 90, 95, 100, 105, 110, 115, 120, 125, 130, 135, or 140 mL/min using the Cockcraft-Gault equation, or mL/min/1.73 m² as estimated glomerular filtration rate (eGFR) per Modification of Diet in Renal Disease (MDRD) equation.

In some embodiments, the patient's total serum bilirubin is less than 5, 4.5, 4.0, 3.5. 3.0, 2.5, 2.0, 1.5, 1.0, 0.5, 0.4, 0.3, 0.2, or 0.1 mg/dL.

In some embodiments, the patient's liver transaminases (aspartate aminotransferase/alanine amino transferase/alkaline phosphatase (AST/ALT/ALP)) is less than or equal to 5, 4.5, 4.0, 3.5, 3.0, 2.5, 2.0, 1.5, 1.0, 0.5, or 0 times the upper limit of normal (ULN) for the test.

Also provided herein are methods comprising administering NK cells to a patient in need thereof, wherein the NK cells comprise a polynucleotide comprising: a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and b) a nucleic acid encoding an IL-15, wherein the patient meets one or more of the following criteria: (i) the patient does not have active nervous system metastases: (ii) the patient does not have a history or presence of a clinically relevant CNS disorder such as a seizure disorder (e.g., epilepsy), cerebrovascular ischemia/hemorrhage. dementia, cerebellar disease, cerebral edema, posterior reversible encephalopathy syndrome (PRES), or any autoimmune disease with CNS involvement: (iii) the patient does not have active human immunodeficiency virus (HIV) infection, e.g., as evidenced by a positive HIV polymerase chain reaction (PCR) test; (iv) the patient does not have active HBV or hepatitis C virus (HCV) infection, e.g., based on laboratory testing: (v) the patient has a viral HBV load below the institutional limit of quantification (LOQ) and the subject is on stable viral suppressive therapy: or (vi) the patient has a HCV RNA viral load below the institutional LOQ and has completed curative antiviral treatment.

Also provided herein are methods comprising administering NK cells to a patient in need thereof, wherein the NK cells comprise a polynucleotide comprising: a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and b) a nucleic acid encoding an IL-15, wherein the patient meets one or more of the following criteria: (i) the patient is diagnosed with or has been diagnosed with a HER2+ cancer with an IHC score of 2+: (ii) the patient has a cancer that is not HER2-addicted, but shows some degree of HER2-expression: (iii) the patient is diagnosed with or has been diagnosed with a HER2-low cancer: (iv) the patient is diagnosed with or has been diagnosed with a HER2-low cancer with a HER2 expression classification of score 1+ or 2+: (v) the patient is diagnosed with or has been diagnosed with a HER2-low cancer without HER2 amplification: (vi) the patient has a cancer comprising a HER2 activating mutation: or (vii) the patient has been diagnosed with a HER2 expressing cancer that is IHC 1+ or IHC 2+/ISH− and is refractory to or has a recurrence after receiving previous treatment.

In some embodiments, the patient is diagnosed with or has been diagnosed with a HER2-low cancer with a HER2 expression classification of score 1+ or 2+ without HER2 amplification.

In some embodiments, the HER2-low cancer harbors HER2 gain.

In some embodiments, the HER2-low cancer lacks HER2 gain.

In some embodiments, the patient has been diagnosed with a IHC 3+ or IHC 2+/ISH+ cancer and is refractory to or has a recurrence after receiving previous treatment with a HER2-targeting therapy.

In some embodiments, the patient has been diagnosed with a HER2 expressing cancer that is IHC 1+ or IHC 2+/ISH−, and the patient's cancer is unresectable, metastatic, or the patient is refractory to or has a recurrence after receiving previous treatment.

In some embodiments, of any of the methods described herein, the anti-HER2 antibody or antigen binding fragment thereof comprises a light chain complementarity determining region 1 (CDRL1) comprising SEQ ID NO: 34, a light chain complementarity determining region 2 (CDRL2) comprising SEQ ID NO: 36: a light chain complementarity determining region 3 (CDRL3) comprising SEQ ID NO: 38, a heavy chain complementarity determining region 1 (CDRH1 comprising SEQ ID NO: 44: a heavy chain complementarity determining region 2 (CDRH2) comprising SEQ ID NO: 46; and a heavy chain complementarity determining region 3 (CDRH3) comprising SEQ ID NO: 48.

In some embodiments, of any of the methods described herein, the nucleic acid encoding the anti-HER2 antibody or antigen binding fragment thereof encodes a CDRL1 encoded by SEQ ID NO: 35, a CDRL2 encoded by SEQ ID NO: 37: a CDRL3 encoded by SEQ ID NO: 39, a CDRH1 encoded by SEQ ID NO: 45: a CDRH2 encoded by SEQ ID NO: 47; and a CDRH3 encoded by SEQ ID NO: 49.

In some embodiments, of any of the methods described herein, the anti-HER2 antibody or antigen binding fragment thereof comprises a light chain variable (VL) region comprising SEQ ID NO: 32 and a heavy chain variable (V_{H}) region comprising SEQ ID NO: 42.

In some embodiments, of any of the methods described herein, the nucleic acid encoding the anti-HER2 antibody or antigen binding fragment thereof comprises a nucleic acid encoding a VL region comprising SEQ ID NO: 33 and a nucleic acid encoding a V_{H} region comprising SEQ ID NO: 37.

In some embodiments, of any of the methods described herein, the anti-HER2 antibody or antigen binding fragment thereof comprises a VL region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 32 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 42.

In some embodiments, of any of the methods described herein, the anti-HER2 antibody or antigen binding fragment thereof is an antigen binding fragment. In some embodiments, the antigen binding fragment comprises a single chain Fv (scFv). In some embodiments, the VL region is amino-terminal to the V_{H} region. In some embodiments, the VL region is carboxy-terminal to the V_{H} region. In some embodiments, the VL region is joined to the V_{H} region via a flexible linker. In some embodiments, the flexible linker comprises the amino acid sequence set forth in SEQ ID NO: 40. In some embodiments, the flexible linker is encoded by a nucleic acid comprising SEQ ID NO: 41. In some embodiments, the scFv comprises the amino acid sequence set forth in SEQ ID NO: 30. In some embodiments, the scFv is encoded by a nucleic acid comprising SEQ ID NO: 31. In some embodiments, the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 30.

In some embodiments, of any of the methods described herein, the anti-HER2 CAR specifically binds to a human epidermal growth factor receptor 2 (HER2) protein. In some embodiments, the HER2 protein comprises the amino acid sequence of SEQ ID NO: 62.

In some embodiments, of any of the methods described herein, the CAR comprises a transmembrane domain, optionally a CD28 transmembrane domain. In some embodiments, the CD28 transmembrane domain comprises the amino acid sequence set forth in SEQ ID NO: 53. In some embodiments, the CD28 transmembrane domain is encoded by a nucleic acid comprising the nucleic acid sequence set forth in SEQ ID NO: 54 or SEQ ID NO: 55.

In some embodiments, of any of the methods described herein, the CAR further comprises a hinge domain between the extracellular antigen binding domain and the transmembrane domain. In some embodiments, the hinge domain comprises at least a portion of a CD8a hinge domain. In some embodiments, the CD8a hinge domain comprises an amino acid sequence set forth in SEQ ID NO: 50. In some embodiments, the CD8a hinge domain is encoded by a nucleic acid comprising SEQ ID NO: 51 or SEQ ID NO: 52. In some embodiments, the CD8a hinge domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 50.

In some embodiments, of any of the methods described herein, the CAR comprises an intracellular signaling region, optionally where the intracellular signaling region comprises a CD28 intracellular signaling domain, an OX40L intracellular signaling domain, and a CD3-zeta (CD35) signaling domain. In some embodiments, the intracellular signaling region comprises a CD28 intracellular signaling domain and a CD3-zeta signaling domain.

In some embodiments, the intracellular signaling region comprises an OX40L intracellular signaling domain. In some embodiments, the OX40L intracellular signaling domain comprises an amino acid sequence set forth in SEQ ID NO: 8, SEQ ID NO: 9, or SEQ ID NO: 10. In some embodiments, the OX40L intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 8, SEQ ID NO: 9, or SEQ ID NO: 10. In some embodiments, the OX40L intracellular signaling domain is encoded by a nucleic acid comprising SEQ ID NO: 11 or SEQ ID NO: 12.

In some embodiments, the intracellular signaling region comprises a CD28 intracellular signaling domain. In some embodiments, the CD28 intracellular signaling domain comprises an amino acid sequence set forth in SEQ ID NO: 5. In some embodiments, the CD28 intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 5. In some embodiments, the CD28 intracellular signaling domain is encoded by a nucleic acid comprising SEQ ID NO: 6 or SEQ ID NO: 7.

In some embodiments, the intracellular signaling region comprises an CD3-zeta intracellular signaling domain. In some embodiments, the CD3-zeta intracellular signaling domain comprises an amino acid sequence set forth in SEQ ID NO: 13. In some embodiments, the CD3-zeta intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 13. In some embodiments, the CD3-zeta intracellular signaling domain is encoded by a nucleic acid comprising SEQ ID NO: 14 or SEQ ID NO: 15.

In some embodiments, the intracellular signaling region comprises an amino acid sequence set forth in SEQ ID NO: 25. In some embodiments, the intracellular signaling region comprises an amino acid sequence having or having at least 85%, 86%, 87%, 88%, 89%, 90%, 91%, 92%, 93%, 94%, 95%, 96%, 97%, 98%, or 99% identity to SEQ ID NO: 25.

In some embodiments, of any of the methods described herein, the CAR comprises an amino sequence set forth in SEQ ID NO: 56. In some embodiments, the CAR is encoded by a nucleic acid comprising SEQ ID NO: 57.

In some embodiments, of any of the methods described herein, the IL-15 comprises the amino acid sequence set forth in SEQ ID NO: 22. In some embodiments, the IL-15 is encoded by a nucleic acid comprising SEQ ID NO: 23 or SEQ ID NO: 24.

In some embodiments, of any of the methods described herein, the polynucleotide encodes a polyprotein comprising the CAR and the IL-15.

In some embodiments, of any of the methods described herein, the polynucleotide further comprises a nucleic acid encoding a self-cleaving peptide, optionally a T2A self-cleaving peptide. In some embodiments, the CAR is joined to the IL-15 by the self-cleaving peptide. In some embodiments, the self-cleaving peptide is capable of inducing ribosomal skipping between the CAR and the IL-15.

In some embodiments, of any of the methods described herein, the polynucleotide further comprises a nucleic acid encoding a signal sequence. In some embodiments, the signal sequence comprises the amino acid sequence set forth in SEQ ID NO: 27. In some embodiments, the nucleic acid encoding the signal sequence comprises the nucleic acid sequence set forth in SEQ ID NO: 28.

In some embodiments, of any of the methods described herein, the polynucleotide encodes a polyprotein comprising the amino acid sequence set forth in SEQ ID NO: 59.

In some embodiments, of any of the methods described herein, the polynucleotide comprises the nucleic acid sequence set forth in SEQ ID NO: 60 or SEQ ID NO: 61.

In some embodiments, of any of the methods described herein, the NK cells are cord blood NK cells.

In some embodiments, of any of the methods described herein, the NK cells comprise a KIR-B haplotype.

In some embodiments, of any of the methods described herein, the NK cells express CD16 having the V/V polymorphism at F158.

In some embodiments, of any of the methods described herein, the NK cells are administered as part of a pharmaceutical composition. In some embodiments, the pharmaceutical composition comprises a pharmaceutically acceptable excipient. In some embodiments, the pharmaceutical composition comprises: (a) human albumin: (b) dextran: (c) glucose: (d) DMSO; and (e) a buffer.

In some embodiments, the pharmaceutical composition comprises from 30 to 50 mg/mL human albumin. In some embodiments, the pharmaceutical composition comprises 40 mg/mL human albumin.

In some embodiments, the pharmaceutical composition comprises 20 to 30 mg/mL dextran. In some embodiments, the pharmaceutical composition comprises 25 mg/mL dextran. In some embodiments, the dextran is Dextran 40.

In some embodiments, the pharmaceutical composition comprises from 12 to 15 mg/mL glucose. In some embodiments, the pharmaceutical composition comprises 12.5 mg/mL glucose. In some embodiments, the pharmaceutical composition comprises less than 27.5 g/L glucose.

In some embodiments, the pharmaceutical composition comprises from 50 to 60 ml/mL DMSO. In some embodiments, the pharmaceutical composition comprises 55 mg/mL DMSO. In some embodiments, the pharmaceutical composition comprises 40 to 60% v/v buffer. In some embodiments, the buffer is phosphate buffered saline.

In some embodiments, the pharmaceutical composition comprises: (a) about 40 mg/mL human albumin: (b) about 25 mg/mL Dextran 40; (c) about 12.5 mg/mL glucose: (d) about 55 mg/mL DMSO; and (e) about 0.5 mL/mL phosphate buffered saline.

In some embodiments, the pharmaceutical composition further comprises 0.5 mL/mL water.

In some embodiments, of any of the methods described herein, the subject has a disease or condition associated with HER2. In some embodiments, the disease or condition associated with HER2 is cancer. In some embodiments, the cancer expresses HER2. In some embodiments, the cancer is a HER2+ cancer. In some embodiments, the cancer is or comprises a solid tumor. In some embodiments, the cancer is or comprises a bladder cancer, breast adenocarcinoma, colorectal adenocarcinoma, non-small cell lung cancer, esophageal cancer, cervix squamous cancer, stomach adenocarcinoma, cholangiocarcinoma, ovary cancer, renal papillary cell carcinoma, and combinations thereof.

In some embodiments, the cancer is or comprises a breast cancer. In some embodiments, the breast cancer is noninvasive. In some embodiments, the breast cancer is invasive. In some embodiments, the breast cancer is metastatic.

In some embodiments, the cancer is or comprises a gastric cancer.

In some embodiments, the cancer is or comprises an ovarian cancer.

In some embodiments, the cancer is or comprises a gastroesophageal cancer.

In some embodiments, the cancer is or comprises a lung cancer. In some embodiments, the lung cancer is non-small cell lung cancer (NSCLC). In some embodiments, the patient has a HER2 activating mutation.

In some embodiments, of any of the methods described herein, the method further comprising administering a lymphodepleting chemotherapy to the subject prior to treatment. In some embodiments, the lymphodepleting chemotherapy is non-myeloablative chemotherapy. In some embodiments, the lymphodepleting chemotherapy comprises treatment with at least one of cyclophosphamide and fludarabine. In some embodiments, the lymphodepleting chemotherapy comprises treatment with cyclophosphamide and fludarabine. In some embodiments. between 100 and 500 mg/m2 cyclophosphamide is administered per day. In some embodiments, 250 mg/m2 cyclophosphamide is administered per day. In some embodiments, 500 mg/m2 cyclophosphamide is administered per day. In some embodiments, between 10 and 50 mg/m2 of fludarabine is administered per day. In some embodiments. 30 mg/m2 of fludarabine is administered per day.

In some embodiments, of any of the methods described herein, the method further comprises administering IL-2 to the subject. In some embodiments, the patient is administered 1×10⁶ IU/m² of IL-2. In some embodiments, the patient is administered 1×10⁷ IU of IL-2. In some embodiments, the patient is administered 6×10⁷ IU of IL-2. In some embodiments, administration of IL-2 occurs within 1-4 hours of administration of the cells. In some embodiments, administration of IL-2 occurs at least 1-4 hours after the administration of the cells.

In some embodiments, of any of the methods described herein, the method further comprises administering the cells a plurality of times. In some embodiments, the method comprises administering the cells three times, four times, or eight times. In some embodiments, the method comprises administering the cells every week, every two weeks, every three weeks, or every four weeks.

In some embodiments, of any of the methods described herein, the method further comprises administering pertuzumab to the subject. In some embodiments, of any of the methods described herein, the method further comprises administering trastuzumab to the subject. In some embodiments, of any of the methods described herein, the method further comprises administering necitumumab to the subject. In some embodiments, of any of the methods described herein, the method further comprises administering margetuximab to the subject. In some embodiments, of any of the methods described herein, the method further comprises administering taxane to the subject. In some embodiments, the taxane is at least one of paclitaxel, docetaxel, and cabazitaxel. In some embodiments, of any of the methods described herein, the method further comprises administering an endocrine therapy to the subject. In some embodiments, the endocrine therapy comprises at least one of an aromatase inhibitor, fulvestrant, and tamoxifen.

In some embodiments, of any of the methods described herein, the method further comprises administering a checkpoint inhibitor to the subject. In some embodiments, the checkpoint inhibitor inhibits CTLA-4, PD-1, or PD-L1. In some embodiments, the checkpoint inhibitor is or comprises ipilimumab. In some embodiments, the checkpoint inhibitor is or comprises nivolumab. In some embodiments, the checkpoint inhibitor is or comprises pembrolizumab. In some embodiments, the checkpoint inhibitor is or comprises cemiplimab. In some embodiments, the checkpoint inhibitor is or comprises atezolizumab. In some embodiments, the checkpoint inhibitor is or comprises avelumab. In some embodiments, the checkpoint inhibitor is or comprises durvalumab.

In some embodiments, of any of the methods described herein, the method further comprises administering a second therapeutic moiety to the subject. In some embodiments, the second therapeutic moiety comprises a lymphodepleting chemotherapy agent. In some embodiments, the second therapeutic moiety comprises IL-2. In some embodiments, the second therapeutic moiety comprises at least one of pertuzumab, trastuzumab, necitumumab, and margetuximab. In some embodiments, the second therapeutic moiety comprises a taxane. In some embodiments, the second therapeutic moiety comprises a checkpoint inhibitor.

Also provided herein are polynucleotides comprising a nucleic acid encoding an anti-human epidermal growth factor receptor 2 (HER2) chimeric antigen receptor (CAR) and natural killer cells expressing the polynucleotides.

Provided herein are polynucleotide(s) comprising: a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and b) a nucleic acid encoding an IL-15.

In some embodiments, the anti-HER2 antibody or antigen binding fragment thereof comprises a light chain complementarity determining region 1 (CDRL1) comprising SEQ ID NO: 34, a light chain complementarity determining region 2 (CDRL2) comprising SEQ ID NO: 36: a light chain complementarity determining region 3 (CDRL3) comprising SEQ ID NO: 38, a heavy chain complementarity determining region 1 (CDRH1 comprising SEQ ID NO: 44: a heavy chain complementarity determining region 2 (CDRH2) comprising SEQ ID NO: 46; and a heavy chain complementarity determining region 3 (CDRH3) comprising SEQ ID NO: 48.

In some embodiments, the nucleic acid encoding the anti-HER2 antibody or antigen binding fragment thereof encodes a CDRL1 encoded by SEQ ID NO: 35, a CDRL2 encoded by SEQ ID NO: 37: a CDRL3 encoded by SEQ ID NO: 39, a CDRH1 encoded by SEQ ID NO: 45: a CDRH2 encoded by SEQ ID NO: 47; and a CDRH3 encoded by SEQ ID NO: 49.

In some embodiments, the anti-HER2 antibody or antigen binding fragment thereof comprises a light chain variable (VL) region comprising SEQ ID NO: 32 and a heavy chain variable (V_{H}) region comprising SEQ ID NO: 42.

In some embodiments, the nucleic acid encoding the anti-HER2 antibody or antigen binding fragment thereof comprises a nucleic acid encoding a VL region comprising SEQ ID NO: 33 and a nucleic acid encoding a V_{H} region comprising SEQ ID NO: 42.

In some embodiments, the anti-HER2 antibody or antigen binding fragment thereof comprises a VL region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 32 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 42.

In some embodiments, the anti-HER2 antibody or antigen binding fragment thereof is an antigen binding fragment.

In some embodiments, the antigen binding fragment comprises a single chain Fv (scFv).

In some embodiments, the VL region is amino-terminal to the V_{H} region.

In some embodiments, the VL region is carboxy-terminal to the V_{H} region.

In some embodiments, the VL region is joined to the V_{H} region via a flexible linker.

In some embodiments, the flexible linker comprises the amino acid sequence set forth in SEQ ID NO: 40.

In some embodiments, the flexible linker is encoded by a nucleic acid comprising SEQ ID NO: 41.

In some embodiments, the scFv comprises the amino acid sequence set forth in SEQ ID NO: 30.

In some embodiments, the scFv is encoded by a nucleic acid comprising SEQ ID NO: 31.

In some embodiments, the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 30.

In some embodiments, the anti-HER2 CAR specifically binds to a human epidermal growth factor receptor 2 (HER2) protein.

In some embodiments, the HER2 protein comprises the amino acid sequence of SEQ ID NO: 62.

In some embodiments, the CAR comprises a transmembrane domain, optionally a CD28 transmembrane domain.

In some embodiments, the CD28 transmembrane domain comprises the amino acid sequence set forth in SEQ ID NO: 53.

In some embodiments, the CD28 transmembrane domain is encoded by a nucleic acid comprising the nucleic acid sequence set forth in SEQ ID NO: 54 or SEQ ID NO: 55.

In some embodiments, the polynucleotide further comprises a hinge domain between the extracellular antigen binding domain and the transmembrane domain.

In some embodiments, the hinge domain comprises at least a portion of a CD8a hinge domain.

In some embodiments, the CD8a hinge domain comprises an amino acid sequence set forth in SEQ ID NO: 50.

In some embodiments, the CD8a hinge domain is encoded by a nucleic acid comprising SEQ ID NO: 51 or SEQ ID NO: 52.

In some embodiments, the CD8a hinge domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 50.

In some embodiments, the CAR comprises an intracellular signaling region, optionally where the intracellular signaling region comprises a CD28 intracellular signaling domain, an OX40L intracellular signaling domain, and a CD3-zeta (CD32) signaling domain.

In some embodiments, the intracellular signaling region comprises a CD28 intracellular signaling domain and a CD3-zeta signaling domain.

In some embodiments, the intracellular signaling region comprises an OX40L intracellular signaling domain.

In some embodiments, the OX40L intracellular signaling domain comprises an amino acid sequence set forth in SEQ ID NO: 8, SEQ ID NO: 9, or SEQ ID NO: 10.

In some embodiments, the OX40L intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 8, SEQ ID NO: 9, or SEQ ID NO: 10.

In some embodiments, the OX40L intracellular signaling domain is encoded by a nucleic acid comprising SEQ ID NO: 11 or SEQ ID NO: 12.

In some embodiments, the intracellular signaling region comprises a CD28 intracellular signaling domain.

In some embodiments, the CD28 intracellular signaling domain comprises an amino acid sequence set forth in SEQ ID NO: 5.

In some embodiments, the CD28 intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 5.

In some embodiments, the CD28 intracellular signaling domain is encoded by a nucleic acid comprising SEQ ID NO: 6 or SEQ ID NO: 7.

In some embodiments, the intracellular signaling region comprises an CD3-zeta intracellular signaling domain.

In some embodiments, the CD3-zeta intracellular signaling domain comprises an amino acid sequence set forth in SEQ ID NO: 13.

In some embodiments, the CD3-zeta intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 13.

In some embodiments, the CD3-zeta intracellular signaling domain is encoded by a nucleic acid comprising SEQ ID NO: 14 or SEQ ID NO: 15.

In some embodiments, the intracellular signaling region comprises an amino acid sequence set forth in SEQ ID NO: 25.

In some embodiments, the intracellular signaling region comprises an amino acid sequence having or having at least 85%, 86%, 87%, 88%, 89%, 90%, 91%, 92%, 93%, 94%, 95%, 96%, 97%, 98%, or 99% identity to SEQ ID NO: 25.

In some embodiments, the CAR comprises an amino sequence set forth in SEQ ID NO: 56.

In some embodiments, the CAR is encoded by a nucleic acid comprising SEQ ID NO: 57.

In some embodiments, the IL-15 comprises the amino acid sequence set forth in SEQ ID NO: 22.

In some embodiments, the IL-15 is encoded by a nucleic acid comprising SEQ ID NO: 23 or SEQ ID NO: 24.

In some embodiments, the polynucleotide encodes a polyprotein comprising the CAR and the IL-15.

In some embodiments, the polynucleotide further comprises a nucleic acid encoding a self-cleaving peptide, optionally a T2A self-cleaving peptide.

In some embodiments, the CAR is joined to the IL-15 by the self-cleaving peptide.

In some embodiments, the self-cleaving peptide is capable of inducing ribosomal skipping between the CAR and the IL-15.

In some embodiments, the polynucleotide further comprises a nucleic acid encoding a signal sequence.

In some embodiments, the signal sequence comprises the amino acid sequence set forth in SEQ ID NO: 27.

In some embodiments, the nucleic acid encoding the signal sequence comprises the nucleic acid sequence set forth in SEQ ID NO: 28.

In some embodiments, the polynucleotide encodes a polyprotein comprising the amino acid sequence set forth in SEQ ID NO: 59.

In some embodiments, the polynucleotide comprises the nucleic acid sequence set forth in SEQ ID NO: 60 or SEQ ID NO: 61.

Also provided herein are vector(s) comprising the polynucleotide(s) described herein. In some embodiments, the vector is a viral vector.

In some embodiments, the viral vector is a retroviral vector or a lentiviral vector.

Also provided herein are cell(s) comprising the polynucleotide(s) and/or vector(s) described herein.

Also provided herein are cell(s) expressing the chimeric antigen receptor(s) and IL-15 encoded by the polynucleotide(s) described herein and/or or the vector(s) described herein

In some embodiments, the cell is a lymphocyte.

In some embodiments, the lymphocyte is a natural killer (NK) cell.

In some embodiments, the lymphocyte is a T cell.

In some embodiments, the cell is a human cell.

In some embodiments, the cell is a primary cell obtained from a subject.

In some embodiments, the cell is a primary cell obtained from cord blood.

In some embodiments, the cell comprises a KIR-B haplotype.

In some embodiments, the cell express CD16 having the V/V polymorphism at F158.

Also provided herein are population(s) of cells comprising a plurality of the cell(s) described herein.

In some embodiments, at least 10%, 20%, 30%, 40%, 50%, 60%, 70%, 80%, 85%, 90%: 95%, 96%, 97%, 98%, or 99% of the cells comprise the polynucleotide(s) and/or vector(s) described herein.

In some embodiments, at least 10%, 20%, 30%, 40%, 50%, 60%, 70%, 80%, 85%, 90%: 95%, 96%, 97%, 98%, or 99% of the cells express the chimeric antigen receptor(s) and the IL-15 encoded by the polynucleotide(s) and/or vector(s) described herein.

Also provided herein are pharmaceutical composition(s) comprising the population(s) of cells described herein.

In some embodiments, the pharmaceutical composition further comprises a pharmaceutically acceptable excipient.

In some embodiments, the pharmaceutical composition further comprises: (a) human albumin: (b) dextran: (c) glucose: (d) DMSO; and (e) a buffer.

In some embodiments, the pharmaceutical composition comprises from 30 to 50 mg/mL human albumin.

In some embodiments, the pharmaceutical composition comprises 50 mg/mL human albumin.

In some embodiments, the pharmaceutical composition comprises 20 to 30 mg/mL dextran.

In some embodiments, the pharmaceutical composition comprises 25 mg/mL dextran. In some embodiments, the dextran is Dextran 40.

In some embodiments, the pharmaceutical composition comprises from 12 to 15 mg/mL glucose.

In some embodiments, the pharmaceutical composition comprises 12.5 mg/ml glucose.

In some embodiments, the pharmaceutical composition comprises less than 27.5 g/L glucose.

In some embodiments, the pharmaceutical composition comprises from 50 to 60 ml/mL DMSO.

In some embodiments, the pharmaceutical composition comprises 55 mg/mL DMSO.

In some embodiments, the pharmaceutical composition comprises 40 to 60% v/v buffer.

In some embodiments, the buffer is phosphate buffered saline.

In some embodiments, the pharmaceutical composition comprises: (a) about 40 mg/mL human albumin: (b) about 25 mg/mL Dextran 40; (c) about 12.5 mg/mL glucose: (d) about 55 mg/mL DMSO; and (e) about 0.5 mL/mL phosphate buffered saline.

In some embodiments, the pharmaceutical composition further comprises 0.5 mL/mL water.

Also provided herein are frozen vial(s) comprising the composition(s) described herein.

Also provided herein are methods of treatment comprising administering the cell(s) described herein, the population(s) of cells described herein, or the composition(s) described herein to a subject having a disease or condition associated with HER2.

Also provided herein are uses of the cell(s) described herein, the population(s) of cells described herein, or the composition(s) described herein in the manufacture of a medicament for treating a disease or condition associated with HER2.

Also provided herein are uses of the cell(s) described herein, the population(s) of cells described herein, or the composition(s) described herein for treating a disease or condition associated with HER2.

In some embodiments, the disease or condition associated with HER2 is cancer.

In some embodiments, the cancer is a HER2+ cancer.

In some embodiments, the HER2+ cancer is or comprises a solid tumor expressing HER2.

In some embodiments, the HER2+ is or comprises a bladder cancer, breast adenocarcinoma, colorectal adenocarcinoma, non-small cell lung cancer, esophageal cancer, cervix squamous cancer, stomach adenocarcinoma, cholangiocarcinoma, ovary cancer, renal papillary cell carcinoma, and combinations thereof.

In some embodiments, the HER2+ is or comprises a breast cancer.

In some embodiments, the HER2+ is or comprises a gastric cancer.

In some embodiments, the HER2+ is or comprises an ovarian cancer.

In some embodiments, the method or use further comprises administering a lymphodepleting chemotherapy to the subject prior to treatment.

In some embodiments, the lymphodepleting chemotherapy is non-myeloablative chemotherapy.

In some embodiments, the lymphodepleting chemotherapy comprises treatment with at least one of cyclophosphamide and fludarabine.

In some embodiments, the lymphodepleting chemotherapy comprises treatment with cyclophosphamide and fludarabine.

In some embodiments, between 100 and 500 mg/m2 cyclophosphamide is administered per day.

In some embodiments, 250 mg/m2 cyclophosphamide is administered per day.

In some embodiments, 500 mg/m2 cyclophosphamide is administered per day.

In some embodiments, between 10 and 50 mg/m2 of fludarabine is administered per

day.

In some embodiments, 30 mg/m2 of fludarabine is administered per day.

In some embodiments, the method or use further comprises administering IL-2 to the subject.

In some embodiments, the patient is administered 1×10⁶IU/m² of IL-2.

In some embodiments, the patient is administered 1×10⁷ IU of IL-2.

In some embodiments, the patient is administered 6×10⁷ IU of IL-2.

In some embodiments, administration of IL-2 occurs within 1-4 hours of

administration of the cell(s), population(s) of cell(s), and/or pharmaceutical composition(s).

In some embodiments, administration of IL-2 occurs at least 1-4 hours after the administration of the cell(s), population(s) of cell(s), and/or pharmaceutical composition(s).

In some embodiments, the method or use comprises administering the cell(s), population(s) of cells, and/or pharmaceutical composition(s) a plurality of times.

In some embodiments, the method or use comprises administering the cell(s), population(s) of cells, and/or pharmaceutical composition(s) three, four times, or eight times.

In some embodiments, the method or use comprises administering the cell(s), population(s) of cells, and/or pharmaceutical composition(s) every week, every two weeks, every three weeks, or every four weeks.

In some embodiments, the method or use further comprises administering pertuzumab to the subject.

In some embodiments, the method or use further comprises administering trastuzumab to the subject.

In some embodiments, the method or use further comprises administering necitumumab to the subject.

In some embodiments, the method or use further comprises administering margetuximab to the subject.

In some embodiments, the method or use further comprises administering taxane to the subject.

In some embodiments, the taxane is at least one of paclitaxel, docetaxel, and

cabazitaxel

In some embodiments, the method or use further comprises administering an endocrine therapy to the subject.

In some embodiments, the endocrine therapy comprises at least one of an aromatase inhibitor, fulvestrant, and tamoxifen.

In some embodiments, the method or use further comprises administering a checkpoint inhibitor to the subject.

In some embodiments, the checkpoint inhibitor inhibits CTLA-4, PD-1, or PD-L1.

In some embodiments, the checkpoint inhibitor is or comprises ipilimumab.

In some embodiments, the checkpoint inhibitor is or comprises nivolumab.

In some embodiments, the checkpoint inhibitor is or comprises pembrolizumab.

In some embodiments, the checkpoint inhibitor is or comprises cemiplimab.

In some embodiments, the checkpoint inhibitor is or comprises atezolizumab.

In some embodiments, the checkpoint inhibitor is or comprises avelumab.

In some embodiments, the checkpoint inhibitor is or comprises durvalumab.

Also provided herein are methods of treatment comprising: administering to a subject having a disease or condition associated with HER2 the cell(s) described herein, the population(s) of cells described herein, and/or the pharmaceutical composition(s) described herein; and a second therapeutic moiety.

In some embodiments, the second therapeutic moiety comprises a lymphodepleting chemotherapy agent.

In some embodiments, the second therapeutic moiety comprises IL-2.

In some embodiments, the second therapeutic moiety comprises at least one of pertuzumab, trastuzumab, necitumumab, and margetuximab.

In some embodiments, the second therapeutic moiety comprises a taxane.

In some embodiments, the second therapeutic moiety comprises a checkpoint inhibitor.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative and are not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary embodiment of a method for NK cell expansion and stimulation.

FIG. 2 shows examples of different manufacturing schemes for master cell bank (MCB) and drug product (DP) manufacturing.

FIG. 3 shows phenotypes of expanded and stimulated population of NK cells.

FIG. 4 shows that CAR-NKs comprising a co-stimulatory domain comprising OX40L exhibited greater cytotoxic potential than those without OX40L.

FIG. 5 shows schematics of CAR constructs.

FIG. 6 shows proliferation of the CAR constructs of FIG. 5.

FIG. 7 shows CAR expression of the CAR constructs of FIG. 5.

FIG. 8 shows CD107a expression of the CAR constructs of FIG. 5. Bars, from left to right: Mock, 2nd-CAR, 3rd_CAR.

FIG. 9 shows IFN-γ expression of the CAR constructs of FIG. 5. Bars, from left to right: Mock, 2nd-CAR, 3rd-CAR.

FIG. 10 shows TNF-α expression of the CAR constructs of FIG. 5. Bars, from left to right: Mock, 2nd-CAR. 3rd-CAR.

FIG. 11 shows short term cytotoxicity of the CAR constructs of FIG. 5.

FIG. 12 shows schemes of CAR constructs. From top to bottom: mock GFP expressing NK (mock-NK); CAR without IL-15 (CAR-NK); truncated CAR with IL-15 (CAR (t)-IL-15-NK): CAR with IL-15 (CAR-IL-15-NK, AB-201)

FIG. 13 shows CAR expression on NK cells cultured in the presence of IL-2.

FIG. 14 shows CAR expression on NK cells cultured in the absence of IL-2.

FIG. 15 shows proliferation of NK cells cultured in the presence of IL-2 and in the absence of IL-2.

FIG. 16 shows viability of NK cells cultured in the presence of IL-2 and in the absence of IL-2.

FIG. 17 shows cytotoxicity of NK cells.

FIG. 18 shows IFNg production of NK cells.

FIG. 19 shows IL-15 production of NK cells.

FIG. 20 demonstrates that secretion of IL-15 Maintains the Survival of Bystander NK Cells.

FIG. 21 is a schematic of two different CAR structures.

FIG. 22 shows CAR expression of the CAR structures shown in FIG. 21 over time

(in days).

FIG. 23 shows survival of cells expressing the CAR structures shown in FIG. 21 (cell numbers).

FIG. 24 shows survival of cells expressing the CAR structures shown in FIG. 21 (percent viability).

FIG. 25 shows the number of viable NK cells after 7 days of co-culture of cells expressing the CAR structures shown in FIG. 21 with target cells without IL-2 support. Bars, from left to right: cord blood NK cells (CBNK): 3rd CAR-NK: 4th CAR-NK.

FIG. 26 shows in vitro killing activity of AB-201 against the breast carcinoma (HER2-) cell line MDA-MB-468.

FIG. 27 shows in vitro killing activity of AB-201 against the ovarian carcinoma (HER2+) cell line SKOV3.

FIG. 28 shows in vitro killing activity of AB-201 against the gastric carcinoma (HER2+) cell line NCI-N87.

FIG. 29 shows in vitro killing activity of AB-201 against the breast carcinoma (HER2+) cell line HCC1954.

FIG. 30 shows in vitro killing activity of AB-201 against the breast carcinoma

(HER2+) cell line K562.

FIG. 31 shows in vitro characterization of AB-201.

FIG. 32 shows in vitro characterization of AB-201.

FIG. 33 shows in vitro characterization of AB-201.

FIG. 34 shows in vitro characterization of AB-201.

FIG. 35 shows in vitro characterization of AB-201.

FIG. 36 shows in vitro characterization of AB-201.

FIG. 37 shows the results of a long-term killing assay on NCI-N87 gastric carcinoma cells in culture using Incucyte live cell imaging. Effector to target ratio (E: T 0.3:1).

FIG. 38 shows that a single AB-201 administration of one million cells in a HER2+HCC1954 Breast Carcinoma model conferred a substantial survival benefit over trastuzumab.

FIG. 39 shows that a single AB-201 administration on day four after the establishment of a HER2+ trastuzumab-resistant breast cancer model resulted in tumor regression.

FIG. 40 shows in vivo characterization of AB-201.

FIG. 41 shows in vivo characterization of AB-201.

FIG. 42 shows in vivo characterization of AB-201.

FIG. 43 shows in vivo characterization of AB-201.

FIG. 44 shows cytotoxicity of primary cells (non-tumor) measured following co-culture of AB-201 or control CB-NK cells with pulmonary artery endothelial cells. keratinocytes, renal epithelial cells, cardiac myocytes and small airway epithelial cells for 4 hours at Effector: Target (E: T) ratios of 3:1, 1:1, or 0.3:1.

FIG. 45 shows tumor volume measurements of NSG mice that received SKOV3-Luc tumor cells (IP) and either not treated (open circles, dashed line) or treated with AB-201 (closed circles, solid line). The vertical line on Day 11 depicts the date of the AB-201 injection.

FIG. 46 shows measurements of body weight in NSG mice that received SKOV3-Luc tumor cells (IP) and either not treated (open circles, dashed line) or treated with AB-201 (closed circles, solid line).

FIG. 47 shows that AB-201 cells persisted in AB-201-treated mice at detectable levels until at least day 52.

FIG. 48 shows tumor volume of irradiated mice inoculated with NCI-N87 cells and administered no treatment, cord blood NK cells, or AB-201 cells.

FIG. 49 shows tumor volume of unconditioned mice inoculated with NCI-N87 cells and administered no treatment, cord blood NK cells, or AB-201 cells.

FIG. 50 shows body weight measurements of mice depicted in FIG. 48 and FIG. 49.

FIG. 51 shows that AB-201 infiltrates tumors, as depicted by detection of CD56 by immunofluorescence.

FIG. 52 shows an experimental design: NSG mice received 1×10⁶ SK-OV-3-Luc tumor cells intraperitoneally (IP) on day 0 and were randomized 4 days later. A single injection of CB-NK (5×10⁶ dose only) or AB-201 (1×10⁶ or 5×10⁶) was administered (IP) on day 5 or on day 5 and 12.

FIG. 53 shows efficacy of AB-201 in a SK-OV-3-Luc xenograft tumor model.

FIG. 54 shows body weight change in SK-OV-3-Luc Tumor-Bearing Mice. Body weight change was calculated based on the BW of the mouse on the day the NK cells were injected.

FIG. 55 shows presence of AB-201 in peripheral lymphoid tissues. AB-201 and CB-NK were measured in peripheral blood and spleen by flow cytometry at indicated timepoints post-tumor inoculation (gated on human CD45+CD56+).

FIG. 56 shows cell surface marker expression on CBNK or AB-201 after thawing.

FIG. 57 shows specific cytotoxicity of AB-201 and control CB-NK cells against K562. SK-OV-3. HCC1954, and NCI-N87 at different E: T ratios.

FIG. 58 shows kinetic analysis of cellular cytotoxicity of AB-201 and CBNK against SK-OV-3, HCC1954, and NCI-N87. Long-term cellular cytotoxicity of AB-201 and control CB-NK cells against HER2+ target tumor cell lines representing different solid tumor malignancies was measured using the Incucyte LiveCell analysis system for 120 hours.

FIG. 59 shows degranulation and cytokine secretion of AB-201 and CBNK following stimulation by K562, SK-OV-3, HCC1954, and NCI-N87. AB-201 or control CB-NK cells were stimulated for 24 hours with multiple target tumor cells at a 1:1 Effector: Target (E: T) ratio. Following stimulation, cells were collected and characterized for degranulation (CD107a) and cytokine secretion (IFNγ, TNFα) by flow cytometry. Data are expressed as percent positive within the CD56+ gated population. (n=3) (*p<0.05, ** p<0.01, compared with CBNK, two-tailed t-test).

FIG. 60 Secretion of IFN-γ by AB-201 following stimulation with K562, SK-OV-3,

HCC1954, and NCI-N87. AB-201 or control CB-NK cells were stimulated for 24 hours with target tumor cells at a 3:1 Effector: Target (E: T) ratio. Following collection of cell-free supernatants, soluble cytokine levels were measured by ELISA for IFNγ. (*p<0.05, ** p<0.01, compared with CBNK, two-tailed t-test).

FIG. 61 shows secretion of IL-15 by AB-201 following stimulation with K562, SK-OV-3, HCC1954, and NCI-N87. AB-201 or control CB-NK cells were stimulated for 24 hours with target tumor cells at a 3:1 Effector: Target (E: T) ratio. Following collection of cell-free supernatants, soluble cytokine levels were measured by ELISA for IL-15. (*p<0.05, compared with CBNK, two-tailed t-test).

### DETAILED DESCRIPTION

Provided herein are, amongst other things, Natural Killer (NK) cells, e.g., CAR-NK cells, methods for producing the NK cells, pharmaceutical compositions comprising the NK cells, and methods of treating patients suffering, e.g., from cancer, with the NK cells. EXPANSION AND STIMULATION OF NATURAL KILLER CELLS

In some embodiments, natural killer cells are expanded and stimulated, e.g., by culturing and stimulation with feeder cells.

NK cells can be expanded and stimulated as described, for example, in US 2020/0108096 or WO 2020/101361. both of which are incorporated herein by reference in their entirety. Briefly, the source cells can be cultured on modified HuT-78 (ATCCR TIB-16] ™) cells that have been engineered to express 4-1BBL, membrane bound IL-21, and a mutant TNFα as described in US 2020/0108096.

Suitable NK cells can also be expanded and stimulated as described herein.

In some embodiments, NK cells are expanded and stimulated by a method comprising: (a) providing NK cells, e.g., a composition comprising NK cells, e.g., CD3 (−) cells; and (b) culturing in a medium comprising feeder cells and/or stimulation factors, thereby producing a population of expanded and stimulated NK cells.

### Natural Killer Cell Sources

In some embodiments, the NK cell source is selected from the group consisting of peripheral blood, peripheral blood lymphocytes (PBLs), peripheral blood mononuclear cells (PBMCs), bone marrow, umbilical cord blood (cord blood), isolated NK cells, NK cells derived from induced pluripotent stem cells, NK cells derived from embryonic stem cells, and combinations thereof.

In some embodiments, the NK cell source is a single unit of cord blood.

In some embodiments, the natural killer cell source, e.g., single unit of cord blood, comprises from or from about 1×10⁷ to or to about 1×10⁹ total nucleated cells. In some embodiments, the natural killer cell source, e.g., single unit of cord blood, comprises from or from about 1×10⁸ to or to about 1.5×10⁸ total nucleated cells. In some embodiments, the natural killer cell source, e.g., single unit of cord blood, comprises 1×10⁸ total nucleated cells. In some embodiments, the natural killer cell source, e.g., single unit of cord blood, comprises about 1×10⁸ total nucleated cells. In some embodiments, the natural killer cell source, e.g., single unit of cord blood, comprises 1×10⁹ total nucleated cells. In some embodiments, the natural killer cell source, e.g., single unit of cord blood, comprises about 1×10⁹ total nucleated cells.

In some embodiments, the NK cell source, e.g., the cord blood unit, comprises from about 20% to about 80% CD16+ cells. In some embodiments, the NK cell source, e.g., the cord blood unit, comprises from or from about 20% to or to about 80%, from about 20% to or to about 70%, from about 20% to or to about 60%, from about 20% to or to about 50%, from about 20% to or to about 40%, from about 20% to or to about 30%, from about 30% to or to about 80%, from about 30% to or to about 70%, from about 30% to or to about 60%, from about 30% to or to about 50%, from about 30% to or to about 40%, from about 40% to or to about 80%, from about 40% to or to about 70%, from about 40% to or to about 60%, from about 40% to or to about 50%, from about 50% to or to about 80%, from about 50% to or to about 70%, from about 50% to or to about 60%, from about 60% to or to about 80%, from about 60% to or to about 70%, or from about 70% to or to about 80% CD16+ cells. In some embodiments, the NK cell source, e.g., the cord blood unit, comprises less than or equal to 80% CD16+ cells. Alternately, some NK cell sources may comprise CD16+ cells at a concentration of greater than 80%.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% MLG2A+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKG2C+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKG2D+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp46+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp30+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% DNAM-1+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp44+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD25+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD62L+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD69+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CXCR3+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD57+ cells.

In some embodiments, NK cells in the NK cell source comprise a KIR B allele of the KIR receptor family. See, e.g., . Hsu et al., “The Killer Cell Immunoglobulin-Like Receptor (KIR) Genomic Region: Gene-Order, Haplotypes and Allelic Polymorphism,” Immunological Review 190:40-52 (2002); and Pyo et al., “Different Patterns of Evolution in the Centromeric and Telomeric Regions of Group A and B Haplotypes of the Human Killer Cell Ig-like Receptor Locus,” PLOS One 5: e15115 (2010).

In some embodiments, NK cells in the NK cell source comprise the 158 V/V variant of CD16 (i.e. homozygous CD16 158V polymorphism). See, e.g., . Koene et al., “FcγRIIIa-158V/F Polymorphism Influences the Binding of IgG by Natural Killer Cell FcgammaRIIIa, Independently of the FcgammaRIIIa-48L/R/H Phenotype,” Blood 90:1109-14 (1997).

In some embodiments, NK cells in the cell source comprises both the KIR B allele of the KIR receptor family and the 158 V/V variant of CD16.

In some embodiments, the NK cells in the cell source are not genetically engineered.

In some embodiments, the NK cells in the cell source do not comprise a CD16 transgene.

In some embodiments, the NK cells in the cell source do not express an exogenous CD16 protein.

In some embodiments, the NK cell source is CD3 (+) depleted. In some embodiments, the method comprises depleting the NK cell source of CD3 (+) cells. In some embodiments, depleting the NK cell source of CD3 (+) cells comprises contacting the NK cell source with a CD3 binding antibody or antigen binding fragment thereof. In some embodiments, the CD3 binding antibody or antigen binding fragment thereof is selected from the group consisting of OKT3, UCHT1, and HIT3a, and fragments thereof. In some embodiments, the CD3 binding antibody or antigen binding fragment thereof is OKT3 or an antigen binding fragment thereof. In some embodiments, the antibody or antigen binding fragment thereof is attached to a bead, e.g., a magnetic bead. In some embodiments, the depleting the composition of CD3 (+) cells comprises contacting the composition with a CD3 targeting antibody or antigen binding fragment thereof attached to a bead and removing the bead-bound CD3 (+) cells from the composition. The composition can be depleted of CD3 cells by immunomagnetic selection, for example, using a CliniMACS T cell depletion set ((LS Depletion set (162-01) Miltenyi Biotec).

In some embodiments, the NK cell source CD56+ enriched, e.g., by gating on CD56 expression.

In some embodiments, the NK cell source is both CD56+ enriched and CD3 (+) depleted, e.g., by selecting for cells with CD56+CD3-expression.

In some embodiments, the NK cell source comprises both the KIR B allele of the KIR receptor family and the 158 V/V variant of CD16 and is + enriched and CD3 (+) depleted, e.g., by selecting for cells with CD56+CD3-expression.

### Feeder Cells

Disclosed herein are feeder cells for the expansion of NK cells. These feeder cells advantageously allow NK cells to expand to numbers suitable for the preparation of a pharmaceutical composition as discussed herein. In some cases, the feeder cells allow the expansion of NK cells without the loss of CD16 expression, which often accompanies cell expansion on other types of feeder cells or using other methods. In some cases, the feeder cells make the expanded NK cells more permissive to freezing such that a higher proportion of NK cells remain viable after a freeze/thaw cycle or such that the cells remain viable for longer periods of time while frozen. In some cases, the feeder cells allow the NK cells to retain high levels of cytotoxicity, including ADCC, extend survival, increase persistence, and enhance or retain high levels of CD16. In some cases, the feeder cells allow the NK cells to expand without causing significant levels of exhaustion or senescence.

Feeder cells can be used to stimulate the NK cells and help them to expand more quickly, e.g., by providing substrate, growth factors, and/or cytokines.

NK cells can be stimulated using various types of feeder cells, including, but not limited to peripheral blood mononuclear cells (PBMC). Epstein-Barr virus-transformed B-lymphoblastoid cells (e.g., EBV-LCL), myelogenous leukemia cells (e.g., K562), and CD4 (+) T cells (e.g., HuT), and derivatives thereof.

In some embodiments, the feeder cells are inactivated, e.g., by y-irradiation or mitomycin-c treatment.

Suitable feeder cells for use in the methods described herein are described, for example, in US 2020/0108096, which is hereby incorporated by reference in its entirety.

In some embodiments, the feeder cell(s) are inactivated CD4 (+) T cell(s). In some embodiments, the inactivated CD4 (+) T cell(s) are HuT-78 cells (ATCCR TIB-161™) or variants or derivatives thereof. In some embodiments, the HuT-78 derivative is H9 (ATCCR) HTB-176™).

In some embodiments, the inactivated CD4 (+) T cell(s) express OX40L. In some embodiments, the inactivated CD4 (+) T cell(s) are HuT-78 cells or variants or derivatives thereof that express OX40L (SEQ ID NO: 4) or a variant thereof.

In some embodiments, the feeder cells are HuT-78 cells engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof.

In some embodiments, the inactivated CD4 (+) T cell(s) are HuT-78 (ATCCR TIB-161™) cells or variants or derivatives thereof that express an ortholog of OX40L, or variant thereof. In some embodiments, the feeder cells are HuT-78 cells engineered to express at least one gene selected from the group consisting of an 4-1BBL ortholog or variant thereof, a membrane bound IL-21 ortholog or variant thereof, and mutant TNFalpha ortholog, or variant thereof.

In some embodiments, the feeder cells are HuT-78 cell(s) that express OX40L (SEQ ID NO: 4) and are engineered to express 4-1BBL (SEQ ID NO: 1), membrane bound IL-21 (SEQ ID NO: 2), and mutant TNFalpha (SEQ ID NO: 3) (“eHut-78 cells”) or variants or derivatives thereof.

In some embodiments, the feeder cells are expanded, e.g., from a frozen stock, before culturing with NK cells, e.g., as described in Example 2.

### Stimulating Factors

NK cells can also be stimulated using one or more stimulation factors other than feeder cells, e.g., signaling factors, in addition to or in place of feeder cells.

In some embodiments, the stimulating factor, e.g., signaling factor, is a component of the culture medium, as described herein. In some embodiments, the stimulating factor, e.g., signaling factor, is a supplement to the culture medium, as described herein.

In some embodiments, the stimulation factor(s) are cytokine(s). In some embodiments, the cytokine(s) are selected from the group consisting of IL-2, IL-12, IL-15, IL-18, IL-21, IL-23, IL-27, IFN-α, IFNβ, and combinations thereof.

In some embodiments, the cytokine is IL-2.

In some embodiments, the cytokines are a combination of IL-2 and IL-15.

In some embodiments, the cytokines are a combination of IL-2, IL-15, and IL-18.

In some embodiments, the cytokines are a combination of IL-2, IL-18, and IL-21.

### Culturing

The NK cells can be expanded and stimulated by co-culturing an NK cell source and feeder cells and/or other stimulation factors. Suitable NK cell sources, feeder cells, and stimulation factors are described herein.

In some cases, the resulting population of expanded natural killer cells is enriched and/or sorted after expansion. In some cases, the resulting population of expanded natural killer cells is not enriched and/or sorted after expansion

Also described herein are compositions comprising the various culture compositions described herein, e.g., comprising NK cells. For example, a composition comprising a population of expanded cord blood-derived natural killer cells comprising a KIR-B haplotype and homozygous for a CD16 158V polymorphism and a plurality of engineered HuT78 cells.

Also described herein are vessels, e.g., vials, cryobags, and the like, comprising the resulting populations of expanded natural killer cells. In some cases, a plurality of vessels comprising portions of the resulting populations of expanded natural killer cells, e.g., at least 10, e.g., 20. 30. 40, 50, 60, 70, 80, 90, 100, 125, 150, 175, 200, 250, 300, 400, 500, 600, 700, 800. 900, 1000, 1100, or 1200 vessels.

Also described herein are bioreactors comprising the various culture compositions described herein, e.g., comprising NK cells. For example, a culture comprising natural killer cells from a natural killer cell source, e.g., as described herein, and feeder cells, e.g., as described herein. Also described herein are bioreactors comprising the resulting populations of expanded natural killer cells.

### Culture Medium

Disclosed herein are culture media for the expansion of NK cells. These culture media advantageously allow NK cells to expand to numbers suitable for the preparation of a pharmaceutical composition as discussed herein. In some cases, the culture media allows NK cells to expand without the loss of CD16 expression that often accompanies cell expansion on other helper cells or in other media.

In some embodiments, the culture medium is a basal culture medium, optionally supplemented with additional components, e.g., as described herein.

In some embodiments, the culture medium, e.g., the basal culture medium, is a serum-free culture medium. In some embodiments, the culture medium, e.g., the basal culture medium, is a serum-free culture medium supplemented with human plasma and/or serum.

Suitable basal culture media include, but are not limited to, DMEM, RPMI 1640, MEM, DMEM/F12, SCGM (CellGenix (R), 20802-0500 or 20806-0500), LGM-3™ (Lonza, CC-3211), TexMACS™ (Miltenyi Biotec, 130-097-196), AlyS™ 505NK-AC (Cell Science and Technology Institute, Inc., 01600P02), AlyS™ 505NK-EX (Cell Science and Technology Institute, Inc., 01400P10), CTS™ AIM-V™ SFM (ThermoFisher Scientific, A3830801), CTS™ OpTmizer™ (ThermoFisher Scientific, A1048501, ABS-001, StemXxVivoand combinations thereof.

The culture medium may comprise additional components, or be supplemented with additional components, such as growth factors, signaling factors, nutrients, antigen binders, and the like. Supplementation of the culture medium may occur by adding each of the additional component or components to the culture vessel either before, concurrently with, or after the medium is added to the culture vessel. The additional component or components may be added together or separately. When added separately, the additional components need not be added at the same time.

In some embodiments, the culture medium comprises plasma, e.g., human plasma. In some embodiments, the culture medium is supplemented with plasma, e.g., human plasma. In some embodiments, the plasma, e.g., human plasma, comprises an anticoagulant, e.g., trisodium citrate.

In some embodiments, the medium comprises and/or is supplemented with from or from about 0.5% to or to about 10% v/v plasma, e.g., human plasma. In some embodiments, the medium is supplemented with from or from about 0.5% to or to about 9%, from or from about 0.5% to or to about 8%, from or from about 0.5% to or to about 7%, from or from about 0.5% to or to about 6%, from or from about 0.5% to or to about 5%, from or from about 0.5% to or to about 4%, from or from about 0.5% to or to about 3%, from or from about 0.5% to or to about 2%, from or from about 0.5% to or to about 1%, from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 2% to or to about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 9%, from or from about 4% to or to about 8%, from or from about 5% to or to about 7%, from or from about 5% to or to about 6%, from or from about 6% to or to about 10%, from or from about 6% to or to about 9%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v plasma, e.g., human plasma. In some embodiments, the culture medium comprises and/or is supplemented with from 0.8% to 1.2% v/v human plasma. In some embodiments, the culture medium comprises and/or is supplemented with 1.0% v/v human plasma. In some embodiments, the culture medium comprises and/or is supplemented with about 1.0% v/v human plasma.

In some embodiments, the culture medium comprises serum, e.g., human serum. In some embodiments, the culture medium is supplemented with serum, e.g., human serum. In some embodiments, the serum is inactivated, e.g., heat inactivated. In some embodiments, the serum is filtered, e.g., sterile-filtered.

In some embodiments, the culture medium comprises glutamine. In some embodiments, the culture medium is supplemented with glutamine. In some embodiments, the culture medium comprises and/or is supplemented with from or from about 2.0 to or to about 6.0 mM glutamine. In some embodiments, the culture medium comprises and/or is supplemented with from or from about 2.0 to or to about 5.5, from or from about 2.0 to or to about 5.0, from or from about 2.0 to or to about 4.5, from or from about 2.0 to or to about 4.0, from or from about 2.0 to or to about 3.5, from or from about 2.0 to or to about 3.0, from or from about 2.0 to or to about 2.5, from or from about 2.5 to or to about 6.0, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3.0 to or to about 6.0, from or from about 3.0 to or to about 5.5, from or from about 3.0 to or to about 5.0, from or from about 3.0 to or to about 4.5, from or from about 3.0 to or to about 4.0, from or from about 3.0 to or to about 3.5, from or from about 3.5 to or to about 6.0, from or from about 3.5 to or to about 5.5, from or from about 3.5 to or to about 5.0, from or from about 3.5 to or to about 4.5, from or from about 3.5 to or to about 4.0, from or from about 4.0 to or to about 6.0, from or from about 4.0 to or to about 5.5, from or from about 4.0 to or to about 5.0, from or from about 4.0 to or to about 4.5, from or from about 4.5 to or to about 6.0, from or from about 4.5 to or to about 5.5, from or from about 4.5 to or to about 5.0, from or from about 5.0 to or to about 6.0, from or from about 5.0 to or to about 5.5, or from or from about 5.5 to or to about 6.0 mM glutamine. In some embodiments, the culture medium comprises and/or is supplemented with from 3.2 mM glutamine to 4.8 mM glutamine. In some embodiments, the culture medium comprises and/or is supplemented with 4.0 mM glutamine. In some embodiments, the culture medium comprises and/or is supplemented with about 4.0 mM glutamine.

In some embodiments, the culture medium comprises one or more cyotkines. In some embodiments, the culture medium is supplemented with one or more cyotkines.

In some embodiments, the cytokine is selected from IL-2, IL-12, IL-15, IL-18, and combinations thereof.

In some embodiments, the culture medium comprises and/or is supplemented with IL-2. In some embodiments, the culture medium comprises and/or is supplemented with from or from about 150 to or to about 2,500 IU/mL IL-2. In some embodiments, the culture medium comprises and/or is supplemented with from or from about 200 to or to about 2,250, from or from about 200 to or to about 2,000, from or from about 200 to or to about 1,750, from or from about 200 to or to about 1,500, from or from about 200 to or to about 1,250, from or from 200 to or to about 1,000, from or from about 200 to or to about 750, from or from about 200 to or to about 500, from or from about 200 to or to about 250, from or from about 250 to or to about 2,500, from or from about 250 to or to about 2,250, from or from about 250 to or to about 2,000, from or from about 250 to or to about 1,750, from or from about 250 to or to about 1,500, from or from about 250 to or to about 1,250, from or from about 250 to or to about 1,000, from or from about 250 to or to about 750, from or from about 250 to or to about 500, from or from about 500 to or to about 2,500, from or from about 500 to or to about 2,250, from or from about 500 to or to about 2,000, from or from about 500 to or to about 1,750, from or from about 500 to or to about 1,500, from or from about 500 to or to about 1,250, from or from about 500 to or to about 1,000, from or from about 500 to or to about 750, from or from about 750 to or to about 2,250, from or from about 750 to or to about 2,000, from or from about 750 to or to about 1,750, from or from about 750 to or to about 1,500, from or from about 750 to or to about 1,250, from or from about 750 to or to about 1,000, from or from about 1,000 to or to about 2,500, from or from about 1,000 to or to about 2,250, from or from about 1,000 to or to about 2,000, from or from about 1,000 to or to about 1,750, from or from about 1,000 to or to about 1,500, from or from about 1,000 to or to about 1,250, from or from about 1,250 to or to about 2,500, from or from about 1,250 to or to about 2,250, from or from about 1,250 to or to about 2,000, from or from about 1.250 to or to about 1.750, from or from about 1.250 to or to about 1.500, from or from about 1,500 to or to about 2,500, from or from about 1,500 to or to about 2,250, from or from about 1,500 to or to about 2,000, from or from about 1,500 to or to about 1,750, from or from about 1,750 to or to about 2,500, from or from about 1,750 to or to about 2,250, from or from about 1,750 to or to about 2,000, from or from about 2,000 to or to about 2,500, from or from about 2,000 to or to about 2,250, or from or from about 2,250 to or to about 2,500 IU/mL IL-2.

In some embodiments, the culture medium comprises and/or is supplemented with from 64 μg/L to 96 μg/L IL-2. In some embodiments, the culture medium comprises and/or is supplemented with 80 μg/L IL-2 (approximately 1,333 IU/mL). In some embodiments, the culture medium comprises and/or is supplemented with about 80 μg/L.

In some embodiments, the culture medium comprises and/or is supplemented with a combination of IL-2 and IL-15.

In some embodiments, the culture medium comprises and/or is supplemented with a combination of IL-2, IL-15, and IL-18.

In some embodiments, the culture medium comprises and/or is supplemented with a combination of IL-2, IL-18, and IL-21.

In some embodiments, the culture medium comprises and/or is supplemented with glucose. In some embodiments, the culture medium comprises and/or is supplemented with from or from about 0.5 to or to about 3.5 g/L glucose. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L glucose. In some embodiments, the culture medium comprises and/or is supplemented with from 1.6 to 2.4 g/L glucose. In some embodiments, the culture medium comprises and/or is supplemented with 2.0 g/L glucose. In some embodiments, the culture medium comprises about 2.0 g/L glucose.

In some embodiments, the culture medium comprises and/or is supplemented with sodium pyruvate. In some embodiments, the culture medium comprises and/or is supplemented with from or from about 0.1 to or to about 2.0 mM sodium pyruvate. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 mM sodium pyruvate. In some embodiments, the culture medium comprises from 0.8 to 1.2 mM sodium pyruvate. In some embodiments, the culture medium comprises 1.0 mM sodium pyruvate. In some embodiments, the culture medium comprises about 1.0 mM sodium pyuruvate.

In some embodiments, the culture medium comprises and/or is supplemented with sodium hydrogen carbonate. In some embodiments, the culture medium comprises and/or is supplemented with from or from about 0.5 to or to about 3.5 g/L sodium hydrogen carbonate. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L sodium hydrogen carbonate. In some embodiments, the culture medium comprises and/or is supplemented with from 1.6 to 2.4 g/L sodium hydrogen carbonate. In some embodiments, the culture medium comprises and/or is supplemented with 2.0 g/L sodium hydrogen carbonate. In some embodiments, the culture medium comprises about 2.0 g/L sodium hydrogen carbonate.

In some embodiments, the culture medium comprises and/or is supplemented with albumin, e.g., human albumin, e.g., a human albumin solution described herein. In some embodiments, the culture medium comprises and/or is supplemented with from or from about 0.5% to or to about 3.5% v/v of a 20% albumin solution, e.g., a 20% human albumin solution. In some embodiments, the culture medium comprises and/or is supplemented with from or from about 0.5% to or to about 3.0%, from or from about 0.5% to or to about 2.5%, from or from about 0.5% to or to about 2.0%, from or from about 0.5% to or to about 1.5%, from or from about 0.5% to or to about 1.0%, from or from about 1.0% to or to about 3.0%, from or from about 1.0% to or to about 2.5%, from or from about 1.0% to or to about 2.0%, from or from about 1.0% to or to about 1.5%, from or from about 1.5% to or to about 3.0%, from or from about 1.5% to or to about 2.5%, from or from about 1.5% to or to about 2.0%, from or from about 2.0% to or to about 3.0%, from or from about 2.0% to or to about 2.5%, or from or from about 2.5% to or to about 3.0% v/v of a 20% albumin solution, e.g., a 20% human albumin solution. In some embodiments, the culture medium comprises and/or is supplemented with from 1.6% to 2.4% v/v of a 20% albumin solution, e.g., a 20% human albumin solution. In some embodiments, the culture medium comprises and/or is supplemented with 2.0% v/v of a 20% albumin solution, e.g., a 20% human albumin solution. In some embodiments, the culture medium comprises about 2.0% v/v of a 20% albumin solution, e.g., a 20% human albumin solution.

In some embodiments, the culture medium comprises and/or is supplemented with from or from about 2 to or to about 6 g/L albumin, e.g., human albumin. In some embodiments, the culture medium comprises and/or is supplemented with from or from about 2 to or to about 5.5, from or from about 2 to or to about 5.0, from or from about 2 to or to about 4.5, from or from about 2 to or to about 4, from or from about 2 to or to about 3.5, from or from about 2 to or to about 3, from or from about 2 to or to about 2.5, from or from about 2.5 to or to about 6, from or from about 2.5 to or to about 5.5, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3 to or to about 6, from or from about 3 to or to about 5.5, from or from about 3 to or to about 5, from or from about 3 to or to about 4.5, from or from about 3 to or to about 4, from or from about 3 to or to about 3.5, from or from about 3.5 to or to about 6, from or from about 3.5 to or to about 5.5, from or from about 3.5 to or to about 5, from or from about 3.5 to or to about 4.5, from or from about 3.5 to or to about 4, from or from about 4 to or to about 6, from or from about 4 to or to about 5.5, from or from about 4 to or to about 5, from or from about 4 to or to about 4.5, from or from about 4.5 to or to about 6, from or from about 4.5 to or to about 5.5, from or from about 4.5 to or to about 5, from or from about 5 to or to about 6, from or from about 5 to or to about 5.5, or from or from about 5.5 to or to about 6 g/L albumin, e.g., human albumin. In some embodiments, the culture medium comprises and/or is supplemented with from 3.2 to 4.8 g/L albumin, e.g., human albumin. In some embodiments, the culture medium comprises 4 g/L albumin, e.g., human albumin. In some embodiments, the culture medium comprises about 4 g/L albumin, e.g., human albumin

In some embodiments, the culture medium is supplemented with Poloxamer 188. In some embodiments, the culture medium comprises and/or is supplemented with from or from about 0.1 to or to about 2.0 g/L Poloxamer 188. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0. 1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0).2 to or to about 0.6, from or from about 0).2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 g/L Poloxamer 188. In some embodiments, the culture medium comprises from 0.8 to 1.2 g/L Poloxamer 188. In some embodiments, the culture medium comprises 1.0 g/L Poloxamer 188. In some embodiments, the culture medium comprises about 1.0 g/L Poloxamer 188.

In some embodiments, the culture medium comprises and/or is supplemented with one or more antibiotics.

A first exemplary culture medium is set forth in Table 1.

TABLE 1
Exemplary Culture Medium #1

| | Exemplary | | | |
|---|---|---|---|---|
| | Concentration | | Exemplary | |
| Component | Range | | Concentration | |
| CellgroSCGM liquid medium | undiluted | | undiluted | |
| Human Plasma | 0.8-1.2% | (v/v) | 1.0% | v/v |
| Glutamine | 3.2-4.8 | mM | 4.0 | mM |
| IL-2 | 64-96 | μg/L | 80 | μg/L |

A second exemplary culture medium is set forth in Table 2.

TABLE 2
Exemplary Culture Medium #2

| | Exemplary | | | |
|---|---|---|---|---|
| | Concentration | | Exemplary | |
| Component | Range | | Concentration | |
| RPMI1640 | 7.6-13.2 | g/L | 10.4 | g/L |
| Human Plasma | 0.8-1.2% | (v/v) | 1.0% | v/v |
| Glucose | 1.6-2.4 | g/L | 2.0 | g/L |
| Glutamine | 3.2-4.8 | mM | 4.0 | mM |
| Sodium Pyruvate | 0.8-1.2 | mM | 1.0 | mM |
| Sodium Hydrogen Carbonate | 1.6-2.4 | g/L | 2.0 | g/L |
| IL-2 | 64-96 | μg/L | 80 | μg/L |
| Albumin 20% solution | 1.6-2.5% v/v | | 2.0% v/v | |
| | (3.2 to 4.8 g/L) | | (4.0 g/L) | |
| Poloxamer 188 | 0.8-1.2 | g/L | 1.0 | g/L |

### CD3 Binding Antibodies

In some embodiments, the culture medium comprises and/or is supplemented with a CD3 binding antibody or antigen binding fragment thereof. In some embodiments, the CD3 binding antibody or antigen binding fragment thereof is selected from the group consisting of OKT3, UCHT1, and HIT3a, or variants thereof. In some embodiments, the CD3 binding antibody or antigen binding fragment thereof is OKT3 or an antigen binding fragment thereof.

In some embodiments, the CD3 binding antibody or antigen binding fragment thereof and feeder cells are added to the culture vessel before addition of NK cells and/or culture medium.

In some embodiments, the culture medium comprises and/or is supplemented with from or from about 5 ng/mL to or to about 15 ng/mL OKT3. In some embodiments, the culture medium comprises and/or is supplemented with from or from about 5 to or to about 12.5, from or from about 5 to or to about 10, from or from about 5 to or to about 7.5, from or from about 7.5 to or to about 15, from or from about 7.5 to or to about 12.5, from or from about 7.5 to or to about 10, from or from about 10 to or to about 15, from or from about 10 to or to about 12.5, or from or from about 12.5 to or to about 15 ng/mL OKT3. In some embodiments, the culture medium comprises and/or is supplemented with 10 ng/mL OKT3. In some embodiments, the culture medium comprises and/or is supplemented with about 10 ng/ml OKT3.

### Culture Vessels

A number of vessels are consistent with the disclosure herein. In some embodiments, the culture vessel is selected from the group consisting of a flask, a bottle, a dish, a multiwall plate, a roller bottle, a bag, and a bioreactor.

In some embodiments, the culture vessel is treated to render it hydrophilic. In some embodiments, the culture vessel is treated to promote attachment and/or proliferation. In some embodiments, the culture vessel surface is coated with serum, collagen, laminin, gelatin, poy-L-lysine, fibronectin, extracellular matrix proteins, and combinations thereof.

In some embodiments, different types of culture vessels are used for different stages of culturing.

In some embodiments, the culture vessel has a volume of from or from about 100 mL to or to about 1,000 L. In some embodiments, the culture vessel has a volume of or about 125 mL, of or about 250 mL, of or about 500 mL, of or about 1 L, of or about 5 L, of about 10 L, or of or about 20 L.

In some embodiments, the culture vessel is a bioreactor.

In some embodiments, the bioreactor is a rocking bed (wave motion) bioreactor. In some embodiments, the bioreactor is a stirred tank bioreactor. In some embodiments, the bioreactor is a rotating wall vessel. In some embodiments, the bioreactor is a perfusion bioreactor. In some embodiments, the bioreactor is an isolation/expansion automated system. In some embodiments, the bioreactor is an automated or semi-automated bioreactor. In some embodiments, the bioreactor is a disposable bag bioreactor.

In some embodiments, the bioreactor has a volume of from about 100 mL to about 1,000 L. In some embodiments, the bioreactor has a volume of from about 10 L to about 1,000 L. In some embodiments, the bioreactor has a volume of from about 100 L to about 900 L. In some embodiments, the bioreactor has a volume of from about 10 L to about 800 L. In some embodiments, the bioreactor has a volume of from about 10 L to about 700 L, about 10 L to about 600 L, about 10 L to about 500 L, about 10 L to about 400 L, about 10 L to about 300 L, about 10 L to about 200 L, about 10 L to about 100 L, about 10 L to about 90 L, about 10 L to about 80 L, about 10 L to about 70 L, about 10 L to about 60 L, about 10 L to about 50 L, about 10 L to about 40 L, about 10 L to about 30 L, about 10 L to about 20 L, about 20 L to about 1,000 L, about 20 L to about 900 L, about 20 L to about 800 L, about 20 L to about 700 L, about 20 L to about 600 L, about 20 L to about 500 L, about 20 L to about 400 L, about 20 L to about 300 L, about 20 L to about 200 L, about 20 L to about 100 L, about 20 L to about 90 L, about 20 L to about 80 L, about 20 L to about 70 L, about 20 L to about 60 L, about 20 L to about 50 L, about 20 L to about 40 L, about 20 L to about 30 L, about 30 L to about 1,000 L, about 30 L to about 900 L, about 30 L to about 800 L, about 30 L to about 700 L, about 30 L to about 600 L. about 30 L to about 500 L, about 30 L to about 400 L, about 30 L to about 300 L, about 30 L to about 200 L, about 30 L to about 100 L, about 30 L to about 90 L, about 30 L to about 80 L, about 30 L to about 70 L, about 30 L to about 60 L, about 30 L to about 50 L, about 30 L to about 40 L, about 40 L to about 1,000 L, about 40 L to about 900 L, about 40 L to about 800 L, about 40 L to about 700 L, about 40 L to about 600 L, about 40 L to about 500 L, about 40 L to about 400 L, about 40 L to about 300 L, about 40 L to about 200 L, about 40 L to about 100 L, about 40 L to about 90 L, about 40 L to about 80 L, about 40 L to about 70 L, about 40 L to about 60 L, about 40 L to about 50 L, about 50 L to about 1.000 L, about 50 L to about 900 L, about 50 L to about 800 L, about 50 L to about 700 L, about 50 L to about 600 L, about 50 L to about 500 L, about 50 L to about 400 L, about 50 L to about 300 L, about 50 L to about 200 L. about 50 L to about 100 L, about 50 L to about 90 L, about 50 L to about 80 L, about 50 L to about 70 L, about 50 L to about 60 L, about 60 L to about 1.000 L, about 60 L to about 900 L, about 60 L to about 800 L, about 60 L to about 700 L, about 60 L to about 600 L, about 60 L to about 500 L, about 60 L to about 400 L, about 60 L to about 300 L, about 60 L to about 200 L, about 60 L to about 100L, about 60 L to about 90 L, about 60 L to about 80 L, about 60 L to about 70 L, about 70 L to about 1,000 L, about 70 L to about 900 L, about 70 L to about 800 L, about 70 L to about 700 L, about 70 L to about 600 L, about 70 L to about 500 L, about 70 L to about 400 L, about 70 L to about 300 L, about 70 L to about 200 L, about 70 L to about 100 L, about 70 L to about 90 L, about 70 L to about 80 L, about 80 L to about 1,000 L, about 80 L to about 900 L, about 80 L to about 800 L, about 80 L to about 700 L, about 80 L to about 600 L, about 80 L to about 500 L, about 80 L to about 400 L, about 80 L to about 300 L, about 80 L to about 200 L, about 80 L to about 100 L, about 80 L to about 90 L, about 90 L to about 1,000 L, about 90 L to about 900 L, about 90 L to about 800 L, about 90 L to about 700 L, about 90 L to about 600 L, about 90 L to about 500 L, about 90 L to about 400 L, about 90 L to about 300 L, about 90 L to about 200 L, about 90 L to about 100 L, about 100 L to about 1,000 L, about 100 L to about 900 L, about 100 L to about 800 L, about 100 L to about 700 L, about 100 L toa bout 600 L, about 100 L to about 500 L, about 100 L to about 400 L, about 100 L to about 300 L, about 100 L to about 200 L, about 200 L to about 1,000 L, about 200 L to about 900 L, about 200 L to about 800 L, about 200 L to about 700 L, about 200 L to about 600 L, about 200 L to about 500 L, about 200 L to about 400 L, about 200 L to about 300 L, about 300 L to about 1,000 L, about 300 L to about 900 L, about 300 L to about 800 L, about 300 L to about 700 L, about 300 L to about 600 L, about 300 L to about 500 L, about 300 L to about 400 L, about 400 L to about 1,000 L, about 400 L to about 900 L, about 400 L to about 800 L, about 400 L to about 700 L, about 400 L to about 600 L, about 400 L to about 500 L, about 500 L to about 1,000 L, about 500 L to about 900 L, about 500 L to about 800 L, about 500 L to about 700 L, about 500 L to about 600 L, about 600 L to about 1,000 L, about 600 L to about 900 L, about 600 L to about 800 L, about 600 L to about 700 L, about 700 L to about 1,000 L, about 700 L to about 900 L, about 700 L to about 800 L, about 800 L to about 1,000 L, about 800 L to about 900 L, or about 900 L to about 1,000 L. In some embodiments, the bioreactor has a volume of about 50 L.

In some embodiments, the bioreactor has a volume of from 100 mL to 1,000 L. In some embodiments, the bioreactor has a volume of from 10 L to 1.000 L. In some embodiments, the bioreactor has a volume of from 100 L to 900 L. In some embodiments, the bioreactor has a volume of from 10 L to 800 L. In some embodiments, the bioreactor has a volume of from 10 L to 700 L, 10 L to 600 L, 10 L to 500 L, 10 L to 400 L, 10 L to 300 L, 10 L to 200 L, 10 L to 100 L, 10 L to 90 L, 10 L to 80 L, 10 L to 70 L, 10 L to 60 L, 10 L to 50 L, 10 L to 40 L, 10 L to 30 L, 10 L to 20 L, 20 L to 1,000 L, 20 L to 900 L, 20 L to 800 L, 20 L to 700 L, 20 L to 600 L, 20 L to 500 L, 20 L to 400 L, 20 L to 300 L, 20 L to 200 L, 20 L to 100 L, 20 L to 90 L, 20 L to 80

L, 20 L to 70 L, 20 L to 60 L, 20 L to 50 L, 20 L to 40 L, 20 L to 30 L, 30 L to 1,000 L, 30 L to 900 L, 30 L to 800 L. 30 L to 700 L, 30 L to 600 L, 30 L to 500 L, 30 L to 400 L. 30 L to 300 L, 30 L to 200 L, 30 L to 100 L, 30 L to 90 L, 30 L to 80 L, 30 L to 70 L, 30 L to 60 L, 30 L to 50 L, 30 L to 40 L, 40 L to 1,000 L, 40 L to 900 L, 40 L to 800 L, 40 L to 700 L, 40 L to 600 L, 40 L to 500 L, 40 L to 400 L, 40 L to 300 L, 40 L to 200 L, 40 L to 100 L, 40 L to 90 L, 40 L to 80 L, 40 L to 70 L, 40 L to 60 L, 40 L to 50 L, 50 L to 1,000 L, 50 L to 900 L, 50 L to 800 L, 50 L to 700 L, 50 L to 600 L, 50 L to 500 L, 50 L to 400 L, 50 L to 300 L, 50 L to 200 L, 50 L to 100 L, 50 L to 90 L, 50 L to 80 L, 50 L to 70 L, 50 L to 60 L, 60 L to 1,000 L, 60 L to 900 L, 60 L to 800 L, 60 L to 700 L. 60 L to 600 L, 60 L to 500 L, 60 L to 400 L, 60 L to 300 L. 60 L to 200 L, 60 L to 100L, 60 L to 90 L, 60 L to 80 L, 60 L to 70 L, 70 L to 1,000 L, 70 L to 900 L, 70 L to 800 L, 70 L to 700 L. 70 L to 600 L, 70 L to 500 L, 70 L to 400 L, 70 L to 300 L. 70 L to 200 L, 70 L to 100 L, 70 L to 90 L, 70 L to 80 L, 80 L to 1,000 L, 80 L to 900 L, 80 L to 800 L, 80 L to 700 L, 80 L to 600 L. 80 L to 500 L, 80 L to 400 L, 80 L to 300 L, 80 L to 200 L, 80 L to 100 L, 80 L to 90 L, 90 L to 1,000 L, 90 L to 900 L, 90 L to 800 L, 90 L to 700 L, 90 L to 600 L, 90 L to 500 L, 90 L to 400 L, 90 L to 300 L, 90 L to 200 L, 90 L to 100 L, 100 L to 1,000 L, 100 L to 900 L. 100 L to 800 L. 100 L to 700 L, 100 L to 600 L. 100 L to 500 L. 100 L to 400 L. 100 L to 300 L, 100 L to 200 L, 200 L to 1,000 L, 200 L to 900 L, 200 L to 800 L, 200 L to 700 L, 200 L to 600 L, 200 L to 500 L, 200 L to 400 L, 200 L to 300 L, 300 L to 1,000 L, 300 L to 900 L, 300 L to 800 L, 300 L to 700 L, 300 L to 600 L, 300 L to 500 L, 300 L to 400 L, 400 L to 1,000 L, 400 L to 900 L, 400 L to 800 L, 400 L to 700 L, 400 L to 600 L, 400 L to 500 L, 500 L to 1,000 L, 500 L to 900 L, 500 L to 800 L, 500 L to 700 L, 500 L to 600 L, 600 L to 1,000 L, 600 L to 900 L, 600 L to 800 L, 600 L to 700 L, 700 L to 1,000 L, 700 L to 900 L, 700 L to 800 L, 800 L to 1,000 L, 800 L to 900 L, or 900 L to 1,000 L. In some embodiments, the bioreactor has a volume of 50 L.

### Cell Expansion and Stimulation

In some embodiments, the natural killer cell source, e.g., single unit of cord blood, is co-cultured with feeder cells to produce expanded and stimulated NK cells.

In some embodiments, the co-culture is carried out in a culture medium described herein, e.g., exemplary culture medium #1 (Table 1) or exemplary culture medium #2 (Table 2).

In some embodiments, the natural killer cell source, e.g., single unit of cord blood, comprises from or from about 1×10⁷ to or to about 1×10⁹ total nucleated cells prior to expansion. In some embodiments, the natural killer cell source, e.g., single unit of cord blood, comprises from or from about 1×10⁸ to or to about 1.5×10⁸ total nucleated cells prior to expansion. In some embodiments, the natural killer cell source, e.g., single unit of cord blood, comprises 1×10⁸ total nucleated cells prior to expansion. In some embodiments, the natural killer cell source, e.g., single unit of cord blood, comprises about 1×10⁸ total nucleated cells prior to expansion. In some embodiments, the natural killer cell source, e.g., single unit of cord blood, comprises 1×10⁹ total nucleated cells prior to expansion. In some embodiments, the natural killer cell source, e.g., single unit of cord blood, comprises about 1×10⁹ total nucleated cells prior to expansion.

In some embodiments, cells from the co-culture of the natural killer cell source, e.g., single unit of cord blood and feeder cells are harvested and frozen, e.g., in a cryopreservation composition described herein. In some embodiments, the frozen cells from the co-culture are an infusion-ready drug product. In some embodiments, the frozen cells from the co-culture are used as a master cell bank (MCB) from which to produce an infusion-ready drug product, e.g., through one or more additional co-culturing steps, as described herein. Thus, for example, a natural killer cell source can be expanded and stimulated as described herein to produce expanded and stimulated NK cells suitable for use in an infusion-ready drug product without generating any intermediate products. A natural killer cell source can also be expanded and stimulated as described herein to produce an intermediate product, e.g., a first master cell bank (MCB). The first MCB can be used to produce expanded and stimulated NK cells suitable for use in an infusion-ready drug product, or, alternatively, be used to produce another intermediate product, e.g., a second MCB. The second MCB can be used to produce expanded and stimulated NK cells suitable for an infusion-ready drug product, or alternatively, be used to produce another intermediate product, e.g., a third MCB, and so on.

In some embodiments, the ratio of feeder cells to cells of the natural killer cell source or MCB cells inoculated into the co-culture is from or from about 1:1 to or to about 4:1. In some embodiments, the ratio of feeder cells to cells of the natural killer cell source or MCB cells is from or from about 1:1 to or to about 3.5:1, from or from about 1:1 to or to about 3:1, from or from about 1:1 to or to about 2.5:1, from or from about 1.1 to or to about 2:1, from or from about 1:1 to or to about 1.5:1, from or from about 1.5:1 to or to about 4:1, from or from about 1.5:1 to or to about 3.5:1, from or from about 1.5:1 to or to about 3:1, from or from about 1.5:1 to or to about 2.5:1, from or from about 1.5:1 to or to about 2:1, from or from about 2:1 to or to about 4:1, from or from about 2:1 to or to about 3.5:1, from or from about 2:1 to or to about 3:1, from or from about 2:1 to or to about 2.5:1, from or from about 2.5:1 to or to about 4:1, from or from about 2.5:1 to or to about 3.5:1, from or from about 2.5:1 to or to about 3:1, from or from about 3:1 to or to about 4:1, from or from about 3:1 to or to about 3.5:1, or from or from about 3.5:1 to or to about 4:1. In some embodiments, the ratio of feeder cells to cells of the natural killer cell source or MCB inoculated into the co-culture is 2.5:1. In some embodiments, the ratio of feeder cells to cells of the natural killer cell source or MCB inoculated into the co-culture is about 2.5:1.

In some embodiments, the co-culture is carried out in a disposable culture bag, e.g., a IL disposable culture bag. In some embodiments, the co-culture is carried out in a bioreactor, e.g., a 50L bioreactor. In some embodiments, culture medium is added to the co-culture after the initial inoculation.

In some embodiments, the co-culture is carried out for 1, 2, 3, 4, 5, 6, 7, 8, 9, 10, 11, 12, 13, 14, 15, 16, 17, 18, 19, 20. 21, 22, 23, 24 or more days. In some embodiments, the co-culture is carried out for a maximum of 16 days.

In some embodiments, the co-culture is carried out at 37° C., or about 37° C.

In some embodiments, the co-culture is carried out at pH 7.9 or about pH 7.9.

In some embodiments, the co-culture is carried out at a dissolved oxygen (DO) level of 50% or more.

In some embodiments, exemplary culture medium #1 (Table 1) is used to produce a MCB and exemplary culture medium #2 (Table 2) is used to produce cells suitable for an infusion-ready drug product.

In some embodiments, the co-culture of the natural killer cell source, e.g., single unit of cord blood, with feeder cells yields from or from about 50×10⁸ to or to about 50×10¹²cells, e.g., MCB cells or infusion-ready drug product cells. In some embodiments, the expansion yields from or from about 50×10⁸ to or to about 25×10¹⁰, from or from about 10×10⁸ to or to about 1×10¹⁰, from or from about 50×10⁸ to or to about 75×10⁹ , from or from about 50×10⁸ to or to about 50×10⁹, from or from about 50×10⁸ to or to about 25×10⁹, from or from about 50×10⁸ to or to about 1×10⁹, from or from about 50×10⁸ to or to about 75×10⁸, from or from about 75×10⁸ to or to about 50×10¹⁰, from or from about 75×10⁸ to or to about 25×10¹⁰, from or from about 75×10⁸ to or to about 1×10¹⁰, from or from about 75×10⁸ to or to about 75×109, from or from about 75×10⁸ to or to about 50×10⁹, from or from about 75×10⁸ to or to about 25×10⁹, from or from about 75×10⁸ to or to about 1×10⁹, from or from about 1×10⁹ to or to about 50×10¹⁰, from or from about 1×10⁹ to or to about 25×10¹⁰, from or from about 1×109 to or to about 1×10¹⁰, from or from about 1×10⁹ to or to about 75×10⁹ , from or from about 1×10⁹ to or to about 50×10⁹, from or from about 1×10⁹ to or to about 25×10⁹, from or from about 25×10⁹ to or to about 50×10¹⁰, from or from about 25×10⁹ to or to about 25×10¹⁰, from or from about 25×10⁹ to or to about 1×10¹⁰, from or from about 25×10⁹ to or to about 75×10⁹, from or from about 25×10⁹ to or to about 50×10⁹, from or from about 50×10⁹ to or to about 50×10¹⁰, from or from about 50×10⁹ to or to about 25×10¹⁰, from or from about 50×10⁹ to or to about 1×10¹⁰, from or from about 50×10⁹ to or to about 75×10⁹, from or from about 75×10⁹ to or to about 50×10¹⁰, from or from about 75×10⁹ to or to about 25×10¹⁰, from or from about 75×10⁹ to or to about 1×10¹⁰, from or from about 1×10¹⁰ to or to about 50×10¹⁰, from or from about 1×10¹⁰ to or to about 25×10¹⁰, or from or from about 25×1010 to or to about 50×10¹⁰ cells, e.g., e.g., MCB cells or infusion-ready drug product cells.

In some embodiments, the expansion yields from or from about 60 to or to about 100 vials, each comprising from or from about 600 million to or to about 1 billion cells, e.g., MCB cells or infusion-ready drug product cells. In some embodiments, the expansion yields 80 or about 80 vials, each comprising or consisting of 800 million or about 800 million cells, e.g., MCB cells or infusion-ready drug product cells.

In some embodiments, the expansion yields from or from about a 100 to or to about a 500 fold increase in the number of cells, e.g., the number of MCB NK cells relative to the number of cells, e.g., NK cells, in the natural killer cell source. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of MCB cells relative to the number of cells, e.g., NK cells, in the natural killer cell source.

In some embodiments, the expansion yields from or from about a 100 to or to about a 70,000 fold increase in the number of cells, e.g., the number of MCB NK cells relative to the number of cells, e.g., NK cells, in the natural killer cell source. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of MCB NK cells relative to the number of cells, e.g., NK cells, in the natural killer cell source.

In some embodiments, the co-culture of the MCB cells and feeder cells yields from or from about 500 million to or to about 1.5 billion cells, e.g., NK cells suitable for use in an MCB and/or in an infusion-ready drug product. In some embodiments, the co-culture of the MCB cells and feeder cells yields from or from about 500 million to or to about 1.5 billion, from or from about 500 million to or to about 1.25 billion, from or from about 500 million to or to about 1 billion, from or from about 500 million to or to about 750 million, from or from about 750 million to or to about 1.5 billion, from or from about 500 million to or to about 1.25 billion, from or from about 750 million to or to about 1 billion, from or from about 1 billion to or to about 1.5 billion, from or from about 1 billion to or to about 1.25 billion, or from or from about 1.25 billion to or to about 1.5 billion cells, e.g., NK cells suitable for use in an MCB and/or an infusion-ready drug product.

In some embodiments, the co-culture of the MCB cells and feeder cells yields from or from about 50 to or to about 150 vials of cells, e.g., infusion-ready drug product cells, each comprising from or from about 750 million to or to about 1.25 billion cells, e.g., NK cells suitable for use in an MCB and/or an infusion-ready drug product. In some embodiments, the co-culture of the MCB cells and feeder cells yields 100 or about 100 vials, each comprising or consisting of 1 billion or about 1 billion cells, e.g., NK cells suitable for use in an MCB and/or an infusion-ready drug product.

In some embodiments, the expansion yields from or from about a 100 to or to about a 500 fold increase in the number of cells, e.g., the number of NK cells suitable for use in an MCB and/or an infusion-ready drug product relative to the number of starting MCB cells. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of NK cells suitable for use in an MCB and/or an infusion-ready drug product relative to the number of starting MCB cells.

In some embodiments, the expansion yields from or from about a 100 to or to about a 70,000 fold increase in the number of cells, e.g., the number of NK cells suitable for use in an MCB and/or an infusion-ready drug product relative to the number of starting MCB NK cells. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold. 65.000 fold, or 70.000 fold increase in the number of cells, e.g., the number of NK cells suitable for use in an MCB and/or an infusion-ready drug product relative to the number of starting MCB NK cells.

In embodiments where the cells are engineered during expansion and stimulation, as described herein, not all of the expanded and stimulated cells will necessarily be engineered successfully, e.g., transduced successfully, e.g., transduced successfully with a vector comprising a heterologous protein, e.g., a heterologous protein comprising a CAR and/or IL-15 as described herein. Thus, the methods described herein can further comprise sorting engineered cells, e.g., engineered cells described herein, away from non-engineered cells.

In some embodiments, the engineered cells, e.g., transduced cells, are sorted from the non-engineered cells, e.g., the non-transduced cells using a reagent specific to an antigen of the engineered cells, e.g., an antibody that targets an antigen of the engineered cells but not the non-engineered cells. In some embodiments, the antigen of the engineered cells is a component of a CAR, e.g., a CAR described herein.

Systems for antigen-based cell separation of cells are available commercially, e.g., the CliniMACSR sorting system (Miltenyi Biotec).

In some embodiments, the engineered cells, e.g., transduced cells, are sorted from the non-engineered cells, e.g., the non-transduced cells using flow cytometry.

In some embodiments, the sorted engineered cells are used as an MCB. In some embodiments, the sorted engineered cells are used as a component in an infusion-ready drug product.

In some embodiments, the engineered cells, e.g., transduced cells, are sorted from the non-engineered cells, e.g., the non-transduced cells using a microfluidic cell sorting method. Microfluidic cell sorting methods are described, for example, in Dalili et al., “A Review of Sorting, Separation and Isolation of Cells and Microbeads for Biomedical Applications: Microfluidic Approaches,” Analyst 144:87 (2019).

In some embodiments, from or from about 1% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein. In some embodiments, from or from about 1% to or to about 90%, from or from about 1% to or to about 80%, from or from about 1% to or to about 70%, from or from about 1% to or to about 60%, from or from about 1% to or to about 50%, from or from about 1% to or to about 40%, from or from about 1% to or to about 30%. from or from about 1% to or to about 20%, from or from about 1% to or to about 10%, from or from about 1% to or to about 5%, from or from about 5% to or to about 99%, from or from about 5% to or to about 90%, from or from about 5% to or to about 80%, from or from about 5% to or to about 70%, from or from about 5% to or to about 60%, from or from about 5% to or to about 50%, from or from about 5% to or to about 40%, from or from about 5% to or to about 30%, from or from about 5% to or to about 20%, from or from about 5% to or to about 10%, from or from about 10% to or to about 99%, from or from about 10% to or to about 90%, from or from about 10% to or to about 80%, from or from about 10% to or to about 70%, from or from about 10% to or to about 60%, from or from about 10% to or to about 50%, from or from about 10% to or to about 40%, from or from about 10% to or to about 30%, from or from about 10% to or to about 20%, from or from about 20% to or to about 99%, from or from about 20% to or to about 90%, from or from about 20% to or to about 80%, from or from about 20% to or to about 70%, from or from about 20% to or to about 60%, from or from about 20% to or to about 50%, from or from about 20% to or to about 40%, from or from about 20% to or to about 30%, from or from about 30% to or to about 99%, from or from about 30% to or to about 90%, from or from about 30% to or to about 80%, from or from about 30% to or to about 70%, from or from about 30% to or to about 60%, from or from about 30% to or to about 50%, from or from about 30% to or to about 40%, from or from about 40% to or to about 99%, from or from about 40% to or to about 90%, from or from about 40% to or to about 80%, from or from about 40% to or to about 70%, from or from about 40% to or to about 70%, from or from about 40% to or to about 60%, from or from about 40% to or to about 50%, from or from about 50% to or to about 99%, from or from about 50% to or to about 90%, from or from about 50% to or to about 80%, from or from about 50% to or to about 70%, from or from about 50% to or to about 60%, from or from about 60% to or to about 99%, from or from about 60% to or to about 90%, from or from about 60% to or to about 80%, from or from about 60% to or to about 70%, from or from about 70% to or to about 99%, from or from about 70% to or to about 90%, from or from about 70% to or to about 80%, from or from about 80% to or to about 99%, from or from about 80% to or to about 90%, or from or from about 90% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein.

In some embodiments, frozen cells of a first or second MCB are thawed and cultured. In some embodiments, a single vial of frozen cells of the first or second MCB e.g., a single vial comprising 800 or about 800 million cells, e.g., first or second MCB cells, are thawed and cultured. In some embodiments, the frozen first or second MCB cells are cultured with additional feeder cells to produce cells suitable for use either as a second or third MCB or in an infusion-ready drug product. In some embodiments, the cells from the co-culture of the first or second MCB are harvested and frozen.

In some embodiments, the cells from the co-culture of the natural killer cell source, a first MCB, or a second MCB are harvested, and frozen in a cryopreservation composition, e.g., a cryopreservation composition described herein. In some embodiments, the cells are washed after harvesting. Thus, provided herein is a pharmaceutical composition comprising activated and stimulated NK cells, e.g., activated and stimulated NK cells produced by the methods described herein, e.g., harvested and washed activated and stimulated NK cells produced by the methods described herein and a cryopreservation composition, e.g., a cryopreservation composition described herein.

In some embodiments, the cells are mixed with a cryopreservation composition, e.g., as described herein, before freezing. In some embodiments, the cells are frozen in cryobags. In some embodiments, the cells are frozen in cryovials.

In some embodiments, the method further comprises isolating NK cells from the population of expanded and stimulated NK cells.

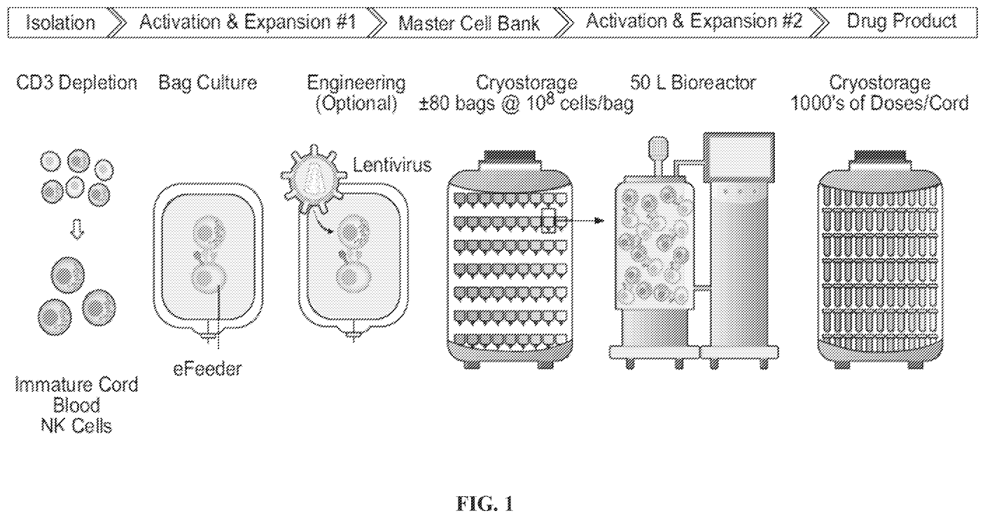

An exemplary process for expanding and stimulating NK cells is shown in FIG. 1.

### Engineering

In some embodiments, the method further comprises engineering NK cell(s), e.g., to express a heterologous protein, e.g., a heterologous protein described herein, e.g., a heterologous protein comprising a CAR and/or IL-15.

In some embodiments, engineering the NK cell(s) to express a heterologous protein described herein comprises transforming, e.g., stably transforming the NK cells with a vector comprising a polynucleic acid encoding a heterologous protein described herein. Suitable vectors are described herein.

In some embodiments, engineering the NK cell(s) to express a heterologous protein described herein comprises introducing the heterologous protein via gene editing (e.g., zinc finger nuclease (ZFN) gene editing, ARCUS gene editing. CRISPR-Cas9 gene editing, or megaTAL gene editing) combined with adeno-associated virus (AAV) technology.

In some embodiments, the NK cell(s) are engineered to express a heterologous protein described herein, e.g., during or after culturing the composition in a medium comprising feeder cells. For example, in some cases, engineering (e.g., transduction) occurs during the expansion and stimulation process described herein, e.g., during co-culturing NK cell source(s) and feeder cell(s) as described herein, e.g., at day 1. 2. 3. 4. 5. 6. 7. 8. 9. 10. 11. 12. 13. 14. 15. 16, 17, or 18 of co-culture.

In some embodiments, the method further comprises engineering NK cell(s), e.g., to express, over-express, knock-out, or knock-down gene(s) or gene product(s).

In some embodiments, the natural killer cells are not genetically engineered.

In some embodiments, the NK cell(s) are engineered (e.g., transduced) in a culture medium supplemented with a stimulating factor (e.g., as described herein). Such cytokines can be used to provide growth or survival signals to the NK cells during the engineering process or to increase transduction efficiency. In some embodiments, the stimulation factor(s) are cytokine(s). In some embodiments, the cytokine(s) are selected from the group consisting of IL-2. IL-12, IL-15, IL-18, IL-21, IL-23, IL-27, IFN-α, IFNβ, and combinations thereof.

In some embodiments, the cytokine is IL-21. IL-21 can be used at a final concentration of between 10 and 100 ng/mL, including, for example, at or at about 10, 15, 20, 25, 30, 34, 40, 45, 50, 55, 60, 70. 80, 90, or 100 ng/mL. In some embodiments, the cytokine is IL-2. In some embodiments, the cytokines are a combination of IL-2 and IL-21. In some embodiments, the cytokines are a combination of IL-2, IL-18, and IL-21.

In some embodiments, the stimulating factor is added to the culture medium at the time of engineering (e.g., transduction). In some embodiments, the stimulating factor is added to the culture medium after the time of engineered (e.g., transducing), e.g., from 1 to 48 hours after engineering, e.g., from 1 to 36, 1 to 24, 1 to 12, 12 to 28, 12 to 36, 12 to 24. 24 to 48, 24 to 36, or 36 to 48 hours after engineering. In some embodiments, the stimulating factor is added to the culture medium both at the time of transduction and after the time of engineering (e.g., from 1 to 48 hours after transduction).

In some embodiments, the culture is supplemented with the stimulating factor after culturing in a medium comprising feeder cells. Thus, in some cases, the culture medium will contain feeder cells at the time of engineering (e.g., transduction). In some cases, the feeder cells are removed from the culture prior to supplementation with the stimulating factor or engineering. In some cases, the feeder cells are not removed from the culture prior to supplementation with the stimulating factor or engineering. In some cases, no additional feeder cells are added to the culture during engineering, whether or not any residual feeder cells are removed. In some cases, both additional feeder cells and a stimulating factor are added to the culture during engineering. In some cases, additional feeder cells are not added to the culture during engineering but stimulating factors are added to the culture during engineering.

### Properties of Expanded and Stimulated NK Cells

After having been ex vivo expanded and stimulated, e.g., as described herein, the expanded and stimulated NK cell populations not only have a number/density (e.g., as described above) that could not occur naturally in the human body, but they also differ in their phenotypic characteristics. (e.g., gene expression and/or surface protein expression) with the starting source material or other naturally occurring populations of NK cells.

In some cases, the starting NK cell source is a sample derived from a single individual, e.g., a single cord blood unit that has not been ex vivo expanded. Therefore, in some cases, the expanded and stimulated NK cells share a common lineage, i.e., they all result from expansion of the starting NK cell source, and, therefore, share a genotype via clonal expansion of a population of cells that are, themselves, from a single organism. Yet, they could not occur naturally at the density achieved with ex vivo expansion and also differ in phenotypic characteristics from the starting NK cell source.

In some cases, the population of expanded and stimulated NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the expanded and stimulated NK cells comprise at least 80%, e.g., at least 90%, at least 95%, at least 99%, or 100% CD56+CD3-cells.

In some embodiments, the expanded and stimulated NK cells are not genetically engineered.

In some embodiments, the expanded and stimulated NK cells do not comprise a CD16 transgene.

In some embodiments, the expanded and stimulated NK cells do not express an exogenous CD16 protein.

The expanded and stimulated NK cells can be characterized, for example, by surface expression, e.g., of one or more of CD16, CD56, CD3, CD38, CD14, CD19, NKG2D, NKp46, NKp30. DNAM-1, and NKp44.

The surface protein expression levels stated herein, in some cases are achieved without positive selection on the particular surface protein referenced. For example, in some cases, the NK cell source, e.g., a single cord unit, comprises both the KIR B allele of the KIR receptor family and the 158 V/V variant of CD16 and is + enriched and CD3 (+) depleted, e.g., by gating on CD56+CD3-expression. but no other surface protein expression selection is carried out during expansion and stimulation.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKG2D+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp46+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp30+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% DNAM-1+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp44+ cells. In some embodiments, the expanded and stimulated NK cells, e.g., from expansion

and stimulation of a single cord blood unit, e.g., as described above, comprise at least 60%, e.g., at least 70%, at least 80%, at least 90% at least 95%, at least 99%, or 100% CD94+ (KLRD1) cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD3+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD14+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD19+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CXCR+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD122+ (IL2RB) cells.

As described herein, the inventors have demonstrated that, surprisingly, the NK cells expanded and stimulated by the methods described herein express CD16 at high levels throughout the expansion and stimulation process, resulting in a cell population with high CD16 expression. The high expression of CD16 obviates the need for engineering the expanded cells to express CD16, which is important for initiating ADCC, and, therefore, a surprising and unexpected benefit of the expansion and stimulation methods described herein. Thus, in some embodiments, the expanded and stimulated NK cells, e.g., from expansion and stimulation of a single cord blood unit, e.g., as described above, comprise 50% or more, e.g., 55%, 60%, 65%, 70%, 75%, 80%, 85%, 90%, or 95% CD16+NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise 50% or more, e.g., 55%, 60%, 65%, 70%, 75%, 80%, 85%, 90%, or 95% CD16+NK cells.

In some embodiments, the percentage of expanded and stimulated NK cells, e.g., from expansion and stimulation of a single cord blood unit, e.g., as described above, expressing CD16 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKG2D is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp30 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing DNAM-1 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp44 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing

NKp46 is the same or higher than the percentage of natural killer cells in the seed cells from umbilical cord blood.

As described herein, the inventors have also demonstrated that, surprisingly, the NK cells expanded and stimulated by the methods described herein express CD38 at low levels. CD38 is an effective target for certain cancer therapies (e.g., multiple myeloma and acute myeloid leukemia). See, e.g., . Jiao et al., “CD38: Targeted Therapy in Multiple Myeloma and Therapeutic Potential for Solid Cancerrs,” Expert Opinion on Investigational Drugs 29 (11): 1295-1308 (2020).

Thus, in some embodiments, the expanded and stimulated NK cells, e.g., from expansion and stimulation of a single cord blood unit, e.g., as described above, comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells, and 50% or more, e.g., 55%, 60%, 65%, 70%, 75%, 80%, 85%, 90%, or 95% CD16+NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise: i) 50% or more, e.g., 55%, 60%, 65%, 70%, 75%, 80%, 85%, 90%, or 95% CD16+NK cells; and/or ii) less than or equal to 80% CD38+ cells, e.g., less than or equal to 75%, 70%, 65%, 60%, 55%, 50%, 45%, 40%, 35%, 30%, 25%, or 20% CD38+ cells; and/or iii) at least 60%, e.g., at least 70%, at least 80%, at least 90% at least 95%, at least 99%, or 100% NKG2D+ cells; and/or iv) at least 60%, e.g., at least 70%, at least 80%, at least 90% at least 95%, at least 99%, or 100%

NKp46+ cells; and/or v) at least 60%, e.g., at least 70%, at least 80%, at least 90% at least 95%, at least 99%, or 100% NKp30+ cells; and/or vi) at least 60%, e.g., at least 70%, at least 80%, at least 90% at least 95%, at least 99%, or 100% DNAM-1+ cells; and/or vii) at least 60%, e.g., at least 70%, at least 80%, at least 90% at least 95%, at least 99%, or 100% NKp44+ cells; and/or viii) at least 60%, e.g., at least 70%, at least 80%, at least 90% at least 95%, at least 99%, or 100% CD94+ (KLRD1) cells; and/or ix) less than or equal to 20%, e.g., less than or equal to 10%, less than or equal to 5%, less than or equal to 1% or 0% CD3+ cells; and/or x) less than or equal to 20%, e.g., less than or equal to 10%, less than or equal to 5%, less than or equal to 1% or 0% CD14+ cells; and/or xi) less than or equal to 20%, e.g., less than or equal to 10%, less than or equal to 5%, less than or equal to 1% or 0% CD19+ cells; and/or xii) less than or equal to 20%, e.g., less than or equal to 10%, less than or equal to 5%, less than or equal to 1% or 0% CXCR+ cells; and/or xiii) less than or equal to 20%, e.g., less than or equal to 10%, less than or equal to 5%, less than or equal to 1% or 0% CD122+ (IL2RB) cells.

In some embodiments, feeder cells do not persist in the expanded and stimulated NK cells, though, residual signature of the feeder cells may be detected, for example, by the presence of residual cells (e.g., by detecting cells with a particular surface protein expression) or residual nucleic acid and/or proteins that are expressed by the feeder cells.

For example, in some cases, the methods described herein include expanding and stimulating natural killer cells using engineered feeder cells, e.g., eHuT-78 feeder cells described above, which are engineered to express sequences that are not expressed by cells in the natural killer cell source, including the natural killer cells. For example, the engineered feeder cells can be engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof.

While these feeder cells may not persist in the expanded and stimulated NK cells, the expanded and stimulated NK cells may retain detectable residual amounts of cells, proteins, and/or nucleic acids from the feeder cells. Thus, their residual presence in the expanded and stimulated NK cells may be detected, for example by detecting the cells themselves (e.g., by flow cytometry), proteins that they express, and/or nucleic acids that they express.

Thus, also described herein is a population of expanded and stimulated NK cells comprising residual feeder cells (live cells or dead cells) or residual feeder cell cellular impurities (e.g., residual feeder cell proteins or portions thereof, and/or genetic material such as a nucleic acid or portion thereof). In some cases, the expanded and stimulated NK cells comprise more than 0) % and, but 0).3% or less residual feeder cells, e.g., eHuT-78 feeder cells.

In some cases, the expanded and stimulated NK cells comprise residual feeder cell nucleic acids, e.g., encoding residual 4-1BBL (UniProtKB P41273, SEQ ID NO: 1), membrane bound IL-21 (SEQ ID NO: 2), and/or mutant TNFalpha (SEQ ID NO: 3) or portion(s) thereof. In some cases, the membrane bound IL-21 comprises a CD8 transmembrane domain

In some cases, the expanded and stimulated NK cells comprise a % residual feeder cells of more than 0% and less than or equal to 0.2%, as measured, e.g., by the relative proportion of a feeder cell specific protein or nucleic acid sequence (that is, a protein or nucleic acid sequence not expressed by the natural killer cells) in the sample. For example, by qPCR, e.g., as described herein.

In some embodiments, the residual feeder cells are CD4 (+) T cells. In some embodiments, the residual feeder cells are engineered CD4 (+) T cells. In some embodiments, the residual feeder cell cells are engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“el lut-78 cells”), or variants thereof. Thus, in some cases, the feeder cell specific protein is 4-1BBL (UniProtKB P41273, SEQ ID NO: 1).

membrane bound IL-21 (SEQ ID NO: 2), and/or mutant TNFalpha (SEQ ID NO: 3). And, therefore, the feeder cell specific nucleic acid is a nucleic acid encoding 4-1BBL (UniProtKB P41273, SEQ ID NO: 1), membrane bound IL-21 (SEQ ID NO: 2), and/or mutant TNFalpha (SEQ ID NO: 3), or portion thereof. In some cases, the membrane bound IL-21 comprises a

CD8 transmembrane domain.

A wide variety of different methods can be used to analyze and detect the presence of nucleic acids or protein gene products in a biological sample. As used herein, “detecting” can refer to a method used to discover, determine, or confirm the existence or presence of a compound and/or substance (e.g., a cell, a protein and/or a nucleic acid). In some embodiments, a detecting method can be used to detect a protein. In some embodiments, detecting can include chemiluminescence or fluorescence techniques. In some embodiments, detecting can include immunological-based methods (e.g., quantitative enzyme-linked immunosorbent assay's (ELISA), Western blotting, or dot blotting) wherein antibodies are used to react specifically with entire proteins or specific epitopes of a protein. In some embodiments, detecting can include immunoprecipitation of the protein (Jungblut et al., J Biotechnol.31: 41 (2-3): 111-20 (1995); Franco et al., Eur.J Morphol. 39 (1): 3-25 (2001)). In some embodiments, a detecting method can be used to detect a nucleic acid (e.g., DNA and/or RNA). In some embodiments, detecting can include Northern blot analysis, nuclease protection assays (NPA), in situ hybridization, or reverse transcription-polymerase chain reaction (RT-PCR) (Raj et al., Nat. Methods 5, 877-879 (2008): Jin et al., J Clin Lab Anal. 11 (1): 2-9 (1997): Ahmed, J Environ Sci Health C Environ Carcinog Ecotoxicol Rev. 20 (2): 77-116 (2002)).

Thus, also described herein, are methods for detecting a population of expanded and stimulated NK cells, e.g., expanded and stimulated using the methods described herein, that have been co-cultured with engineered feeder cells, e.g., eHuT-78 feeder cells described herein.

### Anti-Her2 Car-Nk

Provided herein are engineered cells, e.g., engineered natural killer cells, e.g., CAR-NK cells, e.g., anti-HER2 CAR-NK cells. In some embodiments, the CAR-NK cells are engineered to express IL-15.

In some embodiments, the natural killer cells are engineered, e.g., transduced, during expansion and stimulation, e.g., expansion and stimulation described herein. In some embodiments, the natural killer cells are engineered during expansion and stimulation, e.g., during production of a MCB, as described herein. In some embodiments, the natural killer cells are engineered during expansion and stimulation, e.g., during production of NK cells suitable for use in an injection-ready drug product and/or during production of a MCB, as described above. Thus, in some embodiments, the NK cell(s) are host cells and provided herein are NK host cell(s) expressing a heterogeneous protein, e.g., as described herein.

In some embodiments, the natural killer cells are engineered prior to expansion and stimulation. In some embodiments, the natural killer cells are engineered after expansion and stimulation.

In some embodiments, the NK cells are engineered by transducing with a vector. Suitable vectors are described herein, e.g., lentiviral vectors, e.g., a lentiviral vectors comprising a heterologous protein, e.g., as described herein. In some embodiments, the NK cells are transduced during production of a first MCB, as described herein.

In some embodiments, the NK cell(s) are transduced at a multiplicity of infection of from or from about 1 to or to about 40 viral particles per cell. In some embodiments, the NK cell(s) are transduced at a multiplicity of infection of or of about 1, of or of about 5, of or of about 10, of or of about 15, of or of about 20, of or of about 25, of or of about 30, of or of about 35, or of or of about 40 viral particles per cell.

### Chimeric Antigen Receptors

In some embodiments, the heterologous protein is a fusion protein, e.g., a fusion protein comprising a chimeric antigen receptor (CAR) is introduced into the NK cell, e.g., during the expansion and stimulation process.

In some embodiments, the CAR comprises one or more of: a signal sequence, an extracellular domain, a hinge, a transmembrane domain, and one or more intracellular signaling domain sequences. In some embodiments, the CAR further comprises a spacer sequence.

In some embodiments, the CAR comprises (from N- to C-terminal): a signal sequence, an extracellular domain, a hinge, a spacer, a transmembrane domain, a first signaling domain sequence, a second signaling domain sequence, and a third signaling domain sequence.

In some embodiments, the CAR comprises (from N- to C-terminal): a signal sequence, an extracellular domain, a hinge, a transmembrane domain, a first signaling domain sequence, a second signaling domain sequence, and a third signaling domain sequence.

The signal sequence can be cleaved from a mature CAR protein. Such cleavage can be mediated by a signal peptidase and can occur either during or after completion of translocation to generate the mature protein. Thus, in some embodiments, the CAR comprises (from N- to C-terminal): an extracellular domain, a hinge, a spacer, a transmembrane domain, a first signaling domain sequence, a second signaling domain sequence, and a third signaling domain sequence.

In some embodiments, the CAR comprises (from N- to C-terminal): an extracellular domain, a hinge, a transmembrane domain, a first signaling domain sequence, a second signaling domain sequence, and a third signaling domain sequence.

In some embodiments the extracellular domain comprises an antibody or antigen-binding portion thereof.

In some embodiments, one or more of the intracellular signaling domain sequence(s) is a CD28 intracellular signaling sequence. In some embodiments, the CD28 intracellular signaling sequence comprises or consists of SEQ ID NO: 5.

In some embodiments, one or more of the intracellular signaling domain sequence(s) is an OX40L signaling sequence. Sec, e.g., . Matsumura et al., “Intracellular Signaling of gp34, the OX40 Ligand: Induction of c-jun and c-fos mRNA Expression Through gp34 upon Binding of Its Receptor, OX40,” J. Immunol 163:3007-11 (1999), which is hereby incorporated by reference in its entirety. In some embodiments, the OX40L signaling sequence comprises or consists of SEQ ID NO: 8. SEQ ID NO: 9, or SEQ ID NO: 10.

In some embodiments, one or more of the intracellular signaling sequence(s) is a CD3 {intracellular signaling domain sequence. In some embodiments, the CD3} intracellular signaling sequence comprises of consists of SEQ ID NO: 13.

In some embodiments, the CAR comprises a CD28 intracellular signaling sequence (SEQ ID NO: 5), an OX40L intracellular signaling sequence (SEQ ID NO: 8, SEQ ID NO: 9, or SEQ ID NO: 10), and a CD3} intracellular signaling sequence (SEQ ID NO: 13).

In some embodiments, the CAR comprises an intracellular signaling domain comprising or consisting of SEQ ID NO: 25.

In some embodiments, the CAR does not comprise an OX40L intracellular signaling domain sequence.

In some embodiments, the CAR comprises a CD28 intracellular signaling sequence (SEQ ID NO: 5), and a CD35 intracellular signaling sequence (SEQ ID NO: 13), but not an OX40L intracellular signaling domain sequence.

In some embodiments, the signal sequence is a CD8a signal sequence. In some embodiments, the signal sequence comprises or consists of SEQ ID NO: 27.

In some embodiments, the extracellular domain comprises a single-chain variable fragment (scFv). In some embodiments, the extracellular domain comprises an anti-HER2 antibody or antigen binding fragment thereof. In some embodiments, the extracellular domain comprises an anti-HER2 scFv.

In some embodiments, the anti-HER2 scFv comprises a CDRL1 domain comprising or consisting of SEQ ID NO: 34, a CDRL2 domain comprising or consisting of SEQ ID NO: 36, a CDRL3 domain comprising or consisting of SEQ ID NO: 38, a CDRH1 domain comprising or consisting of SEQ ID NO: 44, a CDRH2 domain comprising or consisting of SEQ ID NO: 46, and a CDRH3 domain comprising or consisting of SEQ ID NO: 48.

In some embodiments, the anti-HER2 scFv comprises a VL domain comprising or consisting of SEQ ID NO: 32 and a V_{H} domain comprising or consisting of SEQ ID NO: 42.

In some embodiments, the anti-HER2 scFv comprises a VL domain comprising or consisting of SEQ ID NO: 32, a linker comprising or consisting of SEQ ID NO: 40, and a V_{H} domain comprising or consisting of SEQ ID NO: 42.

In some embodiments, the anti-HER2 scFv comprises or consists of SEQ ID NO: 30.

In some embodiments, the hinge comprises or consists of a CD8a hinge. In some embodiments, the CD8a hinge comprises or consists of SEQ ID NO: 50.

In some embodiments, the transmembrane domain is a CD28 transmembrane domain.

In some embodiments, the CD28 transmembrane domain comprises of consists of SEQ ID NO: 53.

In some embodiments, the fusion protein comprises or consists of SEQ ID NO: 56. IL-15

In some embodiments, the NK cell is engineered to express IL-15, e.g., human IL-15 (UniProtKB #P40933: NCBI Gene ID #3600), e.g., soluble human IL-15 or an ortholog thereof, or a variant of any of the foregoing. In some embodiments, the IL-15 is expressed as part of a fusion protein further comprising a cleavage site. In some embodiments, the IL-15 is expressed as part of a polyprotein comprising a self-cleaving peptide such as a T2A ribosomal skip sequence site (sometimes referred to as a self-cleaving site). See, e.g., . Radcliffe & Mitrophanous. “Multiple Gene Products from a Single Vector: “Self-Cleaving” 2A Peptides.”

Gene Therapy 11:1673-4 (2004); see also Liu et al., “Systematic Comparison of 2A Peptides for Cloning Multi-Genes in a Polycistronic Vector,” Scientific Reports 7 (1): 2193 (2017). In some embodiments, the IL-15 comprises or consists of SEQ ID NO: 22.

In some embodiments, the self-cleaving peptide is a 2A self-cleaving peptide. In some embodiments, the self-cleaving peptide is a T2A. P2A. E2A, or F2A self-cleaving peptide. In some embodiments, the self-cleaving peptide comprises SEQ ID NO: 16. In some embodiments, the self-cleaving peptide comprises or consists of SEQ ID NO: 16, SEQ ID NO:

17, SEQ ID NO: 19, SEQ ID NO: 20, or SEQ ID NO: 21.

In some embodiments, the T2A cleavage site comprises or consists of SEQ ID NO: 17.

In some embodiments, the IL-15 is expressed as part of a fusion protein comprising a CAR, e.g., a CAR described herein.

In some embodiments, the fusion protein comprises (oriented from N-terminally to C-terminally): a CAR comprising, a cleavage site, and IL-15.

In some embodiments, the fusion protein comprises SEQ ID NO: 26.

In some embodiment, the fusion protein comprises or consists of SEQ ID NO: 59.

### Inhibitory Receptors

In some embodiments, the NK cell is engineered to alter, e.g., reduce, expression of one or more inhibitor receptor genes.

In some embodiments, the inhibitory receptor gene is a HLA-specific inhibitory receptor. In some embodiments, the inhibitory receptor gene is a non-HLA-specific inhibitory receptor.

In some embodiments, the inhibitor receptor gene is selected from the group consisting of KIR, CD94/NKG2A, LILRBI, PD-1, Irp60, Siglec-7, LAIR-1, and combinations thereof.

### Polynucleic Acids, Vectors, and Host Cells

Also provided herein are polynucleic acids encoding the fusion protein(s) or portions thereof, e.g., the polynucleotide sequences encoding the polypeptides described herein, as shown in the Table of sequences provided herein

Also provided herein are vector(s) comprising the polynucleic acids, and cells, e.g., NK cells, comprising the vector(s).

In some embodiments, the vector is a lentivirus vector. See, e.g., . Milone et al.,

“Clinical Use of Lentiviral Vectors,” Leukemia 32:1529-41 (2018). In some embodiments, the vector is a retrovirus vector. In some embodiments, the vector is a gamma retroviral vector. In some embodiments, the vector is a non-viral vector, e.g., a piggy back non-viral vector (PB transposon, see, e.g., . Wu et al . . . “piggyback is a Flexible and Highly Active Transposon as Compared to Sleeping Beauty, Tol2, and Mosl in Mammalian Cells,” PNAS 103 (41): 15008-13 (2006)), a sleeping beauty non-viral vector (SB transposon, see, e.g., Hudecek et al., “Going Non-Viral: the Sleeping Beauty Transposon System Breaks on Through to the Clinical Side,” Critical Reviews in Biochemistry and Molecular Biology 52 (4): 355-380 (2017)), or an mRNA vector.

### Cryopreservation

### Cryopreservation Compositions

Provided herein are cryopreservation compositions, e.g., cryopreservation compositions suitable for intravenous administration, e.g., intravenous administration of NK cells, e.g., the NK cells described herein. In some embodiments, a pharmaceutical composition comprises the cryopreservation composition and cells, e.g., the NK cells described herein.

### Albumin

In some embodiments, the cryopreservation composition comprises albumin protein, e.g., human albumin protein (UniProtKB Accession P0278, SEQ ID NO: 63) or variant thereof. In some embodiments, the cryopreservation composition comprises an ortholog of an albumin protein, e.g., human albumin protein, or variant thereof. In some embodiments, the cryopreservation composition comprises a biologically active portion of an albumin protein, e.g., human albumin, or variant thereof.

In some embodiments, the albumin, e.g., human albumin, is provided as a solution, also referred to herein as an albumin solution or a human albumin solution. Thus, in some embodiments, the cryopreservation composition is or comprises an albumin solution, e.g., a human albumin solution. In some embodiments, the albumin solution is a serum-free albumin solution.

In some embodiments, the albumin solution is suitable for intravenous use.

In some embodiments, the albumin solution comprises from or from about 40 to or to about 200 g/L albumin. In some embodiments, the albumin solution comprises from or from about 40 to or to about 50 g/L albumin, e.g., human albumin. In some embodiments, the albumin solution comprises about 200 g/L albumin, e.g., human albumin. In some embodiments, the albumin solution comprises 200 g/L albumin, e.g., human albumin.

In some embodiments, the albumin solution comprises a protein composition, of which 95% or more is albumin protein, e.g., human albumin protein. In some embodiments. 96%, 97%, 98%, or 99% or more of the protein is albumin, e.g., human albumin.

In some embodiments, the albumin solution further comprises sodium. In some embodiments, the albumin solution comprises from or from about 100 to or to about 200 mmol sodium. In some embodiments, the albumin solution comprises from or from about 130 to or to about 160 mmol sodium.

In some embodiments, the albumin solution further comprises potassium. In some embodiments, the albumin solution comprises 3 mmol or less potassium. In some embodiments, the albumin solution further comprises 2 mmol or less potassium.

In some embodiments, the albumin solution further comprises one or more stabilizers. In some embodiments, the stabilizer(s) are selected from the group consisting of sodium caprylate, caprylic acid, (2S)-2-acetamido-3-(1H-indol-3-yl) propanoic acid (also referred to as acetyl tryptophan, N-Acetyl-L-tryptophan and Acetyl-L-tryptophan), 2-acetamido-3-(1H-indol-3-yl) propanoic acid (also referred to as N-acetyltryptophan, DL-Acetyltroptohan and N-Acetyl-DL-tryptophan). In some embodiments, the solution comprises less than. 1 mmol of each of the one or more stabilizers per gram of protein in the solution. In some embodiments, the solution comprises from or from about 0.05 to or to about 0.1, e.g., from or from about 0.064 to or to about 0.096 mmol of each of the stabilizers per gram of protein in the solution. In some embodiments, the solution comprises less than 0.1 mmol of total stabilizer per gram of protein in the solution. In some embodiments, the solution comprises from or from about 0.05 to or to about 0.1, e.g., from or from about 0.064 to or to about 0.096 mmol of total stabilizer per gram of protein in the solution.

In some embodiments, the albumin solution consists of a protein composition, of which 95% or more is albumin protein, sodium, potassium, and one or more stabilizers selected from the group consisting of sodium caprylate, caprylic acid, (2S)-2-acetamido-3-(1H-indol-3-yl) propanoic acid (also referred to as acetyl tryptophan, N-Acetyl-L-tryptophan and Acetyl-L-tryptophan), 2-acetamido-3-(1H-indol-3-yl) propanoic acid (also referred to as N-acetyltryptophan, DL-Acetyltroptohan and N-Acetyl-DL-tryptophan) in water.

In some embodiments, the cryopreservation composition comprises from or from about 10% v/v to or to about 50% v/v of an albumin solution, e.g., an albumin solution described herein. In some embodiments, the cryopreservation composition comprises from or from about 10% to or to about 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% v/v of an albumin solution described herein. In some embodiments, the cryopreservation composition comprises about 10%, about 15%, about 20%, about 25%, about 30%, about 35%, about 40%, about 45%, or about 50% v/v of an albumin solution described herein. In some embodiments, the cryopreservation composition comprises 10%, 15%, 20%, 25%, 30%, 35%, 40%, 45%, or 50% v/v of an albumin solution described herein.

In some embodiments, the cryopreservation composition comprises from or from about 20 to or to about 100 g/L albumin, e.g., human albumin. In some embodiments, the cryopreservation composition comprises from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 100, from or from about 80 to or to about 90, or from or from about 90 to or to about 100 g/L albumin, e.g., human albumin.

In some embodiments, the cryopreservation composition comprises 20 g/L albumin, e.g., human albumin. In some embodiments, the cryopreservation composition comprises 40 g/L albumin, e.g., human albumin. In some embodiments, the cryopreservation composition comprises 70 g/L albumin, e.g., human albumin. In some embodiments, the cryopreservation composition comprises 100 g/L albumin, e.g., human albumin.

In some embodiments, the cryopreservation composition comprises about 20 g/L albumin, e.g., human albumin. In some embodiments, the cryopreservation composition comprises about 40 g/L albumin, e.g., human albumin. In some embodiments, the cryopreservation composition comprises about 70 g/L albumin, e.g., human albumin. In some embodiments, the cryopreservation composition comprises about 100 g/L albumin, e.g., human albumin.

In some embodiments, the cryopreservation composition further comprises a stabilizer, e.g., an albumin stabilizer. In some embodiments, the stabilizer(s) are selected from the group consisting of sodium caprylate, caprylic acid, (2S)-2-acetamido-3-(1H-indol-3-yl) propanoic acid (also referred to as acetyl tryptophan. N-Acetyl-L-tryptophan and Acetyl-L-tryptophan), 2-acetamido-3-(1H-indol-3-yl) propanoic acid (also referred to as N-acetyltryptophan, DL-Acetyltroptohan and N-Acetyl-DL-tryptophan). In some embodiments, the cryopreservation composition comprises less than. 1 mmol of each of the one or more stabilizers per gram of protein, e.g., per gram of albumin protein, in the composition. In some embodiments, the cryopreservation composition comprises from or from about 0.05 to or to about 0.1, e.g., from or from about 0.064 to or to about 0.096 mmol of each of the stabilizers per gram of protein, e.g., per gram of albumin protein in the composition. In some embodiments, the cryopreservation composition comprises less than 0.1 mmol of total stabilizer per gram of protein, e.g., per gram of albumin protein in the cryopreservation composition. In some embodiments, the cryopreservation composition comprises from or from about 0.05 to or to about 0.1, e.g., from or from about 0.064 to or to about 0.096 mmol of total stabilizer per gram of protein, e.g., per gram of albumin protein, in the cryopreservation composition.

### Dextran

In some embodiments, the cryopreservation composition comprises Dextran, or a derivative thereof.

Dextran is a polymer of anhydroglucose composed of approximately 95% a-D-(1-6) linkages (designated (C6H1005) n). Dextran fractions are supplied in molecular weights of from about 1,000 Daltons to about 2,000,000 Daltons. They are designated by number (Dextran X), e.g., Dextran 1, Dextran 10, Dextran 40, Dextran 70, and so on, where X corresponds to the mean molecular weight divided by 1,000 Daltons. So, for example, Dextran 40 has an average molecular weight of or about 40,000 Daltons.

In some embodiments, the average molecular weight of the dextran is from or from about 1,000 Daltons to or to about 2,000,000 Daltons. In some embodiments, the average molecular weight of the dextran is or is about 40,000 Daltons. In some embodiments, the average molecular weight of the dextran is or is about 70,000 Daltons.

In some embodiments, the dextran is selected from the group consisting of Dextran

40, Dextran 70, and combinations thereof. In some embodiments, the dextran is Dextran 40.

In some embodiments, the dextran, e.g., Dextran 40, is provided as a solution, also referred to herein as a dextran solution or a Dextran 40 solution. Thus, in some embodiments, the composition comprises a dextran solution, e.g., a Dextran 40 solution.

In some embodiments, the dextran solution is suitable for intravenous use.

In some embodiments, the dextran solution comprises about 5% to about 50% w/w

dextran, e.g., Dextran 40. In some embodiments, the dextran solution comprises from or from about 5% to or to about 50%, from or from about 5% to or to about 45%, from or from about 5% to or to about 40%, from or from about 5% to or to about 35%, from or from about 5% to or to about 30%, from or from about 5% to or to about 25%, from or from about 5% to or to about 20%, from or from about 5% to or to about 15%, from or from about 5% to or to about 10%.

from or from about 10% to or to about 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% w/w dextran, e.g.,

Dextran 40. In some embodiments, the dextran solution comprises 5%, 10%, 15%, 20%, 25%, 30%, 35%, 40%, 45%, or 50% w/w dextran, e.g., Dextran 40. In some embodiments, the dextran solution comprises about 5%, about 10%, about 15%, about 20%, about 25%, about 30%, about 35%, about 40%, about 45%, or about 50% w/w dextran, e.g., Dextran 40.

In some embodiments, the dextran solution comprises from or from about 25 g/L to or to about 200 g/L dextran, e.g., Dextran 40. In some embodiments, the dextran solution comprises from or from about 35 to or to about 200, from or from about 25 to or to about 175, from or from about 25 to or to about 150, from or from about 25 to or to about 125, from or from about 25 to or to about 100, from or from about 25 to or to about 75, from or from about 25 to or to about 50, from or from about 50 to or to about 200, from or from about 50 to or to about 175. from or from about 50 to or to about 150, from or from about 50 to or to about 125, from or from about 50 to or to about 100, from or from about 50 to or to about 75, from or from about 75 to or to about 200, from or from about 75 to or to about 175, from or from about 75 to or to about 150, from or from about 75 to or to about 125, from or from about 75 to or to about 100, from or from about 100 to or to about 200, from or from about 100 to or to about 175, from or from about 100 to or to about 150, from or from about 100 to or to about 125, from or from about 125 to or to about 200, from or from about 125 to or to about 175, from or from about 125 to or to about 150, from or from about 150 to or to about 200, from or from about 150 to or to about 175, or from or from about 175 to or to about 200 g/L dextran e.g., Dextran 40. In some embodiments, the dextran solution comprises 25, 50, 75, 100, 125, 150, 175, or 200 g/L dextran, e.g., Dextran 40. In some embodiments, the dextran solution comprises 100 g/L dextran, e.g., Dextran 40. In some embodiments, the dextran solution comprises about 25, about 50, about 75, about 100, about 125, about 150, about 175, or about 200 g/L dextran, e.g., Dextran 40. In some embodiments, the dextran solution comprises about 100 g/L dextran, e.g., Dextran 40.

In some embodiments, the dextran solution further comprises glucose (also referred to as dextrose). In some embodiments, the dextran solution comprises from or from about 10 g/L to or to about 100 g/L glucose. In some embodiments, the dextran solution comprises from or from about 10 to or to about 100, from or from about 10 to or to about 90, from or from about 10 to or to about 80, from or from about 10 to or to about 70, from or from about 10 to or to about 60, from or from about 10 to or to about 50, from or from about 10 to or to about 40, from or from about 10 to or to about 30, from or from about 10 to or to about 20, from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 90, from or from about 80 to or to about 100, from or from about 80 to or to about 90, or from or from about 90 to or to about 100 g/L glucose. In some embodiments, the dextran solution comprises 10, 20, 30, 40. 50. 60, 70, 80, 90, or 100 g/L glucose. In some embodiments, the dextran solution comprises 50 g/L glucose. In some embodiments, the dextran solution comprises about 10, about 20, about 30, about 40, about 50, about 60, about 70, about 80, about 90, or about 100 g/L glucose. In some embodiments, the dextran solution comprises 50 g/L glucose.

In some embodiments, the dextran solution consists of dextran, e.g., Dextran 40, and glucose in water.

In some embodiments, the cryopreservation composition comprises from or from about 10% v/v to or to about 50% v/v of a dextran solution described herein. In some embodiments, the cryopreservation composition comprises from or from about 10% to 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% v/v of a dextran solution, e.g., a dextran solution described herein. In some embodiments, the cryopreservation composition comprises 10%, 15%, 20%, 25%, 30%, 35%, 40%, 45%, or 50% v/v of a dextran solution, e.g., a dextran solution described herein. In some embodiments, the cryopreservation composition comprises about 10%, about 15%, about 20%, about 25%, about 30%, about 35%, about 40%, about 45%, or about 50% v/v of a dextran solution, e.g., a dextran solution described herein.

In some embodiments, the cryopreservation composition comprises from or from about 10 to or to about 50 g/L dextran, e.g., Dextran 40. In some embodiments, the cryopreservation composition comprises from or from about 10 to or to about 50, from or from about 10 to or to about 45, from or from about 10 to or to about 40, from or from about 10 to or to about 35, from or from about 10 to or to about 30, from or from about 10 to or to about 25, from or from about 10 to or to about 20, from or from about 10 to or to about 15, from or from about 15 to or to about 50, from or from about 15 to or to about 45, from or from about 15 to or to about 40, from or from about 15 to or to about 35, from or from about 15 to or to about 30, from or from about 15 to or to about 25, from or from about 15 to or to about 20, from or from about 20 to or to about 50, from or from about 20 to or to about 45, from or from about 20 to or to about 40, from or from about 20 to or to about 30, from or from about 20 to or to about 25, from or from about 25 to or to about 50, from or from about 25 to or to about 45, from or from about 25 to or to about 40, from or from about 25 to or to about 35, from or from about 25 to or to about 30, from or from about 30 to or to about 50, from or from about 30 to or to about 45, from or from about 30 to or to about 40, from or from about 30 to or to about 35, from or from about 35 to or to about 50, from or from about 35 to or to about 45, from or from about 35 to or to about 40, from or from about 40 to or to about 50, from or from about 40 to or to about 45, or from or from about 45 to or to about 50 g/L dextran, e.g., Dextran 40. In some embodiments, the cryopreservation composition comprises 10, 15, 20, 25, 30, 30, 35, 40, 45, or 50 g/L dextran, e.g., Dextran 40. In some embodiments, the cryopreservation composition comprises about 10, about 15, about 20, about 25, about 30, about 30, about 35, about 40, about 45, or about 50 g/L dextran, e.g., Dextran 40.

### Glucose

In some embodiments, the cryopreservation composition comprises glucose (D-glucose and/or L-glucose).

In some embodiments, as described above, the cryopreservation composition comprises a Dextran solution comprising glucose.

In some embodiments, the cryopreservation composition comprises a Dextran solution that does not comprise glucose. In some embodiments, e.g., when the Dextran solution does not comprise glucose, glucose is added separately to the cryopreservation composition.

In some embodiments, the cryopreservation composition comprises from or from about 5 to or to about 25 g/L glucose. In some embodiments, the cryopreservation composition comprises from or from about 5 to or to about 25, from or from about 5 to or to about 20, from or from about 5 to or to about 15, from or from about 5 to or to about 10, from or from about 10 to or to about 25, from or from about 10 to or to about 20, from or from about 10 to or to about 15, from or from about 15 to or to about 25, from or from about 15 to or to about 20, or from or from about 20 to or to about 25 g/L glucose. In some embodiments, the cryopreservation composition comprises 5, 7.5, 10, 12.5, 15, 17.5, 20, 22.5, or 25 g/L glucose. In some embodiments, the cryopreservation composition comprises 12.5 g/L glucose. In some embodiments, the cryopreservation composition comprises about 5, about 7.5, about 10, about 12.5, about 15, about 17.5, about 20, about 22.5, or about 25 g/L glucose. In some embodiments, the cryopreservation composition comprises about 12.5 g/L glucose.

In some embodiments, the cryopreservation composition comprises less than 2.75% w/v glucose. In some embodiments, the cryopreservation composition comprises less than 27.5 g/L glucose. In some embodiments, the cryopreservation composition comprises less than 2% w/v glucose. In some embodiments, the cryopreservation composition comprises less than 1.5% w/v glucose. In some embodiments, the cryopreservation composition comprises about 1.25% w/v or less glucose.

### Dimethyl Sulfoxide

In some embodiments, the cryopreservation composition comprises dimethyl sulfoxide (DMSO, also referred to as methyl sulfoxide and methylsulfinylmethane).

In some embodiments, the DMSO is provided as a solution, also referred to herein as a DMSO solution. Thus, in some embodiments, the cryopreservation composition comprises a DMSO solution.

In some embodiments, the DMSO solution is suitable for intravenous use.

In some embodiments, the DMSO solution comprises 1.1 g/mL DMSO. In some embodiments, the DMSO solution comprises about 1.1 g/mL DMSO.

In some embodiments, the cryopreservation composition comprises from or from about 1% to or to about 10% v/v of the DMSO solution. In some embodiments, the cryopreservation composition comprises from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 9%, from or from about 5% to or to about 8%, from or from about 5% to or to about 7%, from or from about 5% to or to about 6%, from or from about 6% to or to about 10%, from or from about 6% to or to about 9%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v of the DMSO solution. In some embodiments, the cryopreservation composition comprises 1%, 2%, 3%, 4%, 5%, 6%, 7%, 8%, 9%, or 10% v/v of the DMSO solution. In some embodiments, the cryopreservation composition comprises 5% of the DMSO solution. In some embodiments, the cryopreservation composition comprises about 1%, about 2%, about 3%, about 4%, about 5%, about 6%, about 7%, about 8%, about 9%, or about 10% v/v of the DMSO solution. In some embodiments, the cryopreservation composition comprises about 5% of the DMSO solution.

In some embodiments, the cryopreservation composition comprises from or from about 11 to or to about 110 g/L DMSO. In some embodiments, from or from about the cryopreservation composition comprises from or from about 11 to or to about 110, from or from about 11 to or to about 99, from or from about 11 to or to about 88, from or from about 11 to or to about 77, from or from about 11 to or to about 66, from or from about 11 to or to about 55. from or from about 11 to or to about 44, from or from about 11 to or to about 33, from or from about 11 to or to about 22, from or from about 22 to or to about 110, from or from about 22 to or to about 99, from or from about 22 to or to about 88, from or from about 22 to or to about 77, from or from about 22 to or to about 77, from or from about 22 to or to about 66, from or from about 22 to or to about 55, from or from about 22 to or to about 44, from or from about 22 to or to about 33, from or from about 33 to or to about 110, from or from about 33 to or to about 99, from or from about 33 to or to about 88, from or from about 33 to or to about 77, from or from about 33 to or to about 66, from or from about 33 to or to about 55, from or from about 33 to or to about 44, from or from about 44 to or to about 110, from or from about 44 to or to about 99, from or from about 44 to or to about 88, from or from about 44 to or to about 77, from or from about 44 to or to about 66, from or from about 44 to or to about 55, from or from about 55 to or to about 110, from or from about 55 to or to about 99, from or from about 55 to or to about 88, from or from about 55 to or to about 77, from or from about 55 to or to about 66, from or from about 66 to or to about 110, from or from about 66 to or to about 99, from or from about 66 to or to about 88, from or from about 66 to or to about 77, from or from about 77 to or to about 119, from or from about 77 to or to about 88, from or from about 88 to or to about 110, from or from about 88 to or to about 99, or from or from about 99 to or to about 110 g/L DMSO. In some embodiments, the cryopreservation composition comprises 11, 22, 33, 44, 55, 66, 77, 88, 99, or 110 g/L DMSO. In some embodiments, the cryopreservation composition comprises 55 g/L DMSO. In some embodiments, the cryopreservation composition comprises about 11, about 22, about 33, about 44, about 55, about 66, about 77, about 88, about 99, or about 110 g/L DMSO. In some embodiments, the cryopreservation composition comprises about 55 g/L DMSO.

### Buffers

In some embodiments, the cryopreservation composition comprises a buffer solution, e.g., a buffer solution suitable for intravenous administration.

Buffer solutions include, but are not limited to, phosphate buffered saline (PBS), Ringer's Solution, Tyrode's buffer, Hank's balanced salt solution, Earle's Balanced Salt Solution, saline, and Tris.

In some embodiments, the buffer solution is phosphate buffered saline (PBS).

### Exemplary Cryopreservation Compositions

In some embodiments, the cryopreservation composition comprises or consists of: 1) albumin, e.g., human albumin, 2) dextran, e.g., Dextran 40, 3) DMSO, and 4) a buffer solution. In some embodiments, the cryopreservation composition further comprises glucose. In some embodiments, the cryopreservation composition consists of 1) albumin, e.g., human albumin, 2) dextran, e.g., Dextran 40. 3) glucose, 4) DMSO, and 5) a buffer solution.

In some embodiments, the cryopreservation composition comprises: 1) an albumin solution described herein, 2) a dextran solution described herein, 3) a DMSO solution described herein, and 4) a buffer solution.

In some embodiments, the cryopreservation composition consists of: 1) an albumin solution described herein, 2) a dextran solution described herein, 3) a DMSO solution described herein, and 4) a buffer solution.

In some embodiments, the cryopreservation composition does not comprise a cell culture medium.

In one embodiment, the cryopreservation composition comprises or comprises about 40 mg/mL human albumin, 25 mg/mL Dextran 40, 12.5 mg/mL glucose, and 55 mg/mL DMSO.

In one embodiment, the cryopreservation composition comprises or comprises about or consists of or consists of about 40 mg/mL human albumin, 25 mg/ml Dextran 40, 12.5 mg/mL glucose, 55 mg/mL DMSO, and 0.5 mL/mL 100% phosphate buffered saline (PBS) in water.

In one embodiment, the cryopreservation composition comprises or comprises about 32 mg/mL human albumin, 25 mg/mL Dextran 40, 12.5 mg/mL glucose, and 55 mg/mL DMSO.

In one embodiment, the cryopreservation composition comprises or comprises about or consists of or consists of about 32 mg/mL human albumin, 25 mg/ml Dextran 40, 12.5 mg/mL glucose, 55 mg/mL DMSO, and 0.54 mL/mL 100% phosphate buffered saline (PBS) in water.

Exemplary Cryopreservation Compositions are shown in Table 3, Table 4, Table 5, and Table 6.

TABLE 3
Exemplary Cryopreservation Compositions

| | | | Exemplary Range |
|---|---|---|---|
| | | | v/v % in |
| | Concentration Range | Exemplary Solution | Cryopreservation |
| Excipient Solution | of Solution | Concentration | Composition |
| Albumin Solution | 40-200 g/L albumin | 200 g/L albumin | 10%-50% |
| | in water | | |
| Dextran 40 | 25-200 g/L Dextran | 100 g/L Dextran 40; | 10%-50% |
| Solution | 40; and 0-100 g/L | 50 g/L glucose | |
| | glucose; in water | | |
| DMSO | 11-110 g/L DMSO | 1,100 g/L DMSO | 1%-10% |
| | in water | | |
| Buffer | to volume | to volume | to volume |

TABLE 4
Exemplary Cryopreservation Composition #1

| | | Exemplary | |
|---|---|---|---|
| | | v/v % in | Final Concentration in |
| | | Cryopreservation | Cryopreservation |
| Excipient Solution | Solution Composition | Composition #1 | Composition #1 |
| Albumin Solution | 200 g/L albumin in water | 20% | 40 mg/mL albumin |
| Dextran 40 | 100 g/L Dextran 40; and | 25% | 25 mg/mL Dextran 40; |
| Solution | 50 g/L glucose; in water | | 12.5 mg/mL glucose |
| DMSO | 100% DMSO (1,100 g/L) | 5% | 55 mg/mL |
| Buffer | 100% Phosphate | 50% | 0.5 mL/mL |
| | Buffered Saline (PBS) | | |

TABLE 5
Exemplary Cryopreservation Composition #2

| | | Exemplary | |
|---|---|---|---|
| | | v/v % in | Final Concentration in |
| | | Cryopreservation | Cryopreservation |
| Excipient Solution | Solution Composition | Composition #2 | Composition #2 |
| Albumin Solution | 200 g/L albumin in water | 16% | 32 mg/mL albumin |
| Dextran 40 | 100 g/L Dextran 40; and | 25% | 25 mg/mL Dextran 40; |
| Solution | 50 g/L glucose; in water | | 12.5 mg/mL glucose |
| DMSO | 100% DMSO (1,100 g/L) | 5% | 55 mg/mL |
| Buffer | 100% Phosphate | 54% | 0.54 mL/mL |
| | Buffered Saline (PBS) | | |

TABLE 6
Exemplary Cryopreservation #3

| | | Exemplary | |
|---|---|---|---|
| | | v/v % in | Final Concentration in |
| | | Cryopreservation | Cryopreservation |
| Excipient Solution | Solution Composition | Composition #3 | Composition #3 |
| Albumin Solution | 250 g/L albumin in water | 16% | 40 mg/mL albumin |
| Dextran 40 | 100 g/L Dextran 40; and | 25% | 25 mg/mL Dextran 40; |
| Solution | 50 g/L glucose; in water | | 12.5 mg/mL glucose |
| DMSO | 100% DMSO (1,100 g/L) | 5% | 55 mg/mL |
| Buffer | 100% Phosphate | 54% | 0.54 mL/mL |
| | Buffered Saline (PBS) | | |

### Methods of Cryopreserving

The cryopreservation compositions described herein can be used for cryopreserving cell(s), e.g., therapeutic cells, e.g., natural killer (NK) cell(s), e.g., the NK cell(s) described herein.

In some embodiments, the cell(s) are an animal cell(s). In some embodiments, the cell(s) are human cell(s).

In some embodiments, the cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein. In some embodiments, the NK cell(s) are CAR-NK cell(s), for example CAR-NK cell(s) described herein.

In some embodiments, cryopreserving the cell(s) comprises: mixing the cell(s) with a cryopreservation composition or components thereof described herein to produce a composition, e.g., a pharmaceutical composition; and freezing the mixture.

In some embodiments, cryopreserving the cell(s) comprises: mixing a composition comprising the cell(s) with a cryopreservation composition or components thereof described herein to produce a composition, e.g., a pharmaceutical composition; and freezing the mixture. In some embodiments, the composition comprising the cell(s) comprises: the cell(s) and a buffer. Suitable buffers are described herein.

In some embodiments, cryopreserving the cell(s) comprises: mixing a composition comprising the cell(s) and a buffer, e.g., PBS, with a composition comprising albumin. Dextran. and DMSO, e.g., as described herein; and freezing the mixture.

In some embodiments, cryopreserving the cell(s) comprises: mixing a composition comprising the cell(s) and a buffer, e.g., PBS 1:1 with a composition comprising 40 mg/mL albumin, e.g., human albumin, 25 mg/mL Dextran, e.g., Dextran 40, 12.5 mg/mL glucose and 55 mg/mL DMSO.

In some embodiments, the composition comprising the cell(s) and the buffer, e.g., . PBS, comprises from or from about 2×10⁷ to or to about 2×10⁹ cells/mL. In some embodiments, the composition comprising the cell(s) and the buffer, e.g., PBS, comprises 2×10⁸ cells/mL. In some embodiments, the composition comprising the cell(s) and the buffer, e.g., PBS, comprising about 2×10⁸ cells/mL.

In some embodiments, cryopreserving the cell(s) comprising mixing: the cell(s), a buffer, e.g., PBS, albumin, e.g., human albumin, Dextran, e.g., Dextran 40, and DMSO; and freezing the mixture.

In some embodiments, the mixture comprises from or from about 1×10⁷ to or to about 1×10⁹ cells/mL. In some embodiments, the mixture comprises 1×10⁸ cells/mL. In some embodiments, the mixture comprises about 1×10⁸ cells/mL.

Suitable ranges for albumin, Dextran, and DMSO are set forth above.

In some embodiments, the composition is frozen at or below−135° C.

In some embodiments, the composition is frozen at a controlled rate.

### Pharmaceutical Compositions

Provided herein are pharmaceutical compositions comprising the natural killer cells described herein and dosage units of the pharmaceutical compositions described herein.

In some cases, the dosage unit comprises between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion.

Pharmaceutical compositions typically include a pharmaceutically acceptable carrier. As used herein the language “pharmaceutically acceptable carrier” includes saline, solvents. dispersion media, coatings, antibacterial and antifungal agents, isotonic and absorption delaying agents, and the like, compatible with pharmaceutical administration.

In some embodiments, the pharmaceutical composition comprises: a) natural killer cell(s) described herein; and b) a cryopreservation composition.

Suitable cryopreservation compositions are described herein.

In some embodiments, the composition is frozen. In some embodiments, the composition has been frozen for at least three months, e.g., at least six months, at least nine months, at least 12 months, at least 15 months, at least 18 months, at least 24 months, or at least 36 months.

In some embodiments, at least 60%, e.g., at least 70%, at least 80%, at least 90% at least 95%, at least 99%, or 100% of the natural killer cells are viable after being thawed.

In some embodiments, the pharmaceutical composition comprises: a) a cryopreservation composition described herein; and b) therapeutic cell(s).

In some embodiments, the therapeutic cell(s) are animal cell(s). In some embodiments, the therapeutic cell(s) are human cell(s).

In some embodiments, the therapeutic cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein, e.g., the CAR-NKs described herein.

In some embodiments, the pharmaceutical composition further comprises: c) a buffer solution. Suitable buffer solutions are described herein, e.g., as for cryopreservation compositions.

In some embodiments, the pharmaceutical composition comprises from or from about

1×10⁷ to or to about 1×10⁹ cells/mL. In some embodiments, the pharmaceutical composition comprises 1×10⁸ cells/mL. In some embodiments, the pharmaceutical composition comprises about 1×10⁸ cells/mL.

In some embodiments, the pharmaceutical composition further comprises an antibody or antigen binding fragment thereof, e.g., an antibody described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents: antibacterial agents such as benzyl alcohol or methyl parabens: antioxidants such as ascorbic acid or sodium bisulfite: chelating agents such as ethylenediaminetetraacetic acid: buffers such as acetates, citrates or phosphates and agents for the adjustment of tonicity such as sodium chloride or dextrose, pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required. followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

### Methods of Treatment

The NK cells described herein, e.g., the CAR-NK cells described herein, find use for treating cancer or other proliferative disorders.

Thus, also provided herein are methods of treating a patient suffering from a disorder, e.g., a disorder associated with a cancer, e.g., a HER2+ cancer, comprising administering the NK cells, e.g., the NK cells described herein, e.g., the CAR-NK cells described herein.

Also provided herein are methods of preventing, reducing and/or inhibiting the recurrence, growth, proliferation, migration and/or metastasis of a cancer cell or population of cancer cells in a subject in need thereof, comprising administering the NK cells, e.g., the NK cells described herein, e.g., the CAR-NK cells described herein.

Also provided herein are methods of enhancing, improving, and/or increasing the response to an anticancer therapy in a subject in need thereof, comprising administering the NK cells, e.g., the NK cells described herein, e.g., the CAR-NK cells described herein.

Also provided herein are methods for inducing the immune system in a subject in need thereof comprising administering the NK cells, e.g., the NK cells described herein, e.g., the CAR-NK cells described herein.

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. Generally, the methods include administering a therapeutically effective amount of a treatment as described herein, to a subject who is in need of, or who has been determined to be in need of, such treatment. In some embodiments, the methods include administering a therapeutically effective amount of a treatment comprising NK cells, e.g., CAR-NK cells described herein.

As used herein, the terms “treatment,” “treat.” and “treating” refer to reversing, alleviating, delaying the onset of, or inhibiting the progress of a disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis. increased survival, and/or decreased morbidity and mortality, among other things. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “inhibition”, as it relates to cancer and/or cancer cell proliferation, refer to the inhibition of the growth, division, maturation or viability of cancer cells, and/or causing the death of cancer cells, individually or in aggregate with other cancer cells, by cytotoxicity, nutrient depletion, or the induction of apoptosis.

As used herein, “delaying” development of a disease or disorder, or one or more symptoms thereof, means to defer, hinder, slow, retard, stabilize and/or postpone development of the disease, disorder, or symptom thereof. This delay can be of varying lengths of time, depending on the history of the disease and/or subject being treated. As is evident to one skilled in the art, a sufficient or significant delay can, in effect, encompass prevention, in that the subject does not develop the disease, disorder, or symptom thereof. For example, a method that “delays” development of cancer is a method that reduces the probability of disease development in a given time frame and/or reduces extent of the disease in a given time frame, when compared to not using the method. Such comparisons may be based on clinical studies, using a statistically significant number of subjects.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject and/or before a certain stage of the disease (e.g., administration of a therapeutic substance to a subject with a cancer that has not yet metastasized). The subject may be an individual at risk of developing the disease or disorder, or at risk of disease progression, e.g., cancer metastasis. Such as an individual who has one or more risk factors known to be associated with development or onset of the disease or disorder. For example, an individual may be have mutations associated with the development or progression of a cancer. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

An “increased” or “enhanced” amount (e.g., with respect to antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15. 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5. 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

### Disorders

Methods and manufactured compositions disclosed herein find use in targeting a number of disorders, such as cellular proliferative disorders. A benefit of the approaches herein is that allogenic cells are used to target specific cells. Unlike previous therapies, such as chemotherapy or radiotherapy, using the approaches and pharmaceutical compositions herein, one is able to specifically target cells exhibiting detrimental proliferative activity, potentially without administering a systemic drug or toxin that impacts proliferating cells indiscriminately.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix.

lung, prostate, breast. head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev, in Oncol./Hemotol. 11:267-97): lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

In some embodiments, the cancer is selected from the group consisting of: acute lymphoblastic leukemia (ALL), acute myeloid leukemia (AML), adrenocortical carcinoma. Kaposi sarcoma, AIDS-related lymphoma, primary CNS lymphoma, anal cancer, appendix cancer, astrocytoma, typical teratoid/rhabdoid tumor, basal cell carcinoma, bile duct cancer, bladder cancer, bone cancer, brain tumor, breast cancer, bronchial tumor, Burkitt lymphoma, carcinoid, cardiac tumors, medulloblastoma, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric cancer, gastrointestinal carcinoid tumor, gastrointestinal stromal tumors (GIST), germ cell tumors, gestational trophoblastic disease, hairy cell leukemia, head and neck cancer, heart tumor. hepatocellular cancer, histiocytosis, Hodgkin lymphomas, hypopharyngeal cancer, intraocular melanoma, islet cell tumors, pancreatic neuroendocrine tumors, kidney (renal cell) carcinoma, Langerhans cell histiocytosis, laryngeal cancer, leukemia, lip and oral cavity cancer, liver cancer, lung cancer (e.g., non-small cell lung cancer, small cell lung cancer, pleuropulmonary blastoma, and tracheobronchial tumor), lymphoma, male breast cancer, malignant fibrous histiocytoma of bone, melanoma, Merkel cell carcinoma, mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, myeloproliferative neoplasms, nasal cavity and paranasal sinus cancer, nasopharyngeal cancer, neuroblastoma, non-Hodgkin lymphoma, oral cancer, lip and oral cavity cancer, oropharyngeal cancer, osteosarcoma, malignant fibrous histiocytoma, ovarian cancer, pancreatic cancer, pancreatic neuroendocrine tumors, papillomatosis, paraganglioma, paranasal sinus and nasal cavity cancer, parathyroid cancer, penile cancer, pharyngeal cancer, pheochromocytomas, pituitary tumor, plasma cell neoplasm, multiple myeloma, pleuropulmonary blastoma, pregnancy and breast cancer, primary central nervous system lymphoma, primary peritoneal cancer, prostate cancer, rectal cancer, recurrent cancer, renal cell cancer, retinoblastoma, rhabdomyosarcoma, salivary gland cancer, sarcoma (e.g., childhood rhabdomyosarcoma, childhood vascular tumors, Ewing sarcoma, Kaposi sarcoma, osteosarcoma, soft tissue sarcoma, uterine sarcoma), Sezary syndrome, skin cancer, small intestine cancer, soft tissue sarcoma, squamous cell carcinoma, squamous neck cancer, stomach cancer, T-cell lymphomas, testicular cancer, throat cancer, nasopharyngeal cancer, oropharyngeal cancer, hypopharyngeal cancer, thryomoma and thymic carcinomas, thyroid cancer, tracheobronchial tumors, transitional cell cancer of the renal pelvis and ureter, urethral cancer, uterine cancer, uterine sarcoma, vaginal cancer, vascular tumors, vulvar cancer, and Wilms tumor.

In some embodiments, the cancer is a solid tumor.

In some embodiments, the cancer is metastatic.

In some embodiments, the cancer is a HER2+ cancer.

In some embodiments, the HER2+ cancer is selected from the group consisting of bladder cancer, breast adenocarcinoma, colorectal adenocarcinoma, non-small cell lung cancer, esophageal cancer, cervix squamous cancer, stomach adenocarcinoma, cholangiocarcinoma, ovary cancer, renal papillary cell carcinoma, and combinations thereof.

In some embodiments, the HER2+ cancer is selected from the group consisting of breast cancer, gastric cancer, and ovarian cancer.

In some embodiments, the HER2+ cancer is breast cancer. In some embodiments, the HER2+ cancer is gastric cancer. In some embodiments, the HER2+ cancer is ovarian cancer.

### Patients

Suitable patients for the compositions and methods herein include those who are suffering from, who have been diagnosed with, or who are suspected of having a cellular proliferative and/or differentiative disorder, e.g., a cancer. Patients subjected to technology of the disclosure herein generally respond better to the methods and compositions herein, in part because the pharmaceutical compositions are allogeneic and target cells identified by the antigen binding domain, rather than targeting proliferating cells generally. As a result, there is less off-target impact and the patients are more likely to complete treatment regimens without substantial detrimental off-target effects.

In some embodiments, the methods of treatment provided herein may be used to treat a subject (e.g., human, monkey, dog, cat, mouse) who has been diagnosed with or is suspected of having a cellular proliferative and/or differentiative disorder, e.g., a cancer. In some embodiments, the subject is a mammal. In some embodiments, the subject is a human.

As used herein, a subject refers to a mammal, including, for example, a human.

In some embodiments, the mammal is selected from the group consisting of an armadillo, an ass, a bat, a bear, a beaver, a cat, a chimpanzee, a cow, a coyote, a deer, a dog, a dolphin, an elephant, a fox, a panda, a gibbon, a giraffe, a goat, a gopher, a hedgehog, a hippopotamus, a horse, a humpback whale, a jaguar, a kangaroo, a koala, a leopard, a lion, a llama, a lynx, a mole, a monkey, a mouse, a narwhal, an orangutan, an orca, an otter, an ox, a pig, a polar bear, a porcupine, a puma, a rabbit, a raccoon, a rat, a rhinoceros, a sheep, a squirrel, a tiger, a walrus, a weasel, a wolf, a zebra, a goat, a horse, and combinations thereof.

In some embodiments, the mammal is a human.

The subject, e.g., the human subject, can be a child, e.g., from or from about ( ) to or to about 14 years in age. The subject can be a youth, e.g., from or from about 15 to or to about 24 years in age. The subject can be an adult, e.g., from or from about 25 to or to about 64 years in age. The subject can be a senior, e.g. 65+ years in age.

In some embodiments, the subject may be a human who exhibits one or more symptoms associated with a cellular proliferative and/or differentiative disorder, e.g., a cancer, e.g., a tumor. Any of the methods of treatment provided herein may be used to treat cancer at various stages. By way of example, the cancer stage includes but is not limited to early, advanced, locally advanced, remission, refractory, reoccurred after remission and progressive. In some embodiments, the subject is at an early stage of a cancer. In other embodiments, the subject is at an advanced stage of cancer. In various embodiments, the subject has a stage I, stage II, stage III or stage IV cancer. The methods of treatment described herein can promote reduction or retraction of a tumor, decrease or inhibit tumor growth or cancer cell proliferation, and/or induce, increase or promote tumor cell killing. In some embodiments, the subject is in cancer remission. The methods of treatment described herein can prevent or delay metastasis or recurrence of cancer.

In some embodiments, the subject has an Eastern Cooperative Oncology Group (ECOG) performance status of 0, 1, 2, 3, or 4 at the time of treatment. In some embodiments, the subject has an ECOG performance status of 0 to 4, 0 to 3, 0 to 2, 0 to 1, 1 to 4, 1 to 3, 1 to 2, 2 to 4, 2 to 3, or 3 to 4 at the time of treatment.

In some embodiments, the subject is at risk, or genetically or otherwise predisposed (e.g., risk factor), to developing a cellular proliferative and/or differentiative disorder, e.g., a cancer, that has or has not been diagnosed.

As used herein, an “at risk” individual is an individual who is at risk of developing a condition to be treated, e.g., a cellular proliferative and/or differentiative disorder, e.g., a cancer. Generally, an “at risk” subject may or may not have detectable disease, and may or may not have displayed detectable disease prior to the treatment methods described herein. “At risk” denotes that an individual has one or more so-called risk factors, which are measurable parameters that correlate with development of a disease or condition and are known in the art. For example, an at risk subject may have one or more risk factors, which are measurable parameters that correlate with development of cancer. A subject having one or more of these risk factors has a higher probability of developing cancer than an individual without these risk factor(s). In general, risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced the disease, and those whose risk is determined by analysis of genetic or biochemical markers.

In addition, the subject may be undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

In some embodiments, the subject has a cancer that expresses HER2.

In some embodiments, the patient is diagnosed with or has been diagnosed with a HER2+ cancer.

In some embodiments, the patient is diagnosed with or has been diagnosed with a HER2+ cancer by immunohistochemical staining of a biopsy or surgical sample of the cancer. In some embodiments, the patient is or has been diagnosed with a HER2+ cancer by fluorescent in situ hybridization of a biopsy or surgical sample of the cancer.

In some embodiments, the patient is diagnosed with or has been diagnosed with a HER2+ cancer according to ASCOR Guidelines, e.g., the 2018 ASCOR Guidelines, e.g., as described in Wolff et al., “Human Epidermal Growth Factor Receptor 2 Testing in Breast Cancer,” Arch Pathol Lab Med 142:1364-82 (2018), which is hereby incorporated by reference in its entirety

In some embodiments, the patient is diagnosed with or has been diagnosed with a HER2+ cancer with an IHC score of 2+, according to ASCOR Guidelines, e.g., the 2018

ASCOR Guidelines, e.g., as described in Wolff et al., “Human Epidermal Growth Factor Receptor 2 Testing in Breast Cancer,” Arch Pathol Lab Med 142:1364-82 (2018), which is hereby incorporated by reference in its entirety. In some embodiments, the patient is diagnosed with or has been diagnosed with a HER2+ cancer with an IHC score of 2+ within 12 months (e.g., within 12. 11. 10. 9. 8. 7. 6. 5. 4. 3. 2, or 1 months) of treatment.

In some embodiments, the patient has a cancer that is not HER2-addicted, but shows some degree of HER2-expression. In some embodiments, the patient is diagnosed with or has been diagnosed with a HER2-low cancer. In some embodiments, the HER2-low cancer has a HER2 expression classification of score 1+ or 2+ according to ASCOR Guidelines, e.g., the 2018 ASCOR Guidelines, e.g., as described in Wolff et al., “Human Epidermal Growth Factor Receptor 2 Testing in Breast Cancer,” Arch Pathol Lab Med 142:1364-82 (2018), which is hereby incorporated by reference in its entirety, but lacks HER2 amplification. In some embodiments, the HER2-low cancer harbors HER2 gain. In some embodiments, the HER2-low cancer does not harbor HER2 gain.

In some embodiments, the patient is diagnosed with or has been diagnosed with a HER2+ cancer by genetic analysis, e.g., by identifying a HER2 mutated cancer, e.g., a somatic mutation in the HER2 (ERBB2) gene.

In some embodiments, the patient has a cancer comprising one or more mutations set forth in Table 7, an insertion or deletion polymorphism in the HER2 gene, a copy number variation of the HER2 gene, a methylation mutation of the HER2 gene, or combinations thereof. In some embodiments, the mutation is a HER2 activating mutation.

In some embodiments, the patient has a chromosomal translocation associated with cancer, e.g., a HER2+ cancer. In some embodiments, the patient has a fusion gene associated with cancer, e.g., a HER+ cancer.

TABLE 7
HER2 (ERBB2) Mutations (relative to Human
Genome Assembly Reference Build GRCh38.p13
(ncbi.nlm.nih.gov/assembly/88331)

| Mutation (GRCh38) | Protein Position | Consequence |
|---|---|---|
| 17: 39711955: C > T | 310 | missense_variant |
| 17: 39723405: G > A | 678 | missense_variant |
| 17: 39723967: T > C | 755 | missense_variant |
| 17: 39725079: G > A | 842 | missense_variant |
| 17: 39711955: C > A | 310 | missense_variant |
| 17: 39724747: G > T | 777 | missense_variant |
| 17: 39724008: G > T | 769 | missense_variant |
| 17: 39724004: C > G | 767 | missense_variant |
| 17: 39725139: A > G | 862 | missense_variant |
| 17: 39724745: G > T | 776 | missense_variant |
| 17: 39723650: C > T | 733 | missense_variant |
| 17: 39724008: G > C | 769 | missense_variant |
| 17: 39724747: G > C | 777 | missense_variant |
| 17: 39725161: T > G | 869 | missense_variant |
| 17: 39710410: A > G | 277 | missense_variant |
| 17: 39710414: G > A | 278 | synonymous_variant |
| 17: 39711963: C > G | 313 | missense_variant |
| 17: 39723966: T > C | 755 | synonymous_variant |
| 17: 39724008: G > A | 769 | missense_variant |
| 17: 39726610: C > T | 974 | missense_variant |
| 17: 39727303: T > G | 1056 | synonymous_variant |
| 17: 39727784: C > G | 1170 | missense_variant |
| 17: 39706987: C > T | — | splice_region_variant |
| 17: 39708375: G > A | 94 | missense_variant |
| 17: 39708497: A > T | 134 | synonymous_variant |
| 17: 39708530: C > T | 145 | synonymous_variant |
| 17: 39709376: G > A | 166 | synonymous_variant |
| 17: 39709810: C > T | — | splice_region_variant |
| 17: 39710106: G > T | 222 | missense_variant |
| 17: 39710164: C > T | 241 | missense_variant |
| 17: 39710409: G > T | 277 | missense_variant |
| 17: 39710418: G > C | 280 | missense_variant |
| 17: 39712319: C > T | — | splice_region_variant |
| 17: 39712429: C > T | 377 | synonymous_variant |
| 17: 39715854: C > T | 476 | synonymous_variant |
| 17: 39716421: G > A | 545 | missense_variant |
| 17: 39716586: C > T | 573 | missense_variant |
| 17: 39717397: A > C | 605 | missense_variant |
| 17: 39717446: G > A | 622 | missense_variant |
| 17: 39719819: C > T | 644 | missense_variant |
| 17: 39719827: A > C | 647 | synonymous_variant |
| 17: 39723330: C > G | 653 | missense_variant |
| 17: 39723339: C > T | 656 | missense_variant |
| 17: 39723530: C > T | — | splice_region_variant |
| 17: 39724734: C > T | 772 | synonymous_variant |
| 17: 39724902: G > A | 828 | synonymous_variant |
| 17: 39725057: C > T | 834 | synonymous_variant |
| 17: 39725096: C > T | 847 | synonymous_variant |
| 17: 39725139: A > T | 862 | missense_variant |
| 17: 39725347: G > A | 890 | synonymous_variant |
| 17: 39725357: A > C | 894 | missense_variant |
| 17: 39725750: C > T | 923 | synonymous_variant |
| 17: 39726573: G > A | 962 | missense_variant |
| 17: 39726860: C > T | 1006 | missense_variant |
| 17: 39726905: G > C | 1021 | missense_variant |
| 17: 39727000: C > G | 1052 | synonymous_variant |
| 17: 39727847: G > A | 1191 | missense_variant |
| 17: 39727973: C > T | 1233 | missense_variant |
| 17: 39700256: G > T | 6 | missense_variant |
| 17: 39700298: C > T | 20 | synonymous_variant |
| 17: 39706995: A > T | 27 | missense_variant |
| 17: 39707022: C > T | 36 | missense_variant |
| 17: 39707032: C > T | 39 | missense_variant |
| 17: 39707033: C > T | 39 | synonymous_variant |
| 17: 39707063: C > T | 49 | synonymous_variant |
| 17: 39707070: G > A | 52 | missense_variant |
| 17: 39707076: C > T | 54 | stop_gained |
| 17: 39707093: C > A | 59 | missense_variant |
| 17: 39707114: C > A | 66 | synonymous_variant |
| 17: 39708350: C > G | 85 | synonymous_variant |
| 17: 39708354: G > A | 87 | missense_variant |
| 17: 39708386: G > A | 97 | synonymous_variant |
| 17: 39708397: T > G | 101 | missense_variant |
| 17: 39708403: G > A | 103 | missense_variant |
| 17: 39708406: G > A | 104 | missense_variant |
| 17: 39708459: C > T | 122 | missense_variant |
| 17: 39708460: C > T | 122 | missense_variant |
| 17: 39708507: C > T | 138 | missense_variant |
| 17: 39708508: G > A | 138 | missense_variant |
| 17: 39708510: G > A | 139 | missense_variant |
| 17: 39709322: C > A | 148 | synonymous_variant |
| 17: 39709326: A > G | 150 | missense_variant |
| 17: 39709340: G > C | 154 | missense_variant |
| 17: 39709343: C > T | 155 | synonymous_variant |
| 17: 39709352: C > T | 158 | synonymous_variant |
| 17: 39709375: C > T | 166 | missense_variant |
| 17: 39709391: C > T | 171 | synonymous_variant |
| 17: 39709394: C > G | 172 | missense_variant |
| 17: 39709419: C > T | 181 | missense_variant |
| 17: 39709421: C > G | 181 | synonymous_variant |
| 17: 39709423: C > T | 182 | missense_variant |
| 17: 39709427: G > C | 183 | synonymous_variant |
| 17: 39709447: G > A | 190 | missense_variant |
| 17: 39709449: G > A | 191 | missense_variant |
| 17: 39709824: T > C | 196 | missense_variant |
| 17: 39709825: C > T | 196 | missense_variant |
| 17: 39709845: C > T | 203 | missense_variant |
| 17: 39709850: C > A | 204 | stop_gained |
| 17: 39710087: G > T | 215 | splice_region_variant |
| 17: 39710097: G > A | 219 | missense_variant |
| 17: 39710099: C > A | 219 | synonymous_variant |
| 17: 39710103: G > A | 221 | missense_variant |
| 17: 39710111: C > A | 223 | synonymous_variant |
| 17: 39710120: C > T | 226 | synonymous_variant |
| 17: 39710154: G > A | 238 | missense_variant |
| 17: 39710182: C > T | 247 | missense_variant |
| 17: 39710184: A > G | 248 | missense_variant |
| 17: 39710191: C > T | 250 | missense_variant |
| 17: 39710209: C > T | — | splice_region_variant |
| 17: 39710418: G > A | 280 | missense_variant |
| 17: 39710433: C > T | 285 | missense_variant |
| 17: 39710454: G > C | 292 | missense_variant |
| 17: 39710458: C > T | 293 | missense_variant |
| 17: 39710480: C > T | 300 | splice_region_variant |
| 17: 39711940: C > G | 305 | missense_variant |
| 17: 39711950: G > A | 308 | synonymous_variant |
| 17: 39711952: G > C | 309 | missense_variant |
| 17: 39711954: T > G | 310 | missense_variant |
| 17: 39711962: C > G | 312 | synonymous_variant |
| 17: 39711981: A > T | 319 | missense_variant |
| 17: 39712006: G > A | 327 | missense_variant |
| 17: 39712012: A > T | 329 | missense_variant |
| 17: 39712014: C > T | 330 | missense_variant |
| 17: 39712044: C > G | 340 | missense_variant |
| 17: 39712323: G > T | 341 | splice_region_variant |
| 17: 39712361: G > — | 354 | frameshift_variant |
| 17: 39712431: G > A | 377 | synonymous_variant |
| 17: 39715322: G > C | 395 | missense_variant |
| 17: 39715328: C > T | 397 | synonymous_variant |
| 17: 39715340: G > A | 401 | synonymous_variant |
| 17: 39715344: C > T | 403 | synonymous_variant |
| 17: 39715352: G > C | 405 | missense_variant |
| 17: 39715445: G > T | — | splice_acceptor_variant |
| 17: 39715461: C > T | 413 | missense_variant |
| 17: 39715476: G > C | 418 | missense_variant |
| 17: 39715478: C > T | 419 | synonymous_variant |
| 17: 39715519: G > A | 432 | synonymous_variant |
| 17: 39715519: G > T | 432 | synonymous_variant |
| 17: 39715738: A > T | — | splice_acceptor_variant |
| 17: 39715744: G > A | 440 | missense_variant |
| 17: 39715766: G > C | 447 | missense_variant |
| 17: 39715783: C > A | 453 | missense_variant |
| 17: 39715804: G > A | 460 | missense_variant |
| 17: 39715830: C > T | 468 | synonymous_variant |
| 17: 39715836: T > G | 470 | missense_variant |
| 17: 39715844: A > T | 473 | missense_variant |
| 17: 39715867: C > — | 481 | frameshift_variant |
| 17: 39716306: G > A | 507 | missense_variant |
| 17: 39716354: C > T | 523 | missense_variant |
| 17: 39716365: C > G | 526 | synonymous_variant |
| 17: 39716377: C > T | 530 | synonymous_variant |
| 17: 39716393: C > T | 536 | missense_variant |
| 17: 39716401: G > A | 538 | synonymous_variant |
| 17: 39716418: G > T | 544 | missense_variant |
| 17: 39716552: C > T | 562 | missense_variant |
| 17: 39716592: C > T | 575 | missense_variant |
| 17: 39717326: G > A | 582 | missense_variant |
| 17: 39717351: A > G | 590 | missense_variant |
| 17: 39717358: C > T | 592 | synonymous_variant |
| 17: 39717367: C > A | 595 | missense_variant |
| 17: 39717376: C > T | 598 | synonymous_variant |
| 17: 39717406: C > T | 608 | synonymous_variant |
| 17: 39717439: G > A | 619 | synonymous_variant |
| 17: 39717442: G > — | 620 | frameshift_variant |
| 17: 39717466: C > G | 628 | missense_variant |
| 17: 39717480: C > T | 633 | missense_variant |
| 17: 39719785: A > T | — | splice_acceptor_variant |
| 17: 39719786: G > C | — | splice_acceptor_variant |
| 17: 39719799: G > A | 637 | synonymous_variant |
| 17: 39719811: C > T | 641 | synonymous_variant |
| 17: 39719820: C > T | 644 | synonymous_variant |
| 17: 39719834: G > C | 649 | missense_variant |
| 17: 39723323: C > G | 651 | missense_variant |
| 17: 39723328: G > T | 652 | synonymous_variant |
| 17: 39723334: C > A | 654 | synonymous_variant |
| 17: 39723335: A > G | 655 | missense_variant |
| 17: 39723350: G > C | 660 | missense_variant |
| 17: 39723351: G > A | 660 | missense_variant |
| 17: 39723356: C > G | 662 | missense_variant |
| 17: 39723356: C > T | 662 | synonymous_variant |
| 17: 39723357: T > A | 662 | missense_variant |
| 17: 39723360: T > C | 663 | missense_variant |
| 17: 39723368: G > T | 666 | missense_variant |
| 17: 39723373: G > C | 667 | missense_variant |
| 17: 39723376: G > T | 668 | synonymous_variant |
| 17: 39723397: C > G | 675 | missense_variant |
| 17: 39723402: G > A | 677 | missense_variant |
| 17: 39723412: G > A | 680 | synonymous_variant |
| 17: 39723449: G > A | 693 | missense_variant |
| 17: 39723541: G > T | 697 | missense_variant |
| 17: 39723562: G > A | 704 | missense_variant |
| 17: 39723567: G > A | 705 | synonymous_variant |
| 17: 39723577: C > G | 709 | missense_variant |
| 17: 39723582: G > A | 710 | synonymous_variant |
| 17: 39723594: C > T | 714 | synonymous_variant |
| 17: 39723597: G > T | 715 | synonymous_variant |
| 17: 39723603: G > C | 717 | missense_variant |
| 17: 39723908: C > T | — | splice_region_variant |
| 17: 39723909: C > T | — | splice_region_variant |
| 17: 39723916: T > A | 738 | missense_variant |
| 17: 39723941: G > A | 746 | synonymous_variant |
| 17: 39723966: T > A | 755 | missense_variant |
| 17: 39723967: T > G | 755 | missense_variant |
| 17: 39723970: G > A | 756 | missense_variant |
| 17: 39723974: A > C | 757 | missense_variant |
| 17: 39724002: A > T | 767 | missense_variant |
| 17: 39724010: C > T | 769 | splice_region_variant |
| 17: 39724743: G > ATCT | 775 | inframe_insertion |
| 17: 39724744: G > A | 776 | missense_variant |
| 17: 39724761: T > C | 781 | synonymous_variant |
| 17: 39724768: C > T | 784 | missense_variant |
| 17: 39724774: C > T | 786 | synonymous_variant |
| 17: 39724780: A > T | 788 | missense_variant |
| 17: 39724784: G > A | 789 | missense_variant |
| 17: 39724822: C > T | 802 | missense_variant |
| 17: 39724874: C > T | 819 | missense_variant |
| 17: 39724879: G > C | 821 | missense_variant |
| 17: 39724882: C > T | 822 | synonymous_variant |
| 17: 39725070: G > A | 839 | missense_variant |
| 17: 39725088: G > A | 845 | missense_variant |
| 17: 39725122: C > G | 856 | missense_variant |
| 17: 39725172: G > A | 873 | missense_variant |
| 17: 39725174: C > T | 873 | synonymous_variant |
| 17: 39725177: G > A | 874 | synonymous_variant |
| 17: 39725187: C > T | 878 | missense_variant |
| 17: 39725192: A > G | 879 | synonymous_variant |
| 17: 39725344: G > A | 889 | missense_variant |
| 17: 39725363: C > T | 896 | missense_variant |
| 17: 39725374: C > A | 899 | missense_variant |
| 17: 39725374: C > G | 899 | missense_variant |
| 17: 39725400: A > G | 908 | missense_variant |
| 17: 39725726: G > C | 915 | synonymous_variant |
| 17: 39725729: G > A | 916 | missense_variant |
| 17: 39725756: G > A | 925 | synonymous_variant |
| 17: 39725769: G > A | 930 | missense_variant |
| 17: 39725797: A > G | 939 | missense_variant |
| 17: 39725808: C > T | 943 | stop_gained |
| 17: 39725825: C > A | 948 | synonymous_variant |
| 17: 39725857: C > T | — | splice_region_variant |
| 17: 39726577: C > G | 963 | missense_variant |
| 17: 39726594: T > C | 969 | missense_variant |
| 17: 39726596: C > G | 969 | missense_variant |
| 17: 39726633: G > T | 982 | missense_variant |
| 17: 39726651: G > A | 988 | missense_variant |
| 17: 39726821: G > A | 993 | missense_variant |
| 17: 39726829: C > T | 995 | synonymous_variant |
| 17: 39726881: G > C | 1013 | missense_variant |
| 17: 39726933: T > G | 1030 | missense_variant |
| 17: 39726941: C > — | 1033 | frameshift_variant |
| 17: 39726941: C > G | 1033 | missense_variant |
| 17: 39726958: C > T | 1038 | synonymous_variant |
| 17: 39726959: G > A | 1039 | missense_variant |
| 17: 39726980: A > T | 1046 | missense_variant |
| 17: 39726987: G > A | 1048 | missense_variant |
| 17: 39727294: G > A | — | splice_acceptor_variant |
| 17: 39727294: G > C | — | splice_acceptor_variant |
| 17: 39727305: G > T | 1057 | missense_variant |
| 17: 39727308: A > C | 1058 | missense_variant |
| 17: 39727344: C > T | 1070 | missense_variant |
| 17: 39727366: C > T | 1077 | synonymous_variant |
| 17: 39727370: G > A | 1079 | missense_variant |
| 17: 39727373: G > T | 1080 | missense_variant |
| 17: 39727450: C > — | 1105 | frameshift_variant |
| 17: 39727466: C > T | 1111 | missense_variant |
| 17: 39727475: G > C | 1114 | missense_variant |
| 17: 39727492: C > T | 1119 | synonymous_variant |
| 17: 39727533: G > C | 1133 | missense_variant |
| 17: 39727542: A > G | 1136 | missense_variant |
| 17: 39727728: C > T | 1151 | missense_variant |
| 17: 39727732: C > T | 1152 | synonymous_variant |
| 17: 39727752: C > T | 1159 | missense_variant |
| 17: 39727755: C > T | 1160 | missense_variant |
| 17: 39727805: A > G | 1177 | missense_variant |
| 17: 39727825: C > T | 1183 | synonymous_variant |
| 17: 39727838: G > A | 1188 | missense_variant |
| 17: 39727839: G > A | 1188 | missense_variant |
| 17: 39727846: C > T | 1190 | synonymous_variant |
| 17: 39727867: G > C | 1197 | missense_variant |
| 17: 39727871: C > A | 1199 | missense_variant |
| 17: 39727892: C > T | 1206 | stop_gained |
| 17: 39727898: C > T | 1208 | missense_variant |
| 17: 39727904: C > T | 1210 | missense_variant |
| 17: 39727965: G > — | 1230 | frameshift_variant |
| 17: 39727974: C > T | 1233 | missense_variant |
| 17: 39727976: C > T | 1234 | missense_variant |
| 17: 39728002: G > A | 1242 | synonymous_variant |
| 17: 39728006: G > C | 1244 | missense_variant |
| 17: 39728023: G > C | 1249 | synonymous_variant |
| 17: 39728032: C > T | 1252 | synonymous_variant |

In some embodiments, the HER2+ cancer is a HER2+ breast cancer, gastric cancer, gastroesophageal (GEJ) cancer, or lung cancer.

In some embodiments, the breast cancer is in situ (noninvasive). In some embodiments, the breast cancer is invasive. In some embodiments, the breast cancer is metastatic.

In some embodiments, the breast cancer is a carcinoma (e.g., an adenocarcinoma). In some embodiments, the breast cancer is ductal carcinoma. In some embodiments, the breast cancer is invasive lobular carcinoma. In some embodiments, the breast cancer is inflammatory breast cancer. In some embodiments, the breast cancer is Paget's disease of the breast. In some embodiments, the breast cancer is phyllodes tumor.

In some embodiments, the gastric cancer is an adenocarcinoma. In some embodiments, the gastric cancer is an intestinal adenocarcinoma. In some embodiments, the gastric cancer is a diffuse adenocarcinoma. In some embodiments, the gastric cancer is a lymphoma. In some cases, the gastric cancer is a mucosa-associated lymphoid tissue lymphoma. In some embodiments, the gastric cancer is a gastrointestinal stromal tumor. In some embodiments, the gastric cancer is a gastric carcinoid tumor. In some embodiments, the gastric cancer is a Type I, Type II, or Type III ECL-cell carcinoid. In some embodiments, the gastric cancer is a hereditary diffuse gastric cancer.

In some embodiments, the GEJ cancer is a GEJ adenocarcinoma.

In some embodiments, the lung cancer is non-small cell lung cancer (NSCLC), e.g., metastatic NSCLC. In some embodiments, the patient has a NSCLC with a HER2 activating mutation.

In some embodiments, the patient is refractory to or has a recurrence of after treatment, e.g., with trastuzumab or a biosimilar thereof.

In some embodiments, the patient is refractory to or has a recurrence after receiving 1 or more prior systemic therapies. In some embodiments, the patient is refractory to or has a recurrence after receiving 2 or more prior systemic therapies.

In some embodiments, when the patient has been diagnosed with a IHC 3+ or IHC 2+/ISH+ cancer, according to ASCOR Guidelines, e.g., the 2018 ASCOR Guidelines, e.g., as described in Wolff et al., “Human Epidermal Growth Factor Receptor 2 Testing in Breast Cancer,” Arch Pathol Lab Med 142:1364-82 (2018), which is hereby incorporated by reference in its entirety, the patient is refractory to or has a recurrence after receiving previous treatment with a HER2-targeting therapy.

In some embodiments, when the patient has been diagnosed with a HER2 expressing cancer (e.g., breast cancer) that is IHC 1+ or IHC 2+/ISH−, according to ASCOR Guidelines, e.g., the 2018 ASCOR Guidelines, e.g., as described in Wolff et al., “Human Epidermal Growth Factor Receptor 2 Testing in Breast Cancer,” Arch Pathol Lab Med 142:1364-82 (2018), which is hereby incorporated by reference in its entirety, the patient's cancer is unresectable, metastatic, or the patient is refractory to or has a recurrence after receiving previous treatment.

In some embodiments, the patient is refractory to or has a recurrence after treatment with pertuzumab (or FDA-approved biosimilar thereof), trastuzumab (or FDA-approved biosimilar thereof) and docetaxel (or pharmaceutically acceptable salt thereof). In some embodiments, the pertuzumab (or FDA-approved biosimilar thereof) is administered at 840 mg IV day I followed by 420 mg IV. In some embodiments, the trastuzumab (or FDA-approved biosimilar thereof) is administered at 7 mg/kg IV day I followed by 6 mg/kg IV day 1 every 21 days. In some embodiments, the trastuzumab (or FDA-approved biosimilar thereof) is administered as a trastuzumab (or FDA-approved biosimilar thereof) and hyaluronidase-oysk injection for subcutaneous administration. In some embodiments, the docetaxel (or pharmaceutically acceptable salt thereof) is administered at 75-100 mg/m2 IV day 1 cycled every 21 days.

In some embodiments, the patient is refractory to or has a recurrence after treatment with pertuzumab (or FDA-approved biosimilar thereof), trastuzumab (or FDA-approved biosimilar thereof), and paclitaxel (or pharmaceutically acceptable salt thereof). In some embodiments, the pertuzumab (or FDA-approved biosimilar thereof) is administered at 840 mg IV day I followed by 420 mg IV, cycled every 21 days. In some embodiments, the trastuzumab (or FDA-approved biosimilar thereof) is administered at 4 mg/kg IV day 1 followed by 2 mg/kg IV weekly or 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration. In some embodiments, the paclitaxel (or pharmaceutically acceptable salt thereof) is administered at 80 mg/m2 IV day 1 weekly or 175 mg/m2 day 1 cycled every 21 days.

In some embodiments, the patient is refractory to or has a recurrence after treatment with tucatinib (or pharmaceutically acceptable salt thereof), trastuzumab (or FDA-approved biosimilar thereof), and capecitabine (or pharmaceutically acceptable salt thereof). In some embodiments, the tucatinib (or FDA-approved biosimilar thereof) is administered at 300 mg orally twice daily on days 1-21. In some embodiments, the trastuzumab (or FDA-approved biosimilar thereof) is administered at 8 mg/kg IV day I followed by 6 mg/kg IV day 1 every 21 days. In some embodiments, the trastuzumab (or FDA-approved biosimilar thereof) is administered as a trastuzumab (or FDA-approved biosimilar thereof) and hyaluronidase-oysk injection for subcutaneous administration. In some embodiments, the capecitabine (or FDA-approved biosimilar thereof) is administered at 1000 mg/m2 orally twice daily on days 1-14. In some embodiments, the administration of tucatinib (or FDA-approved biosimilar thereof), trastuzumab (or FDA-approved biosimilar thereof), and capecitabine (or pharmaceutically acceptable salt thereof) is cycled every 21 days.

In some embodiments, the patient is refractory to or has a recurrence after treatment with ado-trastuzumab emtansine (T-DM1) (or FDA-approved biosimilar thereof). In some embodiments, the ado-trastuzumab emtansine (T-DM1) (or FDA-approved biosimilar thereof) is administered at 3.6 mg/kg IV day 1, cycled every 21 days.

In some embodiments, the patient is refractory to or has a recurrence after treatment with fam-trastuzumab deruxtecan-nxki (or FDA-approved biosimilar thereof). In some embodiments, the fam-trastuzumab deruxtecan-nxki (or FDA-approved biosimilar thereof) is administered at 5.4 mg/kg IV day 1, cycled every 21 days.

In some embodiments, the patient is refractory to or has a recurrence after treatment with paclitaxel/carboplatin (or pharmaceutically acceptable salts thereof) and trastuzumab (or FDA-approved biosimilar thereof). In some embodiments, the carboplatin/paclitaxel (or pharmaceutically acceptable salts thereof) is administered at AUC 6 IV day 1 carboplatin and 175 mg/m2 IV day 1 paclitaxel), cycled every 21 days. In some embodiments, the trastuzumab (or FDA-approved biosimilar thereof) is administered 4 mg/kg IV day I followed by 2 mg/kg IV weekly or 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with paclitaxel/carboplatin (or pharmaceutically acceptable salts thereof) and trastuzumab (or FDA-approved biosimilar thereof). In some embodiments, the carboplatin/paclitaxel (or pharmaceutically acceptable salts thereof) is administered at AUC 2 IV carboplatin and 80 mg/m2 IV day I paclitaxel), days 1, 8, and 15, cycled every 28 days. In some embodiments, the trastuzumab (or FDA-approved biosimilar thereof) is administered 4 mg/kg IV day 1 followed by 2 mg/kg IV weekly or 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with trastuzumab (or FDA-approved biosimilar thereof) and paclitaxel (or pharmaceutically acceptable salt thereof). In some embodiments, the paclitaxel (or pharmaceutically acceptable salt thereof) is administered at 175 mg/m2 IV day 1 cycled every 21 days or 80-90 mg/m2 IV day 1 weekly. In some embodiments, the trastuzumab (or FDA-approved biosimilar thereof) is administered 4 mg/kg IV day I followed by 2 mg/kg IV weekly or 8 mg/kg IV day I followed by 6 mg/kg IV day 1 every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with trastuzumab (or FDA-approved biosimilar thereof) and docetaxel (or pharmaceutically acceptable salt thereof). In some embodiments, the docetaxel (or pharmaceutically acceptable salt thereof) is administered at 80-100 mg/m2 IV day 1 cycled every 21 days or 35 mg/m2 IV days 1, 8, and 15 weekly. In some embodiments, the trastuzumab (or FDA-approved biosimilar thereof) is administered 4 mg/kg IV day I followed by 2 mg/kg IV weekly or 8 mg/kg IV day 1 followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with trastuzumab (or FDA-approved biosimilar thereof) and vinorelbine (or pharmaceutically acceptable salt thereof). In some embodiments, the vinorelbine (or pharmaceutically acceptable salt thereof) is administered at 25 mg/m2 IV day I weekly or 20-35 mg/m2 IV days I and 8, cycled every 21 days, or 25-30 mg/m2 IV days 1, 8, and 15, cycled every 28 days. In some embodiments, the trastuzumab (or FDA-approved biosimilar thereof) is administered 4 mg/kg IV day I followed by 2 mg/kg IV weekly or 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with trastuzumab (or FDA-approved biosimilar thereof) and capecitabine (or pharmaceutically acceptable salt thereof). In some embodiments, the capecitabine (or pharmaceutically acceptable salt thereof) is administered at 1000-1250 mg/m2 PO twice daily days 1-14 cycled every 21 days. In some embodiments, the trastuzumab (or FDA-approved biosimilar thereof) is administered 4 mg/kg IV day I followed by 2 mg/kg IV weekly or 8 mg/kg IV day I followed by 6 mg/kg IV day 1 every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with lapatinib (or pharmaceutically acceptable salt thereof) and capecitabine (or pharmaceutically acceptable salt thereof). In some embodiments, the lapatinib (or pharmaceutically acceptable salt thereof) is administered at 1250 mg/m2 PO daily days 1-21. In some embodiments, the capecitabine (or pharmaceutically acceptable salt thereof) is administered at 1000 mg/m2 PO twice daily days 1-14, cycled every 21 days.

In some embodiments, the patient is refractory to or has a recurrence after treatment with trastuzumab (or FDA-approved biosimilar thereof) and lapatinib (or pharmaceutically acceptable salt thereof). In some embodiments, the administered (or pharmaceutically acceptable salt thereof) is administered at 1000 mg/m2 PO daily. In some embodiments, the trastuzumab (or FDA-approved biosimilar thereof) is administered 4 mg/kg IV day 1 followed by 2 mg/kg IV weekly or 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with neratinib (or pharmaceutically acceptable salt thereof) and capecitabine (or pharmaceutically acceptable salt thereof). In some embodiments, the neratinib is administered at 240 mg/m2 PO daily on days 1-21. Ins ome embodiments, the capecitabine is administered at 750 mg/m2 PO twice daily on days 1-14, cycled every 21 days.

In some embodiments, the patient has an oxygen saturation level measured via pulse oximeter of at least 92%, 93%, 94%, 95%, 96%, 97%, 98%, or 99% within 1, 5, 10, 15, 20. 30, 45, 60, or 90 minutes, or within 2, 3, 4, 5, 6, 7, 8, 9, 10, 11, 12, or 18 hours, or within 1 day, 1 week, or 1 month of treatment.

In some embodiments, the patient has a left ventricular ejection fraction (LVEF) of at least 50%, 51%, 52%, 53%, 54%, 55%, 56%, 57%, 58%, 59%, 60%, 61%, 62%, 63%, 64%, or 65%, measured within 1 day, 1 week, or 1 month of treatment.

In some embodiments, the patient has an absolute neutrophil count (ANC) of at least 1000/mm³ (1.0×109/L), 1500/mm³, 2000/mm³, 2500/mm³, 3000/mm³, 3500/mm³, 4000/mm³, 4500/mm³. 5000/mm³, 5500/mm³, or 6000/mm³, measured within 1 day, 1 week, or 1 month of treatment. In some embodiments, the patient has an ANC of 1000/mm³ to 6000/mm³, 1000/mm³ to 5500/mm³, 1000/mm³ to 5000/mm³, 1000/mm³ to 4500/mm³, 1000/mm³ to 4000/mm³, 1000/mm³ to 3500/mm³, 1000/mm³ to 3000/mm³, 1000/mm³ to 2500/mm³. 1000/mm³ to 2000/mm³, 1000/mm³ to 1500/mm³, 1500/mm³ to 6000/mm³, 1500/mm³ to 5500/mm³, 1500/mm³ to 5000/mm³, 1500/mm³ to 4500/mm³, 1500/mm³ to 4000/mm³. 1500/mm³ to 3500/mm³. 1500/mm³ to 3000/mm³. 1500/mm³ to 2500/mm³. 1500/mm³ to 2000/mm³. 2000/mm³ to 6000/mm³, 2000/mm³ to 5500/mm³, 2000/mm³ to 5000/mm³. 2000/mm³ to 4500/mm³, 2000/mm³ to 4000/mm³, 2000/mm³ to 3500/mm³, 2000/mm³ to 3000/mm³. 2000/mm³ to 2500/mm³, 2500/mm³ to 6000/mm³, 2500/mm³ to 5500/mm³. 2500/mm³ to 5000/mm³, 2500/mm³ to 4500/mm³, 2500/mm³ to 4000/mm³, 2500/mm³ to 3500/mm³, 2500/mm³ to 3000/mm³, 3000/mm³ to 6000/mm³, 3000/mm³ to 5500/mm³. 3000/mm³ to 5000/mm³, 3000/mm³ to 4500/mm³, 3000/mm³ to 4000/mm³, 3000/mm³ to 3500/mm³. 3500/mm³ to 6000/mm³, 3500/mm³ to 5500/mm³, 3500/mm³ to 5000/mm³.

3500/mm³ to 4500/mm³, 3500/mm³ to 4000/mm³, 4000/mm³ to 6000/mm³, 4000/mm³ to 5500/mm³, 4000/mm³ to 5000/mm³, 4000/mm³ to 4500/mm³, 4500/mm³ to 6000/mm³. 4500/mm³ to 5500/mm³, 4500/mm³ to 5000/mm³, 5000/mm³ to 6000/mm³, 5000/mm³ to 5500/mm³, or 5500/mm³ to 6000/mm³, measured within 1 day, 1 week, or 1 month of treatment.

In some embodiments, the patient has a platelet count of at least 75000/mm³ (75×109/L), 100000/mm³, 125000/mm³, 150000/mm³, 175000/mm³, 200000/mm³, 225000/mm³, 250000/mm³, 275000/mm³, 300000/mm³, 325000/mm³, 350000/mm³, 375000/mm³, or 400000/mm³, measured within 1 day, 1 week, or 1 month of treatment. In some embodiments, the patient has a platelet count of 75000/mm³ to 400000/mm³, 75000/mm³ to 375000/mm³, 75000/mm³ to 350000/mm³, 75000/mm³ to 300000/mm³, 75000/mm³ to 275000/mm³, 75000/mm³ to 250000/mm³, 75000/mm³ to 225000/mm³, 75000/mm³ to 200000/mm³, 75000/mm³ to 175000/mm³, 75000/mm³ to 150000/mm³, 75000/mm³ to 125000/mm³, 75000/mm³ to 100000/mm³, 100000/mm³ to 400000/mm³, 100000/mm³ to 375000/mm³, 100000/mm³ to 350000/mm³. 100000/mm³ to 325000/mm³. 100000/mm³ to 300000/mm³.

100000/mm³ to 275000/mm³, 100000/mm³ to 250000/mm³, 100000/mm³ to 225000/mm³, 100000/mm³ to 200000/mm³, 100000/mm³ to 175000/mm³, 100000/mm³ to 150000/mm³, 100000/mm³ to 125000/mm³, 125000/mm³ to 400000/mm³, 125000/mm³ to 375000/mm³, 125000/mm³ to 350000/mm³, 125000/mm³ to 325000/mm³, 125000/mm³ to 300000/mm³, 125000/mm³ to 275000/mm³, 125000/mm³ to 250000/mm³, 125000/mm³ to 225000/mm³, 125000/mm³ to 200000/mm³, 125000/mm³ to 175000/mm³, 125000/mm³ to 150000/mm³, 150000/mm³ to 400000/mm³, 150000/mm³ to 375000/mm³, 150000/mm³ to 350000/mm³, 150000/mm³ to 325000/mm³, 150000/mm³ to 300000/mm³, 150000/mm³ to 275000/mm³, 150000/mm³ to 250000/mm³, 150000/mm³ to 225000/mm³, 150000/mm³ to 200000/mm³, 150000/mm³ to 175000/mm³. 175000/mm³ to 400000/mm³. 175000/mm³ to 375000/mm³. 175000/mm³ to 350000/mm³, 175000/mm³ to 325000/mm³, 175000/mm³ to 300000/mm³, 175000/mm³ to 275000/mm³, 175000/mm³ to 250000/mm³, 175000/mm³ to 225000/mm³, 175000/mm³ to 200000/mm³, 200000/mm³ to 400000/mm³, 200000/mm³ to 375000/mm³, 200000/mm³ to 350000/mm³, 200000/mm³ to 325000/mm³, 200000/mm³ to 300000/mm³, 200000/mm³ to 275000/mm³, 200000/mm³ to 250000/mm³, 200000/mm³ to 225000/mm³, 225000/mm³ to 400000/mm³, 225000/mm³ to 375000/mm³, 225000/mm³ to 350000/mm³, 225000/mm³ to 325000/mm³, 225000/mm³ to 300000/mm³, 225000/mm³ to 275000/mm³, 225000/mm³ to 250000/mm³, 250000/mm³ to 400000/mm³, 250000/mm³ to 375000/mm³, 250000/mm³ to 350000/mm³, 275000/mm³ to 325000/mm³, 275000/mm³ to 300000/mm³, 300000/mm³ to 400000/mm³, 300000/mm³ to 375000/mm³, 300000/mm³ to 350000/mm³, 300000/mm³ to 325000/mm³, 325000/mm³ to 400000/mm³, 350000/mm³ to 400000/mm³, 350000/mm³ to 375000/mm³, or 375000/mm³ to 400000/mm³, measured within 1 day, 1 week, or 1 month of treatment.

In some embodiments, the patient's hemoglobin level, with or without prior transfusion, is at least 8.0, 9.0, 10.0, 11.0, 12.0, 130, 14.0, 15.0, 16.0, 17.0, 18.0, 19.0, or 20.0 g/dL, measured within 1 day. 1 week, or 1 month of treatment. In some embodiments, the patient's hemoglobin level, with or without prior transfusion, is 8.0 to 20.0, 8.0 to 19.0, 8.0 to 18.0, 8.0 to 17.0, 8.0 to 16.0, 8.0 to 15.0. 8.0 to 14.0, 8.0 to 13.0, 8.0 to 12.0, 8.0 to 11.0, 8.0 to 10.0, 8.0 to 9.0, 9.0 to 20.0, 9.0 to 19.0, 9.0 to 18.0, 9.0 to 17.0, 9.0 to 16.0, 9.0 to 15.0, 9.0 to 14.0, 9.0 to 13.0, 9.0 to 12.0, 9.0 to 11.0. 9.0 to 10.0, 10.0 to 20.0, 10.0 to 19.0, 10.0 to 18.0, 10.0 to 17.0, 10.0 to 16.0, 10.0 to 15.0, 10.0 to 14.0, 10.0 to 13.0, 10.0 to 12.0, 10.0 to 11.0, 11.0 to 20.0. 11.0 to 19.0, 11.0 to 18.0, 11.0 to 17.0. 11.0 to 16.0. 11.0 to 15.0. 11.0 to 14.0. 11.0 to 13.0. 11.0 to 12.0, 12.0 to 20.0, 12.0 to 19.0, 12.0 to 18.0, 12.0 to 17.0, 12.0 to 16.0, 12.0 to 15.0, 12.0 to 14.0, 12.0 to 13.0, 13.0 to 20.0, 13.0 to 19.0, 13.0 to 18.0, 13.0 to 17.0, 13.0 to 16.0, 13.0 to 15.0, 13.0 to 14.0, 14.0 to 20.0, 14.0 to 19.0. 14.0 to 18.0, 14.0 to 17.0, 14.0 to 16.0, 14.0 to 15.0, 15.0 to 20.0, 15.0 to 19.0, 15.0 to 18.0, 15.0 to 17.0, 15.0 to 16.0, 16.0 to 20.0, 16.0 to 19.0, 16.0 to 18.0, 16.0 to 17.0, 17.0 to 20.0, 17.0 to 19.0, 17.0 to 18.0, 18.0 to 20.0, 18.0 to 19.0, or 19.0 to 20.0, measured within 1 day, 1 week, or 1 month of treatment.

In some embodiments, the patient's creatinine clearance is at least 45, 50, 55, 60, 65, 70, 75, 80, 85, 90, 95, 100, 105, 110, 115, 120, 125, 130, 135, or 140 mL/min using the Cockcraft-Gault equation, or mL/min/1.73 m² as estimated glomerular filtration rate (eGFR) per Modification of Diet in Renal Disease (MDRD) equation, measured within 1 day. I week, or] month of treatment. In some embodiments, the patient's creatinine clearance is 45 to 150, 45 to 125, 45 to 100, 45 to 75, 75 to 150, 75 to 125, 75 to 100, 100 to 150, 100 to 125, or 125 to 150 mL/min using the Cockcraft-Gault equation, or mL/min/1.73 m² as estimated glomerular filtration rate (eGFR) per Modification of Diet in Renal Disease (MDRD) equation, measured within 1 day, 1 week, or 1 month of treatment.

In some embodiments, the patient's total serum bilirubin is less than 5, 4.5, 4.0, 3.5. 3.0, 2.5, 2.0, 1.5, 1.0, 0.5, 0.4, 0.3, 0.2, or 0.1 mg/dL, measured within 1 day, 1 week, or 1 month of treatment. In some embodiments, the patient's total serum bilirubin is 0.1 to 5, 0.1 to 4.5, 0.1 to 4.0, 0.1 to 3.5, 0.1 to 3.0, 0.1 to 2.5, 0.1 to 2.0, 0.1 to 1.5, 0.1 to 0.5, 0.1 to 0.4, 0.1 to 0.3, 0.1 to 0.2, 0.2 to 5.0, 0.2 to 4.5, 0.2 to 4.0, 0.2 to 3.5, 0.2 to 3.0, 0.2 to 2.5, 0.2 to 2.0, 0.2 to 1.5, 0.2 to 1.0, 0.2 to 0.5, 0.2 to 0.4, 0.2 to 0.3, 0.3 to 5.0, 0.3 to 4.5, 0.3 to 4.0, 0.3 to 3.5, 0.3 to 3.0, 0.3 to 2.5, 0.3 to 2.0, 0.3 to 1.5, 0.3 to 1.0, 0.3 to 0.5, 0.3 to 0.4, 0.4 to 5.0, 0.4 to 4.5, 0.4 to 4.0, 0.4 to 3.5, 0.4 to 3.0, 0.4 to 2.5, 0.4 to 2.0, 0.4 to 1.5, 0.4 to 1.0, 0.4 to 0.5, 0.5 to 5.0, 0.5 to 4.5, 0.5 to 4.0, 0.5 to 3.5, 0.5 to 3.0, 0.5 to 2.5, 0.5 to 2.0, 0.5 to 1.5, 0.5 to 1.0, 1.0 to 5.0, 1.0 to 4.5, 1.0 to 4.0, 1.0 to 3.5, 1.0 to 3.0, 1.0 to 2.5, 1.0 to 2.0, 1.0 to 1.5, 1.5 to 5.0, 1.5 to 4.5, 1.5 to 4.0, 1.5 to 4.0, 1.5 to 3.5, 1.5 to 3.0, 1.5 to 2.5, 1.5 to 2.0, 2.0 to 5.0, 2.0 to 4.5, 2.0 to 4.0, 2.0 to 3.5, 2.0 to 3.0, 2.0 to 2.5, 2.5 to 5.0, 2.5 to 4.5, 2.5 to 4.0, 2.5 to 3.5, 2.5 to 3.0, 3.0 to 5.0, 3.0 to 4.5, 3.0 to 4.0, 3.5 to 5.0, 3.5 to 4.5, 3.5 to 4.0, 4.0 to 5.0, 4.0 to 4.5, 4.5 to 5.0 mg/dL, measured within 1 day, 1 week, or 1 month of treatment.

In some embodiments, when the patient has known Gilbert's Syndrome, the patient's total serum bilirubin is less than 5, 4.5, 4.0, 3.5, 3.0, 2.5. 2.0, 1.5, 1.0, 0.5, 0.4, 0.3, 0.2, or 0.1 mg/dL, measured within 1 day, 1 week, or 1 month of treatment. In some embodiments, when the patient has known Gilbert's Syndrome, the patient's total serum bilirubin is 0.1 to 5, 0.1 to 4.5, 0.1 to 4.0, 0.1 to 3.5, 0.1 to 3.0, 0.1 to 2.5, 0.1 to 2.0, 0.1 to 1.5, 0.1 to 0.5, 0.1 to 0.4. 0.1 to 0.3, 0.1 to 0.2, 0.2 to 5.0, 0.2 to 4.5, 0.2 to 4.0, 0.2 to 3.5, 0.2 to 3.0, 0.2 to 2.5, 0.2 to 2.0. 0.2 to 1.5, 0.2 to 1.0, 0.2 to 0.5, 0.2 to 0.4, 0.2 to 0.3, 0.3 to 5.0, 0.3 to 4.5, 0.3 to 4.0, 0.3 to 3.5. 0.3 to 3.0, 0.3 to 2.5, 0.3 to 2.0, 0.3 to 1.5, 0.3 to 1.0, 0.3 to 0.5, 0.3 to 0.4, 0.4 to 5.0, 0.4 to 4.5. 0.4 to 4.0, 0.4 to 3.5, 0.4 to 3.0, 0.4 to 2.5, 0.4 to 2.0, 0.4 to 1.5, 0.4 to 1.0, 0.4 to 0.5, 0.5 to 5.0, 0.5 to 4.5, 0.5 to 4.0, 0.5 to 3.5, 0.5 to 3.0, 0.5 to 2.5, 0.5 to 2.0, 0.5 to 1.5, 0.5 to 1.0, 1.0 to 5.0. 1.0 to 4.5, 1.0 to 4.0, 1.0 to 3.5, 1.0 to 3.0, 1.0 to 2.5, 1.0 to 2.0, 1.0 to 1.5, 1.5 to 5.0, 1.5 to 4.5. 1.5 to 4.0, 1.5 to 4.0, 1.5 to 3.5, 1.5 to 3.0, 1.5 to 2.5, 1.5 to 2.0, 2.0 to 5.0, 2.0 to 4.5, 2.0 to 4.0. 2.0 to 3.5. 2.0 to 3.0. 2.0 to 2.5. 2.5 to 5.0, 2.5 to 4.5. 2.5 to 4.0, 2.5 to 3.5. 2.5 to 3.0, 3.0 to 5.0, 3.0 to 4.5, 3.0 to 4.0, 3.5 to 5.0, 3.5 to 4.5, 3.5 to 4.0, 4.0 to 5.0, 4.0 to 4.5, 4.5 to 5.0 mg/dL, measured within 1 day, 1 week, or 1 month of treatment.

In some embodiments, when the patient does not have known Gilbert's Syndrome, the patient's total serum bilirubin is less than 2.5, 2.0, 1.5, 1.0, 0.5, 0.4, 0.3, 0.2, or 0.1 mg/dL, measured within 1 day, 1 week, or 1 month of treatment. In some embodiments, when the patient does not have known Gilbert's Syndrome, the patient's total serum bilirubin is 0.1 to 2.5, 0.1 to 2.0, 0.1 to 1.5, 0.1 to 1.0, 0.1 to 0.5, 0.1 to 0.4, 0.1 to 0.3, 0.1 to 0.2, 0.2 to 2.5, 0.2 to 2.0, 0.2 to 1.5, 0.2 to 1.0, 0.2 to 0.5, 0.2 to 0.4, 0.2 to 0.3, 0.3 to 2.5, 0.3 to 2.0, 0.3 to 1.5, 0.3 to 1.0, 0.3 to 0.5, 0.3 to 0.4, 0.4 to 2.5, 0.4 to 2.0, 0.4 to 1.5, 0.4 to 1.0, 0.4 to 0.5, 0.5 to 2.5, 0.5 to 2.0, 0.5 to 1.5, 0.5 to 1.0, 1.0 to 2.5, 1.0 to 2.0, 1.0 to 1.5, 1.5 to 2.5, 1.5 to 2.0, or 2.0 to 2.5 mg/dL, measured within 1 day, 1 week, or 1 month of treatment.

In some embodiments, the patient's liver transaminases (aspartate aminotransferase/alanine amino transferase/alkaline phosphatase (AST/ALT/ALP)) is less than or equal to 5, 4.5, 4.0, 3.5, 3.0, 2.5, 2.0, 1.5, 1.0, 0.5, or 0 times the upper limit of normal (ULN) for the test, measured within 1 day, 1 week, or 1 month of treatment. In some embodiments, the patient's liver transaminases (aspartate aminotransferase/alanine amino transferase/alkaline phosphatase (AST/ALT/ALP)) is below the upper limit of normal to 5.0 times the upper limit of normal for the test, measured within 1 day, 1 week, or 1 month of treatment. In some embodiments, the patient's liver transaminases (aspartate aminotransferase/alanine amino transferase/alkaline phosphatase (AST/ALT/ALP)) is 0 to 5.0, 0 to 4.5, 0 to 4.0, 0 to 3.5, 0 to 3.0, 0 to 2.5, 0 to 2.0, 0 to 1.5, 0 to 1.0, 0 to 0.5, 0.5 to 5.0, 0.5 to 4.5, 0.5 to 4.0, 0.5 o 3.5. 0.5 to 3.0, 0.5 to 2.5, 0.5 to 2.0. 0.5 to 1.5. 0.5 to 1.0. 1.0 to 5.0, 1.0 to 4.5. 1.0 to 4.0, 1.0 to 3.5. 1.0 to 3.0, 1.0 to 2.5. 1.0 to 2.0, 1.0 to 1.5, 1.5 to 5.0, 1.5 to 4.5, 1.5 to 4.0, 1.5 to 3.5, 1.5 to 3.0, 1.5 to 2.5, 1.5 to 2.0. 2.0 to 5.0, 2.0 to 4.5, 2.0 to 4.0, 2.0 to 3.5, 2.0 to 3.0, 2.0 to 2.5, 2.5 to 5.0, 2.5, to 4.5, 2.5 to 4.0, 2.5 to 3.5, 2.5 to 3.0, 3.0 to 5.0, 3.0 to 4.5, 3.0 to 4.0, 3.0 to 3.5, 3.5 to 5.0, 3.5 to 4.5, 3.5 to 4.0, 4.0 to 5.0, 4.0 to 4.5, or 4.5 to 5.0 times the upper limit of normal for the test, measured within 1 day, 1 week, or 1 month of treatment.

In some embodiments, when the patient has liver metastases, the patient's liver transaminases (aspartate aminotransferase/alanine amino transferase/alkaline phosphatase (AST/ALT/ALP)) is less than or equal to 5, 4.5, 4.0, 3.5, 3.0, 2.5, 2.0, 1.5, 1.0, 0.5, or 0 times the upper limit of normal (ULN) for the test, measured within 1 day, 1 week, or 1 month of treatment. In some embodiments, when the patient has liver metastases, the patient's liver transaminases (aspartate aminotransferase/alanine amino transferase/alkaline phosphatase (AST/ALT/ALP)) is below the upper limit of normal to 5.0 times the upper limit of normal for the test, measured within 1 day, I week, or 1 month of treatment. In some embodiments, when the patient has liver metastases, the patient's liver transaminases (aspartate aminotransferase/alanine amino transferase/alkaline phosphatase (AST/ALT/ALP)) is 0 to 5.0, 0 to 4.5, 0 to 4.0, 0 to 3.5, 0 to 3.0, 0 to 2.5, 0 to 2.0, 0 to 1.5, 0 to 1.0, 0 to 0.5, 0.5 to 5.0, 0.5 to 4.5, 0.5 to 4.0, 0.5 3.5, 0.5 to 3.0, 0.5 to 2.5, 0.5 to 2.0, 0.5 to 1.5, 0.5 to 1.0, 1.0 to 5.0, 1.0 to 4.5, 1.0 to 4.0, 1.0 to 3.5, 1.0 to 3.0, 1.0 to 2.5, 1.0 to 2.0, 1.0 to 1.5, 1.5 to 5.0, 1.5 to 4.5, 1.5 to 4.0, 1.5 to 3.5, 1.5 to 3.0, 1.5 to 2.5, 1.5 to 2.0, 2.0 to 5.0, 2.0 to 4.5, 2.0 to 4.0, 2.0 to 3.5, 2.0 to 3.0, 2.0 to 2.5, 2.5 to 5.0, 2.5, to 4.5. 2.5 to 4.0, 2.5 to 3.5, 2.5 to 3.0, 3.0 to 5.0, 3.0 to 4.5, 3.0 to 4.0, 3.0 to 3.5, 3.5 to 5.0, 3.5 to 4.5, 3.5 to 4.0, 4.0 to 5.0, 4.0 to 4.5, or 4.5 to 5.0 times the upper limit of normal for the test, measured within 1 day, 1 week, or 1 month of treatment.

In some embodiments, when the patient does not have liver metastases, the patient's liver transaminases (aspartate aminotransferase/alanine amino transferase/alkaline phosphatase (AST/ALT/ALP)) is less than or equal to 2.5, 2.0, 1.5, 1.0, 0.5, or 0 times the upper limit of normal (ULN) for the test, measured within 1 day, 1 week, or 1 month of treatment. In some embodiments, when the patient does not have liver metastases, the patient's liver transaminases (aspartate aminotransferase/alanine amino transferase/alkaline phosphatase (AST/ALT/ALP)) is below the upper limit of normal to 5.0 times the upper limit of normal for the test, measured within 1 day, 1 week, or 1 month of treatment. In some embodiments, when the patient does not have liver metastases, the patient's liver transaminases (aspartate aminotransferase/alanine amino transferase/alkaline phosphatase (AST/ALT/ALP)) is 0 to 2.5. 0 to 2.0, 0 to 1.5. 0 to 1.0.

0 to 0.5, 0.5 to 2.5, 0.5 to 2.0, 0.5 to 1.5, 0.5 to 1.0, 1.0 to 2.5, 1.0 to 2.0, 1.0 to 1.5, 1.5 to 2.5, 1.5 to 2.0, or 2.0 to 2.5 times the upper limit of normal for the test, measured within 1 day, 1 week, or 1 month of treatment.

In some embodiments, the patient does not have active nervous system metastases. In some embodiments, the patient does have active nervous system metastases.

In some embodiments, the patient does not have a history or presence of a clinically relevant CNS disorder such as a seizure disorder (e.g., epilepsy), cerebrovascular ischemia/hemorrhage, dementia, cerebellar disease, cerebral edema, posterior reversible encephalopathy syndrome (PRES), or any autoimmune disease with CNS involvement.

In some embodiments, the patient does not have active human immunodeficiency virus (HIV) infection, e.g., as evidenced by a positive HIV polymerase chain reaction (PCR) test, e.g., within 1 day, 1 week, or 1 month of treatment.

In some embodiments, the patient does not have active HBV or hepatitis C virus (HCV) infection, e.g., based on laboratory testing, e.g., within 1 day, 1 week, or 1 month of treatment. In some embodiments, the patient has a viral HBV load below the institutional limit of quantification (LOQ) and the subject is on stable viral suppressive therapy. In some embodiments, the patient has a HCV RNA viral load below the institutional LOQ and has completed curative antiviral treatment.

### Lymphodepletion

In some embodiments, the patient is lymphodepleted before treatment.

Illustrative lymphodepleting chemotherapy regimens, along with correlative beneficial biomarkers, are described in WO 2016/191756 and WO 2019/079564, hereby incorporated by reference in their entirety. In certain embodiments, the lymphodepleting chemotherapy regimen comprises administering to the patient doses of cyclophosphamide (between 200 mg/m²/day and 2000 mg/m²/day) and doses of fludarabine (between 20 mg/m²/day and 900 mg/m²/day).

In some embodiments, lymphodepletion comprises administration of or of about 250 to about 500 mg/m² of cyclophosphamide, e.g., from or from about 250 to or to about 500, 250, 400, 500, about 250, about 400, or about 500 mg/m² of cyclophosphamide.

In some embodiments, lymphodepletion comprises administration of or of about 20 mg/m²/day to or to about 40 mg/m²/day fludarabine, e.g., 30 or about 30 mg/m²/day.

In some embodiments, lymphodepletion comprises administration of both cyclophosmamide and fludarabine.

In some embodiments, the patient is lymphodepleted by intravenous administration of cyclophosphamide (250 mg/m²/day) and fludarabine (30 mg/m²/day).

In some embodiments, the patient is lymphodepleted by intravenous administration of cyclophosphamide (500 mg/m²/day) and fludarabine (30 mg/m²/day).

In some embodiments, the lymphodepletion occurs no more than 5 days prior to the first dose of NK cells. In some embodiments, the lymphodepletion occurs no more than 7 days prior to the first dose of NK cells.

In some embodiments, lymphodepletion occurs daily for 3 consecutive days, starting 5 days before the first dose of NK cells (i.e., from Day-5 through Day-3).

In some embodiments, the lymphodepletion occurs on day-5, day-4 and day-3.

In some embodiments involving repeat or multiple doses, the patient is lymphodepleted before some or every dose as described above. In some embodiments, the patient is lymphodepleted two, three, four, five, or six times.

### Administration

### NK Cells

In some embodiments, the NK cells, e.g., the NK cells described herein, e.g., the CAR-NK cells described herein are administered to the patient as part of a pharmaceutical composition, e.g., a pharmaceutical composition described herein. Cells are administered after thawing, in some cases without any further manipulation in cases where their cryoprotectant is compatible for immediate administration. For a given individual, a treatment regimen often comprises administration over time of multiple aliquots or doses of NK cells, including from doses drawn from a common batch or donor.

The NK cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, a given dose is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

In the context of adoptive cell therapy, administration of a given “dose” can encompass administration of the given amount or number of cells as a single composition and/or single uninterrupted administration, e.g., as a single injection or continuous infusion. A given “dose” can also encompass administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two day's or by multiple infusions over a single day period.

In some embodiments, the NK cells are administered to the patient at a range of at or about 1 million to at or about 100 billion cells. In some embodiments, the NK cells, e.g., the NK cells described herein, e.g., the CAR-NK cells described herein, are administered at or at about 5×10⁶ to or to about 1×10⁹ NK cells per dose. In some embodiments, the NK cells are administered at or at about 5×10⁶, at or at about 1×10⁷, at or at about 3×10⁷, at or at about 1×108, at or at about 3×10⁸, or at or at about 1×10⁹ cells per dose. In some embodiments, the NK cells are administered at or about 1 million to at or about 20 billion cells (e.g., at or about 5 million cells, at or about 25 million cells, at or about 50 million cells, at or about 75 million cells, at or about 100 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, or a range defined by any two of the foregoing values), at or about 10 million to at or about 20 billion cells (e.g., at or about 25 million cells, at or about 50 million cells, at or about 75 million cells, at or about 100 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, at or about 20 billion cells, or a range defined by any two of the foregoing values), and in some cases at or about 100 million cells to at or about 50 billion cells (e.g., at or about 150 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, at or about 20 billion cells, at or about 30 billion cells, at or about 40 billion cells), or any value in between any of these ranges, per dose.

Thus, in some embodiments, the NK cells are administered in a dose comprising at or at about 1×10⁶, 2×10⁶, 3×10⁶. 4×10⁶, 5×10⁶, 6×10⁶, 7×10⁶, 8×10⁶, or 9×10⁶ cells per dose, at or at about 1×10⁷, 2×10⁷, 3×10⁷, 4×10⁷, 5×10⁷, 6×10⁷, 7×10⁷, 8×10⁷, or 9×10⁷ cells per dose, at or at about 1×10⁸, 2×10⁸, 3×10⁸, 4×10⁸, 5×10⁸, 6×10⁸, 7×10⁸, 8×10⁸, or 9×10⁸ cells per dose, at or at about 1×10⁹, 2×10⁹, 3×10⁹, 4×10⁹, 5×10⁹, 6×10⁹, 7×10⁹, 8×109, or 9×10⁹ cells per dose, at or at about 1×10¹⁰ or 2×10¹⁰ cells per dose.

Thus, in some embodiments, the NK cells are administered in a dose comprising at least or at least about 1×10⁶, 2×10⁶, 3×10⁶, 4×10⁶. 5×10⁶, 6×10⁶, 7×10⁶, 8×10⁶, or 9×10⁶ cells per dose, at least or at least about 1×10⁷, 2×10⁷, 3×10⁷, 4×10⁷, 5×10⁷, 6×10⁷, 7×107, 8×10⁷, or 9×10⁷ cells per dose, at least or at least about 1×10⁸, 2×10⁸, 3×10⁸, 4×10⁸, 5×108. 6×10⁸, 7×10⁸, 8×10⁸, or 9×10⁸ cells per dose, at least or at least about 1×10⁹, 2×10⁹, 3×10⁹, 4×10⁹, 5×10⁹, 6×10⁹, 7×10⁹, 8×10⁹, or 9×10⁹ cells per dose, or at least or at least about 1×10¹⁰, or 2×10¹⁰ cells per dose.

In some embodiments, the NK cells are administered in a dose comprising at or about 1 million to at or about 20 billion CAR-expressing NK cells (e.g., at or about 5 million cells, at or about 25 million CAR-expressing cells, at or about 50 million CAR-expressing cells, at or about 75 million CAR-expressing cells, at or about 100 million CAR-expressing cells, at or about 200 million CAR-expressing cells, at or about 300 million CAR-expressing cells, at or about 400 million CAR-expressing cells, at or about 500 million CAR-expressing cells, at or about 1 billion CAR-expressing cells, at or about 2 billion CAR-expressing cells, at or about 3 billion CAR-expressing cells, at or about 4 billion CAR-expressing cells, at or about 5 billion CAR-expressing cells, at or about 6 billion CAR-expressing cells, at or about 7 billion CAR-expressing cells, at or about 8 billion CAR-expressing cells, at or about 9 billion CAR-expressing cells, at or about 10 billion CAR-expressing cells, or at or about 20 billion CAR-expressing cells, or a range defined by any two of the foregoing values), or any value in between any of these ranges, per dose.

Thus, in some embodiments, the NK cells are administered in a dose comprising at or at about 1×10⁶, 2×10⁶, 3×10⁶, 4×10⁶, 5×10⁶, 6×10⁶, 7×10⁶, 8×10⁶, or 9×10⁶ CAR-expressing cells per dose, a dose comprising at or at about 1×10⁷, 2×10⁷, 3×10⁷, 4×10⁷, 5×107, 6×10⁷, 7×10⁷, 8×10⁷, or 9×10⁷ CAR-expressing cells per dose, a dose comprising at or at about 1×10⁸, 2×10⁸, 3×10⁸, 4×10⁸, 5×10⁸, 6×10⁸, 7×10⁸, 8×10⁸, or 9×10⁸ CAR-expressing cells per dose, a dose comprising at or at about 1×10⁹, 2×10⁹, 3×10⁹, 4×10⁹, 5×109, 6×10⁹, 7×10⁹, 8×10⁹, or 9×10⁹ CAR-expressing cells per dose, or a dose comprising at or at about 1×10¹⁰ or at or at about 2×10¹⁰ CAR-expressing cells per dose.

Thus, in some embodiments, the NK cells are administered in a dose comprising at least or at least about 1×10⁶, 2×10⁶, 3×10⁶, 4×10⁶. 5×10⁶, 6×10⁶, 7×10⁶, 8×10⁶, or 9×10⁶ CAR-expressing cells per dose, at least or at least about 1×10⁷, 2×10², 3×10⁷, 4×10⁷, 5×107, 6×10⁷, 7×10⁷, 8×10⁷, or 9×10⁷ CAR-expressing cells per dose, at least or at least about 1×10⁸, 2×10⁸, 3×10⁸, 4×10⁸, 5×10⁸, 6×10⁸, 7×10⁸, 8×10⁸, or 9×10⁸ CAR-expressing cells per dose, at least or at least about 1×10⁹, 2×10⁹, 3×10⁹, 4×10⁹, 5×10⁹, 6×10⁹, 7×10⁹, 8×10⁹, or 9×10⁹ CAR-expressing cells per dose, at least or at least about 1×10¹⁰ CAR-expressing cells per dose.

In some embodiments, the dose of cells is a flat dose of cells or fixed dose of cells such that the dose of cells is not tied to or based on the body surface area or weight of a patient.

In some embodiments, the dose of cells is administered based on the weight of the patient. For example, the dose can be determined per kilogram of body weight of the patient. In some embodiments, the dose of cells comprises between at or about 1×10⁵ of the cells/kg and at or about 1×10⁸ of the cells/kg, such as between at or about 1.5×10⁵ of the cells/kg and at or about 1.5×10⁷ of the cells/kg, or between at or about 4×10⁵ of the cells/kg and at or about 4×10⁶ of the cells/kg.

Thus, in some embodiments, the NK cells are administered in a dose comprising at or at about 1×10⁵, 1.5×10⁵, 2×10⁵, 2.5×10⁵, 3×10⁵, 3.5×10⁵, 4×10⁵, 4.5×10⁵, 5×10⁵, 5.5×105, 6×10⁵, 6.5×10⁵, 7×10⁵, 7.5×10⁵, 8×10⁵, 8.5×10⁵, 9×10⁵, or 9.5×10⁵ cell/kg, 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, or 9.5×10⁶ cells/kg, at or at about 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷ cells/kg, or 9.5×10⁷, or at or at about 1×10⁸, 1.5×10⁸, or 2×10⁸ cells/kg.

In some embodiments, the NK cells are administered in a dose comprising at least or at least about 1×10⁵. 1.5×10⁵, 2×10⁵, 2.5×10⁵, 3×10⁵, 3.5×10⁵, 4×10⁵, 4.5×10⁵. 5×10⁵, 5.5×10⁵, 6×10⁵, 6.5×10⁵, 7×10⁵, 7.5×10⁵, 8×10⁵, 8.5×10⁵, 9×10⁵, or 9.5×10⁵ cell/kg, at least or at least about 1×10⁶, 1.5×10⁶, 2×10⁶, 2.5×10⁶, 3×10⁶, 3.5×10⁶, 4×10⁶, 4.5×10⁶, 5×10⁶. 5.5×10⁶, 6×10⁶, 6.5×10⁶, 7×10⁶, 7.5×10⁶, 8×10⁶, 8.5×10⁶, 9×10⁶, or 9.5×10⁶cells/kg, at least or at least about 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷ cells/kg, or 9.5×10⁷, or at least or at least about 1×10⁸ or 1.5×10⁸ cells/kg.

In some embodiments, the dose of cells, e.g., recombinant receptor-expressing NK cells, is administered to the subject as a single dose or is administered only one time within a period of two weeks, one month, three months, six months, 1 year or more.

The ability to offer repeat dosing may allow patients to experience or maintain a deeper or prolonged response from the therapy. Thus, in some embodiments, the patient receives multiple doses, e.g., two or more doses or at least one subsequent dose, of the NK cells. In some embodiments, two, three, four, five, six, seven, eight, nine, or ten doses are administered to a subject. In some embodiments, the at least one subsequent dose comprises a second dose. In some embodiments, the at least one subsequent dose comprises a second dose and a third dose. In some embodiments, the at least one subsequent dose comprises a second, third, and fourth dose. In some embodiments, the at least one subsequent dose comprises a second, third, fourth, and fifth dose. In some embodiments, the at least one subsequent dose comprises a second, third, fourth, fifth, and sixth dose. In some embodiments, the at least one subsequent dose comprises a second, third, fourth, fifth, sixth, and seventh dose. In some embodiments, the at least one subsequent dose comprises a second, third, fourth, fifth, sixth, seventh, and eighth dose. In some embodiments, a patient can receive response-based dosing, during which the patient continues to receive doses of CAR-NK cell therapy for as long as the patient derives a benefit. The number of doses and the number of cells administered in each dose can also be tailored to the individual patient. In some embodiments, the number of cells administered to the subject in the additional or subsequent dose or doses are the same as or similar to the first dose. Thus, the CAR-NK cell therapies described herein can be tailored to each patient based on that patient's own response. In some cases, the therapy can be terminated if the patient no longer derives a benefit from the CAR-NK cell therapy. In some cases, the therapy can also be reinitiated if the patient relapses.

In some embodiments involving multiple doses or repeat dosing, the NK cells are administered weekly bi-weekly, once every three weeks, once every four weeks, once every five weeks, once every six weeks, once every seven weeks, once every eight weeks, once every nine weeks, once every ten weeks, once every 11 weeks, once every 12 weeks, once every 13 weeks, once every 14 weeks, once every 15 weeks, or once every 16 weeks. For example, a patient can receive a first dose, a second dose, and a third dose, wherein each dose is separated by or by about six weeks. In some embodiments, the NK cells are administered monthly. In some embodiments, the NK cells are administered every other month or once every three months. In some embodiments, the NK cells are administered for or for about 8 weeks.

In some embodiments, the dosing schedule can vary over the course of the therapy. Thus, for example, the patient can receive a first series of doses every 1, 2, 3, 4, 5, 6, 7, 8, 9, 10, 11, 12, 13, 14, 15, or 16 weeks and a second series of doses every 1, 2, 3, 4, 5, 6, 7, 8, 9, 10, 11, 12, 13, 14, 15, or 16 weeks, wherein the time between the first series of doses and the second series of doses is different. In some embodiments, the patient receives 1, 2, 3, 4, 5, 6, 7, 8, 9, or 10 doses in the first series of doses and 1, 2, 3, 4, 5, 6, 7, 8, 9, or 10 doses in the second series of doses, wherein the number of doses in the first series and the second series of doses can be the same or different. Thus, for example, a patient may receive four doses administered every other week in the first series of doses and four doses administered every 12 weeks in the second series of doses.

In some embodiments, the NK cells are administered between one and four times over the course of nine months.

In some embodiments, the NK cells are cryopreserved in an infusion-ready media. e.g., a cryopreservation composition suitable for intravenous administration, e.g., as described herein.

In some embodiments, the NK cells are cryopreserved in vials containing from or from about 1×10⁷ to or to about 1×10⁹ cells per vial. In some embodiments, the NK cells are cryopreserved in vials containing a single dose.

In some embodiments, the cells are thawed, e.g., in a 37° C., water bath, prior to administration.

In some embodiments, the thawed vial(s) of NK cells are aseptically transferred to a single administration vessel, e.g., administration bag using, e.g., a vial adapter and a sterile syringe. The NK cells can be administered to the patient from the vessel through a Y-type blood/solution set filter as an IV infusion, by gravity.

In some embodiments, the NK cells are administered as soon as practical, preferably less than 90 minutes, e.g., less than 80, 70, 60, 50, 40, 30, 20, or 10 minutes after thawing. In some embodiments, the NK cells are administered within 30 minutes of thawing.

In some embodiments, the pharmaceutical composition is administered intravenously via syringe.

In some embodiments, 1 mL, 4 mL, or 10 mL of drug product is administered to the patient intravenously via syringe.

In some embodiments, the patient is administered acetaminophen prior to being administered the NK cell infusion. In some embodiments, the patient is administered 100, 200, 250, 300, 400, 500, 600, 700, 750, 800, 900, 1000, 1100, 1200, 1250, 1300, 1400, 1500, 1600, 1700, 1750, 1800, 1900, or 2000 mg of acetaminophen. In some embodiments, the acetaminophen is administered to the patient immediately prior to the NK cells. In some embodiments, the acetaminophen is administered 5, 10, 15, 20, 25, 30, 35, 40, 45, 50, 55. 60, 70, 80, 90, 100, 110, 120, 150, or 180 minutes before the NK cells. In some embodiments, the acetaminophen is administered orally.

In some embodiments, the patient is administered diphenhydramine prior to being administered the NK cell infusion. In some embodiments, the patient is administered 5, 10, 12.5, 15, 17.5, 20, 25, 30, 40, 50, 60, 70, 75, 80, 90, or 100 mg of diphenhydramine. In some embodiments, the diphenhydramine is administered to the patient immediately prior to the NK cells. In some embodiments, the diphenhydramine is administered 5. 10. 15. 20. 25. 30. 35. 40.

45, 50, 55, 60, 70, 80, 90, 100, 110, 120, 150, or 180 minutes before the NK cells. In some embodiments, the acetaminophen is administered orally.

In some embodiments, the patient is monitored for a period of time before and after administration of NK cells. For example, the patient's vital signs can be monitored. These can include temperature, respiratory rate. heart rate, blood pressure, and oxygen saturation (SaO2) by pulse oximetry. In some cases, at least one vital sign is measured beginning 5, 10, 15 20, 25, or 30 minutes prior to NK cell administration. After NK cell administration, at least one vital sign can be monitored for 1, 2, 3, 4, or 5 hours continuously or at regular or irregular intervals, including approximately every 5. 10, 15, 20, 25, or 30 minutes. In some cases, vital signs can be monitored after NK cell administration until the patient is stable.

### Cytokines

In some embodiments, a cytokine is administered to the patient.

In some embodiments, the cytokine is administered together with the NK cells as part of a pharmaceutical composition. In some embodiments, the cytokine is administered separately from the NK cells, e.g., as part of a separate pharmaceutical composition.

In some embodiments, the cytokine is IL-2.

In some embodiments, the IL-2 is administered subcutaneously.

In some embodiments, the IL-2 is administered from between 1 to 4 or about 1 to about 4 hours following the conclusion of NK cell administration. In some embodiments, the IL-2 is administered at least 1 hour following the conclusion of NK cell administration. In some embodiments, the IL-2 is administered no more than 4 hours following the conclusion of NK cell administration. In some embodiments, the IL-2 is administered at least 1 hour after and no more than 4 hours following the conclusion of NK cell administration.

In some embodiments, the IL-2 is administered at up to 10 million IU/M², e.g., up to 1 million, 2 million, 3 million, 4 million, 5 million, 6 million, 7 million, 8 million, 9 million, or 10 million IU/m².

In some embodiments, the IL-2 is administered at or at about 1 million, at or at about 2 million, at or at about 3 million, at or at about 4 million, at or at about 5 million, at or at about 6 million, at or at about 7 million, at or at about 8 million, at or at about 9 million, at or at about 10 million IU/M2

In some embodiments, the IL-2 is administered at or at about 1×10⁶IU/M2. In some embodiments, the IL-2 is administered at or at about 2×10⁶IU/M2.

In some embodiments, less than 1×10⁶IU/M2 IL-2 is administered to the patient.

In some embodiments, a flat dose of IL-2 is administered to the patient. In some embodiments, a flat dose of 6 million IU or about 6 million IU is administered to the patient. In some embodiments, IL-2 is not administered to the patient.

### Dosing

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week: including once every other day. The skilled artisan will appreciate that certain factors may influence the dosage and timing required to effectively treat a subject, including but not limited to the severity of the disease or disorder, previous treatments. the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Combination Therapies

In some embodiments, the method comprises administering the NK cells described herein, e.g., the CAR-NK cells described herein, in combination with another therapy, e.g., an antibody, an NK cell engager, an antibody drug conjugate (ADC), a chemotherapy drug, e.g., a small molecule drug, an immune checkpoint inhibitor, and combinations thereof. The other therapy can be administered prior to, subsequent to, or simultaneously with administration of the NK cells.

### Antibodies

In some embodiments, the other therapy is an antibody.

In some embodiments, the antibody binds to a target selected from the group consisting of CD20, HER-2, EGFR, CD38, SLAMF7, GD2, ALK1, AMHR2, CCR2, CD137, CD19, CD26, CD32b, CD33, CD37, CD70, CD73, CD74, CD248, CLDN6, Clever-1, c-MET, CSF-1R, CXCR4, DKK1, DR5, Epha3, FGFR2b, FGFR3, FLT3,, FOLRI, Globo-H, Glypican3, GM1, Grp78, HER-3, HGF, IGF-IR, IL1RAP, IL-8R, ILT4, Integrin alpha V, M-CSF,

Mesothelin, MIF, MUCI, MUC16, MUCSAC, Myostatin, NKG2A, NOTCH, NOTCH2/3, PIGF, PRL3, PSMA, RORI, SEMA4D, Sialyl Lewis A, Siglec15, TGF-b, TNFR3, TRAIL-R2, VEGF, VEGFR1, VEGFR2, Vimentin, and combinations thereof.

Suitable antibodies include but are not limited to those shown in Table 8.

TABLE 8
Antibodies for Combination Therapy

| Target | Drug Name | Brand Name | Indication(s) | Reference |
|---|---|---|---|---|
| CD20 | Rituxan | Rituximab | DLBCL/FL, NHL, | Du et al., Auto Immun Highlights |
| | | | CLL, RA, GPA, | (2017) 8(1): 12 |
| | | | MPA | |
| CD20 | Gazyva | Obinutuzumab | CLL, FL | Gagez et al., Curr Opin Oncol. |
| | | | | 2014 September; 26(5): 484-91 |
| CD20 | Arzerra | Ofatumumab | CLL | Robak, Curr Opin Mol Ther. |
| | | | | 2008 June; 10(3): 294-309 |
| CD20 | Ocrevus | Ocrelizumab | RMS, PPMS | Genovese et al., Arthritis Rheum. |
| | | | | 2008 September; 58(9): 2652-61 |
| CD20 | Zevalin | Ibritumomab | NHL | Wiseman et al., Eur J Nucl Med. |
| | | | | 2000 July; 27(7): 766-77 |
| CD20 | | Veltuzumab | NHL, CLL | Kalaycio et al. Leuk Lymphoma. |
| | | | | 2016; 57(4): 803-11 |
| CD20 | Bexxar | Tositumomab | NHL | Vose et al., J Clin Oncol. 2000 |
| | | and Iodine I | | March; 18(6): 1316-23 |
| | | 131 | | |
| | | tositumomab | | |
| CD20 | | Ublituximab | NHL, CLL, RMS | Sawas et al., Br J Haematol. 2017 |
| | | | | April; 177(2): 243-253 |
| HER-2 | Herceptin | Trastuzumab | Breast, Gastric | Goldenberg, Clin Ther. 1999 |
| | | | | February; 21(2): 309-18 |
| HER-2 | Perjeta | Pertuzumab | Breast | Agus et al., J Clin Oncol. 2005 |
| | | | | Apr. 10; 23(11): 2534-43 |
| HER-2 | Margenza | Margetuximab | Breast | Bang et al., Ann Oncol. 2017 Apr. |
| | | | | 1; 28(4): 855-861 |
| EGFR | Erbitux | Cetuximab | CRC, HNC | Jonker et al., N Engl J Med 2007; |
| | | | | 357: 2040-2048 |
| EGFR | Vectibix | Panitumumab | CRC | Gibson et al., Clin Colorectal |
| | | | | Cancer. 2006 May; 6(1): 29-31 |
| EGFR | Portrazza | Necitumumab | NSCLC | Kuenen et al., Clin Cancer Res. |
| | | | | 2010 Mar. 15; 16(6): 1915-23 |
| CD38 | Darzalex | Daratumumab | MM | de Weers et al., J Immunol. 2011 |
| | | | | Feb. 1; 186(3): 1840-8 |
| CD38 | Sarclisa | Isatuximab | MM | Martin et al., Blood Cancer J. |
| | | | | 2019 Mar. 29; 9(4): 41 |
| SLAMF7 | Empliciti | Elotuzumab | MM | Lonial et al., N Engl J Med 2015; |
| | | | | 373: 621-631 |
| GD2 | Unituxin | Dinutuximab | NB | Hoy, Target Oncol. 2016 |
| | | | | Apr; 11(2): 247-53 |
| GD2 | Danyelza | Naxitamab | NB | Markham, Drugs. 2021 |
| | | | | February; 81(2): 291-296 |
| ALK1 | PF- | Ascrinvacumab | Liver cancer | Simonelli et al., Ann Oncol. 2016 |
| | 03446962 | | | September; 27(9): 1782-7 |
| AMHR2 | GM-102 | Murlentamab | Ovarian Cancer | Leary et al., J Clin Oncol. 2019 |
| | | | | 37: 15_suppl, 2521-2521 |
| CCR2 | TAK-202 | Plozalizumab | Atherosclerosis, | Gilbert et al., Am J Cardiol. 2011 |
| | | | Melanoma | Mar. 15; 107(6): 906-11 |
| CD137 | BMS- | Urelumab | Melanoma, | Segal et al., Clin Cancer Res. |
| | 663513 | | Myeloma, NSCLC | 2017 Apr. 15; 23(8): 1929-1936 |
| CD137 | PF- | Utomilumab | Ovarian Cancer | Segal et al., Clin Cancer Res. |
| | 05082566 | | | 2018 Apr. 15; 24(8): 1816-1823 |
| CD19 | AMG103 | Blinatumomab | ALL, NHL | Nadafi et al., Int J Mol Cell Med |
| | | | | (2015) 4(3): 143-151 |
| CD19 | SAR3419 | Coltuximab | ALL, NHL | Nadafi et al. |
| | | Ravtansine | | |
| CD19 | XmAb 5574 | MOR208 | ALL, NHL, CLL | Nadafi et al. |
| CD19 | MEDI-551 | MEDI-551 | B-cell | Nadafi et al. |
| | | | malignancies, | |
| | | | CLL, Multiple | |
| | | | Myeloma, | |
| | | | Scleroderma | |
| CD19 | SGN-19A | Denintuzumab | NHL | Nadafi et al. |
| | | Mafodotin | | |
| CD19 | | DI-B4 | B-cell | Nadafi et al. |
| | | | malignancies | |
| CD19 | Taplitumom | Taplitumomab | B-cell | Nadafi et al. |
| | abpaptox | paptox | malignancies | |
| CD19 | XmAb 5871 | XmAb 5871 | Autoimmune | Nadafi et al. |
| | | | Diseases | |
| CD19 | MDX-1342 | MDX-1342 | CLL, Rheumatoid | Nadafi et al. |
| | | | Arthritis | |
| CD19 | AFM11 | AFM11 | NHL | Nadafi et al. |
| CD19 | ADCT-402 | Loncastuximab | ALL, NHL | Yu et al., Journal of Hematology |
| | | Tesirine | | & Oncology (2019) 12(94) |
| CD19 | Monjuvi | Tafasitamab | NHL (DLBCL) | Hoy, Drugs. 2020 |
| | | | | November; 80(16): 1731-1737 |
| CD26 | Begedina | Begelomab | Graft versus host | Bacigalupo et al., Bone Marrow |
| | | | disease | Transplant. 2020 |
| | | | | August; 55(8): 1580-1587 |
| CD32b | BI-1206 | BI-1206 | BCL, CLL | Trial ID: NCT04219254 |
| CD33 | Mylotarg | Gemtuzumab | AML | Stasi, Expert Opin Biol Ther. |
| | | Ozogamicin | | 2008 April; 8(4): 527-40 |
| CD33 | SGN-33 | Lintuzumab | AML | Trial ID: NCT02998047 |
| CD37 | BI 836826 | BI 836826 | DLBCL, CLL, | Trial ID: NCT02538614 |
| | | | NHL | |
| CD37 | IMGN529 | Naratuximab | DLBCL, NHL | Yu et al., Journal of Hematology |
| | | emtansine | | & Oncology (2019) 12(94) |
| CD37 | AGS67E | AGS67E | DLBCL, NHL | Yu et al. |
| CD70 | BMS- | MDX-1203 | DLBCL, MCL | Yu et al. |
| | 936561 | | | |
| CD70 | SGN-75 | Vorsetuzumab | NHL | Yu et al. |
| | | mafodotin | | |
| CD73 | MEDI9447 | Oleclumab | Pancreatic cancer | Geoghegan et al., Mabs. |
| | | | | 2016; 8(3): 454-67 |
| CD73 | AK119 | AK119 | Covid-19, Solid | Trial ID: NCT04516564 |
| | | | Tumors | |
| CD74 | hLL1-DOX | Milatuzumab | MM | Yu et al. |
| | | doxorubicin | | |
| CD74 | STRO-001 | STRO-001 | MM, NHL | Trial ID: NCT03424603 |
| CD248 | Ontecizumab | Ontuxizumab | MM, Soft tissue | D'Angelo et al., Invest New |
| | | | sarcoma | Drugs. 2018 February; 36(1): 103-113 |
| CLDN6 | IMAB027 | ASP1650 | Testicular cancer | Trial ID: NCT03760081 |
| Clever-1 | Clevegen | Bexmarilimab | Solid tumors | Trial ID: NCT03733990 |
| c-MET | MetMAb | Onartuzumab | NSCLC | Hughes et al., Trends Cancer |
| | | | | (2018) 4(2): 94-97 |
| c-MET | AMG-102 | Rilotumumab | Gastric cancer | Waddell et al., Immunotherapy. |
| | | | | 2014; 6(12): 1243-53 |
| CSF-1R | FPA-008 | Cabiralizumab | MM, NSCLC | Trial ID: NCT04050462 |
| CSF-1R | RG-7155 | Emactuzumab | Ovarian cancer | Trial ID: NCT03708224 |
| CSF-1R | IMC CS4 | LY3022855 | MM | Trial ID: NCT03153410 |
| CSF-1R | AMB 051 | AMG 820 | Solid tumors | Trial ID: NCT04731675 |
| CSF-1R | SNDX-6352 | Axatilimab | Graft versus host | Trial ID: NCT04710576 |
| | | | disease | |
| CXCR4 | BMS- | Ulocuplumab | Leukemia | Bobkov et al., Mol Pharmacol |
| | 936564 | | | (2019) 96: 753-764 |
| CXCR4 | LY2624587 | LY2624587 | Metastatic Cancer | Bobkov et al. |
| CXCR4 | PF- | PF-06747143 | AML | Bobkov et al. |
| | 06747143 | | | |
| CXCR4 | F50067 | hz515H7 | MM | Bobkov et al. |
| CXCR4 | MEDI3185 | MEDI3185 | Hematologic | Bobkov et al. |
| | | | malignancies | |
| DKK1 | DKN-01 | DKN-01 | Gastric cancer | Wall et al., Expert Opin Investig |
| | | | | Drugs. 2020 July; 29(7): 639-644 |
| DKK1 | BHQ880 | BHQ880 | MM | Fulciniti et al., Blood. 2009 Jul. |
| | | | | 9; 114(2): 371-9 |
| DR5 | AD5-10 | Zaptuzumab | Solid tumors | Zhang et al., Theranostics. 2019 |
| | | | | Jul. 13; 9(18): 5412-5423 |
| DR5 | AMG655 | Conatumumab | Colon, pancreatic | Rosevear et al., Curr Opin |
| | | | cancer | Investig Drugs. 2010 |
| | | | | June; 11(6): 688-98 |
| DR5 | PRO955780 | Drozitumab | NHL, NSCLC | Kang et al., Clin Cancer Res. |
| | | | | 2011 May 15; 17(10): 3181-92 |
| DR5 | ETR2-ST01 | Lexatumumab | Solid tumors | Plummer et al., Clin Cancer Res. |
| | | | | 2007 Oct. 15; 13(20): 6187-94 |
| DR5 | CS-1008 | Tigatuzumab | Solid tumors | Reck et al., Lung Cancer. 2013 |
| | | | | December; 82(3): 441-8 |
| DR5 | | DS-8273a | Solid tumors | Forero et al., Invest New Drugs. |
| | | | | 2017 June; 35(3): 298-306 |
| Epha3 | KB004 | KB004 | Glioblastoma | Swords et al., Leuk Res. 2016 |
| | | | | November; 50: 123-131 |
| FGFR2b | FPA-144 | Bemarituzumab | Gastric cancer | Catenacci et al., J Clin Oncol. |
| | | | | 2020 Jul. 20; 38(21): 2418-2426 |
| FGFR2b | BAY | Aprutumab | Solid tumors | Kim et al., Target Oncol. 2019 |
| | 1187982 | ixadotin | | October; 14(5): 591-601 |
| FGFR2b | BAY- | Aprutumab | Solid tumors | Trial ID: NCT01881217 |
| | 1179470 | | | |
| FGFR3 | LY3076226 | LY3076226 | Solid tumors | Trial ID: NCT02529553 |
| FLT3 | IMC-EB10 | IMC-EB10 | AML | Piloto et al., Cancer Res. 2006 |
| | | | | May 1; 66(9): 4843-51 |
| | AGS 62P1 | ASP1235 | AML | Trial ID: NCT02864290 |
| FOLR1 | MORAb- | Farletuzumab | Ovarian cancer | Sato et al., Onco Targets Ther |
| | 003 | | | 2016 Mar. 7; 9: 1181-8 |
| Globo-H | OBI-833 | OBI-833 | Solid tumors | Trial ID: NCT02310464 |
| Globo-H | OBI-888 | OBI-888 | Solid tumors | Trial ID: NCT03573544 |
| Globo-H | OBI-999 | OBI-999 | Solid tumors | Trial ID: NCT04084366 |
| Glypican3 | GC33 | Codrituzumab | Liver cancer | Abou-Alfa et al., J Hepatol. 2016 |
| | | | | August; 65(2): 289-95 |
| Glypican3 | | ERY974 | Solid tumors | Ishiguro et al., Sci Transl Med. |
| | | | | 2017 Oct. 4; 9(410) |
| GM1 | BMS986012 | BMS-986012 | Lung cancer | Ponath et al., Clin Cancer Res. |
| | | | | 2018 Oct. 15; 24(20): 5178-5189 |
| Grp78 | PAT-SM6 | PAT-SM6 | Multiple myeloma | Hensel et al., Melanoma Res. |
| | | | | 2013 August; 23(4): 264-75 |
| HER-3 | U3-1402 | Patritumab | NSCLC, Solid | Hashimoto et al., Clin Cancer |
| | | deruxtecan | tumors | Res. 2019 Dec. 1; 25(23): 7151- |
| | | | | 7161 |
| HGF | AMG-102 | Rilotumumab | Solid tumors | Waddell et al., Immunotherapy. |
| | | | | 2014; 6(12): 1243-53 |
| HGF | AV-299 | Ficlatuzumab | AML, NSCLC | Bauman et al., Cancers (Basel). |
| | | | | 2020 Jun. 11; 12(6): 1537 |
| HGF | L2G7 | TAK-701 | Solid tumors | Okamoto et al., Mol Cancer Ther. |
| | | | | 2010 October; 9(10): 2785-92 |
| IGF-1R | IMC-A12 | Cixutumumab | EWS, HCC | Chen et al., Chin J Cancer (2013) |
| | | | | 32(5): 242-252 |
| IGF-1R | CP-751 | Figitumumab | EWS, ACC | Chen et al. |
| IGF-1R | MK-0646 | Dalotuzumab | Colorectal cancer | Chen et al. |
| IGF-1R | AMG 479 | Ganitumab | EWS, DRCT | Chen et al. |
| IGF-1R | | R1507 | EWS | Chen et al. |
| IGF-1R | AVE-1642 | VRDN 001 | MM, Breast | Trial ID: NCT01233895 |
| | | | cancer | |
| IL1RAP | CAN04 | Nidanilimab | NSCLC | Awada et al., J Clin Oncol. 2019 |
| | | | | May; 37: 2504-2504 |
| IL-8R | BMS- | HuMax-IL8 | Covid-19, NSCLC | Bilusic et al., J Immunother |
| | 986253 | | | Cancer. 2019 Sep. 5; 7(1): 240 |
| ILT4 | JTX-8064 | JTX-8064 | Solid tumors | Trial ID: NCT04669899 |
| Integrin | IMGN388 | IMGN388 | Solid tumors | Trial ID: NCT00721669 |
| alpha V | | | | |
| Integrin | CNTO-95 | Intetumumab | MM | O'Day et al., Br J Cancer. 2011 |
| alpha V | | | | Jul. 26; 105(3): 346-52 |
| Integrin | EMD525797 | Abituzumab | Colorectal cancer | Jiang et al., Mol Cancer Res. |
| alpha V | | | | 2017 July; 15(7): 875-883 |
| Integrin | MEDI-522 | Etaracizumab | MM, Colorectal | Hersey et al., Cancer. 2010 Mar. |
| alpha V | | | cancer | 15; 116(6): 1526-34 |
| Integrin | VPI-2690B | VPI-2690B | Diabetic | Trial ID: NCT02251067 |
| alpha V | | | nephropathies | |
| M-CSF | MCS-110 | Lacnotuzumab | Breast cancer, | Pognan et al., J Pharmacol Exp |
| | | | Gastric cancer | Ther. 2019 June; 369(3): 428-442 |
| Mesothelin | MORAb- | amatuximab | Mesothelioma | Baldo et al., Onco Targets Ther. |
| | 009 | | | 2017 Nov. 8; 10: 5337-5353 |
| Mesothelin | SS1(dsFv)- | SS1P | Neoplasms | Hassan et al., J Clin Oncol. 2016 |
| | PE38 | | | December; 34(34): 4171-4179 |
| Mesothelin | BAY 94- | Anetumab | Mesothelioma | Hassan et al., J Clin Oncol. 2020 |
| | 9343 | ravtansine | | Jun. 1; 38(16): 1824-1835 |
| Mesothelin | RG7600 | DMOT4039A | Pancreatic cancer, | Hassan et al., J Clin Oncol. 2016 |
| | | | ovarian cancer | December; 34(34): 4171-4179 |
| Mesothelin | BMS- | BMS-986148 | Solid Tumors | Hassan et al., J Clin Oncol. 2016 |
| | 986148 | | | December; 34(34): 4171-4179 |
| MIF | BAX69 | Imalumab | Colorectal cancer | Mahalingham et al., Br J Clin |
| | | | | Pharmacol. 2020 September; 86(9): |
| | | | | 1836-1848 |
| MUC1 | huC242- | Cantuzumab | Pancreatic cancer | Tolcher et al., J Clin Oncol. 2003 |
| | DM1 | mertansine | | Jan. 15; 21(2): 211-22 |
| MUC1 | hPAM4 | Clivatuzumab | Pancreatic cancer | Liu et al., Oncotarget. 2015 Feb. |
| | | | | 28; 6(6): 4274-85 |
| MUC1 | GT-MAB | Gatipotuzumab | Ovarian cancer | Heublin et al., Int J Mol Sci. 2019 |
| | 2.5-GEX ™ | | | Jan. 12; 20(2): 295 |
| MUC1 | mAb- | AR20.5 | Pancreatic cancer | de Bono et al., Ann Oncol. 2004 |
| | AR20.5 | | | December; 15(12): 1825-33 |
| MUC16 | ACA 125 | Abagovomab | Ovarian cancer | Sabbatini et al., J Clin Oncol. |
| | | | | 2013 Apr. 20; 31(12): 1554-61 |
| MUC16 | DMUC5754A | Sofituzumab | Ovarian cancer | Liu et al., Ann Oncol. 2016 |
| | | vedotin | | November; 27(11): 2124-2130 |
| MUC16 | DMUC4064A | THIOMAB ™ | Ovarian cancer | Trial ID: NCT02146313 |
| MUC5AC | PAM4 | Clivatuzumab | PDAC | Gold et al., Molecular Cancer |
| | | | | (2013) 12: 143 |
| MUC5AC | NPC-1C | Ensituximab | Pancreatic cancer | Kim et al., Clin Cancer Res. 2020 |
| | | | | Jul. 15; 26(14): 3557-3564 |
| Myostatin | MYO-029 | Stamulumab | Muscular atrophy, | Trial ID: NCT00563810 |
| | | | Muscular | |
| | | | dystrophies | |
| Myostatin | PF- | Domagrozumab | Duchenne | Wagner et al., Neuromuscul |
| | 06252616 | | muscular | Disord. 2020 June; 30(6): 492-502 |
| | | | dystrophy | |
| Myostatin | LY- | Landogrozumab | Muscular atrophy, | Golan et al., J Cachexia |
| | 2495655 | | Pancreatic cancer | Sarcopenia Muscle. 2018 |
| | | | | October; 9(5): 871-879 |
| Myostatin | REGN-1033 | Trevogrumab | Muscular atrophy | Trial ID: NCT01720576 |
| Myostatin | SRK-015 | Apitegromab | Spinal muscular | Trial ID: NCT03921528 |
| | | | atrophy | |
| NKG2A | IPH2201 | Monalizumab | Breast cancer; | Andre et al., Cell. 2018 Dec. |
| | | | NSCLC | 13: 175(7): 1731-1743 |
| NOTCH | OMP- | Demcizumab | NSCLC | Takebe et al., Pharmacol Ther |
| | 21M18 | | | (2014) 141(2): 140-149 |
| NOTCH | REGN421/ | Enoticumab | NSCLC, Ovarian | Takebe et al. |
| | SAR153192 | | cancer | |
| NOTCH | OPM- | Brontictuzumab | Solid tumors | Takebe et al. |
| | 52M51 | | | |
| NOTCH2/3 | OMP-59R5 | Tarextumab | Sarcomas, Rectal | Takebe et al. |
| | | | cancer | |
| PIGF | RO5323441 | TB-403 | Solid tumors | Martinsson-Niskanen et al., Clin |
| | | | | Ther. 2011 September; 33(9): |
| | | | | 1142-9 |
| PRL3 | PRL3- | PRL3-zumab | Solid tumors | Trial ID: NCT04452955 |
| | ZUMAB | | | |
| PSMA | Capromab | Capromab | Prostate cancer | Trial ID: NCT00992745 |
| | | pendetide | | |
| PSMA | MT112 | Pasotuxizumab | Prostate cancer | Hummel et al., Immunotherapy. |
| | | | | 2021 February; 13(2): 125-141 |
| PSMA | | MDX1201- | Prostate cancer | Trial ID: NCT02048150 |
| | | A488 | | |
| PSMA | APVO 414 | MOR209/ES414 | Prostate cancer | Hernandez-Hoyos et al., Mol |
| | | | | Cancer Ther. 2016 |
| | | | | September; 15(9): 2155-65 |
| PSMA | ARX-517 | ARX517 | Prostate cancer | Trial ID: NCT04662580 |
| PSMA | ADCT 401 | MEDI3726 | Prostate cancer | Cho et al., Mol Cancer Ther. |
| | | | | 2018 October.; 17(10): 2176-2186 |
| PSMA | | JNJ-63898081 | Prostate cancer | Trial ID: NCT03926013 |
| PSMA | PSMA TTC | BAY 2315497 | Prostate cancer | Hammer et al., Clin Cancer Res. |
| | | | | 2020 Apr. 15; 26(8): 1985-1996 |
| PSMA | | TLX592 | Prostate cancer | Trial ID: NCT04726033 |
| PSMA | DOTA- | Rosopatamab | Prostate cancer | Vallabhajosula et al., Curr |
| | HUJ-591 | tetraxetan | | Radiopharm. 2016; 9(1): 44-53 |
| PSMA | | PSMA ADC | Prostate cancer | Petrylak et al., Prostate. 2020 |
| | | | | January; 80(1): 99-108 |
| ROR1 | UC-961 | Cirmtuzumab | CLL, MCL | Choi et al., Cell Stem Cell. 2018 |
| | | | | Jun. 1; 22(6): 951-959 |
| SEMA4D | VX15/2503 | Pepinemab | NSCLC, MM | |
| Sialyl | MVT-5873 | MVT-5873 | Colorectal cancer | Gupta et al., J Gastrointest Oncol. |
| Lewis A | | | | 2020 April; 11(2): 231-235 |
| Sialyl | AbGn-7 | AbGn-7 | Gastric cancer | Trial ID: NCT01466569 |
| Lewis A | | | | |
| Siglec15 | NC318 | NC318 | Solid tumors | Trial ID: NCT03665285 |
| TGF-b | | SRK-181 | Solid tumors | Trial ID: NCT04291079 |
| TGF-b | M-7824 | Bintrafusp alfa | NSCLC, Solid | Yoo et al., J Immunother Cancer. |
| | | | tumors | 2020 May; 8(1): e000564 |
| TGF-b | GC-1008 | Fresolimumab | MM | Rice et al., J Clin Invest. 2015 Jul. |
| | | | | 1; 125(7): 2795-807 |
| TGF-b | | LY2382770 | Diabetic | Trial ID: NCT01113801 |
| | | | nephropathies | |
| TGF-b | NIS-793 | NIS793 | Pancreatic cancer | Trial ID: NCT04390763: |
| TGF-b | | SAR439459 | Solid tumors | Trial ID: NCT03192345 |
| TGF-b | | Metelimumab | Cancer, | Lord et al., Mabs. 2018 |
| | | | Scleroderma | April; 10(3): 444-452 |
| TGF-b | IMC TR1 | LY3022859 | Solid tumors | Tolcher et al., Cancer Chemother |
| | | | | Pharmacol. 2017 April; 79(4): 673- |
| | | | | 680 |
| TNFR3 | Baminercept | BG9924 | Rheumatoid | Trial ID: NCT00664716 |
| | | | arthritis | |
| TRAIL-R2 | CS-1008 | Tigatuzumab | Breast cancer, | Cheng et al., J Hepatol. 2015 |
| | | | NSCLC | October; 63(4): 896-904 |
| TRAIL-R2 | AMG-655 | Conatumumab | Solid tumors | Bajaj et al., Expert Opin Biol |
| | | | | Ther. 2011 November; 11(11): |
| | | | | 1519-24 |
| TRAIL-R2 | PRO-95780 | Drozitumab | NHL, NSCLC | Lima et al., Cancer Invest. 2012 |
| | | | | December; 30(10): 727-31 |
| TRAIL-R2 | HGS-ETR2 | Lexatumumab | Solid tumors | Plummer et al., Clin Cancer Res. |
| | | | | 2007 Oct. 15; 13(20): 6187-94 |
| TRAIL-R2 | TAS-266 | TAS266 | Solid tumors | Trial ID: NCT01529307 |
| TRAIL-R2 | GEN1029 | Benufutamab | Solid tumors | Overdijk et al., Mol Cancer Ther. |
| | | | | 2020 October; 19(10): 2126-2138 |
| TRAIL-R2 | RO- | RG7386 | Solid tumors | Brunker et al., Mol Cancer Ther. |
| | 6874813 | | | 2016 May; 15(5): 946-57 |
| TRAIL-R2 | JCT-205 | INBRX-109 | Solid tumors | Trial ID: NCT03715933 |
| VEGF | Avastin | Bevacizumab | NSCLC, MM | Garcia et al., Cancer Treat Rev. |
| | | | | 2020 June; 86: 102017 |
| VEGF | Lucentis | Ranibizumab | Macular | Gross et al., JAMA Ophthalmol. |
| | | | de generation | 2018 Oct. 1; 136(10): 1138-1148 |
| VEGFR1 | IMC-18F1 | Icrucumab | Breast cancer | LoRusso et al., Invest New |
| | | | | Drugs. 2014 April; 32(2): 303-11 |
| VEGFR2 | Cyramza | Ramucirumab | NSCLC, | Khan et al., Expert Opin Biol |
| | | | Colorectal cancer | Ther. 2019 November; 19(11): |
| | | | | 1135-1141 |
| VEGFR2 | Tanibirumab | Olinvacimab | Glioblastoma | Lee et al., Drug Des Devel Ther. |
| | | | | 2018 Mar. 8; 12: 495-504 |
| VEGFR2 | | Gentuximab | Solid tumors | Chamie et al., JAMA Oncol. |
| | | | | 2017 Jul. 1; 3(7): 913-920 |
| VEGFR2 | CDP-791 | Alacizumab | NSCLC | Trial ID: NCT00152477 |
| | | pegol | | |
| VEGFR2 | HLX-06 | Vulinacimab | Solid tumors | Trial ID: NCT03494231 |
| VEGFR2 | | MSB0254 | Solid tumors | Trial ID: NCT04381325 |
| VEGFR2 | | AK109 | Solid tumors | Trial ID: NCT04547205 |
| Vimentin | CLNH11 | Pritumumab | Glioma | Babic et al., Hum Antibodies. |
| | | | | 2018 Feb. 5; 26(2): 95-101 |
| Vimentin | | 86C | Glioblastoma | Stouhalova et al., Cancers (2020) |
| | | | | 12(1): 184 |

### Small Molecule/Chemotherapy Drugs

In some embodiments, the additional therapy is a small molecule drug. In some embodiments, the additional therapy is a chemotherapy drug. In some embodiments, the additional therapy is a small molecule chemotherapy drug. Such small molecule drugs can include existing standard-of-care treatment regimens to which adoptive NK cell therapy is added. In some cases, the use of the NK cells described herein can enhance the effects of small molecule drugs, including by enhancing the efficacy, reducing the amount of small molecule drug necessary to achieve a desired effect, or reducing the toxicity of the small molecule drug.

In some embodiments, the drug is selected from the group consisting of

In some embodiments, the drug is [(1S,2S,3R,4S,7R,9S,10S,12R,15S)-4-acetyloxy-1,9,12-trihydroxy-15-[(2R,3S)-2-hydroxy-3-[(2-methylpropan-2-yl)oxycarbonylamino]-3-phenylpropanoyl] oxy-10,14,17,17-tetramethyl-11-oxo-6-oxatetracyclo[11.3.1.03,10.04,7] heptadec-13-en-2-yl]benzoate (docetaxel) or a pharmaceutically acceptable salt thereof.

In some embodiments, the drug is [(1S,2S,3R,4S,7R,9S,10S,12R,15S)-4,12-diacetyloxy-15-[(2R,3S)-3-benzamido-2-hydroxy-3-phenylpropanoyl] oxy-1,9-dihydroxy-10,14,17,17-tetramethyl-11-oxo-6-oxatetracyclo[11.3.1.03,10.04.7] heptadec-13-en-2-yl]benzoate (paclitaxel) or a pharmaceutically acceptable salt thereof.

In some embodiments, the drug is 6-N-(4,4-dimethyl-5H-1,3-oxazol-2-yl)-4-N-[3-methyl-4-([1.2.4]triazolo[1,5-a]pyridin-7-yloxy)phenyl] quinazoline-4.6-diamine (tucatinib) or a pharmaceutically acceptable salt thereof.

In some embodiments, the drug is pentyl N-[1-[(2R,3R,4S,5R)-3,4-dihydroxy-5-methyloxolan-2-yl]-5-fluoro-2-oxopyrimidin-4-yl]carbamate (capecitabine) or a pharmaceutically acceptable salt thereof.

In some embodiments, the drug is azanide: cyclobutane-1,1-dicarboxylic acid: platinum (2+) (carboplatin) or a pharmaceutically acceptable salt thereof.

In some embodiments, the drug is methyl (1R,9R, 10S,11R,12R, 19R)-11-acetyloxy-12-ethyl-4-[(12S,14R)-16-ethyl-12-methoxycarbonyl-1,10-diazatetracyclo[12.3.1.03.11.04.9] octadeca-3 (11) , 4,6,8,15-pentaen-12-yl]-10-hydroxy-5-methoxy-8-methyl-8,16-diazapentacyclo[10.6.1.01.9.02.7.016.19] nonadeca-2,4,6, 13-tetraene-10-carboxylate (vinorelbine) or a pharmaceutically acceptable salt thereof.

In some embodiments, the drug is N-[3-chloro-4-[(3-fluorophenyl) methoxy]phenyl]-6-[5-[(2-methylsulfonylethylamino)methyl] furan-2-vl] quinazolin-4-amine (lapatinib) or a pharmaceutically acceptable salt thereof.

In some embodiments, the drug is (E)-N-[4-[3-chloro-4-(pyridin-2-ylmethoxy) anilino]-3-cyano-7-ethoxyquinolin-6-yl]-4-(dimethylamino) but-2-enamide (neratinib) or a pharmaceutically acceptable salt thereof.

In some embodiments, the drug is 6-acetyl-8-cyclopentyl-5-methyl-2-[(5-piperazin-1-ylpyridin-2-yl)amino]pyrido[2,3-d]pyrimidin-7-one (palbociclib) or a pharmaceutically acceptable salt thereof.

In some embodiments, the drug is 7-cyclopentyl-N,N-dimethyl-2-[(5-piperazin-1-ylpyridin-2-yl)amino]pyrrolo[2,3-d]pyrimidine-6-carboxamide (ribociclib) or a pharmaceutically acceptable salt thereof.

In some embodiments, the drug is N-[5-[(4-ethylpiperazin-1-yl)methyl] pyridin-2-yl]-5-fluoro-4-(7-fluoro-2-methyl-3-propan-2-ylbenzimidazol-5-yl)pyrimidin-2-amine (abemaciclib) or a pharmaceutically acceptable salt thereof.

In some embodiments, the drug is (1R,9S,12S,15R,16E, 18R,19R,21R,23S,24E,26E,28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.04.9] hexatriaconta-16,24,26,28-tetraene-2.3.10.14.20-pentone (everolimus) or a pharmaceutically acceptable salt thereof.

In some embodiments, the drug is (2S)-1-N-[4-methyl-5-[2-(1,1,1-trifluoro-2-methylpropan-2-yl) pyridin-4-yl]-1,3-thiazol-2-yl]pyrrolidine-1,2-dicarboxamide (alpelisib) or a pharmaceutically acceptable salt thereof.

In some embodiments, the drug is 4-[[3-[4-(cyclopropanecarbonyl) piperazine-1-carbonyl]-4-fluorophenyl]methyl]-2H-phthalazin-1-one (olaparib) or a pharmaceutically acceptable salt thereof.

In some embodiments, the drug is (11S, 12R)-7-fluoro-11-(4-fluorophenyl)-12-(2-methyl-1,2,4-triazol-3-yl)-2,3,10-triazatricyclo[7.3.1.05.13] trideca-1,5 (13).6,8-tetraen-4-one (talazoparib) or a pharmaceutically acceptable salt thereof.

In some embodiments, the drug is N-[2-[2-(dimethylamino)ethyl-methylamino]-4-methoxy-5-[[4-(1-methylindol-3-yl)pyrimidin-2-yl]amino]phenyl]prop-2-enamid (osimertinib) or a pharmaceutically acceptable salt thereof.

In some embodiments, the drug is N-(3-chloro-4-fluorophenyl)-7-methoxy-6-(3-morpholin-4-ylpropoxy) quinazolin-4-amine (gefitinib) or a pharmaceutically acceptable salt thereof.

In some embodiments, the drug is N-(3-ethynylphenyl)-6.7-bis(2-methoxyethoxy) quinazolin-4-amine (erlotinib) or a pharmaceutically acceptable salt thereof.

In some embodiments, the drug is (E)-N-[4-(3-chloro-4-fluoroanilino)-7-[(3S)-oxolan-3-ylJoxyquinazolin-6-yl]-4-(dimethylamino) but-2-enamide (afatinib) or a pharmaceutically acceptable salt thereof.

In some embodiments, the drug is azane: dichloroplatinum (cisplatin, platinol) or a pharmaceutically acceptable salt thereof.

In some embodiments, the drug is azanide: cyclobutane-1,1-dicarboxylic acid: platinum (2+) (carboplatin) or a pharmaceutically acceptable salt thereof

In some embodiments, the drug is 4-amino-1-[(2R,4R,5R)-3,3-difluoro-4-hydroxy-5-(hydroxymethyl)oxolan-2-yl|pyrimidin-2-one (gemcitabine) or a pharmaceutically acceptable salt thereof.

In some embodiments, the drug is (2S)-2-[[4-[2-(2-amino-4-oxo-3,7-dihydropyrrolo[2,3-d]pyrimidin-5-yl)ethyl]benzoyl]amino]pentanedioic acid (pemetrexed) or a pharmaceutically acceptable salt thereof.

In some embodiments, the drug is N,N-bis(2-chloroethyl)-2-oxo-1,3,2×5-oxazaphosphinan-2-amine (cyclophosphamide) or a pharmaceutically acceptable salt thereof.

In some embodiments, the drug is (2R,3S,4S,5R)-2-(6-amino-2-fluoropurin-9-yl)-5-(hydroxymethyl)oxolane-3,4-diol (fludarabine) or a pharmaceutically acceptable salt thereof.

In some embodiments, the drug is (7S,9S)-7-[(2R,4S.5S,6S)-4-amino-5-hydroxy-6-methyloxan-2-yl|oxy-6,9, 11-trihydroxy-9-(2-hydroxyacetyl)-4-methoxy-8,10-dihydro-7H-tetracene-5,12-dione (doxorubicin) or a pharmaceutically acceptable salt thereof.

In some embodiments, the drug is methyl (1R,9R, 10S, 11R, 12R, 19R)-11-acetyloxy-12-ethyl-4-[(13S,15S, 17S)-17-ethyl-17-hydroxy-13-methoxycarbonyl-1, 11-diazatetracyclo[13.3.1.04.12.05.10] nonadeca-4 (12),5,7,9-tetraen-13-yl]-8-formyl-10-hydroxy-5-methoxy-8, 16-diazapentacyclo[10.6.1.01.9.02.7.016.19] nonadeca-2,4,6, 13-tetraene-10-carboxylate (vincristine) or a pharmaceutically acceptable salt thereof.

In some embodiments, the drug is (8S,9S, 10R,13S,14S, 17R)-17-hydroxy-17-(2-hydroxyacetyl)-10,13-dimethyl-6,7,8,9, 12, 14, 15, 16-octahydrocyclopenta[a]phenanthrene-3,11-dione (prednisone) or a pharmaceutically acceptable salt thereof.

In some embodiments, the drug is N,3-bis(2-chloroethyl)-2-oxo-1,3,225-oxazaphosphinan-2-amine (ifosfamide) or a pharmaceutically acceptable salt thereof.

In some embodiments, the drug is (5S,5aR,8aR,9R)-5-[[(2R,4aR,6R,7R,8R,8aS)-7,8-dihydroxy-2-methyl-4,4a,6, 7,8,8a-hexahydropyrano[3,2-d][1,3]dioxin-6-yl]oxy]-9-(4-hydroxy-3,5-dimethoxyphenyl)-5a,6,8a,9-tetrahydro-5H-[2] benzofuro [6,5-f][1,3]benzodioxol-8-one (etopside) or a pharmaceutically acceptable salt thereof.

In some embodiments, the drug is (8S,9R,10S,11S,13S,14S,16R,17R)-9-fluoro-11,17-dihydroxy-17-(2-hydroxyacetyl)-10,13,16-trimethyl-6,7,8, 11, 12, 14,15, 16-octahydrocyclopenta[a]phenanthren-3-one (dexamethasone) or a pharmaceutically acceptable salt thereof.

In some embodiments, the drug is (8S,9R, 10S,11S,13S,14S,16R,17R)-9-fluoro-11,17-dihydroxy-17-(2-hydroxyacetyl)-10,13,16-trimethyl-6,7,8, 11, 12, 14, 15,16-octahydrocyclopenta [alphenanthren-3-one (cytarabine) or a pharmaceutically acceptable salt thereof.

In some embodiments, the NK cells, e.g., the CAR-NK cells described herein, e.g., AB-201 cells, are administered in combination with an antibody, e.g., a monoclonal antibody, an antibody-drug conjugate (ADC), a kinase inhibitor, a CDK4/5 inhibitor, an mTOR inhibitor, a PI3K inhibitor, a PARP inhibitor, or a combination thereof.

In some embodiments, the antibody is selected from the group consisting of trastuzumab, pertuzumab, margetuximab, and combinations thereof.

In some embodiments, the antibody-drug conjugate is selected from the group consisting of ado-trastuzumab emtansine, fam-trastuzumab deruxtecan, sacituzumab govitecan, and combinations thereof.

In some embodiments, the kinase inhibitor is selected from the group consisting of lapatinib, neratinib, tucatinib, and combinations thereof.

In some embodiments, the CDK4/6 inhibitor is selected from the group consisting of palbociclib, ribociclib, abemaciclib, and combinations thereof.

In some embodiments, the mTOR inhibitor is everolimus.

In some embodiments, the PI3K inhibitor is alpelisib.

In some embodiments, the PARP inhibitor is selected from the group consisting of olaparib, talazoparib, and combinations thereof.

In some embodiments, the NK cells, e.g., the CAR-NK cells described herein, e.g., AB-201 cells, are administered in combination with pertuzumab (or FDA-approved biosimilar thereof), trastuzumab (or FDA-approved biosimilar thereof) and docetaxel (or pharmaceutically acceptable salt thereof). In some embodiments, the pertuzumab (or FDA-approved biosimilar thereof) is administered at 840 mg IV day I followed by 420 mg IV. In some embodiments, the trastuzumab (or FDA-approved biosimilar thereof) is administered at 7 mg/kg IV day 1 followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration. In some embodiments, the docetaxel (or pharmaceutically acceptable salt thereof) is administered at 75-100 mg/m² IV day 1 cycled every 21 days.

In some embodiments, the NK cells, e.g., the CAR-NK cells, e.g., AB-201 cells, are administered in combination with pertuzumab (or FDA-approved biosimilar thereof), trastuzumab (or FDA-approved biosimilar thereof), and paclitaxel (or pharmaceutically acceptable salt thereof). In some embodiments, the pertuzumab (or FDA-approved biosimilar thereof) is administered at 840 mg IV day I followed by 420 mg IV, cycled every 21 days. In some embodiments, the trastuzumab (or FDA-approved biosimilar thereof) is administered at 4 mg/kg IV day 1 followed by 2 mg/kg IV weekly or 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration. In some embodiments, the paclitaxel (or pharmaceutically acceptable salt thereof) is administered at 80 mg/m² IV day 1 weekly or 175 mg/m² day I cycled every 21 days.

In some embodiments, the NK cells, e.g., the CAR-NK cells described herein, e.g., AB-201 cells, are administered in combination with tucatinib (or pharmaceutically acceptable salt thereof), trastuzumab (or FDA-approved biosimilar thereof), and capecitabine (or pharmaceutically acceptable salt thereof). In some embodiments, the tucatinib (or FDA-approved biosimilar thereof) is administered at 300 mg orally twice daily on days 1-21. In some embodiments, the trastuzumab (or FDA-approved biosimilar thereof) is administered at 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration. In some embodiments, the capecitabine (or FDA-approved biosimilar thereof) is administered at 1000 mg/m² orally twice daily on days 1-14. In some embodiments, the administration of tucatinib (or FDA-approved biosimilar thereof), trastuzumab (or FDA-approved biosimilar thereof), and capecitabine (or pharmaceutically acceptable salt thereof) is cycled every 21 days.

In some embodiments, the NK cells, e.g., the CAR-NK cells described herein, e.g., AB-201 cells, are administered in combination with ado-trastuzumab emtansine (T-DM1) (or FDA-approved biosimilar thereof). In some embodiments, the ado-trastuzumab emtansine (T-DM1) (or FDA-approved biosimilar thereof) is administered at 3.6 mg/kg IV day 1, cycled every 21 days.

In some embodiments, the NK cells, e.g., the CAR-NK cells described herein, e.g., AB-201 cells, are administered in combination with fam-trastuzumab deruxtecan-nxki (or FDA-approved biosimilar thereof). In some embodiments, the fam-trastuzumab deruxtecan-nxki (or FDA-approved biosimilar thereof) is administered at 5.4 mg/kg IV day 1, cycled every 21 days. In some embodiments, the NK cells, e.g., the CAR-NK cells described herein, e.g.,

AB-201 cells, are administered in combination with paclitaxel/carboplatin (or pharmaceutically acceptable salts thereof) and trastuzumab (or FDA-approved biosimilar thereof). In some embodiments, the carboplatin/paclitaxel (or pharmaceutically acceptable salts thereof) is administered at AUC 6 IV day 1 carboplatin and 175 mg/m² IV day 1 paclitaxel), cycled every 21 days. In some embodiments, the trastuzumab (or FDA-approved biosimilar thereof) is administered 4 mg/kg IV day I followed by 2 mg/kg IV weekly or 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the NK cells, e.g., the CAR-NK cells described herein, e.g., AB-201 cells, are administered in combination with paclitaxel/carboplatin (or pharmaceutically acceptable salts thereof) and trastuzumab (or FDA-approved biosimilar thereof). In some embodiments, the carboplatin/paclitaxel (or pharmaceutically acceptable salts thereof) is administered at AUC 2 IV carboplatin and 80 mg/m² IV day 1 paclitaxel), days 1, 8, and 15, cycled every 28 days. In some embodiments, the trastuzumab (or FDA-approved biosimilar thereof) is administered 4 mg/kg IV day I followed by 2 mg/kg IV weekly or 8 mg/kg IV day 1 followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the NK cells, e.g., the CAR-NK cells described herein, e.g., AB-201 cells, are administered in combination with trastuzumab (or FDA-approved biosimilar thereof) and paclitaxel (or pharmaceutically acceptable salt thereof). In some embodiments, the paclitaxel (or pharmaceutically acceptable salt thereof) is administered at 175 mg/m² IV day 1 cycled every 21 days or 80-90 mg/m² IV day 1 weekly. In some embodiments, the trastuzumab (or FDA-approved biosimilar thereof) is administered 4 mg/kg IV day I followed by 2 mg/kg IV weekly or 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the NK cells, e.g., the CAR-NK cells described herein, e.g., AB-201 cells, are administered in combination with trastuzumab (or FDA-approved biosimilar thereof) and docetaxel (or pharmaceutically acceptable salt thereof). In some embodiments, the docetaxel (or pharmaceutically acceptable salt thereof) is administered at 80-100 mg/m² IV day 1 cycled every 21 days or 35 mg/m² IV days 1, 8, and 15 weekly. In some embodiments, the trastuzumab (or FDA-approved biosimilar thereof) is administered 4 mg/kg IV day 1 followed by 2 mg/kg IV weekly or 8 mg/kg IV day 1 followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the NK cells, e.g., the CAR-NK cells described herein, e.g., AB-201 cells, are administered in combination with trastuzumab (or FDA-approved biosimilar thereof) and vinorelbine (or pharmaceutically acceptable salt thereof). In some embodiments, the vinorelbine (or pharmaceutically acceptable salt thereof) is administered at 25 mg/m² IV day 1 weekly or 20-35 mg/m² IV days 1 and 8, cycled every 21 days, or 25-30 mg/m² IV days 1. 8, and 15, cycled every 28 days. In some embodiments, the trastuzumab (or FDA-approved biosimilar thereof) is administered 4 mg/kg IV day 1 followed by 2 mg/kg IV weekly or 8 mg/kg IV day 1 followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the NK cells, e.g., the CAR-NK cells described herein, e.g., AB-201 cells, are administered in combination with trastuzumab (or FDA-approved biosimilar thereof) and capecitabine (or pharmaceutically acceptable salt thereof). In some embodiments, the capecitabine (or pharmaceutically acceptable salt thereof) is administered at 1000-1250 mg/m² PO twice daily days 1-14 cycled every 21 days. In some embodiments, the trastuzumab (or FDA-approved biosimilar thereof) is administered 4 mg/kg IV day 1 followed by 2 mg/kg IV weekly or 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the NK cells, e.g., the CAR-NK cells described herein, e.g., AB-201 cells, are administered in combination with lapatinib (or pharmaceutically acceptable salt thereof) and capecitabine (or pharmaceutically acceptable salt thereof). In some embodiments, the lapatinib (or pharmaceutically acceptable salt thereof) is administered at 1250 mg/m² PO daily days 1-21. In some embodiments, the capecitabine (or pharmaceutically acceptable salt thereof) is administered at 1000 mg/m² PO twice daily days 1-14, cycled every 21 days.

In some embodiments, the NK cells, e.g., the CAR-NK cells described herein, e.g., AB-201 cells, are administered in combination with trastuzumab (or FDA-approved biosimilar thereof) and lapatinib (or pharmaceutically acceptable salt thereof). In some embodiments, the administered (or pharmaceutically acceptable salt thereof) is administered at 1000 mg/m² PO daily. In some embodiments, the trastuzumab (or FDA-approved biosimilar thereof) is administered 4 mg/kg IV day I followed by 2 mg/kg IV weekly or 8 mg/kg IV day I followed by 6 mg/kg IV day I every 21 days. In some embodiments, the trastuzumab (or FDA-approved biosimilar thereof) is administered as a trastuzumab (or FDA-approved biosimilar thereof) and hyaluronidase-oysk injection for subcutaneous administration.

In some embodiments, the NK cells, e.g., the CAR-NK cells described herein, e.g., AB-201 cells, are administered in combination with neratinib (or pharmaceutically acceptable salt thereof) and capecitabine (or pharmaceutically acceptable salt thereof). In some embodiments, the neratinib is administered at 240 mg/m² PO daily on days 1-21. Ins ome embodiments, the capecitabine is administered at 750 mg/m² PO twice daily on days 1-14, cycled every 21 days.

### NK Cell Engagers

In some embodiments, the additional therapy is an NK cell engager, e.g., a bispecific or trispecific antibody.

In some embodiments, the NK cell engager is a bispecific antibody against CD16 and a disease-associated antigen, e.g., cancer-associated antigen, e.g., an antigen of cancers described herein, e.g, HER2. In some embodiments, the NK cell engager is a trispecific antibody against CD16 and two disease-associated antigens, e.g., cancer-associated antigens, e.g., antigens of cancers described herein.

### Checkpoint Inhibitors

In some embodiments, the additional therapy is an immune checkpoint inhibitor.

In some embodiments, the immune checkpoint inhibitor is selected from the group consisting of a PD-1 inhibitor, a PD-L1 inhibitor, a CTLA-4 inhibitor, and combinations thereof.

In some embodiments, the immune checkpoint inhibitor is selected from the group consisting of a PD-1 inhibitor, a PD-L1 inhibitor, a CTLA-4 inhibitor, a VISTA inhibitor, a BTLA inhibitor, a TIM-3 inhibitor, a KIR inhibitor, a LAG-3 inhibitor, a TIGIT inhibitor, a CD-96 inhibitor, a SIRPa inhibitor, and combinations thereof.

In some embodiments, the immune checkpoint inhibitor is selected from the group consisting of a PD-1 inhibitor, a PD-L1 inhibitor, a CTLA-4 inhibitor, a LAG-3 (CD223) inhibitor, a TIM-3 inhibitor, a B7-H3 inhibitor, a B7-H4 inhibitor, an A2aR inhibitor, a CD73 inhibitor, a NKG2A inhibitor, a PVRIG/PVRL2 inhibitor, a CEACAMI inhibitor, a CEACAM 5 inhibitor, a CEACAM 6 inhibitor, a FAK inhibitor, a CCL2 inhibitor, a CCR2 inhibitor, a LIF inhibitor, a CD47 inhibitor, a SIRPa inhibitor, a CSF-1 inhibitor, an M-CSF inhibitor, a CSF-1R inhibitor, an IL-1 inhibitor, an IL-1R3 inhibitor, an IL-RAP inhibitor, an IL-8 inhibitor, a SEMA4D inhibitor, an Ang-2 inhibitor, a CELVER-1 inhibitor, an Axl inhibitor, a phsphatidylserine inhibitor, and combinations thereof.

In some embodiments, the immune checkpoint inhibitor is selected from those shown in Table 9, or combinations thereof.

TABLE 9
Exemplary Immune Checkpoint Inhibitors

| Target | Inhibitor |
|---|---|
| LAG-3 (CD223) | LAG525 (IMP701), REGN3767 (R3767), BI |
| | 754.091, tebotelimab (MGD013), eftilagimod alpha |
| | (IMP321), FS118 |
| TIM-3 | MBG453, Sym023, TSR-022 |
| B7-H3, B7-H4 | MGC018, FPA150 |
| A2aR | EOS100850, AB928 |
| CD73 | CPI-006 |
| NKG2A | Monalizumab |
| PVRIG/PVRL2 | COM701 |
| CEACAM1 | CM24 |
| CEACAM 5/6 | NEO-201 |
| FAK | Defactinib |
| CCL2/CCR2 | PF-04136309 |
| LIF | MSC-1 |
| CD47/SIRPα | Hu5F9-G4 (5F9), ALX148, TTI-662, RRx-001 |
| CSF-1 | Lacnotuzumab (MCS110), LY3022855, SNDX-6352, |
| (M-CSF)/CSF-1R | emactuzumab (RG7155), pexidartinib (PLX3397) |
| IL-1 and IL-IR3 | CAN04, Canakinumab (ACZ885) |
| (IL-1RAP) | |
| IL-8 | BMS-986253 |
| SEMA4D | Pepinemab (VX15/2503) |
| Ang-2 | Trebananib |
| CLEVER-1 | FP-1305 |
| Axl | Enapotamab vedotin (EnaV) |
| Phospha- | Bavituximab |
| tidylserine | |

In some embodiments, the immune checkpoint inhibitor is an antibody.

In some embodiments, the PD-1 inhibitor is selected from the group consisting of pembrolizumab, nivolumab, toripalimab, cemiplimab-rwlc, sintilimab, and combinations thereof.

In some embodiments, the PD-L1 inhibitor is selected from the group consisting of atezolizumab, durvalumab, avolumab, and combinations thereof.

In some embodiments, the CTLA-4 inhibitor is ipilimumab.

In some embodiments, the PD-1 inhibitor is selected from the group of inhibitors shown in Table 10.

TABLE 10
Exemplary PD-1 Inhibitor Antibodies

| Name | Internal Name | Antigen | Company |
|---|---|---|---|
| nivolumab | Opdivo, ONO-4538, MDX- | PD-1 | BMS, Medarex, Ono |
| | 1106, BMS-936558, 5C4 | | |
| pembrolizumab | Keytruda, MK-3475, SCH | PD-1 | Merck (MSD), Schering- |
| | 900475, lambrolizumab | | Plough |
| toripalimab | JS001, JS-001, TAB001, | PD-1 | Junmeng Biosciences, Shanghai |
| | Triprizumab | | Junshi, TopAlliance Bio |
| cemiplimab- | Libtayo, cemiplimab, | PD-1 | Regeneron, Sanofi |
| rwlc | REGN2810 | | |
| sintilimab | Tyvyt, IBI308 | PD-1 | Adimab, Innovent, Lilly |
| MEDI0680 | AMP-514 | PD-1 | Amplimmune, Medimmune |
| LZM009 | | PD-1 | Livzon |
| vudalimab | XmAb20717 | CTLA4, PD-1 | Xencor |
| SI-B003 | | CTLA4, PD-1 | Sichuan Baili |
| | | | Pharma, Systimmune |
| Sym021 | Symphogen patent anti-PD-1 | PD-1 | Symphogen |
| LVGN3616 | | PD-1 | Lyvgen Biopharma |
| MGD019 | | CTLA4, PD-1 | MacroGenics |
| MEDI5752 | | CTLA4, PD-1 | Medimmune |
| CS1003 | | PD-1 | Cstone Pharma |
| IBI319 | IBI-319 | PD-1, | Innovent, Lilly |
| | | Undisclosed | |
| IBI315 | IBI-315 | HER2/neu, PD-1 | Beijing Hanmi, Innovent |
| budigalimab | ABBV-181, PR-1648817 | PD-1 | Abbvie |
| Sunshine | 609A | PD-1 | Sunshine Guojian Pharma |
| Guojian patent | | | |
| anti-PD-1 | | | |
| F520 | | PD-1 | Shandong New Time Pharma |
| RO7247669 | | LAG-3, PD-1 | Roche |
| izuralimab | XmAb23104 | ICOS, PD-1 | Xencor |
| LY3434172 | | PD-1, PD-L1 | Lilly, Zymeworks |
| SG001 | | PD-1 | CSPC Pharma |
| QL1706 | PSB205 | CTLA4, PD-1 | Sound Biologics |
| AMG 404 | AMG404 | PD-1 | Amgen |
| MW11 | | PD-1 | Mabwcll |
| GNR-051 | | PD-1 | IBC Generium |
| Ningbo Cancer | HerinCAR-PD1 | PD-1 | Ningbo Cancer Hosp. |
| Hosp. anti-PD- | | | |
| 1 CAR | | | |
| Chinese PLA | | PD-1 | Chinese PLA Gen. Hosp. |
| Gen. Hosp. | | | |
| anti-PD-1 | | | |
| cetrelimab | JNJ-63723283 | PD-1 | Janssen Biotech |
| TY101 | | PD-1 | Tayu Huaxia |
| AK112 | | PD-1, VEGF | Akeso |
| EMB-02 | | LAG-3, PD-1 | EpimAb |
| pidilizumab | CT-011, hBat-1, MDV9300 | PD-1 | CureTech, Medivation, Teva |
| sasanlimab | PF-06801591, RN-888 | PD-1 | Pfizer |
| balstilimab | AGEN2034, AGEN-2034 | PD-1 | Agenus, Ludwig Inst., Sloan- |
| | | | Kettering |
| geptanolimab | CBT-501, GB226, GB 226, | PD-1 | CBT Pharma, Genor |
| | Genolimzumab, Genormab | | |
| RO7121661 | | PD-1, TIM-3 | Roche |
| AK104 | | CTLA4, PD-1 | Akeso |
| pimivalimab | JTX-4014 | PD-1 | Jounce |
| IBI318 | IBI-318 | PD-1, PD-L1 | Innovent, Lilly |
| BAT1306 | | PD-1 | Bio-Thera Solutions |
| ezabenlimab | BI754091, BI 754091 | PD-1 | Boehringer |
| Henan Cancer | Teripalimab | PD-1 | Henan Cancer Hospital |
| Hospital anti- | | | |
| PD-1 | | | |
| tebotelimab | | LAG-3, PD-1 | MacroGenics |
| sindelizumab | | PD-1 | Nanjing Medical U. |
| dostarlimab | ANB011, TSR-042, ABT1 | PD-1 | AnaptysBio, Tesaro |
| tislelizumab | BGB-A317 | PD-1 | BeiGene, Celgene |
| spartalizumab | PDR001, BAP049 | PD-1 | Dana-Farber, Novartis |
| retifanlimab | MGA012, INCMGA00012 | PD-1 | Incyte, MacroGenics |
| camrelizumab | SHR-1210 | PD-1 | Incyte, Jiangsu |
| | | | Hengrui, Shanghai Hengrui |
| zimberelimab | WBP3055, GLS-010, AB122 | PD-1 | Arcus, Guangzhou Gloria |
| | | | Bio, Harbin Gloria |
| | | | Pharma, WuXi Biologics |
| penpulimab | AK105 | PD-1 | Akeso, HanX Bio, Taizhou |
| | | | Hanzhong Bio |
| prolgolimab | BCD-100 | PD-1 | Biocad |
| HX008 | | PD-1 | Taizhou Hanzhong |
| | | | Bio, Taizhou HoudeAoke Bio |
| SCT-I10A | | PD-1 | Sinocelltech |
| serplulimab | HLX10 | PD-1 | Henlix |

In some embodiments, the PD-L1 inhibitor is selected from the group of inhibitors shown in Table 11.

TABLE 11
Exemplary PD-L1 Inhibitor Antibodies

| Name | Internal Name | Antigen | Company |
|---|---|---|---|
| durvalumab | Imfinzi, MEDI-4736, | PD-L1 | AstraZeneca, Celgene, Medimmune |
| | MEDI4736 | | |
| atezolizumab | Tecentriq, | PD-L1 | Genentech |
| | MPDL3280A, RG7446, | | |
| | YW243.55.S70, | | |
| | RO5541267 | | |
| avelumab | Bavencio, | PD-L1 | Merck Serono, Pfizer |
| | MSB0010718C, A09- | | |
| | 246-2 | | |
| AMP-224 | | PD-L1 | Amplimmune, GSK, Medimmune |
| cosibelimab | CK-301, TG-1501 | PD-L1 | Checkpoint Therapeutics, Dana- |
| | | | Farber, Novartis, TG Therapeutics |
| lodapolimab | LY3300054 | PD-L1 | Lilly |
| MCLA-145 | | 4-1BB, PD-L1 | Merus |
| FS118 | | LAG-3, PD-L1 | f-star, Merck Serono |
| INBRX-105 | ES101 | 4-1BB, PD-L1 | Elpiscience, Inhibrx |
| Suzhou | | PD-L1 | Suzhou Nanomab |
| Nanomab | | | |
| patent anti-PD- | | | |
| L1 | | | |
| MSB2311 | | PD-L1 | Mabspace |
| BCD-13 | | PD-L1 | Biocad |
| opucolimab | HLX20, HLX09 | PD-L1 | Henlix |
| IBI322 | IBI-322 | CD47, PD-L1 | Innovent |
| LY3415244 | | PD-L1, TIM-3 | Lilly, Zymeworks |
| GR1405 | | PD-L1 | Genrix Biopharma |
| LY3434172 | | PD-1, PD-L1 | Lilly, Zymeworks |
| CDX-527 | | CD27, PD-L1 | Celldex |
| FS222 | | 4-1BB, PD-L1 | f-star |
| LDP | | PD-L1 | Dragonboat Biopharma |
| ABL503 | | 4-1BB, PD-L1 | ABL Bio |
| HB0025 | | PD-L1, VEGF | Huabo Biopharm |
| MDX-1105 | BMS-936559, 12A4 | PD-L1 | Medarex |
| garivulimab | BGB-A333 | PD-L1 | BeiGene |
| GEN1046 | | 4-1BB, PD-L1 | BioNTech, Genmab |
| NM21-1480 | | 4-1BB, PD- | Numab |
| | | L1, Serum | |
| | | Albumin | |
| bintrafusp alfa | M7824, | PD-L1, TGFβRII | Merck Serono, NCI |
| | MSB0011359C | | |
| pacmilimab | CX-072 | PD-L1 | CytomX |
| A167 | KL-A167 | PD-L1 | Harbour Biomed Ltd., Sichuan Kelun |
| | | | Pharma |
| IBI318 | IBI-318 | PD-1, PD-L1 | Innovent, Lilly |
| KN046 | | CTLA4, PD-L1 | Alphamab |
| STI-3031 | IMC-001 | PD-L1 | Sorrento |
| SHR-1701 | | PD-L1 | Jiangsu Hengrui |
| LP002 | | PD-L1 | Taizhou HoudeAoke Bio |
| STI-1014 | ZKAB001 | PD-L1 | Lee's Pharm, Sorrento |
| envafolimab | KN035 | PD-L1 | Alphamab |
| adebrelimab | SHR-1316 | PD-L1 | Jiangsu Hengrui, Shanghai Hengrui |
| CS1001 | | PD-L1 | Cstone Pharma |
| TQB2450 | CBT-502 | PD-L1 | CBT Pharma, Chia Tai Tianqing |
| | | | Pharma |

In some embodiments, the CTLA-4 inhibitor is selected from the group of inhibitors shown in Table 12.

TABLE 12
Exemplary CTLA4 Inhibitor Antibodies

| Name | Internal Name | Antigen | Company |
|---|---|---|---|
| ipilimumab | Yervoy, MDX-010, | CTLA4 | Medarex |
| | MDX101, 10D1, BMS- | | |
| | 734016 | | |
| ATOR-1015 | ADC-1015 | CTLA4, OX40 | Alligator |
| vudalimab | XmAb20717 | CTLA4, PD-1 | Xencor |
| SI-B003 | | CTLA4, PD-1 | Sichuan Baili |
| | | | Pharma, Systimmune |
| MGD019 | | CTLA4, PD-1 | MacroGenics |
| MEDI5752 | | CTLA4, PD-1 | Medimmune |
| ADU-1604 | | CTLA4 | Aduro |
| BCD-145 | Q3W | CTLA4 | Biocad |
| CS1002 | | CTLA4 | Cstone Pharma |
| REGN4659 | | CTLA4 | Regeneron |
| pavunalimab | XmAb22841 | CTLA4, LAG-3 | Xencor |
| AGEN1181 | | CTLA4 | Agenus |
| QL1706 | PSB205 | CTLA4, PD-1 | Sound Biologics |
| ADG126 | | CTLA4 | Adagene |
| KN044 | | CTLA4 | Changchun Intelli-Crown |
| ONC-392 | | CTLA4 | OncoImmune, Pfizer |
| BMS-986218 | | CTLA4 | BMS |
| BMS-986249 | | CTLA4 | BMS |
| BT-001 | TG6030 | CTLA4 | BioInvent |
| quavonlimab | MK-1308 | CTLA4 | Merck (MSD) |
| zalifrelimab | AGEN1884 | CTLA4 | Agenus, Ludwig Inst., Sloan- |
| | | | Kettering |
| AK104 | | CTLA4, PD-1 | Akeso |
| IBI310 | IBI-310 | CTLA4 | Innovent |
| KN046 | | CTLA4, PD-L1 | Alphamab |
| tremelimumab | ticilimumab, CP- | CTLA4 | Amgen, Medimmune, Pfizer |
| | 675206, clone 11.2.1 | | |

In some embodiments, the immune checkpoint inhibitor is a small molecule drug. Small molecule checkpoint inhibitors are described, e.g., in WO2015/034820A1, WO2015/160641A2, WO2018/009505 A1, WO2017/066227 A1, WO2018/044963 A1, WO2018/026971 A1, WO2018/045142 A1, WO2018/005374 A1, WO2017/202275 A1, WO2017/202273 A1, WO2017/202276 A1, WO2018/006795 A1, WO2016/142852 A1, WO2016/142894 A1, WO2015/033301 A1, WO2015/033299 A1, WO2016/142886 A2, WO2016/142833 A1, WO2018/051255 A1, WO2018/051254 A1, WO2017/205464 A1, US2017/0107216 A1, WO2017/070089A1, WO2017/106634A1, US2017/0174679 A1, US2018/0057486 A1, WO2018/013789 A1, US2017/0362253 A1, WO2017/192961 A1, WO2017/118762 A1, US2014/199334 A1, WO2015/036927 A1, US2014/0294898 A1, US2016/0340391 A1, WO2016/039749 A1, WO2017/176608 A1, WO2016/077518 A1, WO2016/100608 A1, US2017/0252432 A1, WO2016/126646 A1, WO2015/044900 A1, US2015/0125491 A1, WO2015/033303 A1, WO2016/142835 A1, WO2019/008154 A1, WO2019/008152 A1, and WO2019023575A1.

In some embodiments, the PD-1 inhibitor is 2-[[4-amino-1-[5-(1-amino-2-hydroxypropyl)-1,3,4-oxadiazol-2-yl]-4-oxobutyl]carbamoylamino]-3-hydroxypropanoic acid (CA-170).

In some embodiments, the immune checkpoint inhibitor is(S)-1-(3-Bromo-4-((2-bromo-[1, l′-biphenyl]-3-yl) methoxy)benzyl) piperidine-2-carboxylic Acid.

In some embodiments, the immune checkpoint inhibitor is a peptide. See, e.g., . Sasikumar et al., “Peptide and Peptide-Inspired Checkpoint Inhibitors: Protein Fragments to Cancer Immunotherapy,” Medicine in Drug Discovery 8:100073 (2020).

### Variants

In some embodiments, the fusion protein(s) or components thereof described herein, or the NK cell genotypes described herein, are at least 80%, e.g., at least 85%, 90%, 95%, 98%, or 100% identical to the amino acid sequence of an exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e, a query) and a second polypeptide or nucleic acid sequence (i.e, target) is determined in various ways that are within the skill in the art, for instance, using publicly available computer software such as Smith Waterman Alignment (Smith, T. F, and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure. Dayhof. M.O., Ed, pp 353-358: BLAST program (Basic Local Alignment Search Tool: (Altschul, S. F., W. Gish, et al. (1990) J Mol Biol 215:403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine: valine, isoleucine, leucine: aspartic acid, glutamic acid, asparagine, glutamine: serine, threonine: lysine, arginine; and phenylalanine, tyrosine.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the term “buffer solution” refers to an aqueous solution consisting of a mixture of a weak acid and its conjugate base, or vice versa.

As used herein, the term “cell culture medium” refers to a mixture for growth and proliferation of cells in vitro, which contains essential elements for growth and proliferation of cells such as sugars, amino acids, various nutrients, inorganic substances, etc.

A buffer solution, as used herein, is not a cell culture medium.

As used herein, the term “bioreactor” refers to a culture apparatus capable of continuously controlling a series of conditions that affect cell culture, such as dissolved oxygen concentration, dissolved carbon dioxide concentration, pH, and temperature.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule(s) or polynucleotide(s) of the present application. Certain vectors are capable of directing the expression of nucleic acids to which they are operatively linked. Such vectors are referred to herein as expression vectors.

The term “operably linked” refers to two or more nucleic acid sequence or polypeptide elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The terms “host cell,” “host cell line,” and “host cell culture” are used interchangeably and refer to cells into which exogenous nucleic acid has been introduced, including the progeny of such cells. Host cells include “engineered cells,” “transformants,” and “transformed cells,” which include the primary engineered (e.g., transformed) cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding a fusion protein, as described herein.

The section headings used herein are for organizational purposes only and are not to be construed as limiting the subject matter described.

### EXAMPLES

The following examples are included for illustrative purposes only and are not intended to limit the scope of the invention.

### Example 1: Off-the-Shelf NK Cell Therapy Platform

One example of a method by which NK cells were expanded and stimulated is shown in FIG. 1.

As shown in FIG. 1, a single unit of FDA-licensed, frozen cord blood that has a high affinity variant of the receptor CD16 (the 158 V/V variant, see, e.g., Koene et al., “FcγRIIIa-158V/F Polymorphism Influences the Binding of IgG by Natural Killer Cell FcgammaRIIIa, Independently of the FcgammaRIIIa-48L/R/H Phenotype,” Blood 90:1109-14 (1997).) and the KIR-B genotype (KIR B allele of the KIR receptor family, see, e.g., Hsu et al., “The Killer Cell Immunoglobulin-Like Receptor (KIR) Genomic Region: Gene-Order, Haplotypes and Allelic Polymorphism,” Immunological Review 190:40-52 (2002); and Pyo et al., “Different Patterns of Evolution in the Centromeric and Telomeric Regions of Group A and B Haplotypes of the Human Killer Cell Ig-like Receptor Locus,” PloS One 5: e15115 (2010)) was selected as the source of NK cells.

The cord blood unit was thawed and the freezing medium was removed via centrifugation. The cell preparation was then depleted of T cells using the QuadroMACS Cell Selection System (Miltenyi) and CD3 (T cell) MicroBeads. A population of 6×10⁸ total nucleated cells (TNC) were labelled with the MicroBeads and separated using the QuadroMACS device and buffer. Following depletion of T cells, the remaining cells, which were predominantly monocytes and NK cells, were washed and collected in antibiotic-free medium (CellgroSCGM). The cell preparation was then evaluated for total nucleated cell count, viability, and % CD3+ cells. The cord blood NK cells were CD3 depleted.

The CD3-cell preparation was inoculated into a gas permeable cell expansion bag containing growth medium. The cells were co-cultured with replication incompetent engineered HuT-78 (eHUT-78) feeder cells to enhance expansion for master cell bank (MCB) production. The CellgroSCGM growth media was initially supplemented with anti-CD3 antibody (OKT3), human plasma, glutamine, and IL-2.

The NK cells are optionally engineered, e.g., to introduce CARs into the NK cells, e.g., with a lentiviral vector, during one of the co-culturing steps.

The cells were incubated as a static culture for 12-16 days at 37° C., in a 5% CO2 balanced air environment, with additional exchanges of media occurring every 2 to 4 days. After the culture expanded more than 100-fold, the cultured cells were harvested and then suspended in freezing medium and filled into cryobags. In this example, 80 bags or vials were produced during the co-culture. The cryobags were frozen using a controlled rate freezer and stored in vapor phase liquid nitrogen (LN2) tanks below-150° C. These cryopreserved NK cells derived from the FDA-licensed cord blood unit served as the master cell bank (MCB).

To produce the drug product, a bag of frozen cells from the MCB was thawed and the freezing medium was removed. The thawed cells were inoculated into a disposable culture bag and co-cultured with feeder cells, e.g., eHUT78 feeder cells to produce the drug product. In this example, the cells are cultured in a 50 L bioreactor to produce thousands of lots of the drug product per unit of cord blood (e.g., 4,000-8,000 cryovials at 10⁹ cells/vial or 13,500 cryovials at 10⁸ cells/vial), which are mixed with a cryopreservation composition and frozen in a plurality of storage vessels, such as cryovials. The drug product is an off-the-shelf infusion ready product that can be used for direct infusion. Each lot of the drug product can be used to infuse hundreds to thousands of patients (e.g., 100-1,000 patients, e.g, with a target dose of 4×10⁹ cells or 1×10⁸ cells).

### Example 2: Feeder Cell Expansion

As one example, suitable feeder cells, e.g., eHut-78 cells, were thawed from a frozen stock and expanded and cultured in a 125 mL flask in growth medium comprising RPMI1640 (Life Technologies), inactivated fetal bovine serum (FBS) (Life Technologies), and glutamine (Hyclone) at or at about 37° C., and at or at about 3-7% CO2. The cells were split every 2-3 days into 125 mL-2L flasks. The cells were harvested by centrifugation and gamma irradiated. The harvested and irradiated cells were mixed with a cryopreservation medium (Cryostor CS10) in cryovials and frozen in a controlled rate freezer, with a decrease in temperature of about 15° C. every 5 minutes to a final temperature of or of about−90° C., after which they were transferred to a liquid nitrogen tank or freezer to a final temperature of or of about−150° C.

After freezing, cell viability was greater than or equal to 70% of the original number of cells, and 85% or more of the cells expressed tmTNF-α, 85% or more of the cells expressed mbIL-21+, and 85% or more of the cells expressed 4-1 BBL.

### Example 3: NK Cell Expansion and Stimulation

As one example, suitable NK cells can be prepared as follows using HuT-78 cells transduced to express 4-1BBL, membrane bound IL-21 and mutant TNFalpha (“eHut-78P cells”) as feeder cells. The feeder cells are suspended in 1% (v/v) CellGro medium and are irradiated with 20,000 cGy in a gamma-ray irradiator. Seed cells (e.g., CD3-depleted PBMC or CD3-depleted cord blood cells) are grown on the feeder cells in CellGro medium containing human plasma, glutamine, IL-2, and OKT-3 in in static culture at 37° C. The cells are split every 2-4 days. The total culture time was 19 days. The NK cells are harvested by centrifugation and cryopreserved. Thawed NK are administered to patients in infusion medium consisting of:

Phosphate Buffered Saline (PBS 1×, FujiFilm Irvine) (50% v/v), albumin (human) (20% v/v of OctaPharma albumin solution containing: 200 g/L protein, of which≥96% is human albumin, 130-160 mmol sodium: ≤2 mmol potassium. 0.064-0.096 mmol/g protein N-acetyl-DL-tryptophan, 0.064-0.096 mmol/g protein, caprylic acid, ad. 1000 ml water), Dextran 40 in Dextrose (25% v/v of Hospira Dextran 40 in Dextrose Injection, USP containing: 10 g/100 mL

Dextran 40 and 5 g/100 mL dextrose hydrous in water) and dimethyl sulfoxide (DMSO) (5% v/v of Avantor DMSL solution with a density of 1.101 g/cm3 at 20° C.).

In this example, the seed cells are CD3-depleted cord blood cells. A cell fraction can be depleted of CD3 cells by immunomagnetic selection. for example, using a CliniMACS T cell depletion set ((LS Depletion set (162-01) Miltenyi Biotec).

Preferably, the cord blood seed cells are selected to express CD16 having the V/V polymorphism at F158 (Fc gamma RIIIa-158 V/V genotype) (Musolino et al. 2008 J Clin Oncol 26:1789). Preferably, the cord blood seed cells are KIR-B haplotype.

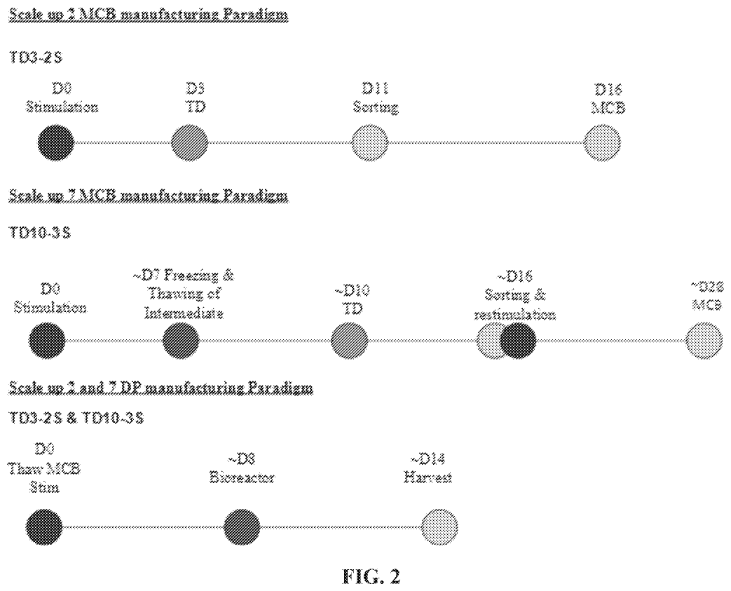

Examples of two different manufacturing timelines are shown in FIG. 2. In one scheme, a master cell bank (MCB) is generated by stimulation of a NK cell source (e.g., a single cord blood unit) with feeder cells (e.g., el luT-78, as described herein) starting at day ( ) (DO), followed by transduction, e.g., with a vector comprising a CAR described herein, e.g., as described in Example 8, at Day 3 (D3), sorting, e.g., for CAR expression, at day 11 (D11), and harvesting and cryopreserving for a MCB at day 16 (D16). In another scheme, a MCB is generated by stimulation of a NK cell source (e.g., a single cord blood unit) with feeder cells (e.g., eHuT-78, as described herein) starting at day 9 (DO), followed by freezing & thawing of an intermediate at around day 7 (D7), transduction, e.g., with a vector comprising a CAR described herein, at around day 10 (D10), sorting and restimulation at around day 16 (D16) and harvesting for a MCB at about day 28 (D28). In some cases, a drug product (DP) is manufactured by thawing and stimulating a MCB (e.g., derived from one of the manufacturing timelines described above) with feeder cells (e.g., eHuT-78, as described here), starting at day 0) (DO), followed by bioreactor culturing at about day 8 (D8) and harvesting and cryopreserving for a drug DP at about day 14. In these examples, the initial NK: feeder cell ratio can be 1:2.5 and incubation can occur, for example as static culture at 37° C., in a 5% CO2 balanced air environment in a growth medium (for example, those described herein). Sorting can be carried out, for example, using an antibody specific for the CAR. The resulting cells can be frozen in a cryopreservation medium (for example, as described herein).

### Example 4: Cord Blood as an NK Cell Source

NK cells make up five to 15% of peripheral blood lymphocytes. Traditionally, peripheral blood has been used as the source for NK cells for therapeutic use. However, as shown herein, NK cells derived from cord blood have a nearly ten-fold greater potential for expansion in the culture systems described herein than those derived from peripheral blood, without premature exhaustion or senescence of the cells. The expression of receptors of interest on the surface of NK cells, such as those involved in the activation of NK cells on engagement of tumor cells, was seen to be more consistent donor-to-donor for cord blood NKs than peripheral-blood NK cells. The use of the manufacturing process described herein consistently activated the NK cells in cord blood in a donor-independent manner, resulting in a highly scaled, active and consistent NK cell product.

### Example 5: Expanded and Stimulated NK-Cell Phenotype

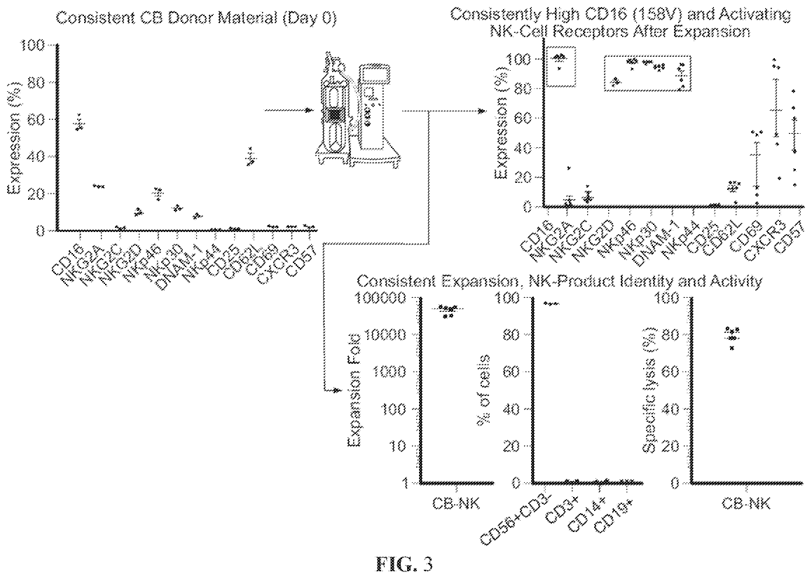

In one example NK cells from a cord blood unit are expanded and stimulated with eHut-78 cells, according to the expansion and stimulation process described in Example 1. As shown in FIG. 3, the resulting expanded and stimulated population of NK cells have consistently high CD16 (158V) and activating NK-cell receptor expression.

### Example 6: CAR Costimulatory Structure Comprising OX40L

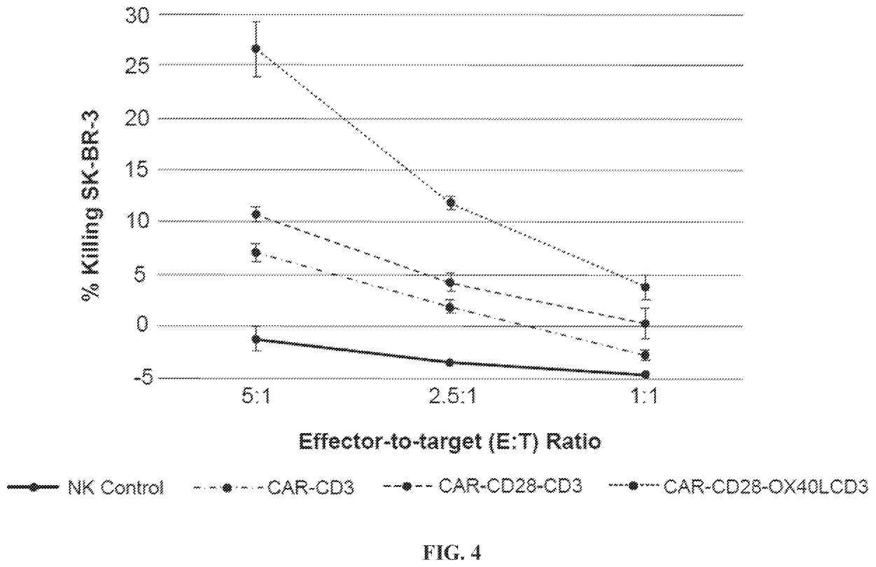

In some embodiments, the NK cells are CAR-NK cells. As shown in FIG. 4, CAR-NKs comprising a co-stimulatory domain comprising OX40L exhibited greater cytotoxic potential than those without OX40L. In this example, the CAR-NK cells comprise an anti-HER2 scFv as described in US20200399397A1, which is hereby incorporated by reference in its entirety.

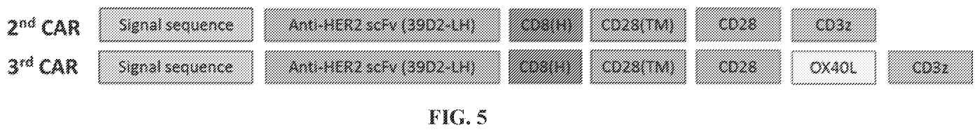

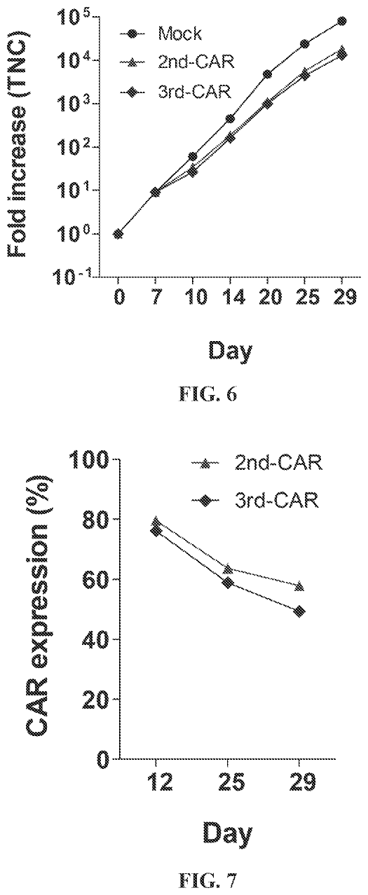

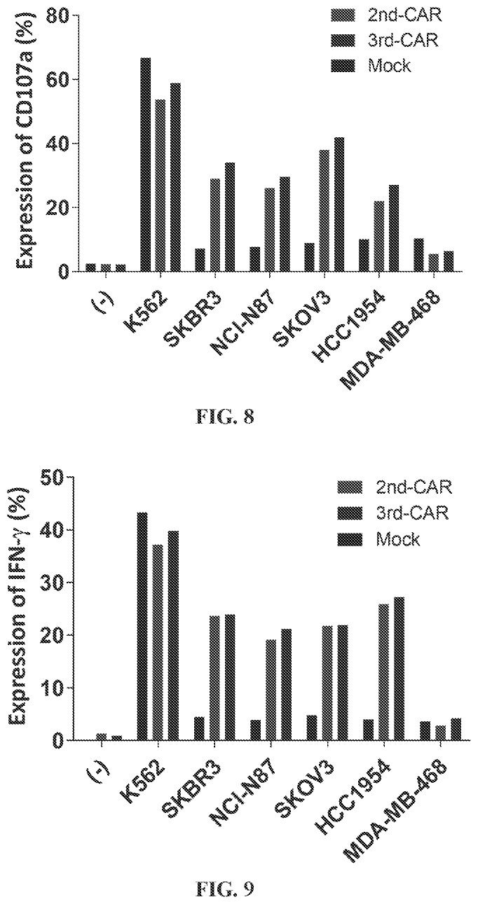

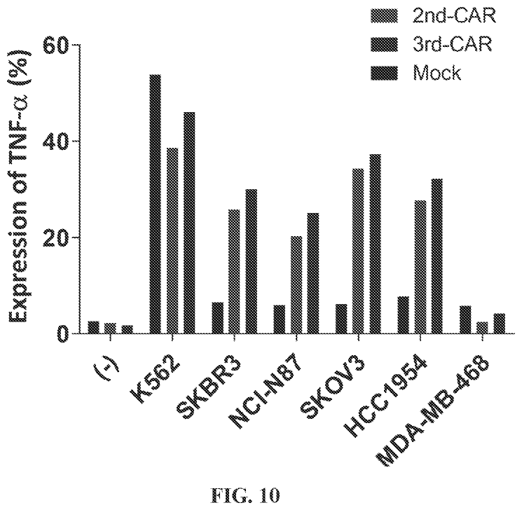

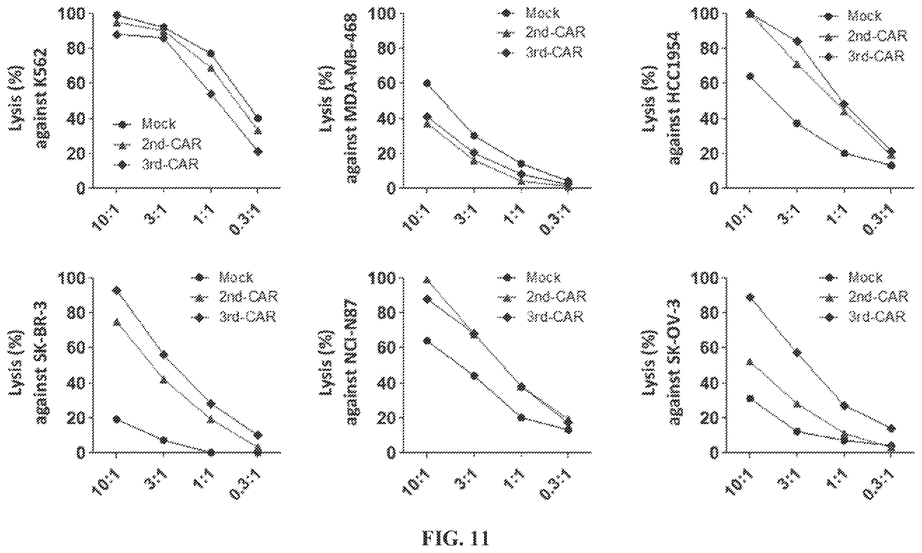

In vitro efficacy, proliferation, CAR expression, and in vitro efficacy was compared for NK-CARs comprising the CARs with anti-HER2 scFy with (SEQ ID NO: 64) and without OX40L (SEQ ID NO: 66) (FIG. 5). As shown in FIG. 6 and FIG. 7, both CAR-NK structures proliferated in tumor negative control cells and expressed the CAR. In vitro efficacy (CD107a expression, cytokine production, and percent lysis) is shown for various cell lines (HER2 positive and trastuzumab sensitive target cells (SKBR3, NCI-N87, and SKOV-3). HER2 positive and trastuzumab resistant target cells (HCC1954), and HER2 negative target cells (MDA-MB-468) in FIG. 8, FIG. 9, FIG. 10, and FIG. 11). The OX40L containing CAR showed greater cytotoxic potential than that the CAR without OX40L against HER2 positive cell lines.

### Example 7: AB-201

AB-201 is comprised of ex vivo expanded allogeneic cord blood derived natural killer (NK) cells that have been genetically modified to express a Human epidermal growth factor receptor 2 positive (HER2) directed chimeric antigen receptor (CAR) and IL-15 in a cryopreserved infusion ready suspension medium.

AB-201 is a cell suspension for infusion in buffered saline (with albumin, Dextran 40, and 5% DMSO), formulated as shown in Table 13.

TABLE 13
Components and Compositions of AB-201

| Component | Solution | | | Quantity per |
|---|---|---|---|---|
| Solution | Composition | Conc | Conc | Unit (11 mL fill) |
| AB-201 drug | Approximately | 54% v/v | 0.54 mL/mL | 5.94 mL |
| substance (ex | 1 × 10⁷, 1 × 10⁸, or | | | |
| vivo- expanded | 1 × 10⁹ viable cells | | | |
| allogeneic CAR- | | | | |
| NK cells) | | | | |
| PBS | 100% Phosphate | | | |
| | Buffered Saline | | | |
| | (PBS) | | | |
| Albumin | 250 g/L albumin in | 16% v/v | 40 mg/mL albumin | 1.76 mL |
| Solution | water | | | |
| Dextran 40 | 100 g/L Dextran 40; | 25% v/v | 25 mg/mL Dextran | 2.75 mL |
| Solution | and 50 g/L dextrose; | | 40; | |
| | in water | | 12.5 mg/mL dextrose | |
| DMSO | 100% DMSO (1,100 | 5% v/v | 55 mg/mL | 0.55 mL |
| | g/L) | | | |

### Example 8: AB-201 Production

A CAR-NK expressing the fusion protein having SEQ ID NO: 59 was produced by transducing NK cells with a vector comprising SEQ ID NO: 61. The manufacture of AB-201 is conducted over a 2-stage process. Stage 1 produces the AB-201 master cell bank (identified as AB-201M), while stage 2 produces the AB-201 drug product (identified as AB-201P).

### Example 9: CAR Constructs Expressing IL-15 have Increased Cytotoxicity

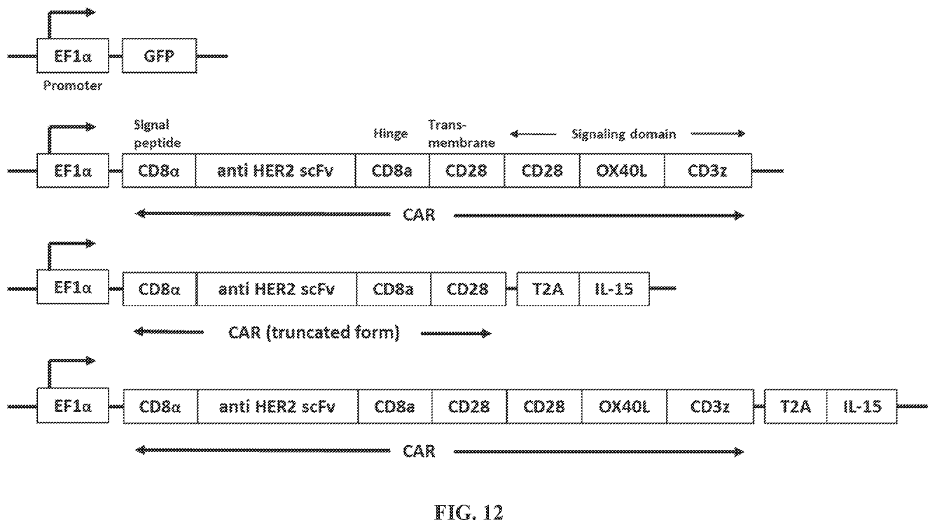

To investigate whether a CAR-NK which expresses both CAR and IL-15 has a synergistic effect on cytotoxicity, CAR-NK structures were generated as shown in FIG. 12.

NK cells including NK, mock-NK, CAR-NK, CAR (t)-IL-15-NK, and CAR-IL-15-NK (AB-201) group were generated from cord blood of a healthy donor. The CD3 negative cells in cord blood unit were purified by using CD3+ cells positive isolation kit, and then they were used as seed cells.

The seed cells included CD56+NK cells were stimulated with irradiated eHuT-78P cells and OKT3 and recombinant IL-2 (Proleukin) in complete serum-free medium (CellGro) on day 0.

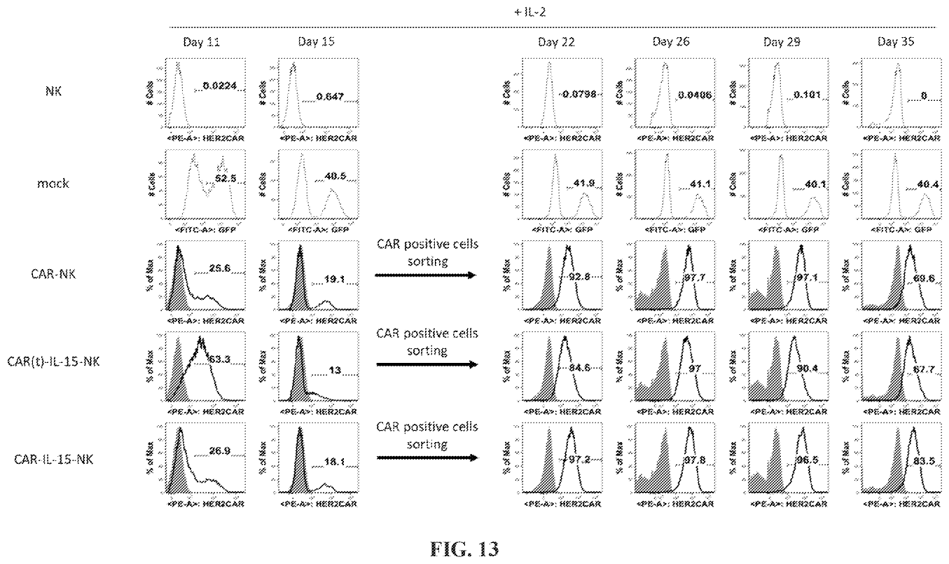

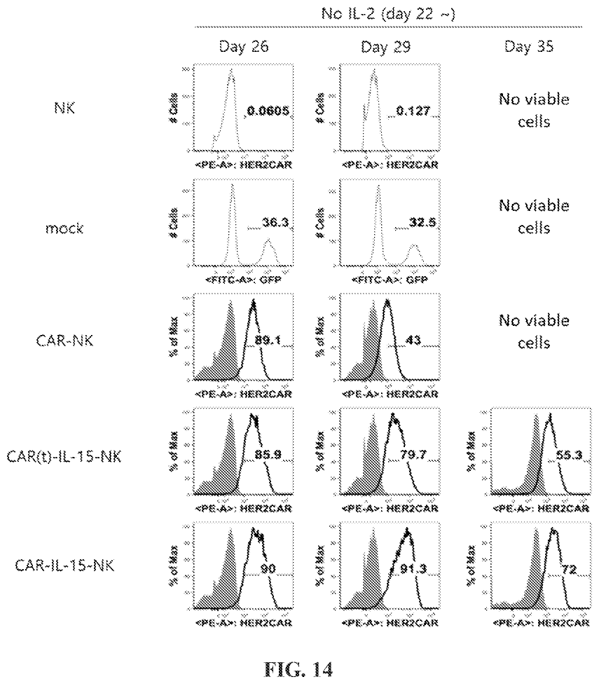

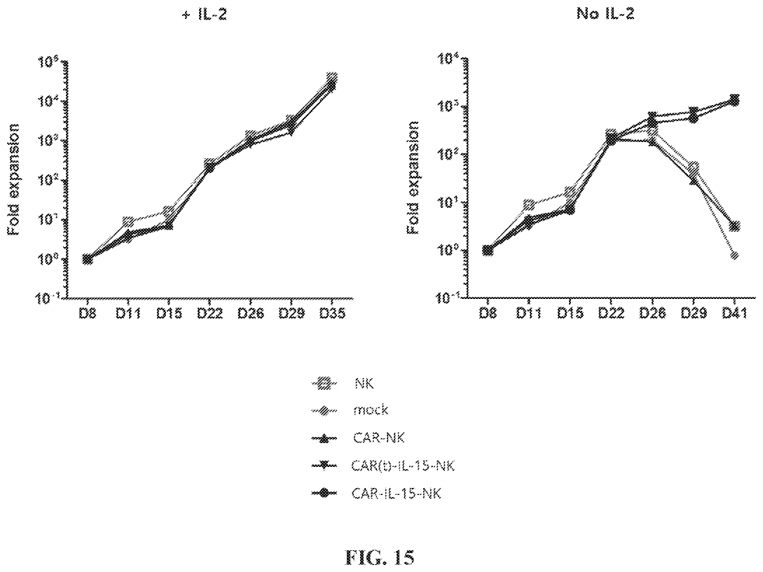

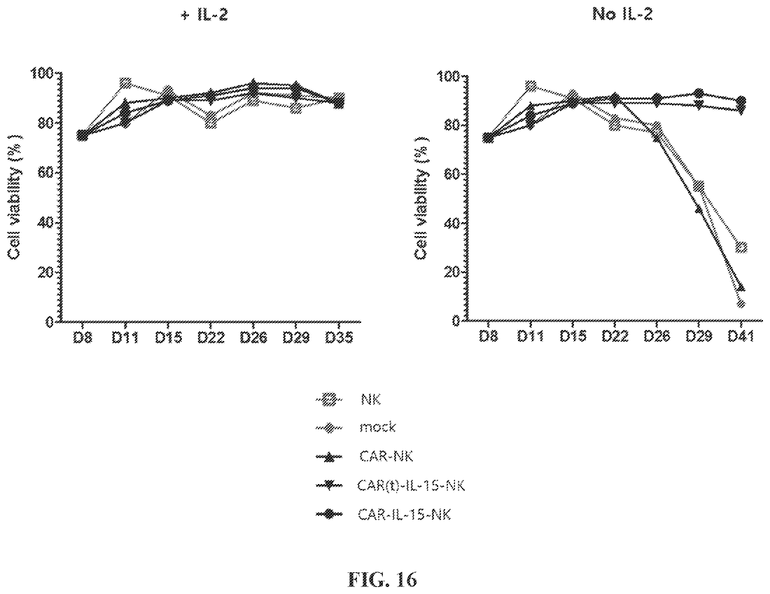

The cultured NK cells were transduced by lentiviral vector on day 6 or 8 and were stimulated again with the irradiated eHuT-78P cells and OKT3 and IL-2 on day 14. At day 22. the cell groups were divided two groups again and cultured in the presence or absence of IL-2, respectively. Both transduced and non-transduced NK cells were cultured for 35 days in the presence of IL-2. As shown in FIG. 13, IL-15 secreting transduced expressed the CAR stably until day 35. As shown in FIG. 14, only IL-15 secreting transduced NK cells survived and expressed the CAR in the absence of IL-2. Moreover, the results show that expression of IL-15 increases the proportion of cells that are CAR+ in the absence of IL-2 (compare CAR-NK (43%) and CAR-IL-15-NK (91.3%) at day 29). As shown in FIG. 15, NK cells not secreting IL-15 did not proliferate after day 22 and, as shown in FIG. 16, their viability decreased rapidly after day 22. The results show that recombinant expression of IL-15 extends survival of NK cells even in the absence of IL-2.

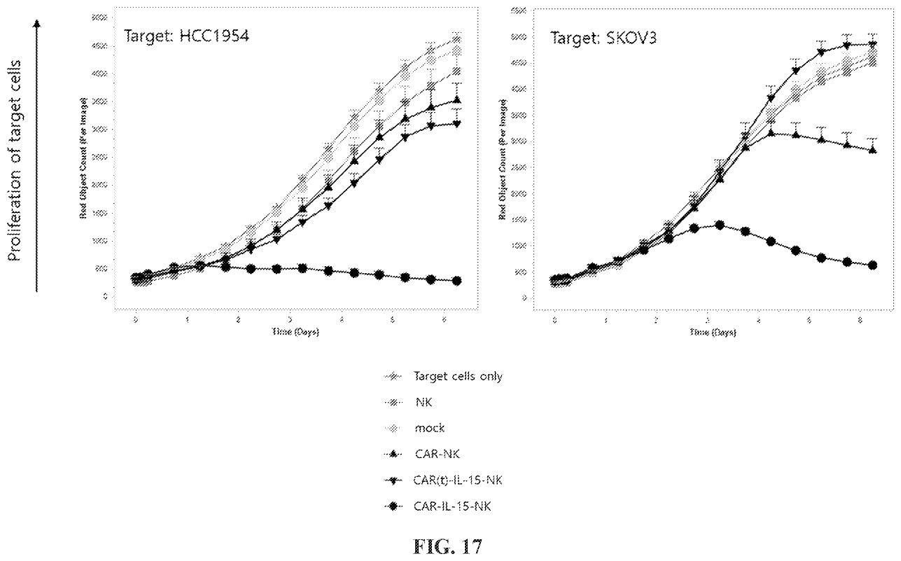

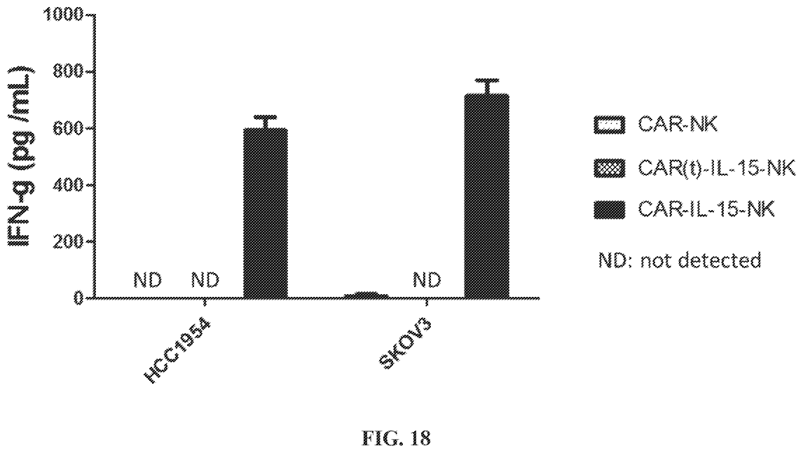

To measure cytotoxicity, NK cells were cultured in the presence of IL-2 until day 22. and then cultured four more days in the absence of IL-2. At day 26, the NK cells were co-cultured with HCC1954 or SKOV3 at the E: T=0.3:1 ratio for long-term killing assay (FIG. 17) or E: T=1:1 ratio for IFNg ELISA (FIG. 18) for 6 days in the absence of IL-2. CAR-IL-15-NK cells had a higher cytotoxicity than that of other NK cells. These results show that the CAR comprising an OX40L costimulatory domain and IL-15 expression exhibited better and more sustained killing activity. The CAR-NK cells lacking IL-15 expression showed significantly reduced killing activity compared to the cells expressing IL-15 under these conditions. On day 32, the amount of IFNg in the culture supernatant was measured. The CAR-IL-15-NK cells produced the highest amount of IFNg, and it was correlated to the cytolytic activity results in FIG. 17.

To measure IL-15 production, the non-transduced or transduced NK cells were cultured in the presence of IL-2 until day 28, and then cultured four more days in the absence of IL-2. At day 32, the indicated NK cells were co-cultured with HCC1954 or SKOV3 for 72 hours in the absence of IL-2, and the IL-15 levels in the cultured supernatant were determined by ELISA, as shown in FIG. 19. These results show that co-culturing CAR-IL-15-NK cells in the presence of HER2+ target cells increased the amount of IL-15 produced by the NK cells. In contrast, the CAR (t)-IL-15-NK cells without costimulatory domains generated relatively constant amounts of basal IL-15 expression in both the absence of and presence of target cells. NK cells that lacked recombinant nucleic acids encoding IL-15 did not generate significant levels of IL-15 expression.

### Example 10: Secretion of IL-15 Maintains the Survival of Bystander NK Cells

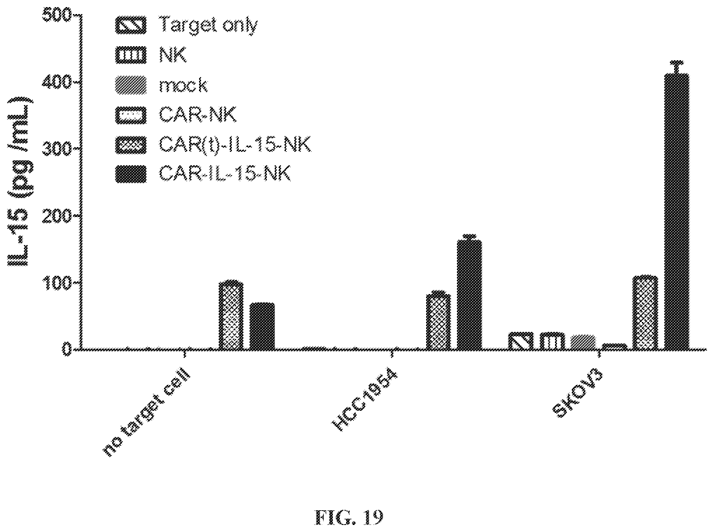

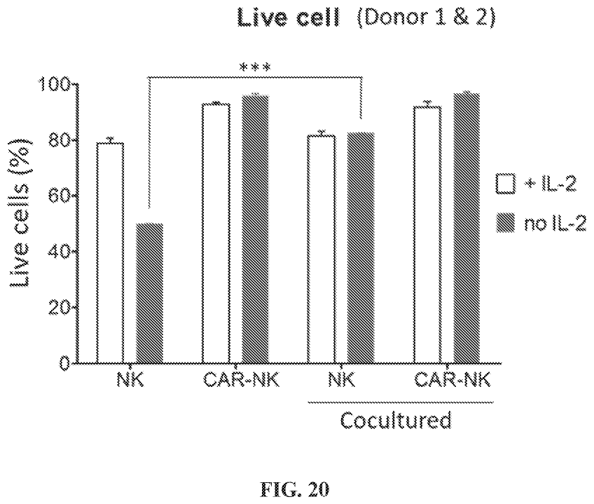

NK cells and CAR-IL-15-NK (AB-201) cells were generated from two different donors. Cells were transduced at day 8 to produce CAR-IL-15-NK. At day 14, the NK cells were re-stimulated and CAR-IL-15-NK cells were re-stimulated and sorted. At day 19, the NK cells were CFSE labeled and co-cultures were created by mixing CFSE NK cells and CAR-IL-15-NK cells at a 1:1 ratio. Cocultures either with or without IL-2 were carried out for 5 days. Fixable viability dye (Invitrogen #65-0865) was used to detect viable cells. As shown in FIG. 20, despite the absence of IL-2, the frequency of living NK cells that co-cultured with CAR-IL-15-NK cells was not decreased in the experiments using two different donors.

### Example 11: Long Term Stability and Survival of CAR-NKs Expressing IL-15

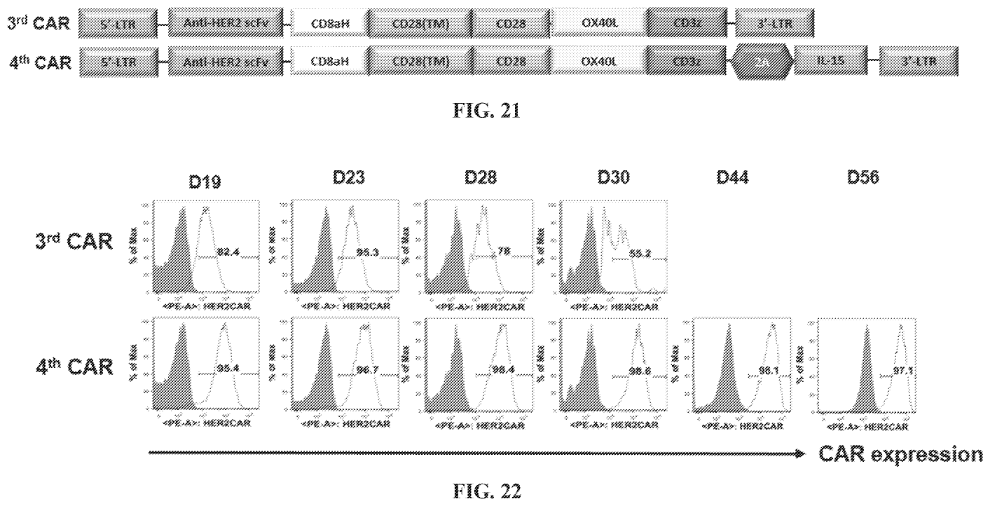

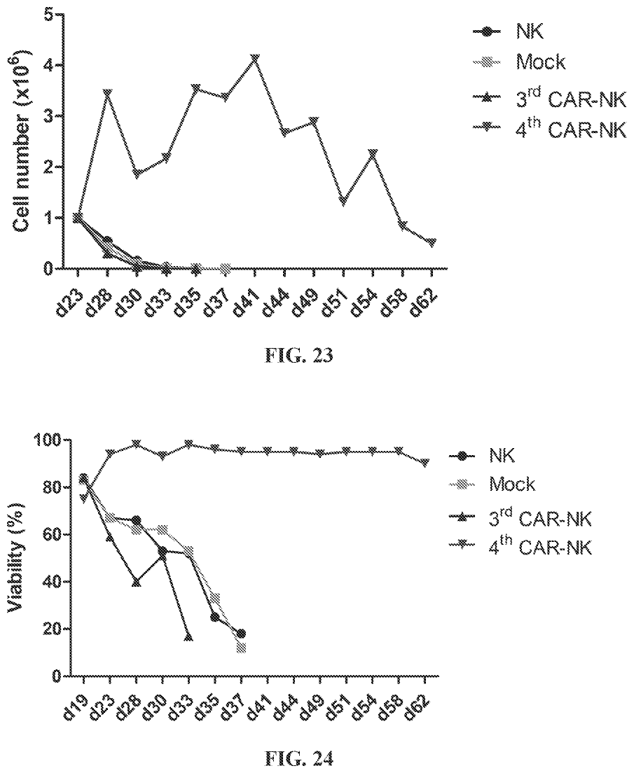

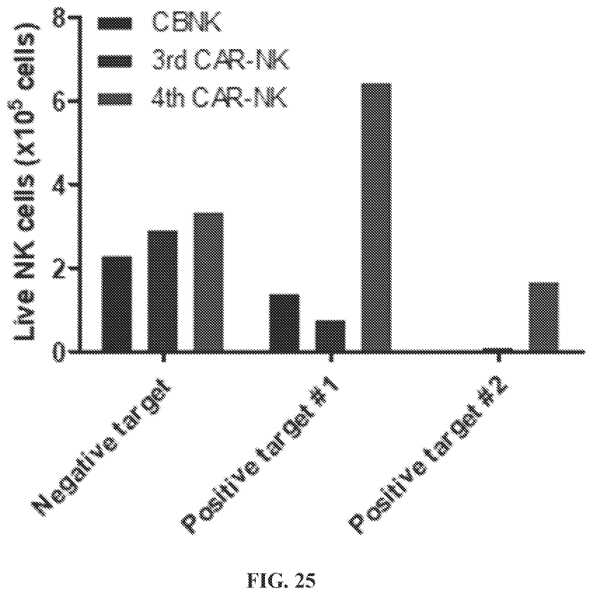

NK cells expressing CARs with and without IL-15 (FIG. 21) were cultured as described in Example 10 to day 19. At day 19, they were cultured without IL-2. As shown in FIG. 22, the CAR-expressing cells lacking IL-15 (3rd CAR) had significantly reduced CAR expression levels (e.g., only 55.2% of max at day 30) as compared to cells expressing the CAR with IL-15 (4th CAR) (e.g., 97.1% of max at day 56). The CAR-expressing cells lacking IL-15 (3rd CAR) also failed to persist as long, as none survived until day 44. As shown in FIG. 23 and FIG. 24, the cells expressing the CAR lacking IL-15 did not survive past day 37, whereas the cells expressing the CAR with IL-15 survived at least up to day 62, and also maintained viability. As shown in FIG. 25, cells expressing IL-15 persisted better than cells lacking heterologous expression of IL-15 in the presence target cells.

### Example 12: AB-201 In vitro Studies

### Characterization of AB-201

Purity and phenotype of AB-201 was evaluated by flow cytometry. NK cell maturation is determined through expression of the markers, CD56 and CD16 while NK cell activity and regulation are conferred through a balance of activating and inhibitory receptor expression. The expression pattern of these receptors was determined by flow cytometry using receptor-specific reagent antibody staining. Cord blood NK (CB-NK) cells were used as a control.

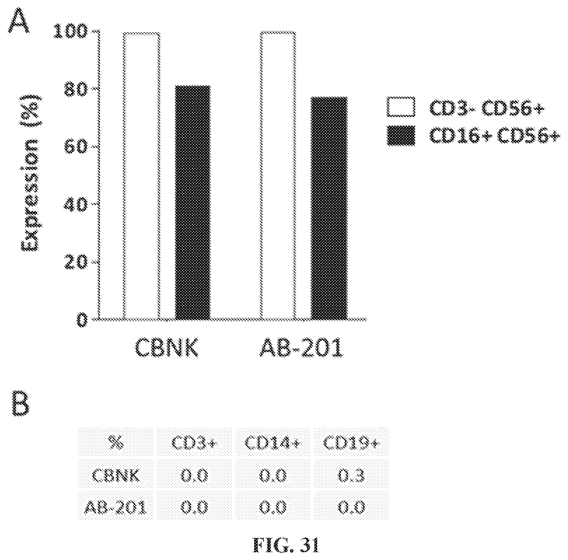

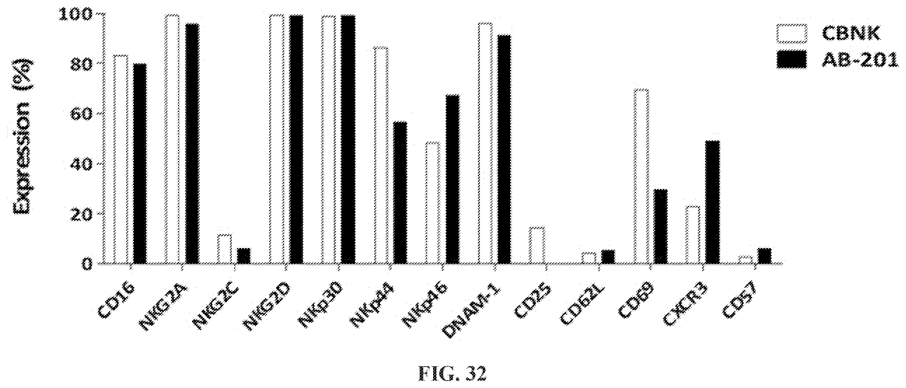

AB-201 purity and identity was determined through the assessment of surface markers CD56, CD3, CD14, and CD19. CD56 is the archetypal phenotypic marker of natural killer maturation whereas CD3, CD14, and CD19 are markers for T cells, monocytes, and B cells, respectively. Expression of CD16 (FcγRIII) is also an indicator of the NK cell maturation state (FIG. 31). Further, CD3, CD14, and CD19 constitute 0% of the AB-201 cell population (FIG. 31). Further characterization of AB-201 demonstrated high expression of activating receptors such as NKG2D, NKp30, NKp46, and DNAM-1 and expression of the chemokine receptor. CXCR3 (FIG. 32). The mean number of cells in the AB-201 sample that expressed the CAR was 92.5%.

### Killing Activity of AB-201

Cytotoxicity of AB-201 against tumor cell lines was assessed using short (4 hr) and long-term (up to 5 day) assays. Cytotoxicity of NK cells can be quantitatively measured at a range of NK cell (effector) to tumor cell (target) ratios. Target cells included SKOV-3, HCC1954, and NCI-N87, HER2+ cancer cell lines of ovarian, breast, and gastric origin, respectively.

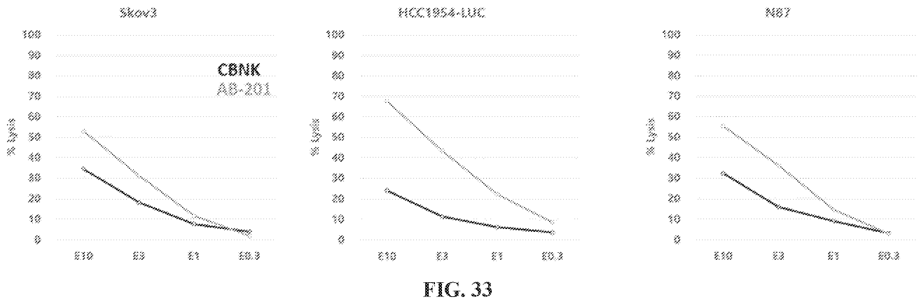

AB-201 demonstrated concentration-dependent cytotoxic activity against the tumor cell lines SKOV-3, HCC1954, and NCI-N87 (FIG. 33). Cytotoxic activity of AB-201 was greater than the donor-matched, non-engineered, eHuT-78-expanded cord-blood derived NK cells (CBNK) in all the cell lines tested. These results indicate that AB-201 has potent cytotoxic activity against HER2+ cancer cell lines.

Long-term cytotoxicity assays were performed using an Incucyte Live-Cell analysis system which images the NK and target cell co-culture over time.

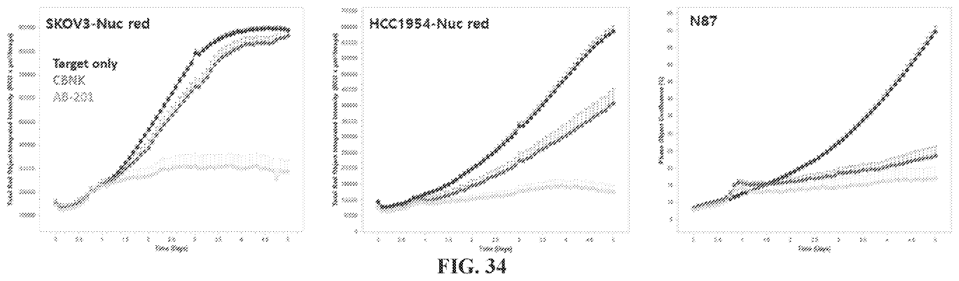

AB-201 demonstrated potent long-term cytotoxic activity against SKOV-3, HCC-1954, and NCI-N87 cancer cell lines over the 5 day timeframe (FIG. 34). In co-cultures with SKOV-3 or HCC-1954 the cytotoxic activity of AB-201 exceeds that observed with non-engineered CBNK cells. When AB-201 is co-cultured with NCI-N87 cells the differential between AB-201 and CBNK cells is less; however, the NCI-N87 experiment was performed utilizing phase contrast analysis of tumor cell confluence which could be a less sensitive measure in this system.

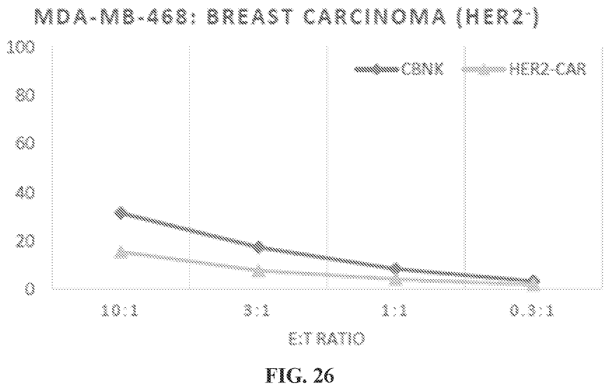

FIG. 26 shows in vitro killing activity of AB-201 against the breast carcinoma (HER2-) cell line MDA-MB-468.

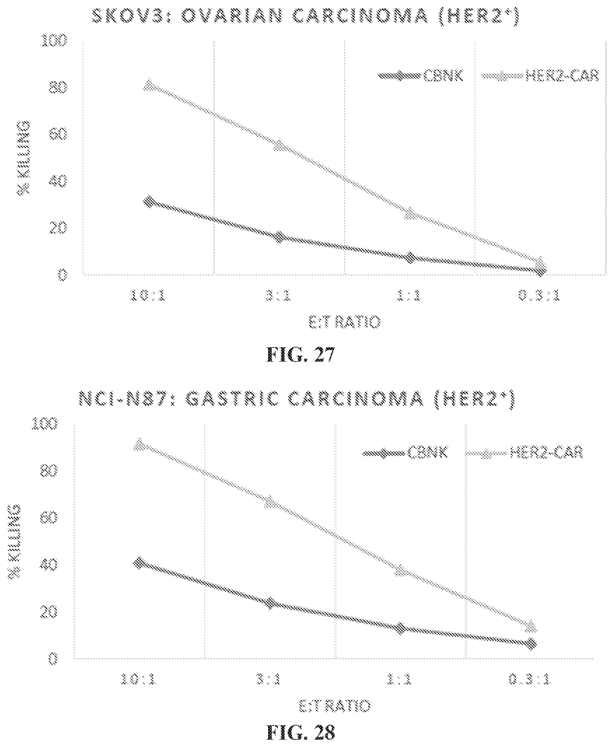

FIG. 27 shows in vitro killing activity of AB-201 against the ovarian carcinoma (HER2+) cell line SKOV3.

FIG. 28 shows in vitro killing activity of AB-201 against the gastric carcinoma (HER2+) cell line NCI-N87.

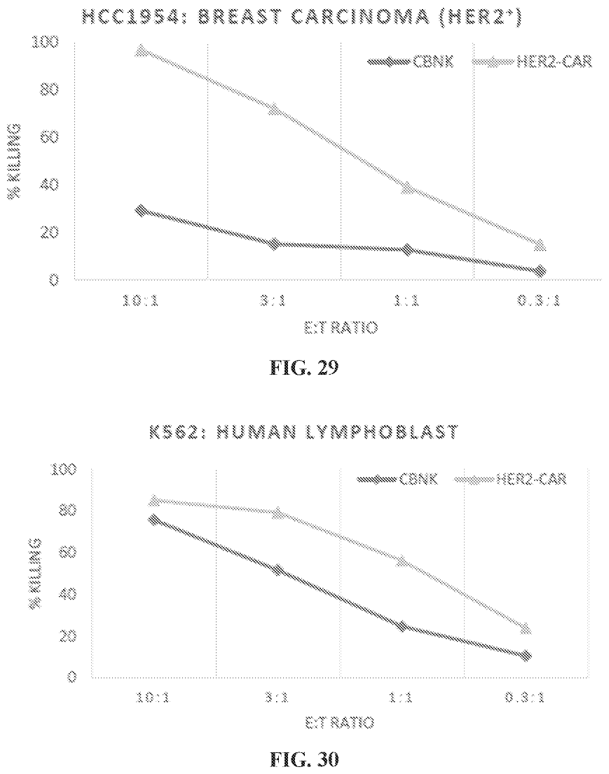

FIG. 29 shows in vitro killing activity of AB-201 against the breast carcinoma (HER2+) cell line HCC1954.

FIG. 30 shows in vitro killing activity of AB-201 against the breast carcinoma (HER2+) cell line K562.

Intracellular Cytokine Staining. Marker of Degranulation and Cytokine Secretion

AB-201 cells were co-cultured with target tumor cells (K562, an immortalized myelogenous leukemia cell line that is widely used in NK cell cytotoxicity assessments, SKOV-3, HCC1954 and NCI-N87). Golgi-plug™ and Golgi-stop™ were used to prevent extracellular secretion of cytokine and CD107a. Production of intracellular cytokines and expression of degranulation markers by AB-201 in response to stimulation with tumor cells was measured by flow cytometry. Cytokine secretion in response to co-culture of AB-201 with target tumor cells (SKOV-3, HCC1954, and NCI-N87) was assessed by ELISA.

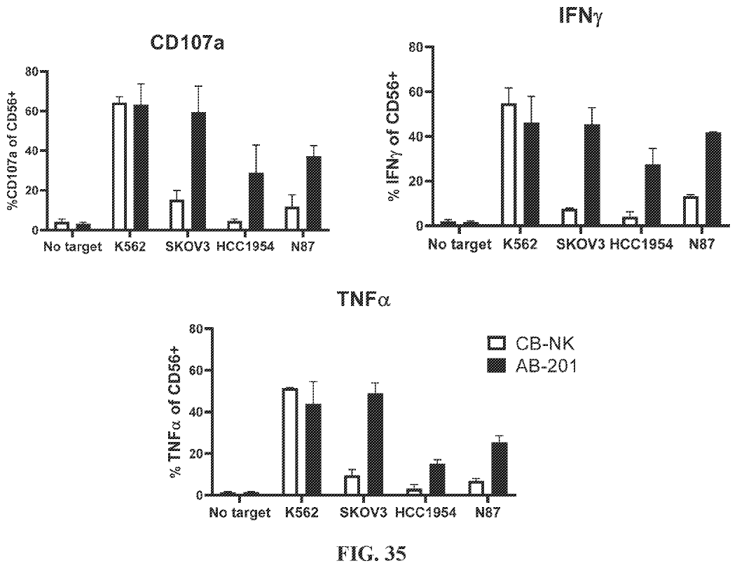

In line with cytotoxic activity, co-culturing of AB-201 with various cancer cell lines (K562, SKOV-3, HCC1954. And NCI-N87) resulted in increased production of effector cytokines (IFN-γ. TNFα) and expression of a marker of degranulation (CD107a) over that observed with the NK cells alone (no target control) (FIG. 35). Further, there was an enhancement of INF-y and TNFa production and CD107a expression in NK cells co-cultured with HER2+ cancer cell lines compared to the non-engineered CBNK cells. This enhancement was not observed with the non-HER2-expressing K562 cancer cell line. In co-culture with K562 the AB-201 results are more comparable to the non-engineered CBNK cells. These results confirm AB-201 activity in response to co-culture with HER2+ tumor cells.

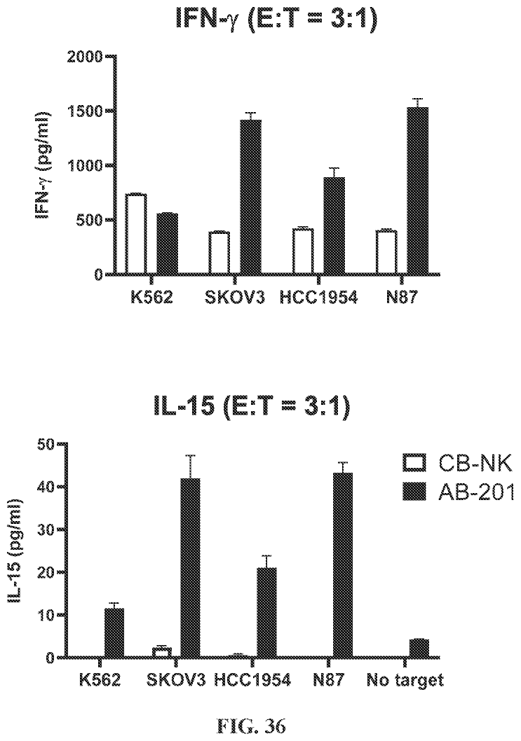

Cytokine levels (INF-y and IL-15) were assessed in the culture media following co-culture of AB-201 with SKOV-3, HCC1954, and NCI-N87 cancer cell lines. Consistent with the intracellular cytokine staining results, an x-y fold elevation in INF-y was observed with AB-201 compared to non-engineered CBNK cells. This enhanced increase in the presence of HER2+ cancer cells was also observed with IL-15 when co-cultures with AB-201 were compared to the non-engineered CBNK and no target control indicating specific activation by cancer cells expressing the HER2 target. FIG. 36.

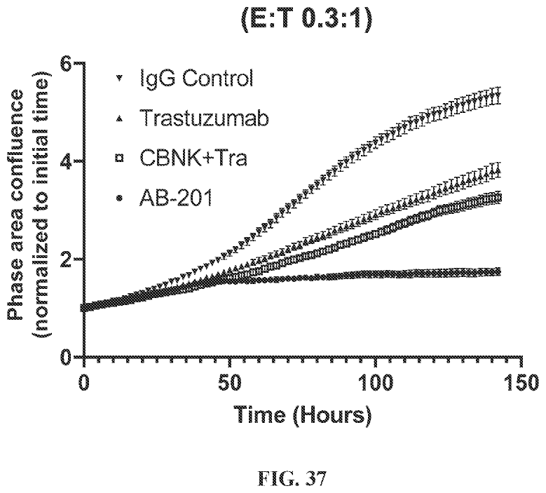

As shown in FIG. 37, the growth of a human HER+ gastric carcinoma cell line. NCI-N87, was monitored by measuring the cell confluence in long term cultures. Trastuzumab was seen to inhibit growth of the culture, whereas trastuzumab combined with the non-CAR NK product AB-101, resulted in further depletion of cell confluence via ADCC cell killing. It was shown that AB-201 had significantly greater cytotoxic killing activity over trastuzumab in combination non-CAR NKs.

HER2-Dependent Cytotoxicity on Primary Cells

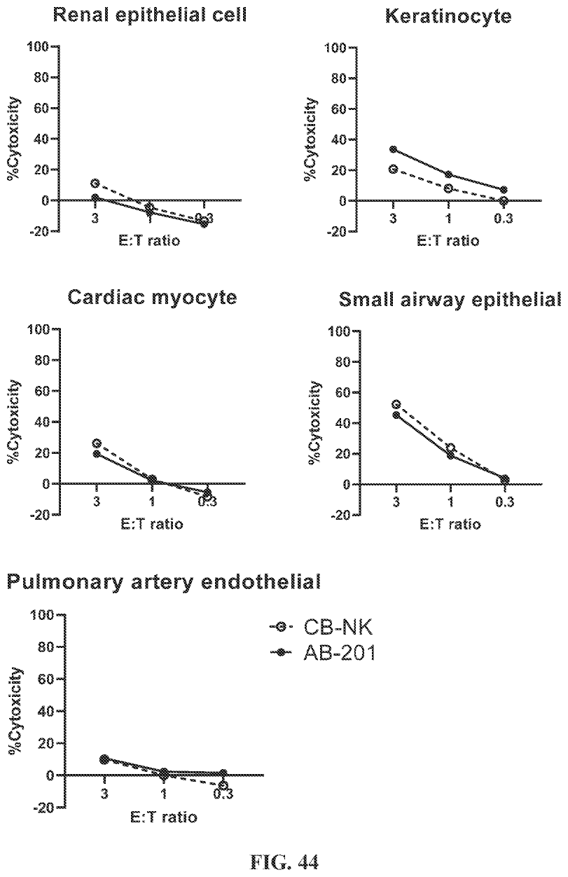

Cytotoxicity of primary cells (non-tumor) was measured following co-culture of AB-201 or control CB-NK cells with pulmonary artery endothelial cells, keratinocytes, renal epithelial cells, cardiac myocytes and small airway epithelial cells for 4 hours at Effector: Target (E: T) ratios of 3:1. 1:1, or 0.3:1. No HER2-dependent cytotoxicity was observed (FIG. 44).

### Example 13: AB-201 In vivo Studies

In vivo efficacy of AB-201 has been evaluated in murine xenograft models bearing HER2+ tumors includingHCC1954, SKOV-3, and NCI-N87.

### HCC1954

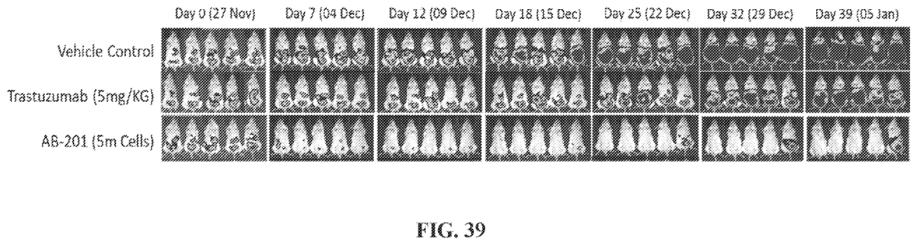

AB-201 demonstrated anti-tumor efficacy in a the human HCC1954 mouse xenograft model of breast cancer using the human HCC1954 breast cancer cell line, which has been characterized as trastuzumab resistant. HCC1954-luc tumor cells were grown in cell culture, harvested, and concentrated to 5×10⁶ cells/mL with PBS (phosphate buffered saline). Mice were injected intraperitoneally (IP) with 1×10⁶ cells/mouse. As shown in FIG. 39, tumors were established in the mice, which were imaged on day zero.

Three days after HCC1954-luc inoculation, mice were randomized to one of 7 groups (Table 14) according to bioluminescence of Day 0 (average bioluminescence signal was 2.49E±08 photons/s). AB101, AB201, TRZ and IL-2 were administered intraperitoneally.

TABLE 14

| | | | Volume | No. of |
|---|---|---|---|---|
| Group | Dose | Route | (μL) | Animals |
| 1 | Freezing medium + IgG 5 | i.p + i.p | 200 + 200 | 8 |
| | mg/kg | | | |
| 2 | TRZ 5 mg/kg | i.p | 200 | 8 |
| 3 | AB101 2 × 10⁷ cells + IL-2 | i.p + i.p | 200 + 100 | 8 |
| 4 | AB101 + TRZ + IL-2 | i.p + | 200 + 200 + | 8 |
| | | i.p + i.p | 100 | |
| 5 | AB201 2 × 10⁷ cells + IL-2 | i.p + i.p | 200 + 100 | 8 |
| 6 | AB201 5 × 10⁶ cells + IL-2 | i.p + i.p | 200 + 100 | 8 |
| 7 | AB201 1 × 10⁶ cells + IL-2 | i.p + i.p | 200 + 100 | 8 |

A single dose of five million AB-201 cells was administered on day four in the appropriate groups. All animals were observed for general symptoms and death two times a day (once a day for weekends and holidays) during the study period. All animals were weighed three times a week. Bioluminescence imaging was performed 8 times (Day 0, 7, 14, 19, 25, 31, 38, 45) using IVIS® Spectrum in vivo imaging system (PerkinElmer).

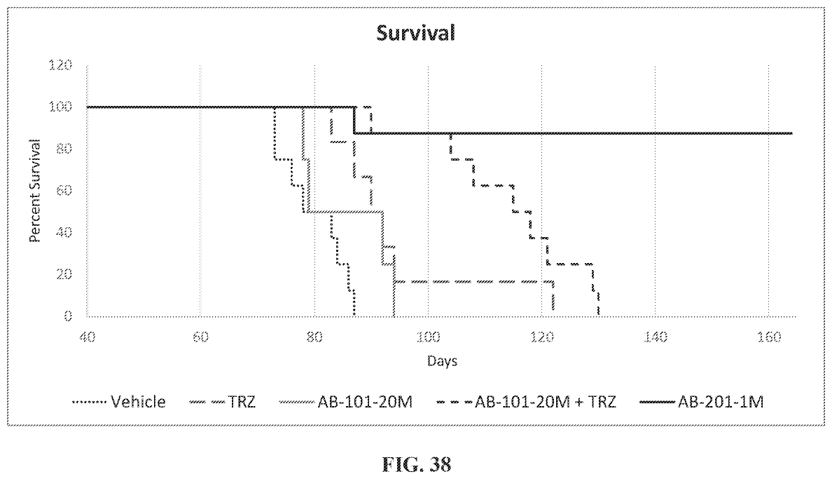

As shown in FIG. 38, survival rates were highest for AB-201, followed by AB-101 with trastuzumab. 87.5% of animals receiving AB-201 were still alive at the end of the experiment. Trastuzumab increased median survival time by 10.5 days and AB-101+ trastuzumab increased median survival time by 38.5 days.

As shown in FIG. 39, AB-201 led to tumor suppression by day seven, and in four out of the five mice complete tumor regression, with no recurrence through the duration of the study (Day 45). The bioluminescence signal of the Vehicle group increased steadily over the course of the study. Compared to the vehicle group, the bioluminescence of AB101+IL-2, AB101+ TRZ +IL-2, and AB201+IL-2 groups decreased by 78.3%, 59.3%, 97.2% and 81.9% at day 45, respectively. Each of these decreases had a p value of less than 0.05.

The results show that AB-201 performed substantially better at controlling HCC1954 tumors than trastuzumab

SK-OV-3

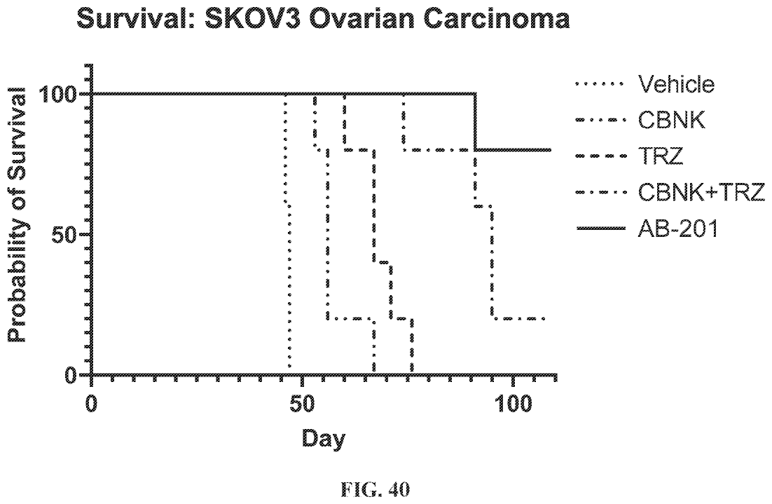

AB-201 demonstrated substantial tumor regression and survival benefits in the SK-OV-3 human ovarian cancer cell line xenograft model system. Three administrations of AB-201 conferred a significant survival benefit (FIG. 40).

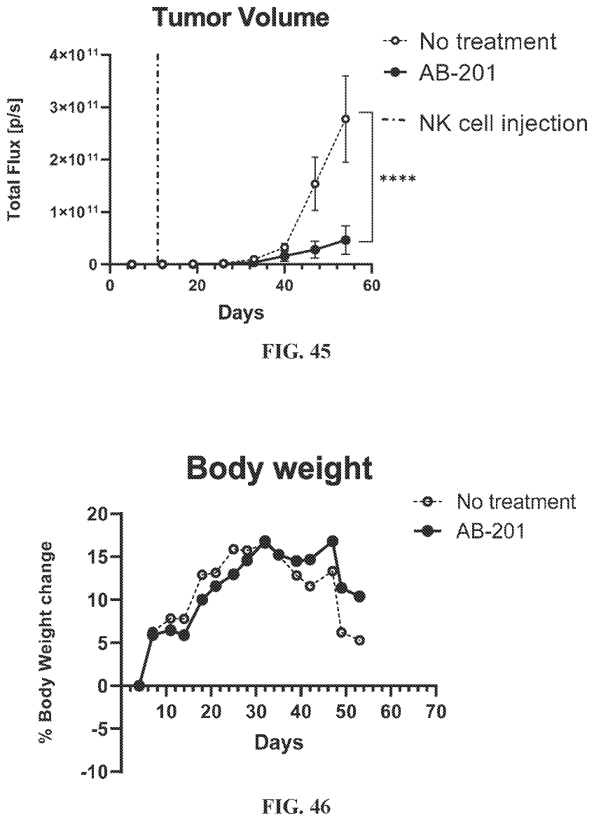

In a separate experiment. NSG mice received 1×10⁶ SKOV3-Luc tumor cells (IP) on day 0 and a single injection of AB-201 (IP) on day 11Bioluminescence (BLI) measurements of SKOV3-Luc, mean total flux+SEM for each group of mice are shown in FIG. 45. The difference in tumor volume between the AB-201 and untreated mice had a p value of <0.0001 as determined by two-way ANOVA (noted as **** in FIG. 45). Differences in body weight were not observed over the course of the study (FIG. 46).

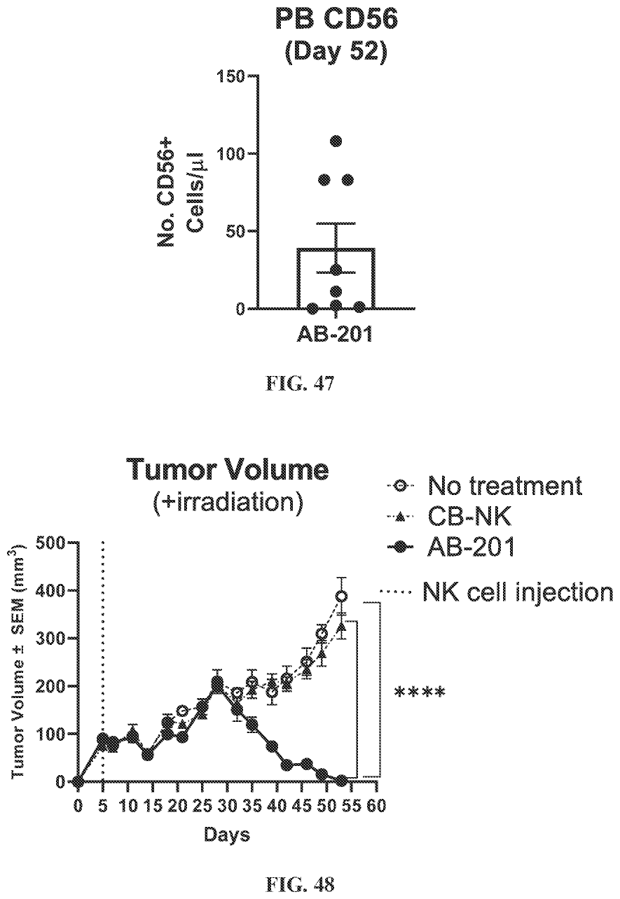

AB-201's ability to persist in NSG mice was also assessed. Blood samples were obtained on day 52. AB-201 cells were still detectable by flow cytometry when gating for human CD45+/CD56+ cells (FIG. 47).

### NCI-N87

AB-201 demonstrated substantial tumor regression and survival benefits in the NCI-N87 human gastric carcinoma cell line xenograft model system.

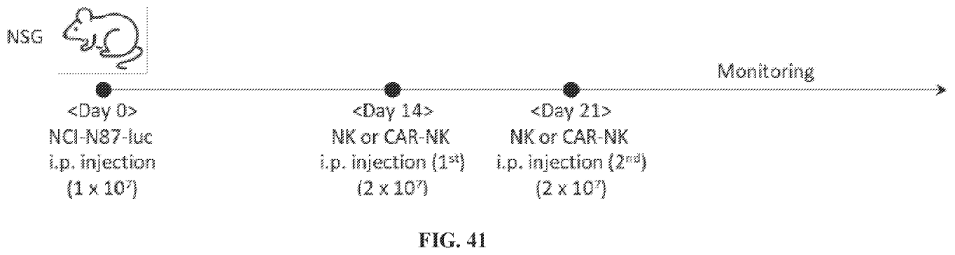

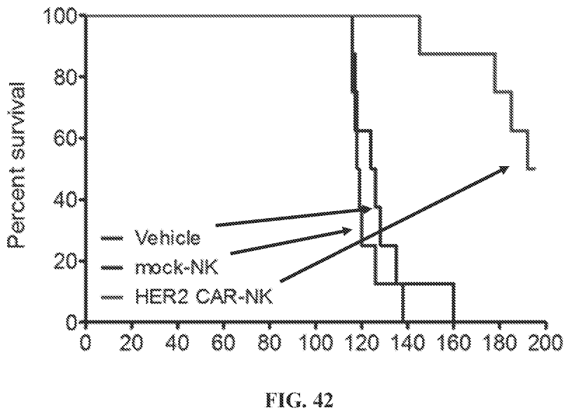

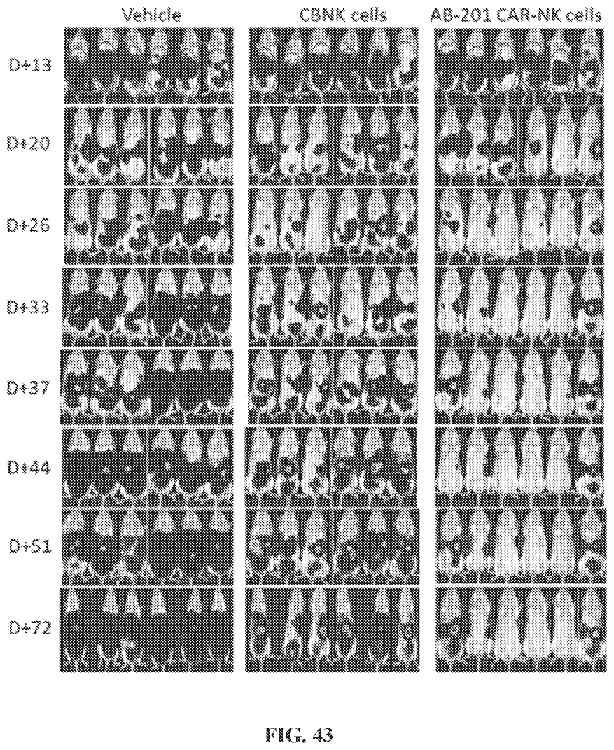

Two administrations of AB-201 confers significant survival benefit and tumor regression. Mice were injected as shown in FIG. 41. FIG. 42 shows percent survival. FIG. 43 shows tumor load.

In a separate experiment, 30 female mice aged six weeks were inoculated subcutaneously in their right flanks with 1×10⁷ NCI-N87 cells/mouse on day 0. Some of the mice also received a single dose of 1.5 Gy (150 rad) of full body irradiation on day-1. Mice were left untreated, administered a single dose of 5×10⁶ AB-201 cells/mouse intravenously, or administered a single dose of 5×10⁶ cord blood NK (CB-NK) cells/mouse intravenously on day 5 post-tumor implantation. Mice were observed daily and tumor volume and body weight were measured twice a week. Mice were euthanized when the No Treatment control group (Group 1) mean tumor volume reached >500 mm³. The study ended on Day 53.

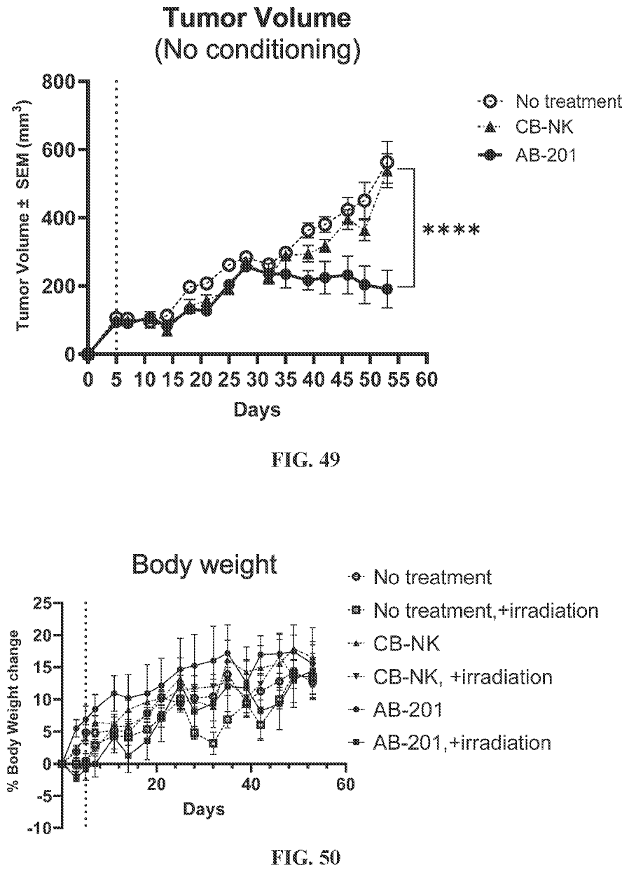

AB-201 demonstrated significant efficacy over no treatment and CB-NK (P<0.0001, Two-way ANOVA) in irradiated mice (FIG. 48) and unconditioned mice (FIG. 49). AB-201 was well tolerated based on no change in body weight across groups (FIG. 50).

At study end, all tumor bearing mice in all groups were euthanized and tumors were excised, and wet weights were recorded in the necropsy inventory. Slits were cut in the tumor every 3-5 mm (to ensure full penetration of fixative), placed in 10% neutral buffered formalin (VWR: Radnor, PAK) for 48-72 hours then transferred to 70% ethanol (EMD Millipore: Billerica, MA) and stored at room temperature.

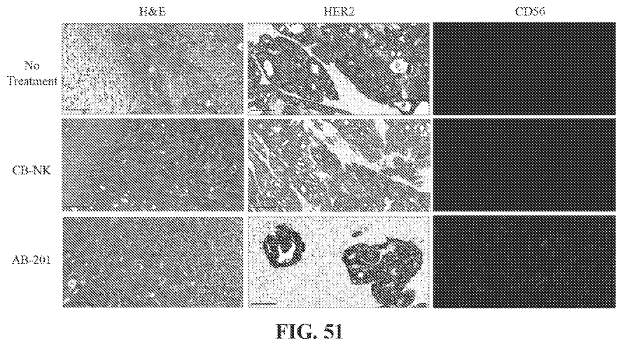

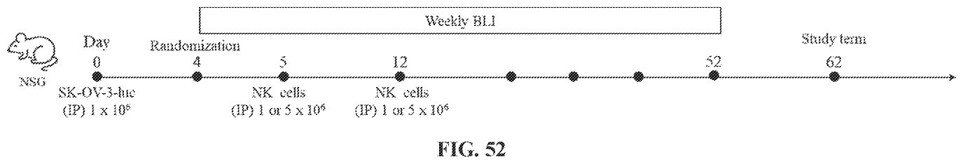

Tissues were assessed for AB-201 infiltration. Samples were trimmed, processed, and embedded as formalin-fixed paraffin embedded blocks. Blocks were then sectioned at 4 μm onto positively-charged slides. Immunofluorescence was performed using a rabbit-anti-CD56 antibody. Heat induced antigen retrieval was performed using Leica Bond Epitope Retrieval Buffer 1 (Citrate solution, pH6.0) for 20 minutes. Non-specific background was blocked with Novocastra Protein Block (Leica, #RE7102-CE, Lot #6055249) for 20 minutes. Primary antibody was applied for overnight incubation at 4° C. A Goat anti-Rabbit IgG Alexa Fluor Plus 647 (red) at a dilution of 1:200 (ThermoFisher, #A31573, Lot #1964354) was applied for 60 minutes at room temperature. Slides were mounted with DAPI in Fluorogel II for nuclear visualization (blue). FIG. 51 depicts representative images of H&E, HER2 IHC and CD56 immunofluorescence staining of tumor section from mice receiving no treatment, CB—NK or AB-201. HER2 immunohistochemistry staining on tumor section (10×magnification), staining visualized in DAB (dark) and hematoxylin was used as nuclear counterstain (blue). CD56 is indicated in red (right) and DAPI used as a nuclear counterstain in blue (40×magnification). The scale bar represents 50 μm. The DC56 staining results demonstrate that AB-201 infiltrated the tumor.

### Example 14: Further In vivo Studies

Here we tested the anti-tumor activity of AB-201, an ex vivo-expanded allogeneic cord blood-derived natural killer cell (CB-NK) that has been genetically modified to express a HER2-directed chimeric antigen receptor (CAR), in a HER2-expressing mouse xenograft model.

Efficacy of AB-201 against HER2+ tumors was evaluated in an NSG mouse (Jackson Laboratories: Bar Harbor, ME) xenograft using the SKOV-3 ovarian adenocarcinoma model. SKOV-3 cells were obtained from ATCC (American Type Culture Collection, Manassas, VA). The SKOV-3 cell line was modified to stably express a luciferase gene (SKOV-3-Luc) which allows for noninvasive bioluminescent imaging (BLI) of tumor cells in vivo in the presence of the substrate d-luciferin. Female NSG mice (n=8/group) were inoculated with 1×10⁶ SKOV-3-Luc tumor cells intraperitoneally on Day 0. On day 4, animals were randomized into seven groups based on tumor burden as defined by BLI signal. Two doses of AB-201 (1×10⁶ and 5×10⁶) were tested in this study and both dose levels were administered IP as either a single administration or as two administrations in tumor-bearing NSG mice starting on day 5 post-tumor inoculation, with the second administration on day 12. Donor-matched cord blood natural killer cells (CB-NK) were administered as a single dose of 5×10⁶ cells/mouse as a control for tumor cell sensitivity to non-engineered NK cells. Tumor cells alone serve as the no treatment control.

Tumors were measured by IVIS starting on Day 4 (once a week). The mice were injected subcutaneously (s.c.) with 150 mg/kg d-Luciferin 15 minutes prior to imaging. Mice were anaesthetized and placed into the imaging chamber (Spectrum CT) ten minutes following administration of d-Luciferin and imaged for luminescence. The BLI is measured in photons/see and is expressed as total flux.

In the intraperitoneal SKOV-3-Luc xenograft model, anti-tumor efficacy of AB-201 was observed at both dose levels tested. AB-201 administration decreased the SKOV-3-Luc tumor burden as assessed by the BLI signal on day 52. Compared to the no treatment control and CB-NK group, AB-201 demonstrated delayed tumor progression as evidenced by reduced luciferase signal (p<0.0001 all AB-201groups). AB-201 administered as a single dose at either 1×10⁶ or 5×10⁶ cells/animal compared to the no treatment control, demonstrated an 82.8% and a 95.6% decrease in tumor burden, respectively. Further, when compared to the CB-NK control group, a 79.4% and 94.8% decrease in tumor burden was observed following a single administration of AB-201 at 1×10⁶ or 5×10⁶, respectively, on day 52. Similar data was obtained with the multiple dose groups. AB-201 was well tolerated at both dose levels with no significant body weight (BW) changes associated with treatment across any of the groups. All animals that received AB-201 survived the duration of study, while four animals in the no treatment and one animal that received CB-NK were found dead or were a moribund sacrifice. AB-201 was detected in peripheral blood on day 7 (2 days post-NK cell administration), day 14 (2 days post-additional administration for AB-201 groups 6 &7), day 21 and at term analysis on day 62, while CB-NK detection peaked early on day 7 and gradually decreased and each subsequent timepoint indicating that both CB-NK and AB-201 were capable of trafficking from the site of injection into the periphery. In conclusion, these results demonstrate significant anti-tumor efficacy of AB-201 in an ovarian SKOV-3 xenograft tumor model and suggest the therapeutic potential of AB-201 against HER2+ tumors.

Spleen and blood samples were collected, processed, and stained for the flow cytometry analysis with a 4-color panel as shown in Table 15 below.

TABLE 15
Flow Cytometry Antibodies

| Markers | Fluorochrome | Clone | Cat # | Isotype | Vendor |
|---|---|---|---|---|---|
| hCD45 | AF700 | HI30 | 304024 | Mouse IgG1, κ | BioLegend |
| hCD56 | BUV395 | NCAM16.2 | 563554 | Mouse IgG2b, κ | BD |
| hCD16 | FITC | 3G8 | 302006 | Mouse IgG1, κ | BioLegend |
| L/D | efluo780 | n/a | 65-0865-18 | n/a | eBioscience |

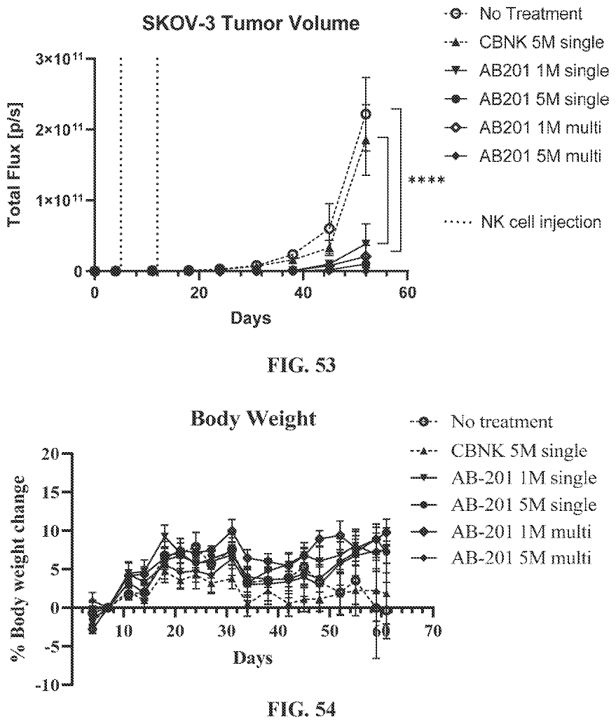

As shown in FIG. 53, BLI measurements of SKOV-3-Luc tumor xenografts were taken at multiple timepoints following administration of tumor cells, mean total [flux photons/second]±SEM for each group of mice is shown. **** p<0.0001, two-way ANOVA with Tukey's multiple comparison test.

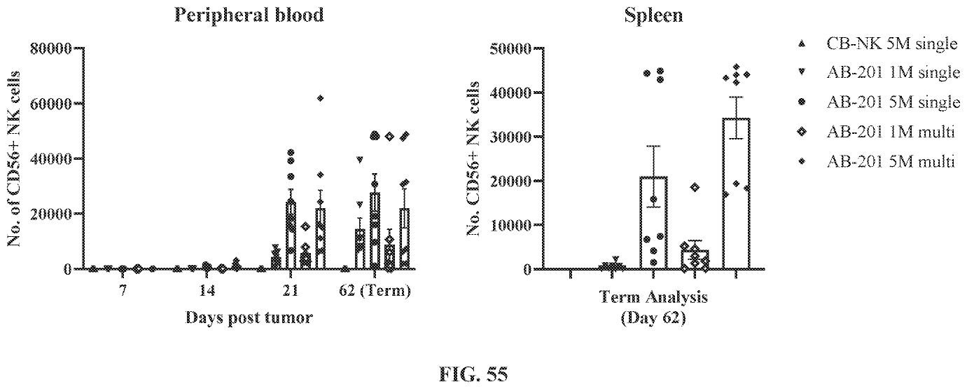

AB-201 tolerability was determined by group percent BW change and is shown in FIG. 54. AB-201 was well tolerated at both dose levels of 1×10⁶ cells/mouse and 5×10⁶ cells/mouse. There was observed body weight gain in the AB-201-treated groups and weight loss in the untreated or CB-NK treated groups by day 55 of study. No adverse events were observed in any AB-201-treated animals in this study. All animals receiving AB-201 survived the duration of the study. Only animals from the no treatment and CB-NK control groups were found dead or sacrificed due to tumor burden.

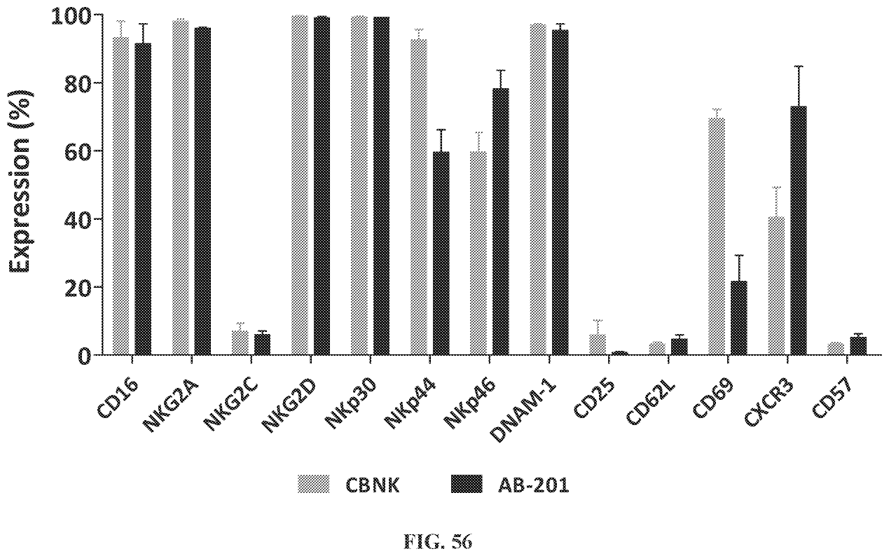

As NK cells were administered by IP injection, we wanted to determine whether there was trafficking of NK cells into the periphery. Peripheral blood was assessed at multiple timepoints post-NK cell infusion and at term by flow cytometry (FIG. 55). AB-201 was detected in peripheral blood on day 7 (2 days post-NK administration), day 14 (2 days post-additional administration for AB-201 group 6 &7), day 21 and at term analysis on day 62 while CB-NK detection peaked early on day 7 and gradually decreased and each subsequent timepoint. These data demonstrate that AB-201 persisted for at least 57 days following a single injection (both dose levels). In addition, at term collection AB-201 was detected in spleen with higher presence correlating with dose level. These data indicate that both CB-NK and AB-201 were capable of trafficking from the site of injection into the periphery.

Efficacy of AB-201 was observed at 1×10⁶ or 5×10⁶ dose levels following a single dose administration on Day 5 or multiple doses administered on Days 5 and 12 post-tumor cell inoculation in the SKOV-3 ovarian carcinoma tumor model. AB-201 was detected in peripheral blood and spleen at term on day 62, indicating trafficking to the periphery in a tumor bearing animal. Both AB-201 dose levels (1×10⁶ or 5×10⁶ cells/mouse) administered either as single or multiple doses were well tolerated as demonstrated by body weight gain in the AB-201-treated groups and weight loss in the untreated and CB-NK treated groups by day 55 of study. In addition, no adverse events were observed in any AB-201 treated animals in this study. In conclusion, these AB-201 data demonstrate significant in vivo anti-tumor activity and good tolerability in the SKOV-3 xenograft model.

### Example 15: Further In vitro studies

AB-201 consists of ex vivo-expanded allogeneic cord blood-derived natural killer cells that have been genetically modified to express a HER2-CAR. This study characterizes the purity, NK cell activity, phenotypic characteristics (i.e., expression of inhibitory receptors), and cytotoxicity and cytokine secretion against tumor cells of AB-201.

The purity of NK cells was determined through CD3-CD56+expression. A high purity of 98.7±0.8% (mean±SD) CD3-CD56+expressing cells was observed in the final AB-201 drug product. In addition, AB-201 demonstrated high expression levels of NK activating receptors (i.e., CD16. NKG2D. NKp30, NKp46, and DNAM-1) and chemokine receptors (i.e., CXCR3).

The short-term (4 hr) cytotoxicity assay was performed to confirm the direct tumor cell killing activity of AB-201. The cytotoxicity against tumor cell lines was compared between AB-201 cells and donor matched, eHuT-78-expanded CBNK incubated with tumor cells at different Effector: target (E: T) ratios. According to the short-term cytotoxicity analysis results, it was confirmed significant cytotoxicity against HER2-positive tumor cell lines (i.e., SKOV-3, HCC1954, and NCI-N87) was observed for AB-201 in comparison to CBNK at an ET ratio of 10:1.

The long-term (5 days) cytotoxicity assay was performed to confirm the direct tumor cell killing activity of AB-201. The long-term cytotoxicity assay evaluates NK cell killing activity through a 5-day measurement of fluorescence reduction following co-culture with tumor cell lines expressing red fluorescent protein. Results indicate a higher tumor cell killing activity for AB-201 compared to CBNK as the co-culture of SKOV-3 cells with CBNK resulted in 95.7±2.5% tumor cells while co-culture of SKOV-3 cells with AB-201 resulted in 33.3±4.5% tumor cells. Similarly, a larger number of HCC1954 tumor cells was reduced when co-cultured with AB-201 as compared to co-culture with CBNK (82.9±6.1% vs 25.3=1.0% tumor cells for CBNK or AB-201, respectively). Regarding the NCI-N87 tumor cell line which does not express fluorescent proteins, the degree of cell killing is assessed through the confluence of the tumor cells. A larger decrease in tumor cell confluence was achieved by AB-201 as 54.5±6.9% and 38.8±4.2% tumor cells were measured for CBNK and AB-201, respectively at the end of the 5 day period. Based on these results, AB-201 demonstrated enhanced anti-tumor activity against HER2±tumor cell lines.

The NK cell activity of AB-201 was evaluated through cytokine secretion and CD107a expression. After co-culture of NK cells (CBNK orAB-201) with HER2-positive tumor cell lines, cytokine secretion and CD107a expression levels were assessed. Compared to CBNK. a 4 to 6-fold expression of CD107a, 2 to 4-fold expression of IFN-γ, and 2 to 4-fold expression of TNF-α was observed for the AB-201.

To confirm IL-15 secretion, which is relevant to persistence of CAR-NK cells in vivo, AB-201 was co-cultured with HER2-positive tumor cell lines, and the IL-15 level within the culture medium was measured. Results confirmed a 3.5 to 6-fold higher IL-15 secretion of AB-201 compared to CBNK.

In summary, AB-201 demonstrated enhanced anti-tumor activity in a HER2-dependent manner.

### Cell Viability

The cell viability of AB-201 post thaw was measured in three separate experiments performed on different days using an automated cell counter (ADAM cell counter). Donor matched CBNK was included as a control. The average cell viability of AB-201 and CBNK post-thaw was 97.3+0.7% (mean±SD) and 91.8+3.9% respectively, indicating high viability for both.

### Purity

The purity of AB-201 and CBNK cells was assessed using FACS. The content of NK cells (CD3 CD56+) , T cells (CD3+), monocytes (CD14+), and B cells (CD19+) were measured in three separate experiments for both AB-201 and CBNK.

Purity results for CBNK and AB-201 confirmed that the content of CD3 CD56+NK cells was 98.64+0.33% and 98.65+0.83%, respectively. CD3+ T cells were not detected in CBNK and AB-201, CD14+ monocytes were 0.01+0.02% and 0.06+0.10%, respectively, and CD19+B cells were 0.49+0.17% and 0.12+0.12%, respectively. In summary, these results demonstrate high purity of NK cells in AB-201.

### NK Cell Phenotypes

Activation/inhibitory receptors, chemokine receptors, and surface molecules related to cytotoxicity are expressed on the surface of NK cells. To confirm the expression of these molecules, the cell phenotype of CBNK and AB-201 was analyzed using FACS.

The activation receptors that were highly expressed at an average of at least 90% in both CBNK and AB-201 include CD16, NKG2D, NKp30, and DNAM-1. The inhibitory receptor NKG2A was also highly expressed at an average of at least 90%. In particular, a higher average expression of ˜20% was observed for NKp46 in AB-201 compared to CBNK. Similarly, a higher average expression of ˜30% was observed for chemokine receptor, CXCR3 (FIG. 56). HER2 CAR Expression

The content of HER2 CAR expressed in AB-201 was analyzed using FACS. The result of thawing AB-201 and measuring the HER2 CAR expression confirmed an average of 90.4±2.9%.

### Short Term Cytotoxicity

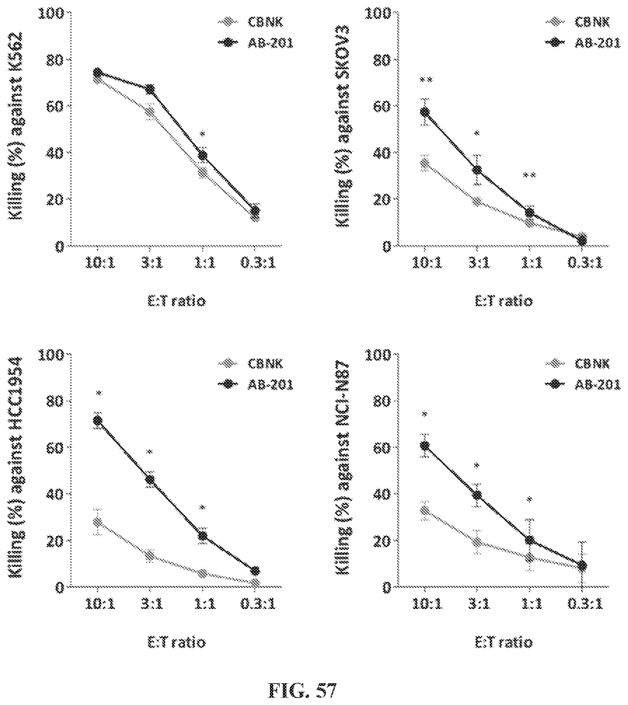

K562: To evaluate the anti-tumor activity of AB-201 against K562 at several E: T ratios (10:1, 3:1, 1:1, 0.3:1) were measured in three separate experiments. At a 10:1 E:T ratio, CBNK and AB-201 yielded measurements of 71.4±2.8% and 74.4±1.1%), respectively. The CBNK and AB-201 cytotoxicity was measured to be 57.5±6.4% and 67.1±2.1% at an E:T ratio of 3:1, 31.4±3.8% and 38.7±3.3% at E:T ratio of 1:1, and 12.1±1.6% and 15.1±2.9% at an E: T ratio of 0.3:1. respectively. Hence, an E:T ratio-dependent anti-tumor activity with no significant differences between the CBNK and AB-201 was observed (FIG. 57, Table 16).

SKOV-3: The anti-tumor activity of AB-201 was evaluated against SKOV-3, which is a HER2+ovarian cancer cell line, in three separate experiments. At an ET ratio of 10:1, CBNK showed a cytotoxicity of 35.4±5.7%, while AB-201 demonstrated a cytotoxicity of 57.4±5.6%. The cytotoxicity for the CBNK and AB-201 was measured to be 18.9+2.2% and 32.5±6.2% at an E:T ratio of 3:1, 9.7±2.2% and 14.2+2.7% at an E:T ratio of 1:1, and 4.0=1.1% and 2.1±2.2%, at an E:T ratio of 0.3:1, respectively. These results demonstrated HER2-dependent anti-tumor activity of AB-201 that was statistically significant in comparison to CBNK (at an E:T ratio of 10:1. ** p<0.01, at an E:T ratio of 3:1, *p<0.05, at an E:T ratio of 1:1, ** p<0.01, two-tailed t-test) (FIG. 57, Table 16).

HCC1954: The anti-tumor activity of AB-201 was evaluated against HCC1954, a HER2+ breast cancer cell line in three separate experiments. At an E:T ratio of 10:1, cytotoxicity for CBNK was 27.9+9.5% while AB-201 mediated cytotoxicity was 71.5±3.5% (statistically significant difference p<0.05, two-tailed t-test). Similarly, at an E:T ratio of 3:1, the measured cytotoxicity for CBNK and AB-201 was 13.3+4.6% and 46.1±3.3%, respectively, which demonstrated that the anti-tumor activity of AB-201 is significantly higher (p<0.05, two-tailed t-test) than that of CBNK. At an E:T ratio of 1:1, AB-201 exhibited significantly greater cytotoxicity compared to CBNK (E: T ratio of 1:1, *p<0.05, two-tailed t-test) (FIG. 57, Table 16).

NCI-N87: The anti-tumor activity of AB-201 was evaluated against NCI-N87, a HER2+ gastric carcinoma cell line in three separate experiments. At an E:T ratio of 10:1, the cytotoxicity of CBNK was 32.916.8% while AB-201 mediated cytotoxicity was 60.7±4.7%. At an E: T ratio of 3:1, the cytotoxicity of CBNK and AB-201 cells was observed to be 19.3+8.4% and 39.4±4.9%, and 12.7±9.8% and 20.1±8.8% respectively at an E:T ratio of 1:1. At an E: T ratio of 0.3:1, CBNK was 8.1±10.3% and AB-201 was 9.4±9.8%. Hence, results demonstrated that the anti-tumor activity of AB-201 was statistically significant compared to CBNK except for the E: T ratio of 0.3:1 (at an ET ratio of 10:1, *p<0.05, at an ET ratio of 3:1, *p<0.05, at an E: T ratio of 1:1, *p<0.05, two-tailed t-test) (FIG. 57, Table 16).

TABLE 16
Summary of direct cytotoxicity of CBNK,
AB-201 against tumor cells (n = 3)

| | Target cell: K562 | | | | Target cell: SKOV-3 | | | |
|---|---|---|---|---|---|---|---|---|
| Specific lysis (%) | CBNK | | AB-201 | | CBNK | | AB-201 | |
| E:T ratio | Mean | SD | Mean | SD | Mean | SD | Mean | SD |
| 10:1 | 71.4 | 2.8 | 74.4 | 1.1 | 35.4 | 5.7 | 57.4 | 5.6 |
| 3:1 | 57.5 | 6.4 | 67.1 | 2.1 | 18.9 | 2.2 | 32.5 | 6.2 |
| 1:1 | 31.4 | 3.8 | 38.7 | 3.3 | 9.7 | 2.2 | 14.2 | 2.7 |
| 0.3:1 | 12.1 | 1.6 | 15.1 | 2.9 | 4.0 | 1.1 | 2.1 | 2.2 |

| | Target cell: HCC1954 | | | | Target cell: NCI-N87 | | | |
|---|---|---|---|---|---|---|---|---|
| Specific lysis (%) | CBNK | | AB-201 | | CBNK | | AB-201 | |
| E:T ratio | Mean | SD | Mean | SD | Mean | SD | Mean | SD |
| 10:1 | 27.9 | 9.5 | 71.5 | 3.5 | 32.9 | 6.8 | 60.7 | 4.7 |
| 3:1 | 13.3 | 4.6 | 46.1 | 3.3 | 19.3 | 8.4 | 39.4 | 4.9 |
| 1:1 | 5.8 | 2.5 | 22.0 | 3.3 | 12.7 | 9.8 | 20.1 | 8.8 |
| 0.3:1 | 1.7 | 0.6 | 6.9 | 1.9 | 8.1 | 10.3 | 9.4 | 9.8 |

### Long Term Cytotoxicity

To test the long-term anti-tumor activity of CBNK and AB-201 against HER2-expressing target cells, CBNK and AB-201 were co-cultured with NCI-N87 and red fluorescent protein-expressing HCC1954 and SKOV-3 cell lines for 5 days.

After co-culturing the target cell lines with NK cells, the anti-tumor activity against the NCI-N87 cell line, which does not express the red fluorescent protein, was determined by confluence (%). The anti-tumor activity against HCC1954 and SKOV-3 cells was measured by the fluorescence intensity.

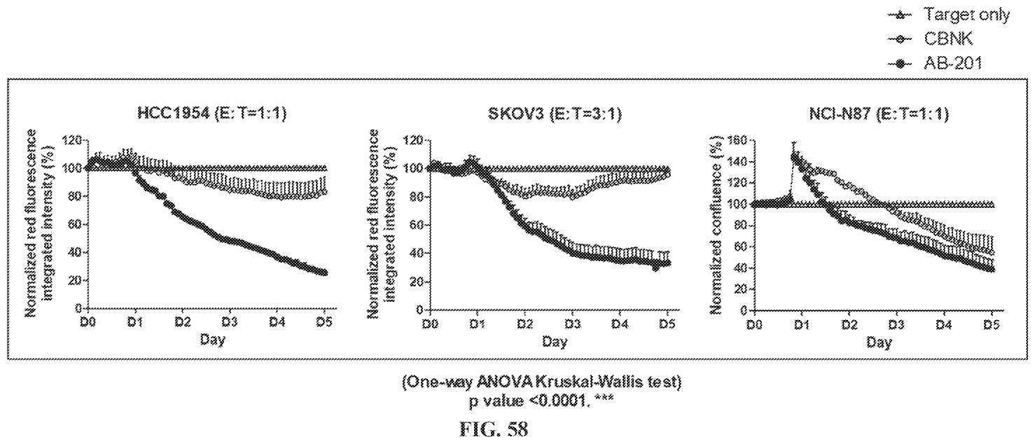

FIG. 58 represents the anti-tumor activity of AB-201 against the HER2-expressing cell lines HCC1954, SKOV-3, and NCI-N87. The data presented within the figure is a representative result from 3 separate tests. The red fluorescence integrated intensity or confluence of target cells for conditions without effector cells were normalized to 100%, and the fluorescence intensity and confluence of each co-culture condition were displayed for each time interval.

An enhanced anti-tumor activity of AB-201 against the HCC1954 cell line was observed at all observed time points compared to CBNK (E: T ratio=1:1, p value <0.0001, One-way ANOVA Kruskal-wallis test). As the fluorescence intensity of untreated HCC1954 cells at day 5 of co-culturing was set as 100%, it was confirmed that the anti-tumor activity of AB-201 against the HCC1954 cell line was high resulting in a reduced fluorescence of 25.3+1.0% (mean±SEM) while the co-culture conditions with CBNK exhibited 82.9+6.1% (mean±SEM) of fluorescence. In addition, it was confirmed that the anti-tumor activity of AB-201 occurred continuously during the co-culture process as the fluorescence intensity of HCC1954 cells continued to decrease during the 5 days. As the fluorescence intensity increased by approximately 6.6 fold over the 5 days for the HCC1954 cell only condition, it was demonstrated that the decrease in target cells were due to the cytotoxicity of AB-201.

An enhanced anti-tumor activity of AB-201 against the SKOV-3 cell line was observed at all time points compared to CBNK (E: T ratio=3:1, p value <0.0001, One-way ANOVA Kruskal-wallis test). As the fluorescence intensity of untreated SKOV3 cells at day 5 was set as 100%, it was shown that the co-culture condition with CBNK was at 95.7±2.5% (mean±SEM) and the co-culture condition with AB-201 was 33.3+4.5% (mean=SEM), which indicates an enhanced anti-tumor activity of AB-201 in comparison to CBNK. In addition, while CBNK demonstrated a trend of increasing fluorescence intensity of the target cells as the anti-tumor activity gradually decreased following day 3 of co-culture, the anti-tumor activity of AB-201 was maintained continuously during all 5 days of co-culture. As the fluorescence intensity increased by approximately 5.9-fold over the 5 days for the SKOV-3 cell only condition, it was demonstrated that the decrease in target cells were due to the cytotoxicity of AB-201.

An enhanced anti-tumor activity of AB-201 against the NCI-N87 cell line was observed at all observed time points compared to CBNK cells (E:T ratio=1:1, p value <0.0001, One-way ANOVA Kruskal-wallis test). As the confluence of untreated NCI-N87 cells at day 5 of co-culturing was set as 100%, the measured confluence of CBNK and AB-201 cells resulted in 54.5±6.9% (mean±SEM) and 38.8+4.2% (mean±SEM), respectively. Hence, it was shown that the anti-tumor activity of AB-201 exceeded the high basal anti-tumor activity of CBNK cells against NCI-N87 cells. The upward pattern of the graph in the groups where NK cells have been added compared to the graph of untreated NCI-N87 at a 20-hour point can be explained by the increased confluence due to the influx of NK cells, and it was confirmed that confluence of the target cell decreased as the anti-tumor activity continued to occur during 5 days of co-culture. For NCI-N87 cell only condition, the confluence increased by approximately 3.8-foldover the 5 days, which confirmed that NCI-N87 cells have decreased due to the cytotoxicity of the NK cells.

### Intracellular Cyotkine Measurement and Cell Surface CD107a Expression Measurement

After co-culturing NK cells and HER2-positive tumor cell lines (SKOV-3, HCC1954, NCI-N87) and a HER2-negative tumor cell line (K562) at an E:T ratio of 1:1 for 4 hours, respectively, the effector cytokines (IFN-γ, TNF-α) and degranulation markers (CD107a) generated from NK cells were measured through flow cytometry. These results were obtained from a total of 3 repeat tests using CBNK and AB-201 generated from the same donor.

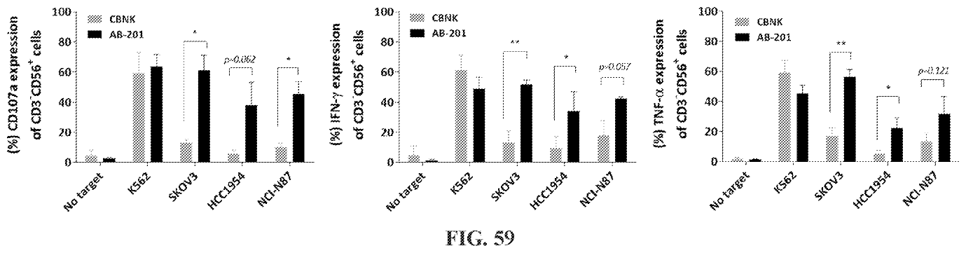

When CBNK or AB-201 was cultured alone without the target cell line, CD107a expressed in each cell was measured as 4.5=3.9% in CBNK and 2.8=0.7% in AB-201 (Mean±SD). Under the co-culture condition with SKOV3. CD107a expression level in CBNK was 13.1±2.2% and in AB-201 61.2+10.1%, and when co-cultured with HCC1954, CD107a expression levels were 6.1=2.3% in CBNK and 38.1±15.1% in AB-201. Under the co-culture condition with NCI-N87, CBNK and AB-201 resulted in expression levels of 18.1±9.9% and 42.6+1.0%, respectively (FIG. 59, Table 17).

The intracellular expression of effector cytokine, IFN-γ was shown to be 4.8+6.3% and 1.4±0.6% for CBNK and AB-201 cells, respectively, in culture conditions without the target cell line. Conversely, IFN-γ expression was 13.4±7.5% and 51.9+2.9% for CBNK and AB-201, respectively, when co-cultured with SKOV-3. These data indicate a significant upregulation of IFN-γ expression in AB-201 compared to CBNK (** p<0.01, two-tailed t-test). When co-cultured with HCC1954, CBNK and AB-201 exhibited IFN-γ expression levels of 9.5±7.5% and 33.9+13.3%, respectively, which indicates a statistically significant difference (*p<0.05, two-tailed t-test). In the NCI-N87 co-culture, IFN-γ expression for CBNK and AB-201 were 18.1±9.9% and 42.6+1.0%, respectively. These data demonstrate a 2.3-fold higher IFN-Y expression for AB-201, however the results were not statistically significant (p=0.057, two-tailed t-test). The co-culture with K562 resulted in 61.4=9.9% in CBNK and 48.9-7.8% in AB-201 (FIG. 59, Table 17).

Through a similar method, the intracellular expression of TNF-α was analyzed, and the protein was barely expressed with 1.7±1.3% in CBNK and 1.4±0.3% in AB-201 in culture conditions without the target cell line. Conversely, when co-cultured with SKOV3, the expression of TNF-α increased to 17.0+5.5% with CBNK and 56.7±2.9% for CBNK and AB-201, respectively. Under the co-culture condition with HCC1954, TNF-α expression was measured to be 5.4±2.0% and 22.3+6.9% for CBNK and AB-201, respectively. Both SKOV3 and HCC1954 co-cultures with AB-201 demonstrated significantly higher TNF-α expression levels compared to co-cultures with CBNK (** p<0.01 and *p<0.05, two-tailed t-test for SKOV3 and HCC1954, respectively). Under the co-culture condition with NCI-N87, CBNK was 13.2+6.0%, and AB-201 was 32.0+11.4%, in which an approximately 2.4-fold higher expression was observed for AB-201 compared to CBNK. The co-culture with K562 resulted in 59.1±8.3% in CBNK and 45.5±5.3% in AB-201 (FIG. 59, Table 17).

Results indicate that AB-201 co-cultured with HER2-positive tumor cell lines expressed 10 to 20-fold higher levels of CD107a, 20 to 40-fold higher levels of IFN-γ, and 15 to 40-fold higher levels of TNF-α compared to unstimulated NK cells (no target). When compared to CBNK, AB-201 co-cultured with HER2-positive tumor cell lines expressed a 4.5 to 6.3-fold higher expression of CD107a, 2.3 to 3.9-fold higher expression of IFN-γ, and 2.4 to 4.1-fold higher expression of TNF-α.

TABLE 17
Summary of ICS data of CBNK, AB-201 against tumor cells (n = 3)

| | CD107a | | | | IFN-γ | | | | TNF-α | | | |
|---|---|---|---|---|---|---|---|---|---|---|---|---|
| Expression | CBNK | | AB-201 | | CBNK | | AB-201 | | CBNK | | AB-201 | |
| (%) | Mean | SD | Mean | SD | Mean | SD | Mean | SD | Mean | SD | Mean | SD |
| No target | 4.5 | 3.9 | 2.8 | 0.7 | 4.8 | 6.3 | 1.4 | 0.6 | 1.7 | 1.3 | 1.4 | 0.3 |
| K562 | 59.5 | 13.8 | 63.7 | 8.1 | 61.4 | 9.9 | 48.9 | 7.8 | 59.1 | 8.3 | 45.5 | 5.3 |
| SKOV-3 | 13.1 | 2.2 | 61.2 | 10.1 | 13.4 | 7.5 | 51.9 | 2.9 | 17.0 | 5.5 | 56.7 | 4.7 |
| HCC1954 | 6.1 | 2.3 | 38.1 | 15.2 | 9.5 | 7.5 | 33.9 | 13.3 | 5.4 | 2.0 | 22.3 | 6.9 |
| NCI-N87 | 10.2 | 2.9 | 45.7 | 7.9 | 18.1 | 9.9 | 42.6 | 1.0 | 13.2 | 6.0 | 32.0 | 11.4 |

### IFN-γ ELISA

The HER2-positive tumor cell lines (SKOV-3, HCC1954, NCI-N87) and a HER2-negative tumor cell line (K562) were co-cultured with CBNK or AB-201 at a ratio of 1:1 for 24 hours, respectively, and the concentration of IFN-γ was measured in the culture medium.

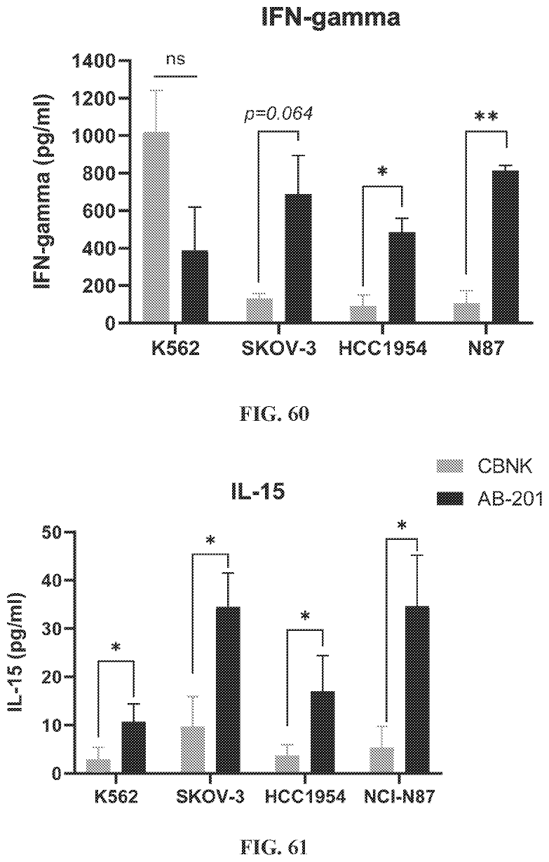

The data in FIG. 60 is an average of two experiments using the same batch of NK cells (run in triplicate), and exhibits the p values obtained through the T.TEST verification.

In co-culture with HER2-positive tumor cell lines and AB-201, the concentration of IFN-γ in the culture medium was approximately 5.3 to 7.8-fold higher than that of CBNK. In addition, HCC1954 and NCI-N87 showed a statistically significant difference. In co-culture with a HER2-negative tumor cell line (K562) and AB-201 the concentration of IFN-γ in culture medium was approximately 2.6-fold lower than that of CBNK.

IL-15 ELISA

The HER2-positive tumor cell lines (SKOV-3, HCC1954, NCI-N87) and a HER2-negative tumor cell line (K562) were co-cultured with CBNK or AB-201 cells at a ratio of 3:1 for 24 hours, respectively, and the concentration of IL-15 was measured in the culture medium. The data in FIG. 61 is an average of three experiments using the same batch of NK cells, and exhibits the p values obtained through the T.TEST verification.

In co-culture with tumor cells and AB-201, the concentration of IL-15 in the culture medium was approximately 3.5 to 6.4 fold higher than that of CBNK, and it demonstrated a statistically significant difference for all HER2+ tumor cell lines. In co-culture with AB-201 and a HER2-negative tumor cell line (K562), the concentration of IL-15 in culture medium was approximately 3.7-fold higher than that of CBNK, demonstrating a statistically significant difference.

### CONCLUSIONS

The results for the short-term (4-hour) cytotoxicity analysis against HER2-positive tumor cells confirm significant anti-tumor activity of AB-201 compared to CBNK (ET ratio 10:1). (*p<0.05 for HCC1954 and NCI-N87, and ** p<0.01 for SKOV-3, compared with CBNK, two-tailed t-test). To confirm the direct anti-tumor activity of AB-201 against tumor cells, the long-term (5-day) cytotoxicity analysis was performed. The anti-tumor activity of AB-201 was higher in comparison to CBNK at all observed time points, and the anti-tumor activity of AB-201 persisted over the 5 days.

The activity of AB-201 against tumor cells was evaluated through cytokine secretion and CD107a expression measurement. Results indicate that AB-201 co-cultured with HER2-positive cell lines expressed 10 to20-fold higher levels of CD107a, 20 to40-fold higher levels of IFN-γ, and 15 to40-fold higher levels TNF-α compared to its non-stimulated state (no target). In addition, a 4.5 to 6.3-fold higher expression of CD107a, a 2.3 to 3.9-fold higher expression of

IFN-γ, and a 2.4 to4.1-fold higher expression of TNF-α was observed in comparison to CBNK cells. These results demonstrate HER2-dependent activity from AB-201.

IL-15 secretion which enhances the persistence of CAR-NK cells was analyzed following co-culture with the HER2-positive tumor cell lines. Results confirm that a 3.5 to 6.4 fold higher IL-15 level was secreted for AB-201 in comparison to CBNK.

In conclusion, this study confirmed specific and significant anti-tumor effects of AB-201 cells against HER2-positive tumor cells.

### Example 16: AB-201 Therapy

### Fixed Dosing

Patients with HER2+solid tumors are selected and treated with varying doses of AB-201 (1×10⁷ AB-201 cells per administration, 3×10⁷ AB-201 cells per administration, 1×108 AB-201 cells per administration, 3×108 AB-201 cells per administration or 1×109 AB-201 cells per administration).

Some patients have advanced breast cancer (3rd line and beyond), some have gastric/GEJ cancer (2nd line and beyond, post trastuzumab).

### Lymphodepleting Chemotherapy

Cyclophosphamide (500 mg/m²/day) and fludarabine (30 mg/m²/day) is administered IV daily for 3 consecutive days followed by two days of rest, starting 5 days before receiving AB-201 (i.e., from Day-5 through Day-3). Fludarabine and cyclophosphamide is administered by IV infusion, including renal dosing, as appropriate.

Each vial of AB-201 contains 11 mL of study drug at two different strengths: approximately 1.1×10⁷, 1.1×10⁸ or 1.1×10⁹ NK cells (e.g., 0.9×10⁹-1.3×10⁹. 0.9×10⁸1.3×10⁸, or 0.9×10⁷-1.3×10⁷). No more than 10 mL (1×10⁷ or 1×10⁹ NK cells) is intended to be drawn from each vial. The prescribed dose of AB-201 is thawed and transferred aseptically into an IV bag for administration as an IV infusion, by gravity. When multiple vials are administered (i.e., Dose Levels 2 and 4), the vials needed for the dose are thawed simultaneously and transferred aseptically into a single IV bag for administration. AB-201 should be administered as soon as practical, preferably within 30 minutes and no longer than 90 minutes after thawing.

Dose levels for AB-201 are shown in Table 18.

TABLE 18
AB-201 Dose Levels

| Dose Level | Dose |
|---|---|
| −1 | 5 × 10⁶ cells |
| 1 | 1 × 10⁷ cells |
| 2 | 3 × 10⁷ cells |
| 3 | 1 × 10⁸ cells |
| 4 | 3 × 10⁸ cells |
| 5 | 1 × 10⁹ cells |

Cytokine support (IL-2) is not administered.

After the initial dose of AB-201, up to 3 additional doses are given (at days 29 and/or 57 or later, e.g., months 2. 3, and 5).

### Weight-Based Dosing

Patients with HER2+solid tumors are selected and treated with varying doses of AB-201. The patients have one of the following: i) advanced/unresectable or metastatic breast cancer with HER2 overexpression (IHC≥2+) that is refractory to or intolerable of standard treatment, or for which no standard treatment is available: ii) advanced/unresectable or metastatic breast cancer with HER2 overexpression (IHC 3+ or IHC 2+/ISH+) that is relapsed to, refractory to, or intolerable of previous trastuzumab deruxtecan (T-DXd) based therapy: or advanced/unresectable or metastatic gastric or gastroesophageal junction adenocarcinoma with HER2 overexpression (IHC≥2+) that is refractory to or intolerable of standard treatment, or for which no standard treatment is available.

Cyclophosphamide (500 mg/m²/day) and fludarabine (30 mg/m²/day) is administered IV daily for 3 consecutive days followed by two days of rest, starting 5 days before receiving AB-201 (i.e., from Day-5 through Day-3). Fludarabine and cyclophosphamide is administered by IV infusion, including renal dosing, as appropriate.

The first dose of AB-201 is administered as a single IV infusion for approximately 5 to 10 minutes starting at 48 hours (but no more than 7 days) after completion of the lymphodepletion regimen. A 28-day observation period will then occur, at the end of which all relevant safety data will be reviewed. Patients may continue to receive second and third doses of AB201 every six weeks (in Week 7 and Week 13 (+1 week)), until unacceptable toxicity, progressive disease (PD), or withdrawal of consent. If the subject's weight changes by #10% of the baseline weight, then the AB-201 dose will be recalculated. Subsequent doses of AB-201 will be administered without prior lymphodepletion therapy.

Dose levels for AB-201 are shown in Table 19.

TABLE 19
AB-201 Dose Levels

| Dose Level | Dose (per Body Weight) |
|---|---|
| −1 | 1.5 × 10⁵ cells/kg |
| 1 (Starting Dose) | 4.0 × 10⁵ cells/kg |
| 2 | 1.5 × 10⁶ cells/kg |
| 3 | 4.0 × 10⁶ cells/kg |
| 4 | 1.5 × 10⁷ cells/kg |

SEQUENCES

| SEQ ID NO: and | |
|---|---|
| DESCRIPTION | SEQUENCE |
| SEQ ID NO: 1 | MEYASDASLDPEAPWPPAPRARACRVLPWALVAGLLLLLLLAAACAVFLACPWAVSGA |
| Sequence of 4-1BBL | RASPGSAASPRLREGPELSPDDPAGLLDLRQGMFAQLVAQNVLLIDGPLSWYSDPGLA |
| that can be expressed | GVSLTGGLSYKEDTKELVVAKAGVYYVFFQLELRRVVAGEGSGSVSLALHLQPLRSAA |
| by feeder cells | GAAALALTVDLPPASSEARNSAFGFQGRLLHLSAGQRLGVHLHTEARARHAWQLTQGA |
| | TVLGLFRVTPEIPAGLPSPRSE |
| SEQ ID NO: 2 | MALPVTALLLPLALLLHAARPQDRHMIRMRQLIDIVDQLKNYVNDLVPEFLPAPEDVE |
| Sequence of a | TNCEWSAFSCFQKAQLKSANTGNNERIINVSIKKLKRKPPSTNAGRRQKHRLTCPSCD |
| membrane bound IL- | SYEKKPPKEFLERFKSLLQKMIHQHLSSRTHGSEDSAKPTTTPAPRPPTPAPTIASQP |
| 21(mbIL-21) that can | LSLRPEACRPAAGGAVHTRGLDFACDIYIWAPLAGTCGVLLLSLVITLY |
| be expressed by feeder | |
| cells | |
| SEQ ID NO: 3 | MSTESMIRDVELAEEALPKKTGGPQGSRRCLFLSLESESIVAGATTLFCLLHEGVIGP |
| Sequence of a mutated | QREEFPRDLSLISPLAQPVRSSSRTPSDKPVAHVVANPQAEGQLQWLNRRANALLANG |
| TNF alpha (mTNF-a) | VELRDNQLVVPSEGLYLIYSQVLFKGQGCPSTHVLLTHTISRIAVSYQTKVNLLSAIK |
| that can be expressed | SPCQRETPEGAEAKPWYEPIYLGGVFQLEKGDRLSAEINRPDYLDFAESGQVYFGIIA |
| by feeder cells | L |
| SEQ ID NO: 4 | MERVQPLEENVGNAARPRFERNKLLLVASVIQGLGLLLCFTYICLHFSALQVSHRYPR |
| Sequence of OX40L | IQSIKVQFTEYKKEKGFILTSQKEDEIMKVQNNSVIINCDGFYLISLKGYFSQEVNIS |
| that can be expressed | LHYQKDEEPLFQLKKVRSVNSLMVASLTYKDKVYLNVTTDNTSLDDFHVNGGELILIH |
| by feeder cells | QNPGEFCVL |
| SEQ ID NO: 5 | RSKRSRLSHSDYMNMTPRRPGPTRKHYQPYAPPRDFAAYRS |
| CD28 intracellular | |
| signaling domain | |
| SEQ ID NO: 6 | AGGAGTAAGAGGAGCAGGCTCCTGCACAGTGACTACATGAACATGACTCCCCGCCGCC |
| CD28 intracellular | CCGGGCCCACCCGCAAGCATTACCAGCCCTATGCCCCACCACGCGACTTCGCAGCCTA |
| signaling domain | TCGCTCC |
| SEQ ID NO: 7 | CGGAGCAAGAGGTCCCGCCTGCTGCACAGCGACTATATGAACATGACCCCACGGAGAC |
| Codon Optimized | CCGGCCCTACACGGAAACATTACCAGCCCTATGCTCCACCCCGGGACTTCGCAGCTTA |
| CD28 intracellular | CAGAAGT |
| signaling domain | |
| SEQ ID NO: 8 | ERVQPLEENVGNAARPRFERNK |
| OX40L intracellular | |
| signaling domain | |
| SEQ ID NO: 9 | LEENVGNAARPRFERNK |
| OX40L intracellular | |
| signaling domain | |
| functional domain | |
| SEQ ID NO: 10 | RPRFERNK |
| OX40L intracellular | |
| signaling domain | |
| functional domain | |
| SEQ ID NO: 11 | GAAAGGGTCCAACCCCTGGAAGAGAATGTGGGAAATGCAGCCAGGCCAAGATTCGAGA |
| OX40L intracellular | GGAACAAG |
| signaling domain | |
| SEQ ID NO: 12 | GAAAGAGTGCAGCCCCTGGAAGAGAATGTCGGGAATGCCGCTCGCCCAAGATTTGAAA |
| Codon optimized | GGAACAAA |
| OX40L intracellular | |
| signaling domain | |
| SEQ ID NO: 13 | RVKESRSADAPAYQQGQNQLYNELNLGRREEYDVLDKRRGRDPEMGGKPRRKNPQEGL |
| CD3ζ signaling domain | YNELQKDKMAEAYSEIGMKGERRRGKGHDGLYQGLSTATKDTYDALHMQALPPR |
| SEQ ID NO: 14 | AGAGTGAAGTTCAGCAGGAGCGCAGACGCCCCCGCGTACCAGCAGGGCCAGAACCAGC |
| CD3ζ signaling domain | TCTATAACGAGCTCAATCTAGGACGAAGAGAGGAGTACGATGTTTTGGACAAGAGACG |
| | TGGCCGGGACCCTGAGATGGGGGGAAAGCCGAGAAGGAAGAACCCTCAGGAAGGCCTG |
| | TACAATGAACTGCAGAAAGATAAGATGGCGGAGGCCTACAGTGAGATTGGGATGAAAG |
| | GCGAGCGCCGGAGGGGCAAGGGGCACGATGGCCTTTACCAGGGTCTCAGTACAGCCAC |
| | CAAGGACACCTACGACGCCCTTCACATGCAGGCCCTGCCCCCTCGC |
| SEQ ID NO: 15 | CGAGTGAAGTTCAGCAGGTCCGCCGACGCTCCTGCATACCAGCAGGGACAGAACCAGC |
| Codon optimized CD3ζ | TGTATAACGAGCTGAATCTGGGCCGGAGAGAGGAATACGACGTGCTGGACAAAAGGCG |
| signaling odmian | GGGCCGGGACCCCGAAATGGGAGGGAAGCCACGACGGAAAAACCCCCAGGAGGGCCTG |
| | TACAATGAGCTGCAAAAGGACAAAATGGCCGAGGCTTATTCTGAAATCGGGATGAAGG |
| | GAGAGAGAAGGCGCGGAAAAGGCCACGATGGCCTGTACCAGGGGCTGAGCACCGCTAC |
| | AAAGGACACCTATGATGCACTGCACATGCAGGCCCTGCCCCCTCGG |
| SEQ ID NO: 16 | GDVEXNPGP |
| 2A cleavage motif | |
| SEQ ID NO: 17 | GSGEGRGSLLTCGDVEENPGP |
| T2A cleavage site | |
| SEQ ID NO: 18 | GGCTCAGGTGAGGGGCGCGGGAGCCTGCTGACTTGTGGGGATGTAGAGGAAAATCCTG |
| T2A cleavage site | GTCCT |
| SEQ ID NO: 19 | GSGATNFSLLKQAGDVEENPGP |
| P2A cleavage site | |
| SEQ ID NO: 20 | GSGQCTNYALLKLAGDVESNPGP |
| E2A cleavage site | |
| SEQ ID NO: 21 | GSGVKQTLNFDLLKLAGDVESNPGP |
| F2A cleavage site | |
| SEQ ID NO: 22 | MRISKPHLRSISIQCYLCLLLNSHFLTEAGIHVFILGCFSAGLPKTEANWVNVISDLK |
| IL-15 | KIEDLIQSMHIDATLYTESDVHPSCKVTAMKCELLELQVISLESGDASIHDTVENLII |
| | LANNSLSSNGNVTESGCKECEELEEKNIKEFLQSFVHIVQMFINTS- |
| SEQ ID NO: 23 | ATGAGAATCAGCAAACCACACCTCCGGAGCATATCAATCCAGTGTTACTTGTGCCTTC |
| IL-15 | TTTTGAACTCCCATTTCCTCACCGAGGCAGGCATTCATGTGTTCATATTGGGGTGCTT |
| | TAGTGCTGGGCTTCCGAAAACGGAAGCTAACTGGGTAAACGTCATCAGTGACCTTAAA |
| | AAAATTGAGGATCTTATCCAATCAATGCACATCGACGCGACTCTCTACACAGAATCTG |
| | ACGTACACCCGTCATGCAAAGTCACGGCAATGAAGTGTTTTCTTCTCGAGCTCCAAGT |
| | AATTTCCCTGGAGTCTGGCGATGCCTCCATCCACGATACGGTTGAAAATCTGATTATA |
| | TTGGCCAACAATAGCCTCAGTTCTAACGGTAACGTGACTGAAAGTGGCTGCAAAGAGT |
| | GCGAAGAGCTCGAAGAAAAGAATATCAAGGAGTTCCTCCAATCATTTGTTCACATTGT |
| | GCAAATGTTTATCAACACCTCTTGA |
| SEQ ID NO: 24 | ATGCGCATAAGTAAGCCTCATCTGCGGTCCATTTCTATACAATGTTATCTGTGCTTGC |
| IL-15 | TTTTGAACTCCCACTTTCTTACGGAAGCAGGCATTCATGTGTTCATTCTGGGTTGTTT |
| | TTCtGCCGGGCTGCCCAAAACCGAGGCCAACTGGGTCAACGTGATCAGCGACCTCAAG |
| | AAGATCGAGGATTTGATTCAAAGTATGCATATAGACGCCACACTCTATACTGAGTCCG |
| | ACGTTCACCCGAGTTGTAAAGTTACGGCTATGAAGTGCTTTTTGTTGGAACTCCAGGT |
| | GATTTCCCTTGAATCCGGCGATGCGAGCATCCACGATACGGTAGAGAATCTTATTATT |
| | CTGGCGAATAATTCTCTGTCTTCAAATGGGAATGTAACTGAGAGCGGTTGTAAAGAAT |
| | GCGAAGAACTTGAAGAAAAGAATATCAAGGAATTTCTTCAGAGTTTCGTGCATATTGT |
| | TCAAATGTTCATCAACACATCCTGA |
| SEQ ID NO: 25 | RSKRSRLLHSDYMNMTPRRPGPTRKHYQPYAPPRDFAAYRSERVQPLEENVGNAARPR |
| CD28/OX40L/CDζ | FERNKRVKFSRSADAPAYQQGQNQLYNELNLGRREEYDVLDKRRGRDPEMGGKPRRKN |
| | PQEGLYNELQKDKMAEAYSEIGMKGERRRGKGHDGLYQGLSTATKDTYDALHMQASPP |
| | R |
| SEQ ID NO: 26 | RSKRSRLLHSDYMNMTPRRPGPTRKHYQPYAPPRDFAAYRSERVQPLEENVGNAARPR |
| CD28/OX40L/CDζ/ | FERNKRVKFSRSADAPAYQQGQNQLYNELNLGRREEYDVLDKRRGRDPEMGGKPRRKN |
| T2A/IL1-5 | PQEGLYNELQKDKMAEAYSEIGMKGERRRGKGHDGLYQGLSTATKDTYDALHMQALPP |
| | RGSGEGRGSLLTCGDVEENPGPMRISKPHLRSISIQCYLCLLLNSHELTEAGIHVFIL |
| | GCFSAGLPKTEANWVNVISDLKKIEDLIQSMHIDATLYTESDVHPSCKVTAMKCELLE |
| | LQVISLESGDASIHDTVENLIILANNSLSSNGNVTESGCKECEELEEKNIKEFLQSFV |
| | HIVQMFINTS- |
| SEQ ID NO: 27 | MALPVTALLLPLALLLHAARP |
| CD8α signal sequence | |
| SEQ ID NO: 28 | ATGGCCTTACCAGTGACCGCCTTGCTCCTGCCGCTGGCCTTGCTGCTCCACGCCGCCA |
| CD8α signal sequence | GGCCG |
| SEQ ID NO: 29 | ATGGCACTTCCTGTTACAGCCCTCCTGCTCCCACTGGCTTTGCTGCTGCATGCTGCAC |
| Codon Optimized | GACCG |
| CD8α signal sequence | |
| SEQ ID NO: 30 | DIQMTQSPSSLSASVGDRVTITCKASQDINSYLSWFQQKPGKAPKTLIYRANRLVDGV |
| anti-HER2 scFv | PSRFSGSGSGQDYTLTISSLQPEDFATYYCLQYDEFPWTFGQGTKVEIKGGGGSGGGG |
| | SGGGGSQVQLVQSGSELKKPGASVKVSCKASGYTFTNYGVNWVRQAPGQGLEWMGWIN |
| | THTGEPTYAEEFKGRFVFSLDTSVSTAYLQISSLKAEDTAVYYCARDDYYVRVDYWGQ |
| | GTTVTVSS |
| SEQ ID NO: 31 | GACATCCAGATGACCCAGAGCCCCAGCAGCCTGAGCGCCAGCGTGGGCGACAGAGTGA |
| anti-HER2 scFv | CCATCACCTGCAAGGCCAGCCAGGACATCAACAGCTACCTGAGCTGGTTCCAGCAGAA |
| | GCCCGGCAAGGCCCCCAAGACCCTGATCTACAGAGCCAACAGACTGGTGGACGGCGTG |
| | CCCAGCAGATTCAGCGGCAGCGGCAGCGGCCAGGACTACACCCTGACCATCAGCAGCC |
| | TGCAGCCCGAGGACTTCGCCACCTACTACTGCCTGCAGTACGACGAGTTCCCCTGGAC |
| | CTTCGGCCAGGGCACCAAGGTGGAGATCAAGGGTGGCGGTGGATCGGGCGGTGGTGGA |
| | TCTGGAGGAGGTGGCTCCCAGGTGCAGCTGGTGCAGAGCGGCAGCGAGCTGAAGAAGC |
| | CCGGCGCCAGCGTGAAGGTGAGCTGCAAGGCCAGCGGCTACACCTTCACCAACTACGG |
| | CGTGAACTGGGTGAGACAGGCCCCCGGCCAGGGCCTGGAGTGGATGGGCTGGATCAAC |
| | ACCCACACCGGCGAGCCCACCTACGCCGAGGAGTTCAAGGGCAGATTCGTGTTCAGCC |
| | TGGACACCAGCGTGAGCACCGCCTACCTGCAGATCAGCAGCCTGAAGGCCGAGGACAC |
| | CGCCGTGTACTACTGCGCCAGAGACGACTACTACGTGAGAGTGGACTACTGGGGCCAG |
| | GGCACCACCGTGACCGTGAGCAGC |
| SEQ ID NO: 32 | DIQMTQSPSSLSASVGDRVTITCKASQDINSYLSWFQQKPGKAPKTLIYRANRLVDGV |
| anti-HER2 scFv VL | PSRFSGSGSGQDYTLTISSLQPEDFATYYCLQYDEFPWTFGQGTKVEIK |
| SEQ ID NO: 33 | GACATCCAGATGACCCAGAGCCCCAGCAGCCTGAGCGCCAGCGTGGGCGACAGAGTGA |
| anti-HER2 scFv VL | CCATCACCTGCAAGGCCAGCCAGGACATCAACAGCTACCTGAGCTGGTTCCAGCAGAA |
| | GCCCGGCAAGGCCCCCAAGACCCTGATCTACAGAGCCAACAGACTGGTGGACGGCGTG |
| | CCCAGCAGATTCAGCGGCAGCGGCAGCGGCCAGGACTACACCCTGACCATCAGCAGCC |
| | TGCAGCCCGAGGACTTCGCCACCTACTACTGCCTGCAGTACGACGAGTTCCCCTGGAC |
| | CTTCGGCCAGGGCACCAAGGTGGAGATCAAG |
| SEQ ID NO: 34 | KASQDINSYLS |
| anti-HER2 scFv | |
| CDRL1 | |
| SEQ ID NO: 35 | AAGGCCAGCCAGGACATCAACAGCTACCTGAGC |
| anti-HER2 scFv | |
| CDRL1 | |
| SEQ ID NO: 36 | RANRLVD |
| anti-HER2 scFv | |
| CDRL2 | |
| SEQ ID NO: 37 | AGAGCCAACAGACTGGTGGAC |
| anti-HER2 scFv | |
| CDRL2 | |
| SEQ ID NO: 38 | LQYDEFPWT |
| anti-HER2 scFv | |
| CDRL3 | |
| SEQ ID NO: 39 | CTGCAGTACGACGAGTTCCCCTGGACC |
| anti-HER2 scFv | |
| CDRL3 | |
| SEQ ID NO: 40 | GGGGSGGGGSGGGGS |
| anti-HER2 scFv linker | |
| SEQ ID NO: 41 | GGTGGCGGTGGATCGGGCGGTGGTGGATCTGGAGGAGGTGGCTCC |
| anti-HER2 scFv linker | |
| SEQ ID NO: 42 | QVQLVQSGSELKKPGASVKVSCKASGYTFTNYGVNWVRQAPGQGLEWMGWINTHTGEP |
| anti-HER2 scFv VH | TYAEEFKGRFVFSLDTSVSTAYLQISSLKAEDTAVYYCARDDYYVRVDYWGQGTTVTV |
| | SS |
| SEQ ID NO: 43 | CAGGTGCAGCTGGTGCAGAGCGGCAGCGAGCTGAAGAAGCCCGGCGCCAGCGTGAAGG |
| anti-HER2 scFv VH | TGAGCTGCAAGGCCAGCGGCTACACCTTCACCAACTACGGCGTGAACTGGGTGAGACA |
| | GGCCCCCGGCCAGGGCCTGGAGTGGATGGGCTGGATCAACACCCACACCGGCGAGCCC |
| | ACCTACGCCGAGGAGTTCAAGGGCAGATTCGTGTTCAGCCTGGACACCAGCGTGAGCA |
| | CCGCCTACCTGCAGATCAGCAGCCTGAAGGCCGAGGACACCGCCGTGTACTACTGCGC |
| | CAGAGACGACTACTACGTGAGAGTGGACTACTGGGGCCAGGGCACCACCGTGACCGTG |
| | AGCAGC |
| SEQ ID NO: 44 | NYGVN |
| anti-HER2 scFv | |
| CDRH1 | |
| SEQ ID NO: 45 | AACTACGGCGTGAAC |
| anti-HER2 scFv | |
| CDRH1 | |
| SEQ ID NO: 46 | WINTHTGEPTYAEEFKG |
| anti-HER2 scFv | |
| CDRH2 | |
| SEQ ID NO: 47 | TGGATCAACACCCACACCGGCGAGCCCACCTACGCCGAGGAGTTCAAGGGC |
| anti-HER2 scFv | |
| CDRH2 | |
| SEQ ID NO: 48 | DDYYVRVDY |
| anti-HER2 scFv | |
| CDRH3 | |
| SEQ ID NO: 49 | GACGACTACTACGTGAGAGTGGACTAC |
| anti-HER2 scFv | |
| CDRH3 | |
| SEQ ID NO: 50 | AKPTTTPAPRPPTPAPTIASQPLSLRPEACRPAAGGAVHTRGLDFACD |
| CD8α hinge | |
| SEQ ID NO: 51 | GCGAAGCCCACCACGACGCCAGCGCCGCGACCACCAACACCGGCGCCCACCATCGCGT |
| CD8α hinge | CGCAGCCCCTGTCCCTGCGCCCAGAGGCGTGCCGGCCAGCGGCGGGGGGCGCAGTGCA |
| | CACGAGGGGGCTGGACTTCGCCTGTGAT |
| SEQ ID NO: 52 | GCAAAACCTACCACAACTCCTGCACCACGCCCCCCTACTCCAGCACCTACCATCGCAT |
| Codon Optimized | CTCAGCCACTGAGTCTGCGACCAGAGGCCTGCCGGCCCGCCGCCGGGGGGCCGTCCA |
| CD8α hinge | TACCAGAGGGCTGGACTTTGCCTGCGAT |
| SEQ ID NO: 53 | FWVLVVVGGVLACYSLLVTVAFIIFWV |
| CD28 transmembrane | |
| domain | |
| SEQ ID NO: 54 | TTTTGGGTGCTGGTGGTGGTTGGTGGAGTCCTGGCTTGCTATAGCTTGCTAGTAACAG |
| CD28 transmembrane | TGGCCTTTATTATTTTCTGGGTG |
| domain | |
| SEQ ID NO: 55 | TTTTGGGTCCTGGTGGTCGTGGGAGGGGTGCTGGCATGTTACTCACTGCTGGTCACCG |
| Codon Optimized | TGGCCTTCATCATCTTCTGGGTG |
| CD28 transmembrane | |
| domain | |
| SEQ ID NO: 56 | MALPVTASLLPLALLLHAARPDIQMTQSPSSLSASVGDRVTITCKASQDINSYLSWFQ |
| HER2 CAR | QKPGKAPKTLIYRANRLVDGVPSRFSGSGSGQDYTLTISSLQPEDFATYYCLQYDEFP |
| | WTFGQGTKVEIKGGGGSGGGGSGGGGSQVQLVQSGSELKKPGASVKVSCKASGYTFTN |
| | YGVNWVRQAPGQGLEWMGWINTHTGEPTYAEEFKGRFVFSLDTSVSTAYLQISSLKAE |
| | DTAVYYCARDDYYVRVDYWGQGTTVTVSSAKPTTTPAPRPPTPAPTIASQPLSLRPEA |
| | CRPAAGGAVHTRGLDFACDFWVLVVVGGVLACYSLLVTVAFIIFWVRSKRSRLLHSDY |
| | MNMTPRRPGPTRKHYQPYAPPRDFAAYRSERVQPLEENVGNAARPRFERNKRVKESRS |
| | ADAPAYQQGQNQLYNELNLGRREEYDVLDKRRGRDPEMGGKPRRKNPQEGLYNELQKD |
| | KMAEAYSEIGMKGERRRGKGHDGLYQGLSTATKDTYDASHMQALPPR |
| SEQ ID NO: 57 | ATGGCCTTACCAGTGACCGCCTTGCTCCTGCCGCTGGCCTTGCTGCTCCACGCCGCCA |
| HER2 CAR | GGCCGGACATCCAGATGACCCAGAGCCCCAGCAGCCTGAGCGCCAGCGTGGGCGACAG |
| | AGTGACCATCACCTGCAAGGCCAGCCAGGACATCAACAGCTACCTGAGCTGGTTCCAG |
| | CAGAAGCCCGGCAAGGCCCCCAAGACCCTGATCTACAGAGCCAACAGACTGGTGGACG |
| | GCGTGCCCAGCAGATTCAGCGGCAGCGGCAGCGGCCAGGACTACACCCTGACCATCAG |
| | CAGCCTGCAGCCCGAGGACTTCGCCACCTACTACTGCCTGCAGTACGACGAGTTCCCC |
| | TGGACCTTCGGCCAGGGCACCAAGGTGGAGATCAAGGGTGGCGGTGGATCGGGCGGTG |
| | GTGGATCTGGAGGAGGTGGCTCCCAGGTGCAGCTGGTGCAGAGCGGCAGCGAGCTGAA |
| | GAAGCCCGGCGCCAGCGTGAAGGTGAGCTGCAAGGCCAGCGGCTACACCTTCACCAAC |
| | TACGGCGTGAACTGGGTGAGACAGGCCCCCGGCCAGGGCCTGGAGTGGATGGGCTGGA |
| | TCAACACCCACACCGGCGAGCCCACCTACGCCGAGGAGTTCAAGGGCAGATTCGTGTT |
| | CAGCCTGGACACCAGCGTGAGCACCGCCTACCTGCAGATCAGCAGCCTGAAGGCCGAG |
| | GACACCGCCGTGTACTACTGCGCCAGAGACGACTACTACGTGAGAGTGGACTACTGGG |
| | GCCAGGGCACCACCGTGACCGTGAGCAGCGCGAAGCCCACCACGACGCCAGCGCCGCG |
| | ACCACCAACACCGGCGCCCACCATCGCGTCGCAGCCCCTGTCCCTGCGCCCAGAGGCG |
| | TGCCGGCCAGCGGCGGGGGGCGCAGTGCACACGAGGGGGCTGGACTTCGCCTGTGATT |
| | TTTGGGTGCTGGTGGTGGTTGGTGGAGTCCTGGCTTGCTATAGCTTGCTAGTAACAGT |
| | GGCCTTTATTATTTTCTGGGTGAGGAGTAAGAGGAGCAGGCTCCTGCACAGTGACTAC |
| | ATGAACATGACTCCCCGCCGCCCCGGGCCCACCCGCAAGCATTACCAGCCCTATGCCC |
| | CACCACGCGACTTCGCAGCCTATCGCTCCGAAAGGGTCCAACCCCTGGAAGAGAATGT |
| | GGGAAATGCAGCCAGGCCAAGATTCGAGAGGAACAAGAGAGTGAAGTTCAGCAGGAGC |
| | GCAGACGCCCCCGCGTACCAGCAGGGCCAGAACCAGCTCTATAACGAGCTCAATCTAG |
| | GACGAAGAGAGGAGTACGATGTTTTGGACAAGAGACGTGGCCGGGACCCTGAGATGGG |
| | GGGAAAGCCGAGAAGGAAGAACCCTCAGGAAGGCCTGTACAATGAACTGCAGAAAGAT |
| | AAGATGGCGGAGGCCTACAGTGAGATTGGGATGAAAGGCGAGCGCCGGAGGGGCAAGG |
| | GGCACGATGGCCTTTACCAGGGTCTCAGTACAGCCACCAAGGACACCTACGACGCCCT |
| | TCACATGCAGGCCCTGCCCCCTCGC |
| SEQ ID NO: 58 | ATGGCACTTCCTGTTACAGCCCTCCTGCTCCCACTGGCTTTGCTGCTGCATGCTGCAC |
| Codon Optimized | GACCGGACATCCAGATGACCCAGAGCCCCAGCAGCCTGAGCGCCAGCGTGGGCGACAG |
| HER2 CAR | AGTGACCATCACCTGCAAGGCCAGCCAGGACATCAACAGCTACCTGAGCTGGTTCCAG |
| | CAGAAGCCCGGCAAGGCCCCCAAGACCCTGATCTACAGAGCCAACAGACTGGTGGACG |
| | GCGTGCCCAGCAGATTCAGCGGCAGCGGCAGCGGCCAGGACTACACCCTGACCATCAG |
| | CAGCCTGCAGCCCGAGGACTTCGCCACCTACTACTGCCTGCAGTACGACGAGTTCCCC |
| | TGGACCTTCGGCCAGGGCACCAAGGTGGAGATCAAGGGTGGCGGTGGATCGGGCGGTG |
| | GTGGATCTGGAGGAGGTGGCTCCCAGGTGCAGCTGGTGCAGAGCGGCAGCGAGCTGAA |
| | GAAGCCCGGCGCCAGCGTGAAGGTGAGCTGCAAGGCCAGCGGCTACACCTTCACCAAC |
| | TACGGCGTGAACTGGGTGAGACAGGCCCCCGGCCAGGGCCTGGAGTGGATGGGCTGGA |
| | TCAACACCCACACCGGCGAGCCCACCTACGCCGAGGAGTTCAAGGGCAGATTCGTGTT |
| | CAGCCTGGACACCAGCGTGAGCACCGCCTACCTGCAGATCAGCAGCCTGAAGGCCGAG |
| | GACACCGCCGTGTACTACTGCGCCAGAGACGACTACTACGTGAGAGTGGACTACTGGG |
| | GCCAGGGCACCACCGTGACCGTGAGCAGCGCAAAACCTACCACAACTCCTGCACCACG |
| | CCCCCCTACTCCAGCACCTACCATCGCATCTCAGCCACTGAGTCTGCGACCAGAGGCC |
| | TGCCGGCCCGCCGCCGGCGGGGCCGTCCATACCAGAGGGCTGGACTTTGCCTGCGATT |
| | TTTGGGTCCTGGTGGTCGTGGGAGGGGTGCTGGCATGTTACTCACTGCTGGTCACCGT |
| | GGCCTTCATCATCTTCTGGGTGCGGAGCAAGAGGTCCCGCCTGCTGCACAGCGACTAT |
| | ATGAACATGACCCCACGGAGACCCGGCCCTACACGGAAACATTACCAGCCCTATGCTC |
| | CACCCCGGGACTTCGCAGCTTACAGAAGTGAAAGAGTGCAGCCCCTGGAAGAGAATGT |
| | CGGGAATGCCGCTCGCCCAAGATTTGAAAGGAACAAACGAGTGAAGTTCAGCAGGTCC |
| | GCCGACGCTCCTGCATACCAGCAGGGACAGAACCAGCTGTATAACGAGCTGAATCTGG |
| | GCCGGAGAGAGGAATACGACGTGCTGGACAAAAGGCGGGGCCGGGACCCCGAAATGGG |
| | AGGGAAGCCACGACGGAAAAACCCCCAGGAGGGCCTGTACAATGAGCTGCAAAAGGAC |
| | AAAATGGCCGAGGCTTATTCTGAAATCGGGATGAAGGGAGAGAGAAGGCGCGGAAAAG |
| | GCCACGATGGCCTGTACCAGGGGCTGAGCACCGCTACAAAGGACACCTATGATGCACT |
| | GCACATGCAGGCCCTGCCCCCTCGG |
| SEQ ID NO: 59 | MALPVTALLLPLALLLHAARPDIQMTQSPSSLSASVGDRVTITCKASQDINSYLSWFQ |
| HER2 CAR with T2A | QKPGKAPKTLIYRANRLVDGVPSRFSGSGSGQDYTLTISSLQPEDFATYYCLQYDEFP |
| and IL-15 | WTFGQGTKVEIKGGGGSGGGGSGGGGSQVQLVQSGSELKKPGASVKVSCKASGYTFTN |
| | YGVNWVRQAPGQGLEWMGWINTHTGEPTYAEEFKGRFVFSLDTSVSTAYLQISSLKAE |
| | DTAVYYCARDDYYVRVDYWGQGTTVTVSSAKPTTTPAPRPPTPAPTIASQPLSLRPEA |
| | CRPAAGGAVHTRGLDFACDFWVLVVVGGVLACYSLLVTVAFIIFWVRSKRSRLLHSDY |
| | MNMTPRRPGPTRKHYQPYAPPRDFAAYRSERVQPLEENVGNAARPRFERNKRVKESRS |
| | ADAPAYQQGQNQLYNELNLGRREEYDVLDKRRGRDPEMGGKPRRKNPQEGLYNELQKD |
| | KMAEAYSEIGMKGERRRGKGHDGLYQGLSTATKDTYDASHMQALPPRGSGEGRGSALT |
| | CGDVEENPGPMRISKPHLRSISIQCYLCLLLNSHFLTEAGIHVFILGCFSAGLPKTEA |
| | NWVNVISDLKKIEDLIQSMHIDATLYTESDVHPSCKVTAMKCFLLELQVISLESGDAS |
| | IHDTVENLIILANNSLSSNGNVTESGCKECEELEEKNIKEFLQSFVHIVQMFINTS- |
| SEQ ID NO: 60 | ATGGCCTTACCAGTGACCGCCTTGCTCCTGCCGCTGGCCTTGCTGCTCCACGCCGCCA |
| HER2 CAR with T2A | GGCCGGACATCCAGATGACCCAGAGCCCCAGCAGCCTGAGCGCCAGCGTGGGCGACAG |
| and IL-15 | AGTGACCATCACCTGCAAGGCCAGCCAGGACATCAACAGCTACCTGAGCTGGTTCCAG |
| | CAGAAGCCCGGCAAGGCCCCCAAGACCCTGATCTACAGAGCCAACAGACTGGTGGACG |
| | GCGTGCCCAGCAGATTCAGCGGCAGCGGCAGCGGCCAGGACTACACCCTGACCATCAG |
| | CAGCCTGCAGCCCGAGGACTTCGCCACCTACTACTGCCTGCAGTACGACGAGTTCCCC |
| | TGGACCTTCGGCCAGGGCACCAAGGTGGAGATCAAGGGTGGCGGTGGATCGGGCGGTG |
| | GTGGATCTGGAGGAGGTGGCTCCCAGGTGCAGCTGGTGCAGAGCGGCAGCGAGCTGAA |
| | GAAGCCCGGCGCCAGCGTGAAGGTGAGCTGCAAGGCCAGCGGCTACACCTTCACCAAC |
| | TACGGCGTGAACTGGGTGAGACAGGCCCCCGGCCAGGGCCTGGAGTGGATGGGCTGGA |
| | TCAACACCCACACCGGCGAGCCCACCTACGCCGAGGAGTTCAAGGGCAGATTCGTGTT |
| | CAGCCTGGACACCAGCGTGAGCACCGCCTACCTGCAGATCAGCAGCCTGAAGGCCGAG |
| | GACACCGCCGTGTACTACTGCGCCAGAGACGACTACTACGTGAGAGTGGACTACTGGG |
| | GCCAGGGCACCACCGTGACCGTGAGCAGCGCGAAGCCCACCACGACGCCAGCGCCGCG |
| | ACCACCAACACCGGCGCCCACCATCGCGTCGCAGCCCCTGTCCCTGCGCCCAGAGGCG |
| | TGCCGGCCAGCGGCGGGGGGCGCAGTGCACACGAGGGGGCTGGACTTCGCCTGTGATT |
| | TTTGGGTGCTGGTGGTGGTTGGTGGAGTCCTGGCTTGCTATAGCTTGCTAGTAACAGT |
| | GGCCTTTATTATTTTCTGGGTGAGGAGTAAGAGGAGCAGGCTCCTGCACAGTGACTAC |
| | ATGAACATGACTCCCCGCCGCCCCGGGCCCACCCGCAAGCATTACCAGCCCTATGCCC |
| | CACCACGCGACTTCGCAGCCTATCGCTCCGAAAGGGTCCAACCCCTGGAAGAGAATGT |
| | GGGAAATGCAGCCAGGCCAAGATTCGAGAGGAACAAGAGAGTGAAGTTCAGCAGGAGC |
| | GCAGACGCCCCCGCGTACCAGCAGGGCCAGAACCAGCTCTATAACGAGCTCAATCTAG |
| | GACGAAGAGAGGAGTACGATGTTTTGGACAAGAGACGTGGCCGGGACCCTGAGATGGG |
| | GGGAAAGCCGAGAAGGAAGAACCCTCAGGAAGGCCTGTACAATGAACTGCAGAAAGAT |
| | AAGATGGCGGAGGCCTACAGTGAGATTGGGATGAAAGGCGAGCGCCGGAGGGGCAAGG |
| | GGCACGATGGCCTTTACCAGGGTCTCAGTACAGCCACCAAGGACACCTACGACGCCCT |
| | TCACATGCAGGCCCTGCCCCCTCGCGGCTCAGGTGAGGGGCGCGGGAGCCTGCTGACT |
| | TGTGGGGATGTAGAGGAAAATCCTGGTCCTATGAGAATTTCGAAACCACATTTGAGAA |
| | GTATTTCCATCCAGTGCTACTTGTGTTTACTTCTAAACAGTCATTTTCTAACTGAAGC |
| | TGGCATTCATGTCTTCATTTTGGGCTGTTTCAGTGCAGGGCTTCCTAAAACAGAAGCC |
| | AACTGGGTGAATGTAATAAGTGATTTGAAAAAAATTGAAGATCTTATTCAATCTATGC |
| | ATATTGATGCTACTTTATATACGGAAAGTGATGTTCACCCCAGTTGCAAAGTAACAGC |
| | AATGAAGTGCTTTCTCTTGGAGTTACAAGTTATTTCACTTGAGTCCGGAGATGCAAGT |
| | ATTCATGATACAGTAGAAAATCTGATCATCCTAGCAAACAACAGTTTGTCTTCTAATG |
| | GGAATGTAACAGAATCTGGATGCAAAGAATGTGAGGAACTGGAGGAAAAAAATATTAA |
| | AGAATTTTTGCAGAGTTTTGTACATATTGTCCAAATGTTCATCAACACTTCT |
| SEQ ID NO: 61 | ATGGCACTTCCTGTTACAGCCCTCCTGCTCCCACTGGCTTTGCTGCTGCATGCTGCAC |
| Codon Optimized | GACCGGACATCCAGATGACCCAGAGCCCCAGCAGCCTGAGCGCCAGCGTGGGCGACAG |
| HER2 CAR with T2A | AGTGACCATCACCTGCAAGGCCAGCCAGGACATCAACAGCTACCTGAGCTGGTTCCAG |
| and IL-15 | CAGAAGCCCGGCAAGGCCCCCAAGACCCTGATCTACAGAGCCAACAGACTGGTGGACG |
| | GCGTGCCCAGCAGATTCAGCGGCAGCGGCAGCGGCCAGGACTACACCCTGACCATCAG |
| | CAGCCTGCAGCCCGAGGACTTCGCCACCTACTACTGCCTGCAGTACGACGAGTTCCCC |
| | TGGACCTTCGGCCAGGGCACCAAGGTGGAGATCAAGGGTGGCGGTGGATCGGGCGGTG |
| | GTGGATCTGGAGGAGGTGGCTCCCAGGTGCAGCTGGTGCAGAGCGGCAGCGAGCTGAA |
| | GAAGCCCGGCGCCAGCGTGAAGGTGAGCTGCAAGGCCAGCGGCTACACCTTCACCAAC |
| | TACGGCGTGAACTGGGTGAGACAGGCCCCCGGCCAGGGCCTGGAGTGGATGGGCTGGA |
| | TCAACACCCACACCGGCGAGCCCACCTACGCCGAGGAGTTCAAGGGCAGATTCGTGTT |
| | CAGCCTGGACACCAGCGTGAGCACCGCCTACCTGCAGATCAGCAGCCTGAAGGCCGAG |
| | GACACCGCCGTGTACTACTGCGCCAGAGACGACTACTACGTGAGAGTGGACTACTGGG |
| | GCCAGGGCACCACCGTGACCGTGAGCAGCGCAAAACCTACCACAACTCCTGCACCACG |
| | CCCCCCTACTCCAGCACCTACCATCGCATCTCAGCCACTGAGTCTGCGACCAGAGGCC |
| | TGCCGGCCCGCCGCCGGCGGGGCCGTCCATACCAGAGGGCTGGACTTTGCCTGCGATT |
| | TTTGGGTCCTGGTGGTCGTGGGAGGGGTGCTGGCATGTTACTCACTGCTGGTCACCGT |
| | GGCCTTCATCATCTTCTGGGTGCGGAGCAAGAGGTCCCGCCTGCTGCACAGCGACTAT |
| | ATGAACATGACCCCACGGAGACCCGGCCCTACACGGAAACATTACCAGCCCTATGCTC |
| | CACCCCGGGACTTCGCAGCTTACAGAAGTGAAAGAGTGCAGCCCCTGGAAGAGAATGT |
| | CGGGAATGCCGCTCGCCCAAGATTTGAWAGGAACAAACGAGTGAAGTTCAGCAGGTCC |
| | GCCGACGCTCCTGCATACCAGCAGGGACAGAACCAGCTGTATAACGAGCTGAATCTGG |
| | GCCGGAGAGAGGAATACGACGTGCTGGACAAAAGGCGGGGCCGGGACCCCGAAATGGG |
| | AGGGAAGCCACGACGGAAAAACCCCCAGGAGGGCCTGTACAATGAGCTGCAAAAGGAC |
| | AAAATGGCCGAGGCTTATTCTGAAATCGGGATGAAGGGAGAGAGAAGGCGCGGAAAAG |
| | GCCACGATGGCCTGTACCAGGGGCTGAGCACCGCTACAAAGGACACCTATGATGCACT |
| | GCACATGCAGGCCCTGCCCCCTCGGGGCTCAGGTGAGGGGCGCGGGAGCCTGCTGACT |
| | TGTGGGGATGTAGAGGAAAATCCTGGTCCTATGAGAATCAGCAAACCACACCTCCGGA |
| | GCATATCAATCCAGTGTTACTTGTGCCTTCTTTTGAACTCCCATTTCCTCACCGAGGC |
| | AGGCATTCATGTGTTCATATTGGGGTGCTTTAGTGCTGGGCTTCCGAAAACGGAAGCT |
| | AACTGGGTAAACGTCATCAGTGACCTTAAAAAAATTGAGGATCTTATCCAATCAATGC |
| | ACATCGACGCGACTCTCTACACAGAATCTGACGTACACCCGTCATGCAAAGTCACGGC |
| | AATGAAGTGTTTTCTTCTCGAGCTCCAAGTAATTTCCCTGGAGTCTGGCGATGCCTCC |
| | ATCCACGATACGGTTGAAAATCTGATTATATTGGCCAACAATAGCCTCAGTTCTAACG |
| | GTAACGTGACTGAAAGTGGCTGCAAAGAGTGCGAAGAGCTCGAAGAAAAGAATATCAA |
| | GGAGTTCCTCCAATCATTTGTTCACATTGTGCAAATGTTTATCAACACCTCT |
| SEQ ID NO: 62 | MELAALCRWGLLLALLPPGAASTQVCTGTDMKLRLPASPETHLDMLRHLYQGCQVVQG |
| P04626 ERBB2 Human | NLELTYLPTNASLSFLQDIQEVQGYVLIAHNQVRQVPLQRLRIVRGTQLFEDNYALAV |
| Receptor tyrosine- | LDNGDPLNNTTPVTGASPGGLRELQLRSLTEILKGGVLIQRNPQLCYQDTILWKDIFH |
| protein kinase (HER2) | KNNQLALTLIDTNRSRACHPCSPMCKGSRCWGESSEDCQSLTRTVCAGGCARCKGPLP |
| | TDCCHEQCAAGCTGPKHSDCLACLHENHSGICELHCPALVTYNTDTFESMPNPEGRYT |
| | FGASCVTACPYNYLSTDVGSCTLVCPLHNQEVTAEDGTQRCEKCSKPCARVCYGLGME |
| | HLREVRAVTSANIQEFAGCKKIFGSLAFLPESFDGDPASNTAPLQPEQLQVFETLEEI |
| | TGYLYISAWPDSLPDLSVFQNLQVIRGRILHNGAYSLTLQGLGISWLGLRSLRELGSG |
| | LALIHHNTHLCFVHTVPWDQLERNPHQALLHTANRPEDECVGEGLACHQLCARGHCWG |
| | PGPTQCVNCSQFLRGQECVEECRVLQGLPREYVNARHCLPCHPECQPQNGSVTCFGPE |
| | ADQCVACAHYKDPPFCVARCPSGVKPDLSYMPIWKEPDEEGACQPCPINCTHSCVDLD |
| | DKGCPAEQRASPLTSIISAVVGILLVVVLGVVFGILIKRRQQKIRKYTMRRLLQETEL |
| | VEPLTPSGAMPNQAQMRILKETELRKVKVLGSGAFGTVYKGIWIPDGENVKIPVAIKV |
| | LRENTSPKANKEILDEAYVMAGVGSPYVSRLLGICLTSTVQLVTQLMPYGCLLDHVRE |
| | NRGRLGSQDLLNWCMQIAKGMSYLEDVRLVHRDLAARNVLVKSPNHVKITDEGLARLL |
| | DIDETEYHADGGKVPIKWMALESILRRRFTHQSDVWSYGVTVWELMTFGAKPYDGIPA |
| | REIPDLLEKGERLPQPPICTIDVYMIMVKCWMIDSECRPRFRELVSEFSRMARDPQRF |
| | VVIQNEDSGPASPLDSTFYRSLLEDDDMGDLVDAEEYLVPQQGFFCPDPAPGAGGMVH |
| | HRHRSSSTRSGGGDLTLGLEPSEEEAPRSPLAPSEGAGSDVFDGDLGMGAAKGLQSLP |
| | THDPSPLQRYSEDPTVPLPSETDGYVAPLTCSPQPEYVNQPDVRPQPPSPREGPLPAA |
| | RPAGATLERPKTLSPGKNGVVKDVFAFGGAVENPEYLTPQGGAAPQPHPPPAESPAFD |
| | NLYYWDQDPPERGAPPSTFKGTPTAENPEYLGLDVPV |
| SEQ ID NO: 63 | MKWVTFISLLFLESSAYSRGVERRDAHKSEVAHRFKDLGEENFKALVLIAFAQYLQQC |
| Human Albumin | PFEDHVKSVNEVTEFAKTCVADESAENCDKSLHTLFGDKLCTVATLRETYGEMADCCA |
| | KQEPERNECFLQHKDDNPNLPRLVRPEVDVMCTAFHDNEETFLKKYLYEIARRHPYFY |
| | APELLFFAKRYKAAFTECCQAADKAACLLPKLDELRDEGKASSAKQRLKCASLQKFGE |
| | RAFKAWAVARLSQRFPKAEFAEVSKLVTDLTKVHTECCHGDLLECADDRADLAKYICE |
| | NQDSISSKLKECCEKPLLEKSHCIAEVENDEMPADLPSLAADEVESKDVCKNYAEAKD |
| | VFLGMFLYEYARRHPDYSVVLLLRLAKTYETTLEKCCAAADPHECYAKVEDEEKPLVE |
| | EPQNLIKQNCELFEQLGEYKFQNALLVRYTKKVPQVSTPTLVEVSRNLGKVGSKCCKH |
| | PEAKRMPCAEDYLSVVLNQLCVLHEKTPVSDRVTKCCTESLVNRRPCFSALEVDETYV |
| | PKEFNAETFTFHADICTLSEKERQIKKQTALVELVKHKPKATKEQLKAVMDDEAAFVE |
| | KCCKADDKETCFAEEGKKLVAASQAALGL |
| SEQ ID NO: 64 | DIQMTQSPSSLSASVGDRVTITCKASQDINSYLSWFQQKPGKAPKTLIYRANRLVDGV |
| HER2 CAR | PSRFSGSGSGQDYTLTISSLQPEDFATYYCLQYDEFPWTFGQGTKVEIKGGGGSGGGG |
| | SGGGGSQVQLVQSGSELKKPGASVKVSCKASGYTFTNYGVNWVRQAPGQGLEWMGWIN |
| | THTGEPTYAEEFKGRFVFSLDTSVSTAYLQISSLKAEDTAVYYCARDDYYVRVDYWGQ |
| | GTTVTVSSAKPTTTPAPRPPTPAPTIASQPLSLRPEACRPAAGGAVHTRGLDEACDFW |
| | VLVVVGGVLACYSLLVTVAFIIFWVRSKRSRLLHSDYMNMTPRRPGPTRKHYQPYAPP |
| | RDFAAYRSERVQPLEENVGNAARPRFERNKRVKFSRSADAPAYQQGQNQLYNELNLGR |
| | REEYDVLDKRRGRDPEMGGKPRRKNPQEGLYNELQKDKMAEAYSEIGMKGERRRGKGH |
| | DGLYQGLSTATKDTYDALHMQALPPR |
| SEQ ID NO: 65 | DIQMTQSPSSLSASVGDRVTITCKASQDINSYLSWFQQKPGKAPKTLIYRANRLVDGV |
| HER2 CAR with T2A | PSRFSGSGSGQDYTLTISSLQPEDFATYYCLQYDEFPWTFGQGTKVEIKGGGGSGGGG |
| and IL-15 | SGGGGSQVQLVQSGSELKKPGASVKVSCKASGYTFTNYGVNWVRQAPGQGLEWMGWIN |
| | THTGEPTYAEEFKGRFVFSLDTSVSTAYLQISSLKAEDTAVYYCARDDYYVRVDYWGQ |
| | GTTVTVSSAKPTTTPAPRPPTPAPTIASQPLSLRPEACRPAAGGAVHTRGLDEACDFW |
| | VLVVVGGVLACYSLLVTVAFIIFWVRSKRSRLLHSDYMNMTPRRPGPTRKHYQPYAPP |
| | RDFAAYRSERVQPLEENVGNAARPRFERNKRVKESRSADAPAYQQGQNQLYNELNEGR |
| | REEYDVLDKRRGRDPEMGGKPRRKNPQEGLYNELQKDKMAEAYSEIGMKGERRRGKGH |
| | DGLYQGLSTATKDTYDALHMQALPPRGSGEGRGSLLTCGDVEENPGPMRISKPHLRSI |
| | SIQCYLCLLLNSHELTEAGIHVFILGCFSAGLPKTEANWVNVISDLKKIEDLIQSMHI |
| | DATLYTESDVHPSCKVTAMKCELLELQVISLESGDASIHDTVENLIILANNSLSSNGN |
| | VTESGCKECEELEEKNIKEFLQSFVHIVQMFINTS- |
| SEQ ID NO: 66 | DIQMTQSPSSLSASVGDRVTITCKASQDINSYLSWFQQKPGKAPKTLIYRANRLVDGV |
| HER2 CAR without | PSRFSGSGSGQDYTLTISSLQPEDFATYYCLQYDEFPWTFGQGTKVEIKGGGGSGGGG |
| OX40L | SGGGGSQVQLVQSGSELKKPGASVKVSCKASGYTFTNYGVNWVRQAPGQGLEWMGWIN |
| | THTGEPTYAEEFKGRFVFSLDTSVSTAYLQISSLKAEDTAVYYCARDDYYVRVDYWGQ |
| | GTTVTVSSAKPTTTPAPRPPTPAPTIASQPLSLRPEACRPAAGGAVHTRGLDEACDEW |
| | VLVVVGGVLACYSLLVTVAFIIFWVRSKRSRLLHSDYMNMTPRRPGPTRKHYQPYAPP |
| | RDFAAYRSRVKFSRSADAPAYQQGQNQLYNELNLGRREEYDVLDKRRGRDPEMGGKPR |
| | RKNPQEGLYNELQKDKMAEAYSEIGMKGERRRGKGHDGLYQGLSTATKDTYDALHMQA |
| | LPPR |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising administering from 1 million to 100 billion natural killer (NK) cells to a patient in need thereof, wherein the NK cells comprise a polynucleotide comprising:
a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and
b) a nucleic acid encoding an IL-15.

2. The method of claim 1, wherein the NK cells are administered in a dose comprising from 1 billion to 100 billion NK cells, optionally in a dose comprising at or about 1 million to at or about 20 billion cells (e.g., at or about 5 million cells, at or about 25 million cells, at or about 50 million cells, at or about 75 million cells, at or about 100 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, or a range defined by any two of the foregoing values), at or about 10 million to at or about 20 billion cells (e.g., at or about 25 million cells, at or about 50 million cells, at or about 75 million cells, at or about 100 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, at or about 20 billion cells, or a range defined by any two of the foregoing values), and in some cases at or about 100 million cells to at or about 50 billion cells (e.g., at or about 150 million cells, at or about 200 million cells, at or about 300 million cells, at or about 400 million cells, at or about 500 million cells, at or about 1 billion cells, at or about 2 billion cells, at or about 3 billion cells, at or about 4 billion cells, at or about 5 billion cells, at or about 6 billion cells, at or about 7 billion cells, at or about 8 billion cells, at or about 9 billion cells, at or about 10 billion cells, at or about 20 billion cells, at or about 30 billion cells, at or about 40 billion cells).

3. The method of claim 1, where the NK cells are administered in a dose comprising at least 1×10⁶, 2×10⁶, 3×10⁶, 4×10⁶, 5×10⁶, 6×10⁶, 7×10⁶, 8×10⁶, or 9×10⁶ cells per dose, at or at least 1×10⁷, 2×10⁷, 3×10⁷, 4×10⁷, 5×10⁷, 6×10⁷, 7×10⁷, 8×10⁷, or 9×10⁷ cells per dose, at or at least 1×10⁸, 2×10⁸, 3×10⁸, 4×10⁸, 5×10⁸, 6×10⁸, 7×10⁸, 8×10⁸, or 9×10⁸ cells per dose, at least 1×10⁹, 2×10⁹, 3×10⁹, 4×10⁹, 5×10⁹, 6×10⁹, 7×10⁹, 8×10⁹, or 9×10⁹ cells per dose, or at least 1×10¹⁰ or 2×10¹⁰ cells per dose.

4. The method of claim 1, where the NK cells are administered in a dose comprising about 1×10⁶, 2×10⁶, 3×10⁶, 4×10⁶, 5×10⁶, 6×10⁶, 7×10⁶, 8×10⁶, or 9×10⁶ cells per dose, about 1×10⁷, 2×10⁷, 3×10⁷, 4×10⁷, 5×10⁷, 6×10⁷, 7×10⁷, 8×10⁷, or 9×10⁷ cells per dose, about 1×10⁸, 2×10⁸, 3×10⁸, 4×10⁸, 5×10⁸, 6×10⁸, 7×10⁸, 8×10⁸, or 9×10⁸ cells per dose, about 1×10⁹, 2×10⁹, 3×10⁹, 4×10⁹, 5×10⁹, 6×10⁹, 7×10⁹, 8×10⁹, or 9×10⁹ cells per dose, or about 1×10¹⁰ or 2×10¹⁰ cells per dose.

5. The method of claim 1, wherein the NK cells are administered in a dose comprising at or about 1 million to at or about 20 billion CAR-expressing NK cells (e.g., at or about 5 million cells, at or about 25 million CAR-expressing cells, at or about 50 million CAR-expressing cells, at or about 75 million CAR-expressing cells, at or about 100 million CAR-expressing cells, at or about 200 million CAR-expressing cells, at or about 300 million CAR-expressing cells, at or about 400 million CAR-expressing cells, at or about 500 million CAR-expressing cells, at or about 1 billion CAR-expressing cells, at or about 2 billion CAR-expressing cells, at or about 3 billion CAR-expressing cells, at or about 4 billion CAR-expressing cells, at or about 5 billion CAR-expressing cells, at or about 6 billion CAR-expressing cells, at or about 7 billion CAR-expressing cells, at or about 8 billion CAR-expressing cells, at or about 9 billion CAR-expressing cells, at or about 10 billion CAR-expressing cells, or at or about 20 billion CAR-expressing cells, or a range defined by any two of the foregoing values) per dose.

6. The method of claim 1, wherein the NK cells are administered in a dose comprising at or at about 1×10⁶, 2×10⁶, 3×10⁶, 4×10⁶, 5×10⁶, 6×10⁶, 7×10⁶, 8×10⁶, or 9×10⁶ CAR-expressing cells per dose, a dose comprising at or at about 1×10⁷, 2×10⁷, 3×10⁷, 4×10⁷, 5×10⁷, 6×10⁷, 7×10⁷, 8×10⁷, or 9×10⁷ CAR-expressing cells per dose, a dose comprising at or at about 1×10⁸, 2×10⁸, 3×10⁸, 4×10⁸, 5×10⁸, 6×10⁸, 7×10⁸, 8×10⁸, or 9×10⁸ CAR-expressing cells per dose, a dose comprising at or at about 1×10⁹, 2×10⁹, 3×10⁹, 4×10⁹, 5×10⁹, 6×10⁹, 7×10⁹, 8×10⁹, or 9×10⁹ CAR-expressing cells per dose, or a dose comprising at or at about 1×10¹⁰ or at or at about 2×10¹⁰ CAR-expressing cells per dose.

7. The method of claim 1, wherein the NK cells are administered in a dose comprising at least or at least about 1×10⁶, 2×10⁶, 3×10⁶, 4×10⁶, 5×10⁶, 6×10⁶, 7×10⁶, 8×10⁶, or 9×10⁶ CAR-expressing cells per dose, at least or at least about 1×10⁷, 2×10⁷, 3×10⁷, 4×10⁷, 5×107, 6×10⁷, 7×10⁷, 8×10⁷, or 9×10⁷ CAR-expressing cells per dose, at least or at least about 1×10⁸, 2×10⁸, 3×10⁸, 4×10⁸, 5×10⁸, 6×10⁸, 7×10⁸, 8×10⁸, or 9×10⁸ CAR-expressing cells per dose, at least or at least about 1×10⁹, 2×10⁹, 3×10⁹, 4×10⁹, 5×10⁹, 6×10⁹, 7×10⁹, 8×10⁹, or 9×10⁹ CAR-expressing cells per dose, at least or at least about 1×10¹⁰ CAR-expressing cells per dose.

8. A method comprising administering NK cells to a patient in need thereof, wherein the NK cells comprise a polynucleotide comprising:
a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and
b) a nucleic acid encoding an IL-15,
wherein the NK cells are administered in a dose comprising 1×10⁵ to 10×10⁸ cells/kg.

9. The method of claim 8, wherein the dose of cells comprises between at or about 1×10⁵ of the cells/kg and at or about 1×10⁸ of the cells/kg, such as between at or about 1.5×10⁵ of the cells/kg and at or about 1.5×10⁷ of the cells/kg, or between at or about 4×10⁵ of the cells/kg and at or about 4×10⁶ of the cells/kg.

10. The method of claim 8, wherein the NK cells are administered in a dose comprising at or at about 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵. 8×10⁵, 8.5×10⁵, 9×10⁵, or 9.5×10⁵ cell/kg, 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, or 9.5×10⁶ cells/kg, at or at about 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷ cells/kg, or 9.5×10⁷, or at or at about 1×10⁸, 1.5×10⁸, or 2×10⁸ cells/kg.

11. The method of claim 8, wherein the NK cells are administered in a dose comprising at least or at least about 1×10⁵, 1.5×10⁵, 2×10⁵, 2.5×10⁵. 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, or 9.5×10⁵ cell/kg, at least or at least about 1×10⁶, 1.5×10⁶, 2×10⁶, 2.5×10⁶, 3×10⁶, 3.5×10⁶, 4×10⁶4.5×10⁶, 5×10⁶, 5.5×10⁶, 6×10⁶, 6.5×10⁶, 7×10⁶, 7.5×10⁶, 8×10⁶, 8.5×10⁶, 9×10⁶, or 9.5×10⁶ cells/kg, at least or at least about 1×10⁷, 1.5×10⁷, 2×10⁷, 2.5×10⁷, 3×10⁷, 3.5×107, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷ cells/kg, or 9.5×10⁷, or at least or at least about 1×10⁸ or 1.5×10⁸ cells/kg.

12. A method comprising administering multiple doses of NK cells to a patient in need thereof, wherein the NK cells comprise a polynucleotide comprising:
a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and
b) a nucleic acid encoding an IL-15, and
wherein the doses are administered once every one to sixteen weeks.

13. The method of claim 12, wherein the NK cells are administered bi-weekly, once every three weeks, once every four weeks, once every five weeks, once every six weeks, once every seven weeks, once every eight weeks, once every nine weeks, once every ten weeks, once every 11 weeks, once every 12 weeks, once every 13 weeks, once every 14 weeks, once every 15 weeks, or once every 16 weeks.

14. A method comprising administering NK cells to a patient in need thereof, wherein the NK cells comprise a polynucleotide comprising:
a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and
b) a nucleic acid encoding an IL-15,
wherein the patient meets one or more of the following criteria:
(i) the patient has an Eastern Cooperative Oncology Group (ECOG) performance status of from 0 to 4 at the time of treatment:
(ii) the patient is refractory to or has a recurrence after receiving 1 or more prior systemic therapies:
(iii) the patient has an oxygen saturation level measured via pulse oximeter of at least 92%:
(iv) the patient has a left ventricular ejection fraction (LVEF) of at least 50%;
(v) the patient has an absolute neutrophil count (ANC) of at least 1000/mm³ (1.0×109/L);
(vi) the patient has a platelet count of at least 75000/mm³ (75×10⁹ /L);
(vii) the patient's hemoglobin level, with or without prior transfusion, is at least 8.0 g/dL:
(viii) the patient's creatinine clearance is at least 45 mL/min using the Cockcraft-Gault equation, or mL/min/1.73 m² as estimated glomerular filtration rate (eGFR) per Modification of Diet in Renal Disease (MDRD) equation:
(ix) the patient's total serum bilirubin is less than 5 mg/dL: or
(x) the patient's liver transaminases (aspartate aminotransferase/alanine amino transferase/alkaline phosphatase (AST/ALT/ALP)) is less than or equal to 5 times the upper limit of normal (ULN) for the test.

15. The method of claim 14, wherein the patient has an Eastern Cooperative Oncology Group (ECOG) performance status of 0, 1, 2, 3, or 4 at the time of treatment.

16. The method of claim 14, wherein the patient is refractory to or has a recurrence after receiving 2 or more prior systemic therapies.

17. The method of claim 14, wherein the patient has an oxygen saturation level measured via pulse oximeter of at least 92%, 93%, 94%, 95%, 96%, 97%, 98%, or 99%.

18. The method of claim 14, wherein the patient has a left ventricular ejection fraction (LVEF) of at least 50%, 51%, 52%, 53%, 54%, 55%, 56%, 57%, 58%, 59%, 60%, 61%, 62%, 63%, 64%, or 65%.

19. The method of claim 14, wherein the patient has an absolute neutrophil count (ANC) of at least 1000/mm³ (1.0×10⁹ /L), 1500/mm³, 2000/mm³, 2500/mm³, 3000/mm³, 3500/mm³. 4000/mm³, 4500/mm³, 5000/mm³, 5500/mm³, or 6000/mm³.

20. The method of claim 14, wherein the patient has a platelet count of at least 75000/mm³ (75×10⁹ /L), 100000/mm³, 125000/mm³, 150000/mm³, 175000/mm³, 200000/mm³, 225000/mm³, 250000/mm³, 275000/mm³, 300000/mm³, 325000/mm³, 350000/mm³, 375000/mm³, or 400000/mm³.

21. The method of claim 14, wherein the patient's hemoglobin level, with or without prior transfusion, is at least 8.0, 9.0, 10.0, 11.0, 12.0, 130, 14.0, 15.0, 16.0, 17.0, 18.0, 19.0, or 20.0 g/dL

22. The method of claim 14, wherein the patient's creatinine clearance is at least 45, 50, 55, 60, 65, 70, 75, 80, 85, 90, 95, 100, 105, 110, 115, 120. 125, 130, 135, or 140 mL/min using the Cockcraft-Gault equation, or mL/min/1.73 m² as estimated glomerular filtration rate (eGFR) per Modification of Diet in Renal Disease (MDRD) equation.

23. The method of claim 14, wherein the patient's total serum bilirubin is less than 5, 4.5, 4.0, 3.5, 3.0, 2.5, 2.0, 1.5, 1.0, 0.5, 0.4, 0.3, 0.2, or 0.1 mg/dL.

24. The method of claim 14, wherein the patient's liver transaminases (aspartate aminotransferase/alanine amino transferase/alkaline phosphatase (AST/ALT/ALP)) is less than or equal to 5, 4.5, 4.0, 3.5, 3.0, 2.5, 2.0, 1.5, 1.0, 0.5, or 0 times the upper limit of normal (ULN) for the test.

25. A method comprising administering NK cells to a patient in need thereof, wherein the NK cells comprise a polynucleotide comprising:
a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and
b) a nucleic acid encoding an IL-15,
wherein the patient meets one or more of the following criteria:
(i) the patient does not have active nervous system metastases:
(ii) the patient does not have a history or presence of a clinically relevant CNS disorder such as a seizure disorder (e.g., epilepsy), cerebrovascular ischemia/hemorrhage, dementia, cerebellar disease, cerebral edema, posterior reversible encephalopathy syndrome (PRES), or any autoimmune disease with CNS involvement:
(iii) the patient does not have active human immunodeficiency virus (HIV) infection, e.g., as evidenced by a positive HIV polymerase chain reaction (PCR) test;
(iv) the patient does not have active HBV or hepatitis C virus (HCV) infection, e.g., based on laboratory testing:
(v) the patient has a viral HBV load below the institutional limit of quantification (LOQ) and the subject is on stable viral suppressive therapy: or
(vi) the patient has a HCV RNA viral load below the institutional LOQ and has completed curative antiviral treatment.

26. A method comprising administering NK cells to a patient in need thereof, wherein the NK cells comprise a polynucleotide comprising:
a) a nucleic acid encoding an anti-human epidermal growth factor receptor 2 (HER2) chimeric antigen receptor (CAR) comprising an extracellular antigen binding domain comprising an anti-HER2 antibody or antigen binding fragment thereof; and
b) a nucleic acid encoding an IL-15,
wherein the patient meets one or more of the following criteria:
(i) the patient is diagnosed with or has been diagnosed with a HER2+ cancer with an IHC score of 2+;
(ii) the patient has a cancer that is not HER2-addicted, but shows some degree of HER2-expression:
(iii) the patient is diagnosed with or has been diagnosed with a HER2-low cancer:
(iv) the patient is diagnosed with or has been diagnosed with a HER2-low cancer with a HER2 expression classification of score 1+ or 2+;
(v) the patient is diagnosed with or has been diagnosed with a HER2-low cancer without HER2 amplification;
(vi) the patient has a cancer comprising a HER2 activating mutation; or
(vii) the patient has been diagnosed with a HER2 expressing cancer that is IHC 1+ or IHC 2+/ISH− and is refractory to or has a recurrence after receiving previous treatment.

27. The method of claim 26, wherein the patient is diagnosed with or has been diagnosed with a HER2-low cancer with a HER2 expression classification of score 1+ or 2+without HER2 amplification.

28. The method of claim 27, wherein the HER2-low cancer harbors HER2 gain.

29. The method of claim 27, wherein the HER2-low cancer lacks HER2 gain.

30. The method of claim 26, wherein the patient has been diagnosed with a IHC 3+ or IHC 2+/ISH+ cancer and is refractory to or has a recurrence after receiving previous treatment with a HER2-targeting therapy.

31. The method of claim 26, wherein the patient has been diagnosed with a HER2 expressing cancer that is IHC 1+ or IHC 2+/ISH−, and the patient's cancer is unresectable, metastatic, or the patient is refractory to or has a recurrence after receiving previous treatment.

32. The method of any one of claims 1 to 31, wherein the anti-HER2 antibody or antigen binding fragment thereof comprises a light chain complementarity determining region 1 (CDRL1) comprising SEQ ID NO: 34, a light chain complementarity determining region 2 (CDRL2) comprising SEQ ID NO: 36; a light chain complementarity determining region 3 (CDRL3) comprising SEQ ID NO: 38, a heavy chain complementarity determining region 1 (CDRH1 comprising SEQ ID NO: 44; a heavy chain complementarity determining region 2 (CDRH2) comprising SEQ ID NO: 46; and a heavy chain complementarity determining region 3 (CDRH3) comprising SEQ ID NO: 48.

33. The method of any one of claims 1-32, wherein the nucleic acid encoding the anti-HER2 antibody or antigen binding fragment thereof encodes a CDRL1 encoded by SEQ ID NO: 35, a CDRL2 encoded by SEQ ID NO: 37; a CDRL3 encoded by SEQ ID NO: 39, a CDRH1 encoded by SEQ ID NO: 45: a CDRH2 encoded by SEQ ID NO: 47; and a CDRH3 encoded by SEQ ID NO: 49.

34. The method of any one of claims 1-33, wherein the anti-HER2 antibody or antigen binding fragment thereof comprises a light chain variable (VL) region comprising SEQ ID NO: 32 and a heavy chain variable (V_{H}) region comprising SEQ ID NO: 42.

35. The method of any one of claims 1-34, wherein the nucleic acid encoding the anti-HER2 antibody or antigen binding fragment thereof comprises a nucleic acid encoding a VL region comprising SEQ ID NO: 33 and a nucleic acid encoding a V_{H} region comprising SEQ ID NO: 37.

36. The method of any one of claims 1-35, wherein the anti-HER2 antibody or antigen binding fragment thereof comprises a VL region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 32 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 42.

37. The method of any one of claims 1-36, wherein the anti-HER2 antibody or antigen binding fragment thereof is an antigen binding fragment.

38. The method of any one of claims 1-37, wherein the antigen binding fragment comprises a single chain Fv (scFv).

39. The method of any one of claims 1-38, wherein the VL region is amino-terminal to the V_{H} region.

40. The method of any one of claims 1-38, wherein the VL region is carboxy-terminal to the V_{H} region.

41. The method of any one of claims 1-40, wherein the VI. region is joined to the V_{H} region via a flexible linker.

42. The method of claim 41, wherein the flexible linker comprises the amino acid sequence set forth in SEQ ID NO: 40.

43. The method of claim 42, wherein the flexible linker is encoded by a nucleic acid comprising SEQ ID NO: 41.

44. The method of any one of claims 38-43, wherein the scFv comprises the amino acid sequence set forth in SEQ ID NO: 30.

45. The method of claim 44, wherein the scFv is encoded by a nucleic acid comprising SEQ ID NO: 31.

46. The method of claim 44, wherein the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 30.

47. The method of any one of claims 1-46, wherein the anti-HER2 CAR specifically binds to a human epidermal growth factor receptor 2 (HER2) protein.

48. The method of claim 47, wherein the HER2 protein comprises the amino acid sequence of SEQ ID NO: 62.

49. The method of any one of claims 1-48, wherein the CAR comprises a transmembrane domain, optionally a CD28 transmembrane domain.

50. The method of claim 49, wherein the CD28 transmembrane domain comprises the amino acid sequence set forth in SEQ ID NO: 53.

51. The method of claim 50, wherein the CD28 transmembrane domain is encoded by a nucleic acid comprising the nucleic acid sequence set forth in SEQ ID NO: 54or SEQ ID NO: 55.

52. The method of any one of claims 1-51, wherein the CAR further comprising a hinge domain between the extracellular antigen binding domain and the transmembrane domain.

53. The method of claim 52, wherein the hinge domain comprises at least a portion of a CD8a hinge domain.

54. The method of claim 53, wherein the CD8a hinge domain comprises an amino acid sequence set forth in SEQ ID NO: 50.

55. The method of claim 54, wherein the CD8a hinge domain is encoded by a nucleic acid comprising SEQ ID NO: 51or SEQ ID NO: 52.

56. The method of claim 54, wherein the CD8a hinge domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 50.

57. The method of any one of claims 1-56, wherein the CAR comprises an intracellular signaling region, optionally where the intracellular signaling region comprises a CD28 intracellular signaling domain, an OX40L intracellular signaling domain, and a CD3-zeta (CD3) signaling domain.

58. The method of claim 57, wherein the intracellular signaling region comprises a CD28 intracellular signaling domain and a CD3-zeta signaling domain.

59. The method of any one of claim 57 or 58, wherein the intracellular signaling region comprises an OX40L intracellular signaling domain.

60. The method of claim 57 or claim 59, wherein the OX40L intracellular signaling domain comprises an amino acid sequence set forth in SEQ ID NO: 8. SEQ ID NO: 9, or SEQ ID NO: 10.

61. The method of claim 57 or claim 59, wherein the OX40L intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 8, SEQ ID NO: 9, or SEQ ID NO: 10.

62. The method of claim 57 or claim 59, wherein the OX40L intracellular signaling domain is encoded by a nucleic acid comprising SEQ ID NO: 11 or SEQ ID NO: 12.

63. The method of any one of claims 57-62, wherein the intracellular signaling region comprises a CD28 intracellular signaling domain.

64. The method of claim 63, wherein the CD28 intracellular signaling domain comprises an amino acid sequence set forth in SEQ ID NO: 5.

65. The method of claim 63, wherein the CD28 intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 5.

66. The method of claim 63, wherein the CD28 intracellular signaling domain is encoded by a nucleic acid comprising SEQ ID NO: 6 or SEQ ID NO: 7.

67. The method of any one of claims 57-66, wherein the intracellular signaling region comprises an CD3-zeta intracellular signaling domain.

68. The method of claim 67, wherein the CD3-zeta intracellular signaling domain comprises an amino acid sequence set forth in SEQ ID NO: 13.

69. The method of claim 67, wherein the CD3-zeta intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 13.

70. The method of claim 67, wherein the CD3-zeta intracellular signaling domain is encoded by a nucleic acid comprising SEQ ID NO: 14 or SEQ ID NO: 15.

71. The method of any one of claims 57-70, wherein the intracellular signaling region comprises an amino acid sequence set forth in SEQ ID NO: 25.

72. The method of any one of claims 57-70, wherein the intracellular signaling region comprises an amino acid sequence having or having at least 85%, 86%, 87%, 88%, 89%, 90%, 91%, 92%, 93%, 94%, 95%, 96%, 97%, 98%, or 99% identity to SEQ ID NO: 25.

73. The method of any one of claims 1-72, wherein the CAR comprises an amino sequence set forth in SEQ ID NO: 56.

74. The method of claim 73, wherein the CAR is encoded by a nucleic acid comprising SEQ ID NO: 57.

75. The method of any one of claims 1-74, wherein the IL-15 comprises the amino acid sequence set forth in SEQ ID NO: 22.

76. The method of claim 75, wherein the IL-15 is encoded by a nucleic acid comprising SEQ ID NO: 23 or SEQ ID NO: 24.

77. The method of any one of claims 1-76, wherein the polynucleotide encodes a polyprotein comprising the CAR and the IL-15.

78. The method of any one of claims 1-77, wherein the polynucleotide further comprises a nucleic acid encoding a self-cleaving peptide, optionally a T2A self-cleaving peptide.

79. The method of claim 78, wherein the CAR is joined to the IL-15 by the self-cleaving peptide.

80. The method of claim 79, wherein the self-cleaving peptide is capable of inducing ribosomal skipping between the CAR and the IL-15.

81. The method of any one of claims 1-80, wherein the polynucleotide further comprises a nucleic acid encoding a signal sequence.

82. The method of claim 81, wherein the signal sequence comprises the amino acid sequence set forth in SEQ ID NO: 27.

83. The method of claim 81, wherein the nucleic acid encoding the signal sequence comprises the nucleic acid sequence set forth in SEQ ID NO: 28.

84. The method of any one of claims 1-83, wherein the polynucleotide encodes a polyprotein comprising the amino acid sequence set forth in SEQ ID NO: 59.

85. The method of any one of claims 1-84, wherein the polynucleotide comprises the nucleic acid sequence set forth in SEQ ID NO: 60 or SEQ ID NO: 61.

86. The method of any one of claims 1-85, wherein the NK cells are cord blood NK cells.

87. The method of any one of claims 1-86, wherein the NK cells comprise a KIR-B haplotype.

88. The method of any one of claims 1-87, wherein the NK cells express CD16 having the V/V polymorphism at F158.

89. The method of any one of claims 1-88, wherein the NK cells are administered as part of a pharmaceutical composition.

90. The method of claim 89, wherein the pharmaceutical composition comprises a pharmaceutically acceptable excipient.

91. The method of claim 89, wherein the pharmaceutical composition comprises:
(a) human albumin;
(b) dextran;
(c) glucose;
(d) DMSO; and
(e) a buffer.

92. The method of claim 91, wherein the pharmaceutical composition comprises from 30 to 50 mg/ml human albumin.

93. The method of claim 91, wherein the pharmaceutical composition comprises 40 mg/ml human albumin.

94. The method of any one of claims 91-93, wherein the pharmaceutical composition comprises 20 to 30 mg/mL dextran.

95. The method of any one of claims 91-94, wherein the pharmaceutical composition comprises 25 mg/mL dextran.

96. The method of any one of claims 91-95, wherein the dextran is Dextran 40.

97. The method of any one of claims 91-96, wherein the pharmaceutical composition comprises from 12 to 15 mg/mL glucose.

98. The method of any one of claims 91-97, wherein the pharmaceutical composition comprises 12.5 mg/mL glucose.

99. The method of any one of claims 91-98, wherein the pharmaceutical composition comprises less than 27.5 g/L glucose.

100. The method of any one of claims 91-99, wherein the pharmaceutical composition comprises from 50 to 60 ml/mL DMSO.

101. The method of any one of claims 91-100, wherein the pharmaceutical composition comprises 55 mg/mL DMSO.

102. The method of any one of claims 91-101, wherein the pharmaceutical composition comprises 40 to 60% v/v buffer.

103. The method of any one of claims 91-102, wherein the buffer is phosphate buffered saline.

104. The method of claim 91, wherein the pharmaceutical composition comprises:
(a) about 40 mg/mL human albumin;
(b) about 25 mg/ml Dextran 40;
(c) about 12.5 mg/mL glucose;
(d) about 55 mg/mL DMSO; and
(e) about 0.5 mL/mL phosphate buffered saline.

105. The method of any one of claims 89-104, wherein the pharmaceutical composition further comprises 0.5 mL/mL water.

106. The method of any one of claims 1-85, wherein the subject has a disease or condition associated with HER2.

107. The method of any one of the preceding claims, wherein the disease or condition associated with HER2 is cancer.

108. The method of claim 107, wherein the cancer expresses HER2.

109. The method of claim 87, wherein the cancer is a HER2+ cancer.

110. The method of claim 108 or 109, wherein the cancer is or comprises a solid tumor.

111. The method of any one of claims 107-109, wherein the cancer is or comprises a bladder cancer, breast adenocarcinoma, colorectal adenocarcinoma, non-small cell lung cancer, esophageal cancer, cervix squamous cancer, stomach adenocarcinoma, cholangiocarcinoma, ovary cancer, renal papillary cell carcinoma, and combinations thereof.

112. The method of any one of claims 107-109, wherein the cancer is or comprises a breast cancer.

113. The method of any one of claims 107-109, wherein the breast cancer is noninvasive.

114. The method of any one of claims 107-109, wherein the breast cancer is invasive.

115. The method of any one of claims 107-109, wherein the breast cancer is metastatic.

116. The method of any one of claims 107-109, wherein the cancer is or comprises a gastric cancer.

117. The method of any one of claims 107-109, wherein the cancer is or comprises an ovarian cancer.

118. The method of any one of claims 107-109, wherein the cancer is or comprises a gastroesophageal cancer.

119. The method of any one of claims 107-109, wherein the cancer is or comprises a lung cancer.

120. The method of claim 119, wherein the lung cancer is non-small cell lung cancer (NSCLC).

121. The method of claim 120, wherein the patient has a HER2 activating mutation.

122. The method or use of any one of the foregoing claims, further comprising administering a lymphodepleting chemotherapy to the subject prior to treatment.

123. The method of claim 122, wherein the lymphodepleting chemotherapy is non-myeloablative chemotherapy.

124. The method of claim 122 or claim 123, wherein the lymphodepleting chemotherapy comprises treatment with at least one of cyclophosphamide and fludarabine.

125. The method of claim 124, wherein the lymphodepleting chemotherapy comprises treatment with cyclophosphamide and fludarabine.

126. The method of any one of claims 124-125, wherein between 100 and 500 mg/m² cyclophosphamide is administered per day.

127. The method of claim 126, wherein 250 mg/m² cyclophosphamide is administered per day.

128. The method of claim 126, wherein 500 mg/m² cyclophosphamide is administered per day.

129. The method of any one of claims 124-128, wherein between 10 and 50 mg/m² of fludarabine is administered per day.

130. The method of claim 129, wherein 30 mg/m² of fludarabine is administered per day.

131. The method or use of any one of claims 106-130, further comprising administering IL-2 to the subject.

132. The method of claim 131, wherein the patient is administered 1×10⁶IU/m² of IL-2.

133. The method of claim 131, wherein the patient is administered 1×10⁷ IU of IL-2.

134. The method of claim 131, wherein the patient is administered 6×10⁷ IU of IL-2.

135. The method of any one of claims 132-134, wherein administration of IL-2 occurs within 1-4 hours of administration of the cells.

136. The method of any one of claims 132-134, wherein administration of IL-2 occurs at least 1-4 hours after the administration of the cells.

137. The method of any one of claims 106-136, comprising administering the cells a plurality of times.

138. The method of any one of claims 106-137, comprising administering the cells three times, four times, or eight times.

139. The method of any one of claims 106-137, comprising administering the cells every week, every two weeks, every three weeks, or every four weeks.

140. The method of any one of claims 106-139, further comprising administering pertuzumab to the subject.

141. The method of any one of claims 106-140, further comprising administering trastuzumab to the subject.

142. The method of any one of claims 106-141, further comprising administering necitumumab to the subject.

143. The method of any one of claims 106-142, further comprising administering margetuximab to the subject.

144. The method of any one of claims 106-143, further comprising administering taxane to the subject.

145. The method of claim 144, wherein the taxane is at least one of paclitaxel, docetaxel, and cabazitaxel

146. The method of any one of claims 106-145, further comprising administering an endocrine therapy to the subject.

147. The method of claim 146, wherein the endocrine therapy comprises at least one of an aromatase inhibitor, fulvestrant, and tamoxifen.

148. The method of any one of claims 106-147, further comprising administering a checkpoint inhibitor to the subject.

149. The method of claim 148, wherein the checkpoint inhibitor inhibits CTLA-4, PD-1, or PD-L1.

150. The method of claim 148 or claim 149, wherein the checkpoint inhibitor is or comprises ipilimumab.

151. The method of any one of claims 148-150, wherein the checkpoint inhibitor is or comprises nivolumab.

152. The method of any one of claims 148-151, wherein the checkpoint inhibitor is or comprises pembrolizumab.

153. The method of any one of claims 148-152, wherein the checkpoint inhibitor is or comprises cemiplimab.

154. The method of any one of claims 148-153, wherein the checkpoint inhibitor is or comprises atezolizumab.

155. The method of any one of claims 148-150, wherein the checkpoint inhibitor is or comprises avelumab.

156. The method of any one of claims 148-150, wherein the checkpoint inhibitor is or comprises durvalumab.

157. The method of any one of claims 1-156, further comprising:
administering a second therapeutic moiety to the subject.

158. The method of claim 157, wherein the second therapeutic moiety comprises a lymphodepleting chemotherapy agent.

159. The method of any one of claims 157-158, wherein the second therapeutic moiety comprises IL-2.

160. The method of any one of claims 157-159, wherein the second therapeutic moiety comprises at least one of pertuzumab, trastuzumab, necitumumab, and margetuximab.

161. The method of any one of claims 157-160, wherein the second therapeutic moiety comprises a taxane.

162. The method of any one of claims 157-161, wherein the second therapeutic moiety comprises a checkpoint inhibitor.
